(19) United States
(12) Patent Application Publication
(10) Pub. No.: US 2023/0148005 A1
(43) Pub. Date: May 11, 2023

(54) MACROCYCLIC COMPOUNDS AND USES THEREOF

(71) Applicant: THESEUS PHARMACEUTICALS, INC., Cambridge, MA (US)

(72) Inventors: Wei-Sheng Huang, Cambridge, MA (US); William C. SHAKESPEARE, Cambridge, MA (US); Charles J. EYERMANN, Cambridge, MA (US); David C. DALGARNO, Cambridge, MA (US)

(21) Appl. No.: 17/800,330

(22) PCT Filed: Feb. 18, 2021
(86) PCT No.: PCT/US2021/018520

§ 371 Date: Aug. 17, 2022

Related U.S. Application Data
(60) Provisional application No. 62/978,202, filed on Feb. 18, 2020.

(51) Int. Cl. C07D 498/22; C07D 513/22; A61P 35/00

(52) U.S. Cl. CPC C07D 498/22; C07D 513/22; A61P 35/00

(57) ABSTRACT

Described herein are macrocyclic compounds of Formula (I), which can inhibit kinases such as EGFR, including mutant forms such as T790M EGFR mutants. Also described herein are pharmaceutical compositions comprising a compound of Formula (I), or any pharmaceutically acceptable form thereof, processes for their preparation, and use in therapy for the prevention or treatment of cancer. In particular, compounds described herein can be effective for treating EGFR-driven cancers including non-small cell lung cancer (NSCLC).

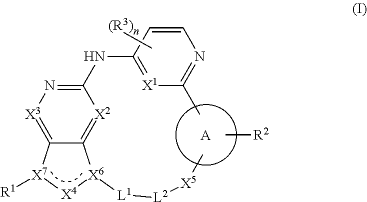

### CROSS-REFERENCE TO RELATED APPLICATIONS

The present application claims benefit of U.S. Provisional Application No. 62/978,202, filed Feb. 18, 2020, which is hereby incorporated by reference in its entirety.

### FIELD OF THE INVENTION

Described herein are macrocyclic compounds that can be used as kinase inhibitors. In particular, compounds described herein can inhibit epidermal growth factor receptor (EGFR), including mutant forms of EGFR. Compounds described herein can be effective for treating various disorders that include cancers such as EGFR-driven cancers (e.g., non-small cell lung cancer (NSCLC) characterized by mutant EGFR).

### BACKGROUND

Signal transduction refers to the transmission of stimulatory or inhibitory signals into and within a cell leading, often via a cascade of signal transmission events, to a biological response within the cell. Defects in various components of signal transduction pathways have been found to account for a large number of diseases, including numerous forms of cancer, inflammatory disorders, metabolic disorders, vascular and neuronal diseases.

Signal transduction is often mediated by certain proteins called kinases. Kinases can generally be classified into protein kinases and lipid kinases, and certain kinases exhibit dual specificities. For example, epidermal growth factor receptor (EGFR) belongs to a family of receptor tyrosine kinases (RTKs) that include EGFR/ERBB1, HER2/ERBB2/NEU, HER3/ERBB3, and HER4/ERBB4. The binding of a ligand, such as epidermal growth factor (EGF), induces a conformational change in EGFR that facilitates receptor homo- or heterodimer formation, leading to activation of EGFR tyrosine kinase activity. Activated EGFR then phosphorylates its substrates, resulting in activation of multiple downstream pathways within the cell, including the PI3K-AKT-mTOR pathway, which is involved in cell survival, and the RAS-RAF-MEK-ERK pathway, which is involved in cell proliferation. (Chong et al. Nature Med. 2013; 19(11):1389-1400).

Certain cancers are characterized by mutations of EGFR, which results in increased cell proliferation. Tyrosine kinase inhibitor (TKI) therapies that inhibit EGFR can lead to clinical responses; however, mutations in EGFR can also confer resistance to such therapies.

New therapeutic methods therefore remain necessary for treating cancers associated with defective signal transduction pathways, including EGFR-driven cancers.

### SUMMARY OF THE INVENTION

Described herein are new compounds that can be effective inhibitors of EGFR. Such compounds can be useful for treating various diseases and disorders, including EGFR-driven cancers such as non-small cell lung cancer (NSCLC) characterized by mutant EGFR.

Accordingly, in one aspect, the invention features a compound having a structure according to

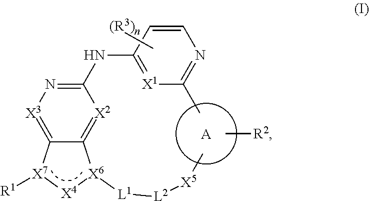

or a pharmaceutically acceptable salt thereof,

wherein:

A is C₆₋₁₀ arylene, 5-12-membered heteroarylene, or 5-12-membered heterocycloalkylene;

X¹ is N or CR^{X};

X² is N or CR^{X};

X³ is N or CR^{X};

X⁴ is N or CR^{X};

X⁶ is N or CR^{X′};

X⁷ is N or CR^{X′};

[symbol] represents an optional double bond between X⁷ and X⁴ or X⁴ and X⁶, wherein one and only one double bond is present;

X⁵ is a covalent bond, CH₂, O, NR⁴, C(O)NR⁴, or NR⁴C(O);

L¹ is a covalent bond or C(R⁵)₂, and L² is C₁₋₄ alkylene, or L¹ and L² combine to form a C₃₋₆ cycloalkyl or a 4- to 6-membered heterocycloalkyl;

R¹ is halogen, C₁₋₆ alkyl, C₃₋₇ cycloalkyl, C₆₋₁₀ aryl, 5- to 10-membered heteroaryl, 3- to 10-membered heterocycloalkyl, CN, NR⁶R⁷, NR⁶C(O)R⁷, NR⁶C(O)NH₂, OR⁸, or C(O)NR⁶R⁷;

R² is absent, H, C₁₋₆ alkyl, halogen, CN, or C₁₋₆ alkoxy;

each R³, when present, is independently OH, CN, halogen, C₁₋₆ alkyl, or C₁₋₆ alkoxy;

n is 0, 1, or 2;

each R^{X} is independently H, OR^{X1}, CN, halogen, or C₁₋₆ alkyl, wherein R^{X1} is H or C₁₋₆ alkyl;

each R^{X′} is independently H, OR^{X1}, CN, halogen, or C₁₋₆ alkyl, wherein R^{X1} is H or C₁₋₆ alkyl, or R^{X′} is absent if the carbon to which it is attached is part of a double bond;

each R⁴ and R⁵ is independently H or C₁₋₆ alkyl;

each R⁶ and R⁷ is independently H, C₁₋₆ alkyl, C₃₋₇ cycloalkyl, or 3- to 10-membered heterocycloalkyl; or R⁶ and R⁷ together with the nitrogen atom to which they are attached form a 3- to 8-membered heterocycloalkyl ring; and

R⁸ is independently H, C₁₋₆ alkyl, or 4- to 6-membered heterocycloalkyl.

In embodiments, the compound has a structure according to Formula I′:

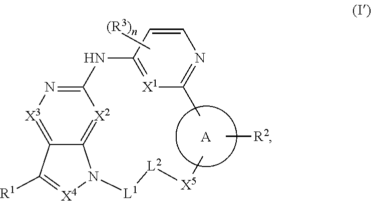

or a pharmaceutically acceptable salt thereof.

In embodiments, n is 0.

In embodiments, X³ is CH.

In embodiments, X² is N or CH.

In embodiments, X¹ is N or CH.

In embodiments, one of X¹ and X² is N and the other is CH.

In embodiments, X⁴ is N or CH.

In embodiments, L¹ is CHR⁵, and R⁵ is H, CH₃, or CH₂CH₃.

In embodiments, L¹ is C(CH₃)₂.

In embodiments, L¹ is CHCH₃.

In embodiments, L² is unsubstituted C₁₋₄ alkylene, or C₁₋₄ alkylene substituted by unsubstituted C₁₋₃ alkyl.

In embodiments, L² is (CH₂)₂, (CH₂)₃, CH(CH₃)CH₂, or CH₂CH(CH₃).

In embodiments, L¹ and L² combine to form cyclopropyl, cyclobutyl, cyclopentyl, or cyclohexyl.

In embodiments, X⁵ is O or NR⁴.

In embodiments, X⁵ is O, NH, or NCH₃.

In embodiments, A is C₆₋₁₀ arylene or 5-12-membered heteroarylene.

In embodiments, A is 5-12-membered heteroarylene or 5-12-membered heterocycloalkylene.

In embodiments, A is pyridyl, pyrazolyl, thiazolyl, oxazolyl, imidazyolyl,

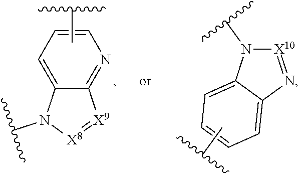

wherein each X⁸, X⁹, and X¹⁰ is CH or N.

In embodiments, A is pyrazolyl optionally substituted by methyl.

In embodiments, A is phenyl.

In embodiments, R² is absent, H, unsubstituted C₁₋₃ alkyl, or C₁₋₃ alkyl substituted by unsubstituted C₃₋₆ cycloalkyl.

In embodiments, R¹ is F, CN, NH₂, O-(oxetan-3-yl), NH-(oxetan-3-yl), O-(tetrahydrofuran-3-yl), O-(1-N,N-dimethylaminocyclohexan-4-yl), NH-(tetrahydrofuran-3-yl), NH(C₁₋₆ alkyl), NCH₃(C₁₋₆ alkyl), and wherein said C₁₋₆ alkyl comprises one or two substituents selected from OH, NH₂, piperidinyl, and CONH₂.

In embodiments, R¹ is an N-linked group that is azetidine, pyrrolidine, pyrrolyl, or piperazinyl, and wherein said N-linked group is unsubstituted or substituted with a substituent that is OH, CN, oxo, C₁₋₄ alkyl, —NR^{1A}R^{1B}, or —C(O)NR^{1A}R^{1B}, wherein
said C₁₋₄ alkyl is unsubstituted or substituted with at least one group that is OH, CN, NH₂, NHCH₃, N(CH₃)₂, N-methylpiperazinyl, C(O)NH₂, C(O)NHCH₃, C(O)N(CH₃)₂,
each R^{1A} and R^{1B} is independently H, C₁₋₆ alkyl, C₃₋₇ cycloalkyl, or 3- to 10-membered heterocycloalkyl; or R^{1A} and R^{1B} together with the nitrogen atom to which they are attached form a 3- to 8-membered heterocycloalkyl ring, wherein said C₁₋₆ alkyl is unsubstituted or substituted with a group that is alkoxy.

In embodiments, R¹ is C(O)NHR⁷, and R⁷ is a cyclic group that is cyclopentyl, cyclohexyl,
wherein said cyclic group is unsubstituted or substituted by a group that is CN, OH, oxo, C₁₋₄ alkyl, —NR^{1A}R^{1B} or —C(O)NR^{1A}R^{1B}, wherein
said C₁₋₄ alkyl is unsubstituted or substituted with a group that is OH, NH₂, NHCH₃, N(CH₃)₂, N-methylpiperazinyl, C(O)NH₂, C(O)NHCH₃, C(O)N(CH₃)₂,
each R^{1A} and R^{1B} is independently H, C₁₋₆ alkyl, C₃₋₇ cycloalkyl, or 3- to 10-membered heterocycloalkyl; or R^{1A} and R^{1B} together with the nitrogen atom to which they are attached form a 3- to 8-membered heterocycloalkyl ring.

In embodiments, R¹ is NR⁶R⁷, wherein
R⁶ is independently H or unsubstituted C₁₋₃ alkyl; and
R⁷ is independently C₁₋₆ alkyl, wherein said C₁₋₆ alkyl is unsubstituted or comprises one or two substituent groups selected from —OH and —C(O)NH₂.

In embodiments, R¹ is a substituted or unsubstituted 5- or 6-membered heteroarylene; a substituted or unsubstituted 5- or 6-membered heterocycloalkyl, C₁₋₆ alkyl substituted by a 5- or 6-membered heteroarylene that is substituted or unsubstituted; or C₁₋₆ alkyl substituted by a 5- or 6-membered heterocycloalkyl that is substituted or unsubstituted, or substituted phenyl.

In embodiments, R⁸ is a substituted C₁₋₆ alkyl (e.g., piperidinyl-substituted C₁₋₆ alkyl such as —CH₂CH₂(piperidinyl)).

In embodiments, a compound has a structure according to Formula (I-A),

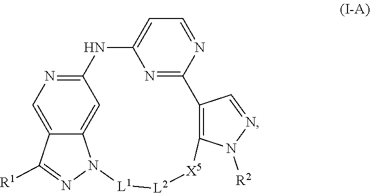

or a pharmaceutically acceptable salt thereof, wherein

R² is unsubstituted C₁₋₆ alkyl or C₁₋₆ alkyl substituted by a group that is unsubstituted C₃₋₆ cycloalkyl; and

L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄, or

L¹-L²-X⁵ is

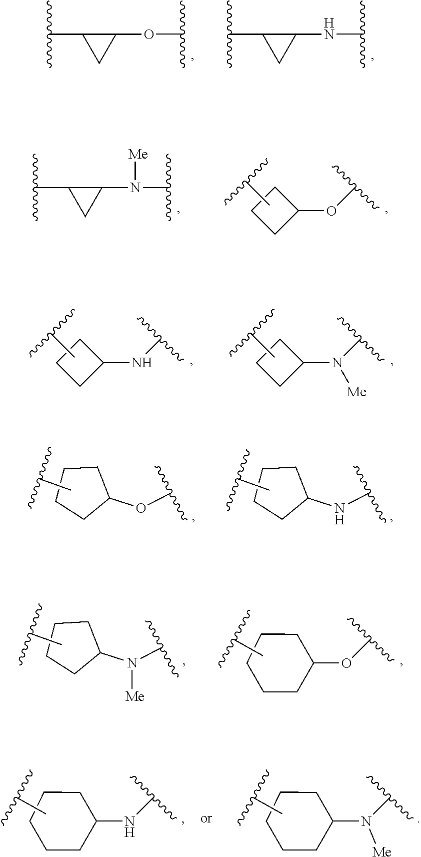

In embodiments, R² is CH₃ or C₁₋₃ alkyl substituted by unsubstituted C₃₋₆ cycloalkyl.

In embodiments, L¹-L²-X⁵ is CH(CH₃)—(CH₂)₂—O, CH(CH₃)—(CH₂)₃—O, or (CH₂)₃—O.

In embodiments, a compound has a structure according to Formula (I-A-1),

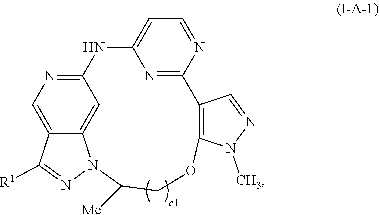

or a pharmaceutically acceptable salt thereof, wherein c1 is 2 or 3.

In embodiments, a compound has a structure according to Formula (I-A-1′),

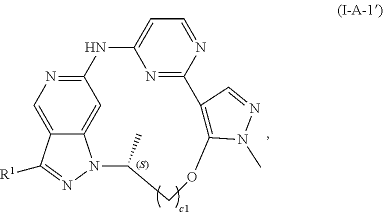

or a pharmaceutically acceptable salt thereof.

In embodiments, a compound has a structure according to Formula (I-A-1″),

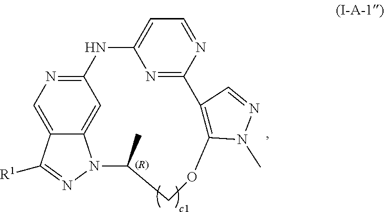

or a pharmaceutically acceptable salt thereof.

In embodiments, a compound has a structure according to Formula (I-A-2),

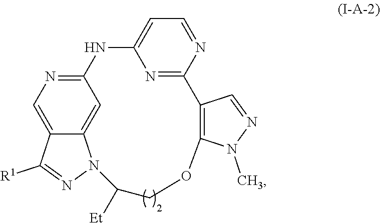

or a pharmaceutically acceptable salt thereof.

In embodiments, a compound has a structure according to Formula (I-A-3),

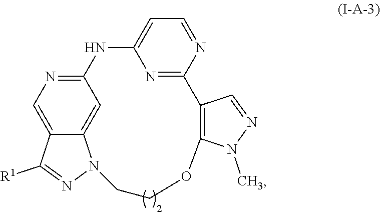

or a pharmaceutically acceptable salt thereof.

In embodiments, a compound has a structure according to Formula (I-B),

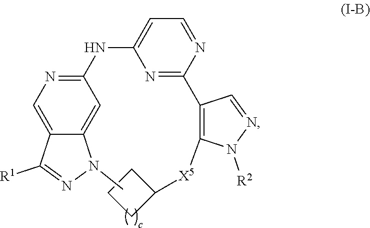

or a pharmaceutically acceptable salt thereof, wherein R² is unsubstituted C₁₋₆ alkyl or C₁₋₆ alkyl substituted by a group that is unsubstituted C₃₋₆ cycloalkyl; X⁵ is O; and c is 0, 1, 2, or 3.

In embodiments, R² is CH₃.

In embodiments, a compound has a structure according to Formula (I-B-1),

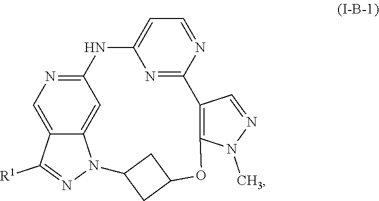

or a pharmaceutically acceptable salt thereof.

In embodiments, a compound has a structure according to Formula (I-B-2),

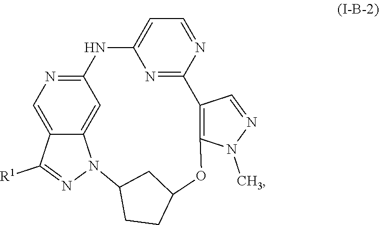

or a pharmaceutically acceptable salt thereof.

In embodiments, a compound has a structure according to Formula (I-B-3),

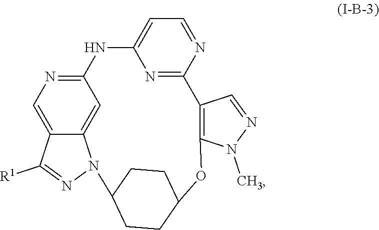

or a pharmaceutically acceptable salt thereof.

In embodiments, a compound has a structure according to Formula (I-C),

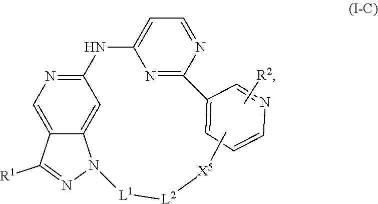

or a pharmaceutically acceptable salt thereof, wherein

R² is H, unsubstituted C₁₋₆ alkyl or C₁₋₆ alkyl substituted by a group that is unsubstituted C₃₋₆ cycloalkyl;

L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, CH(CH₃)—(CH₂)₄, CH(CH₃)—(CH₂)₂—NHC(O), CH(CH₃)—(CH₂)₂—NCH₃C(O), CH(CH₃)—(CH₂)₃—NHC(O), CH(CH₃)—(CH₂)₃—NCH₃C(O), CH(CH₃)—(CH₂)₂—C(O)NH, CH(CH₃)—(CH₂)₂—C(O)NCH₃, CH(CH₃)—(CH₂)₃—C(O)NH, or CH(CH₃)—(CH₂)₃—C(O)NCH₃; or

L¹-L²-X⁵ is,

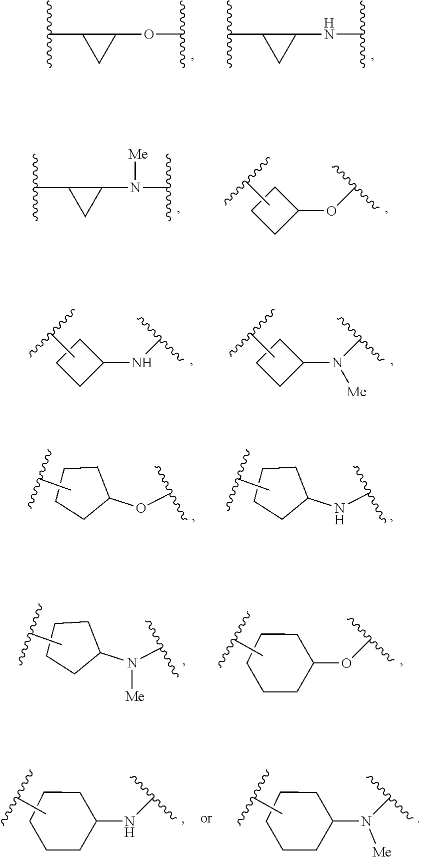

In embodiments, R² is H or CH₃.

In embodiments, L¹-L²-X⁵ is CH(CH₃)—(CH₂)₂—O, CH(CH₃)—(CH₂)₃—O, (CH₂)₃—O, CH(CH₃)—(CH₂)₂—NHC(O), CH(CH₃)—(CH₂)₂—NCH₃C(O), CH(CH₃)—(CH₂)₃—NHC(O), CH(CH₃)—(CH₂)₃—NCH₃C(O), CH(CH₃)—(CH₂)₂—C(O)NH, CH(CH₃)—(CH₂)₂—C(O)NCH₃, CH(CH₃)—(CH₂)₃—C(O)NH, or CH(CH₃)—(CH₂)₃—C(O)NCH₃.

In embodiments, a compound has a structure according to Formula (I-C-1),

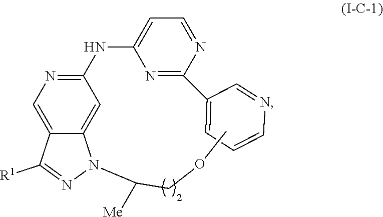

or a pharmaceutically acceptable salt thereof.

In embodiments, a compound has a structure according to Formula (I-C-2),

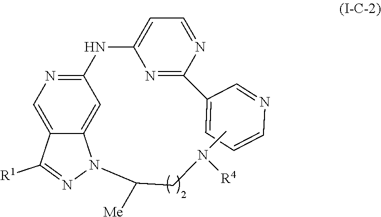

or a pharmaceutically acceptable salt thereof, wherein R⁴ is H or CH₃.

In embodiments, a compound has a structure according to Formula (I-C-3),

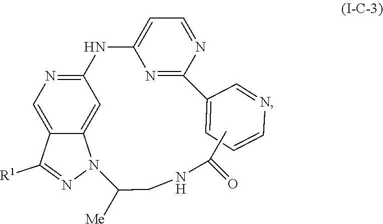

or a pharmaceutically acceptable salt thereof.

In embodiments, a compound has a structure according to Formula (I-C-4),

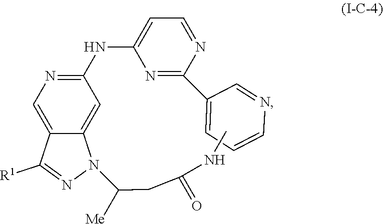

or a pharmaceutically acceptable salt thereof.

In embodiments, a compound has a structure according to Formula (I-D),

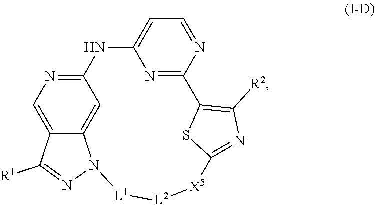

or a pharmaceutically acceptable salt thereof, wherein

R² is H or unsubstituted C₁₋₆ alkyl; and

L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄, or

L¹-L²-X⁵ is

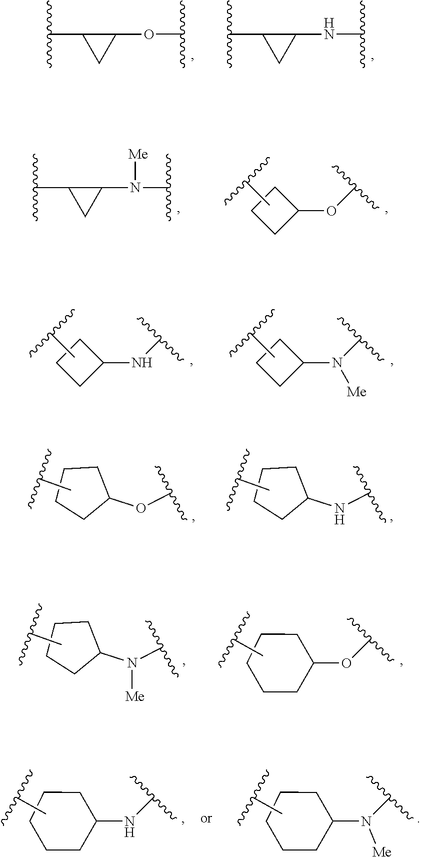

In embodiments, R² is H or CH₃.

In embodiments, L¹-L²-X⁵ is CH(CH₃)—(CH₂)₂—NH, CH(CH₃)—(CH₂)₂—NCH₃, CH(CH₃)—(CH₂)₃—NH, or CH(CH₃)—(CH₂)₃—NCH₃.

In embodiments, a compound has a structure according to Formula (I-D-1),

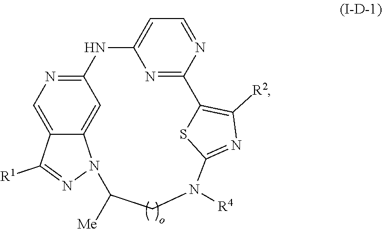

or a pharmaceutically acceptable salt thereof, wherein R² is H or CH₃; R⁴ is H or CH₃; and o is 1 or 2.

In embodiments, a compound has a structure according to Formula (I-E),

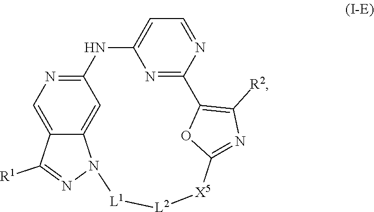

or a pharmaceutically acceptable salt thereof, wherein

R² is H or unsubstituted C₁₋₆ alkyl; and

L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄; or

L¹-L²-X⁵ is

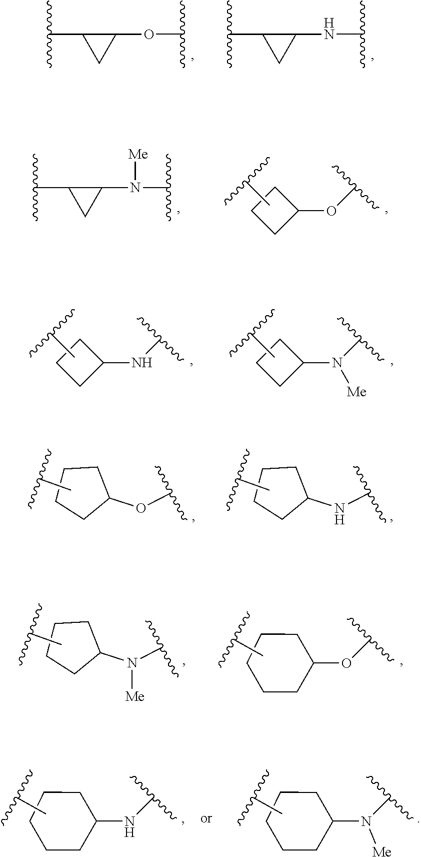

In embodiments, R² is H or CH₃.

In embodiments, L¹-L²-X⁵ is CH(CH₃)—(CH₂)₃, or CH(CH₃)—(CH₂)₄.

In embodiments, a compound has a structure according to Formula (I-E-1),

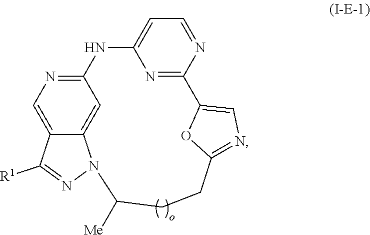

or a pharmaceutically acceptable salt thereof, wherein

o is 2 or 3.

In embodiments, a compound has a structure according to Formula (I-F),

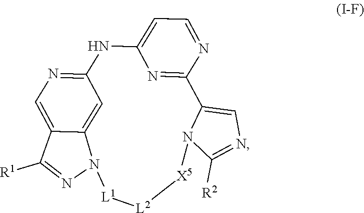

or a pharmaceutically acceptable salt thereof, wherein

R² is H or unsubstituted C₁₋₆ alkyl; and

L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄; or

L¹-L²-X⁵ is

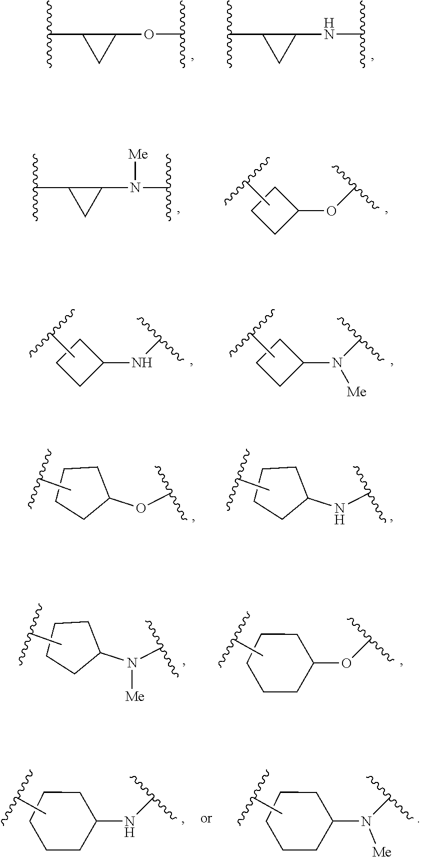

In embodiments, R² is H or CH₃.

In embodiments, L¹-L²-X⁵ is CH(CH₃)—(CH₂)₃, or CH(CH₃)—(CH₂)₄.

In embodiments, a compound has a structure according to Formula (I-F-1),

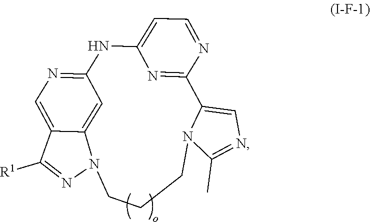

or a pharmaceutically acceptable salt thereof, wherein o is 1 or 2.

In embodiments, a compound has a structure according to Formula (I-G),

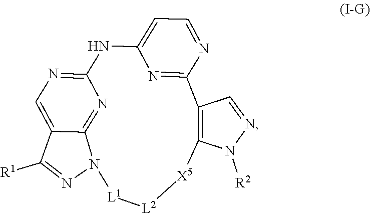

or a pharmaceutically acceptable salt thereof, wherein

R² is unsubstituted C₁₋₆ alkyl or C₁₋₆ alkyl substituted by a group that is unsubstituted C₃₋₆ cycloalkyl; and

L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄; or

L¹-L²-X⁵ is

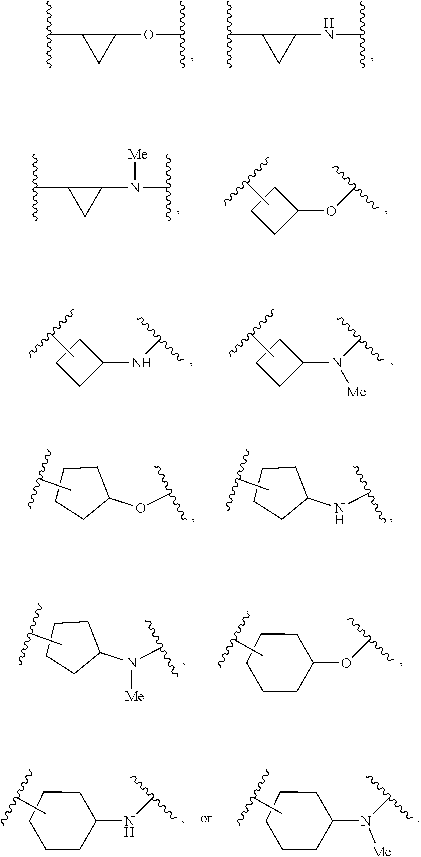

In embodiments, R² is H or CH₃.

In embodiments, L¹-L²-X⁵ is CH(CH₃)—(CH₂)₂—O, CH(CH₃)—(CH₂)₃—O, or (CH₂)₃—O.

In embodiments, a compound has a structure according to Formula (I-G-1),

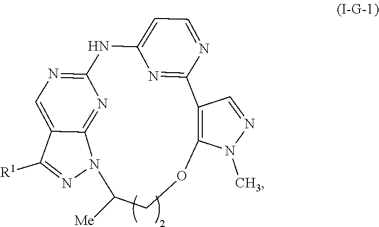

or a pharmaceutically acceptable salt thereof.

In embodiments, a compound has a structure according to Formula (I-H),

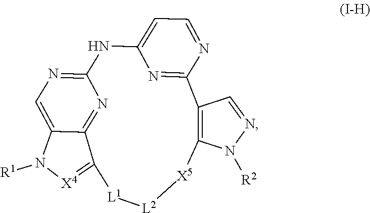

or a pharmaceutically acceptable salt thereof, wherein

X⁴ is CH or N;

R² is unsubstituted C₁₋₆ alkyl or C₁₋₆ alkyl substituted by a group that is unsubstituted C₃₋₆ cycloalkyl; and

L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄; or

L¹-L²-X⁵ is

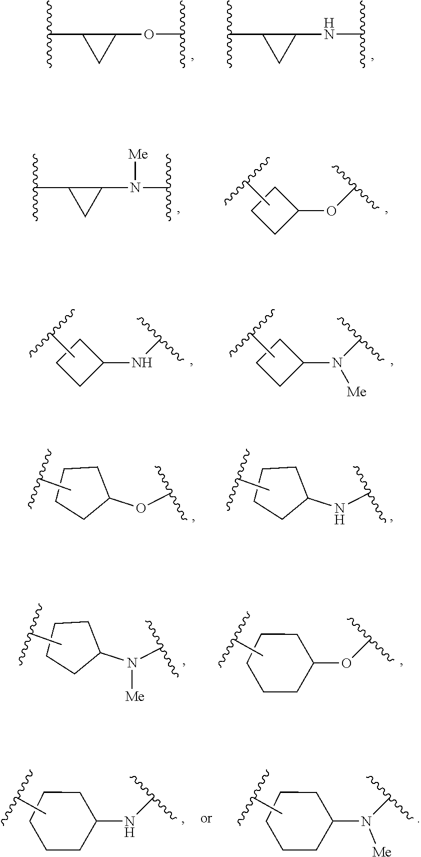

In embodiments, R² is H or CH₃.

In embodiments, L¹-L²-X⁵ is CH(CH₃)—(CH₂)₂—O, CH(CH₃)—(CH₂)₃—O, or (CH₂)₃—O.

In embodiments, a compound has a structure according to Formula (I-H-1),

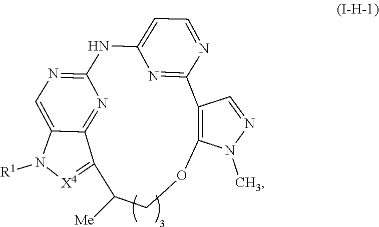

or a pharmaceutically acceptable salt thereof.

In embodiments, a compound has a structure according to Formula (I-I),

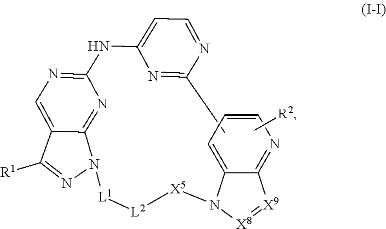

or a pharmaceutically acceptable salt thereof, wherein

R² is H or unsubstituted C₁₋₆ alkyl;

each X⁸ and X⁹ is CH or N; and

L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH₂CH₂, CH(CH₃)—CH₂)₃, or CH(CH₃)—(CH₂)₄; or

L¹-L²-X⁵ is

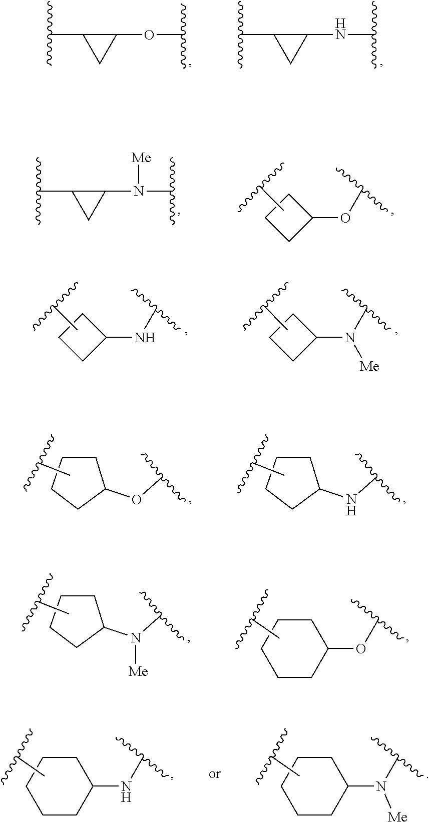

In embodiments, R² is H or CH₃.

In embodiments, L¹-L²-X⁵ is CH₂CH₂, CH(CH₃)—(CH₂)₃, or CH(CH₃)—(CH₂)₄.

In embodiments, a compound has a structure according to Formula (I-I-1),

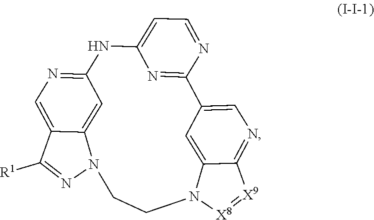

or a pharmaceutically acceptable salt thereof, wherein each X⁸ and X⁹ is CH or N.

In embodiments, a compound has a structure according to Formula (I-J),

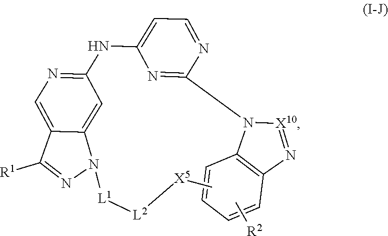

or a pharmaceutically acceptable salt thereof, wherein

R² is H or unsubstituted C₁₋₆ alkyl;

X¹⁰ is CH or N; and

L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH₂CH₂, CH(CH₃)—(CH₂)₃, or CH(CH₃)—(CH₂)₄; or

L¹-L²-X⁵ is

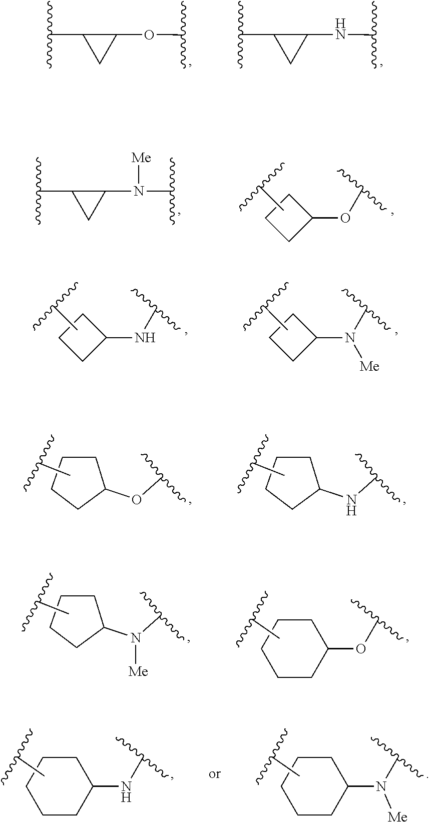

In embodiments, R² is H or CH₃.

In embodiments, L¹-L²-X⁵ is CH₂CH₂, CH(CH₃)—(CH₂)₃, or CH(CH₃)—(CH₂)₄.

In embodiments, a compound has a structure according to Formula (I-J-1),

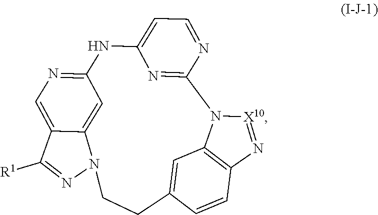

or a pharmaceutically acceptable salt thereof, wherein X¹⁰ is CH or N.

In embodiments, R¹ is F, CN, or NH₂.

In embodiments, R¹ has a structure according to Substructure 1,

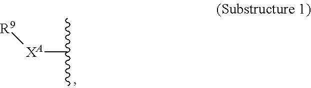

wherein

X^{A} is NH, NCH₃, or O; and R⁹ is a 3- to 6-membered oxygen-containing or nitrogen-containing heterocycloalkyl, C₃₋₇ cycloalkyl, or C₁₋₆ alkyl, and wherein said C₃₋₇ cycloalkyl or C₁₋₆ alkyl comprises one or two substituents selected from OH, NH₂, NMe₂, piperidinyl, and CONH₂.

In embodiments, R¹ is

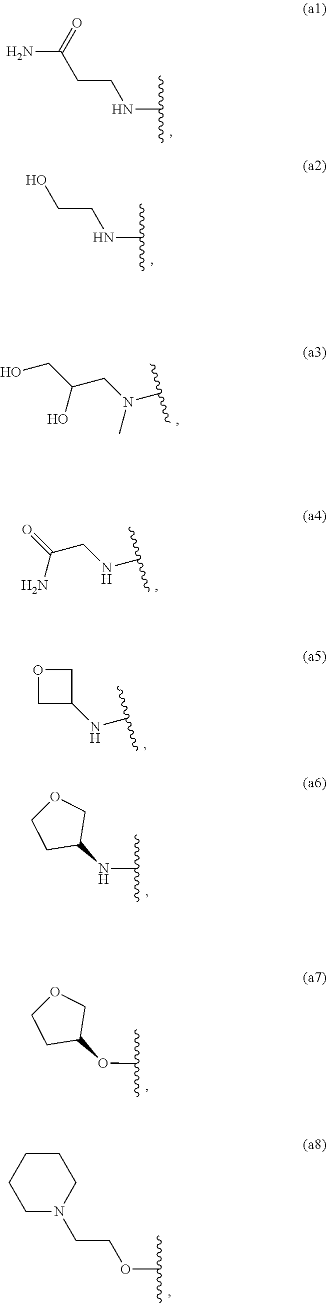

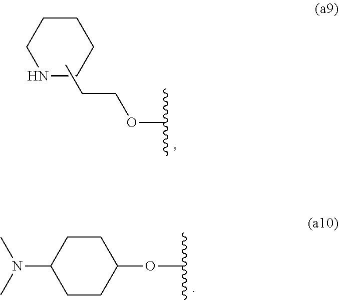

In embodiments, R¹ has a structure according to Substructure 2,

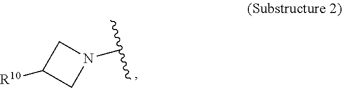

wherein

R¹⁰ is H, OH, C₁₋₆ alkyl, or CONR^{10A}R^{10B} and wherein said C₁₋₆ alkyl comprises one or two substituents selected from OH and CN;

each R^{10A} and R^{10B} is independently H, unsubstituted C₁₋₆ alkyl, C₁₋₆ alkyl substituted by alkoxy, or R^{10A} and R^{10B} together with the nitrogen atom to which they are attached form an unsubstituted 3- to 8-membered heterocycloalkyl ring.

In embodiments, R¹ is

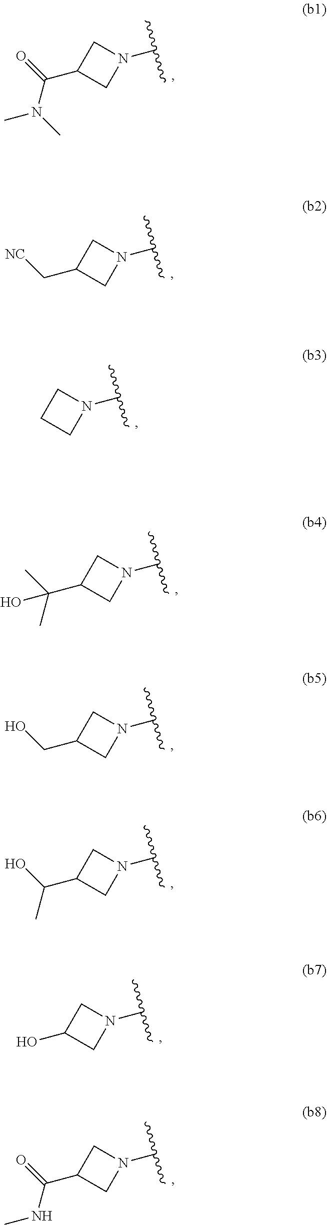

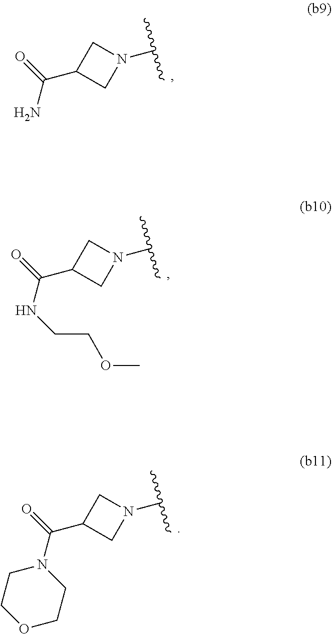 [SMILES: *N1CC(C(N)=O)C1 ; *N1CC(C(=O)NCCOC)C1 ; *N1CC(C(=O)N2CCOCC2)C1]

In embodiments, R¹ has a structure according to Substructure 3,

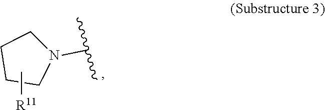

wherein

R¹¹ is H, OH, amino, mono(C₁₋₆ alkyl)amino, di(C₁₋₆ alkyl)amino, —CH₂-[di(C₁₋₆ alkyl)amino], CN, C₁₋₆ alkyl, CONH₂, CONHMe, COOH, CO₂Me, or CONR^{11A}R^{11B}; and wherein said C₁₋₆ alkyl comprises one or two substituents selected from OH, F, and NR^{11A}R^{11B}.

each R^{11A} and R^{11B} is independently unsubstituted C₁₋₆ alkyl, or R^{11A} and R^{11B} together with the nitrogen atom to which they are attached form a methyl or isopropyl substituted 3- to 8-membered heterocycloalkyl ring.

In embodiments, R¹ is

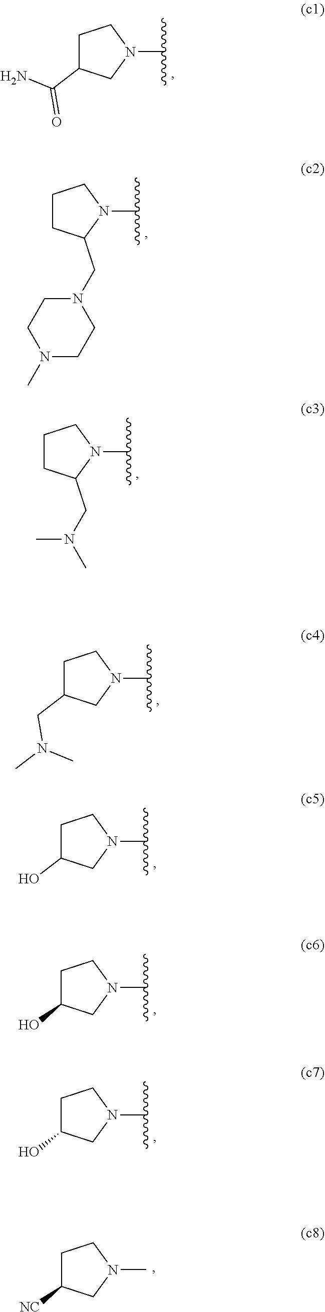

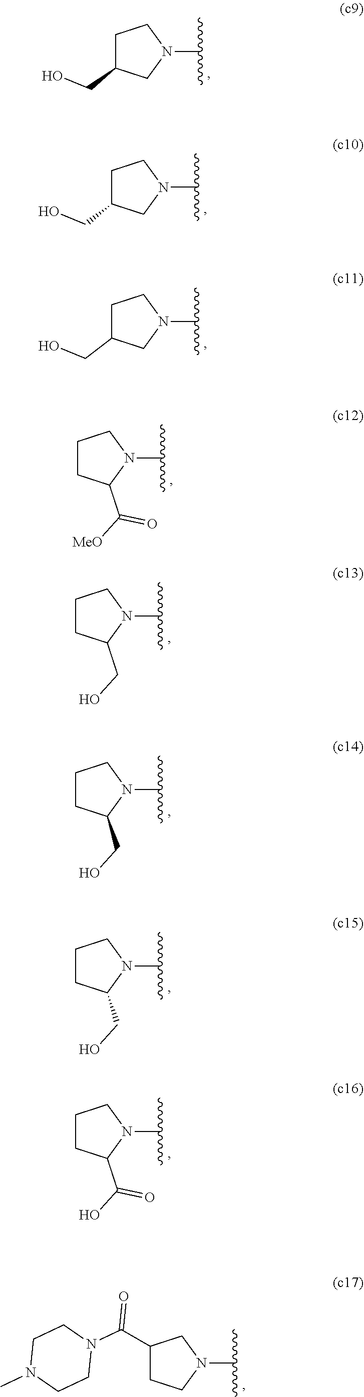

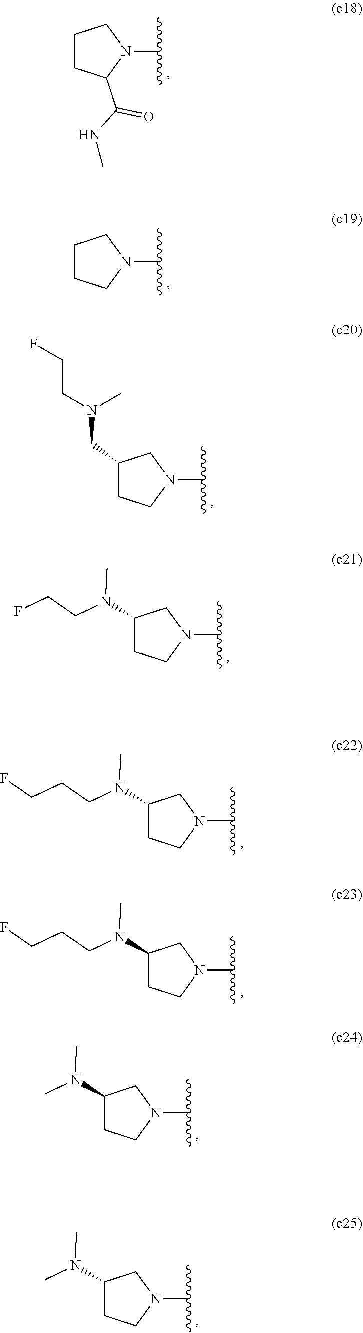

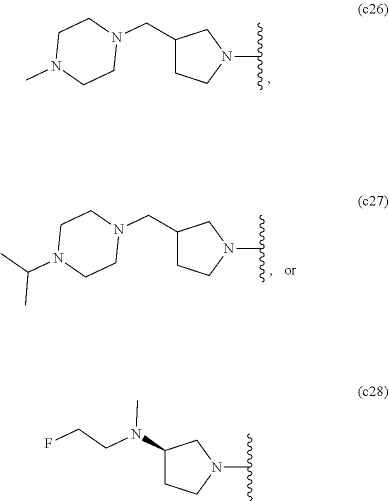 [SMILES: *N1CCC(CN2CCN(C)CC2)C1 ; *N1CCC(CN2CCN(C(C)C)CC2)C1 ; *N1CC[C@@H](N(C)CCF)C1]

In embodiments, R¹ has a structure according to Substructure 4,

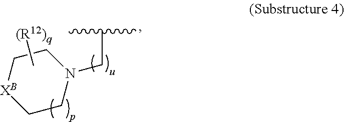

wherein

X^{B} is N, O, S, SO, or SO₂; each R¹², when present, is oxo, methyl, or cyclopropyl; p is 0 or 1; q is 0, 1, or 2; and u is 0 or 1.

In embodiments, R¹ is

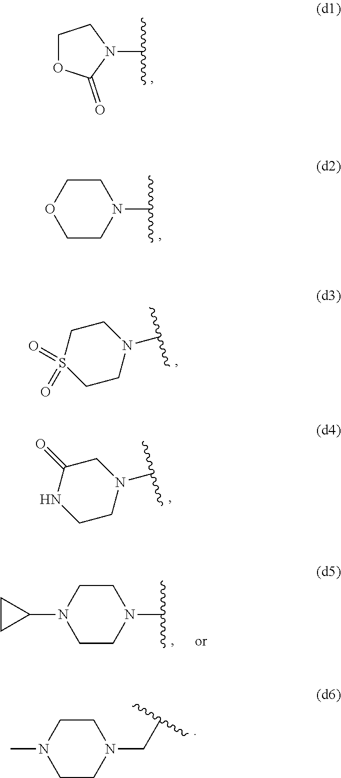

In embodiments, R¹ has a structure according to Substructure 5,

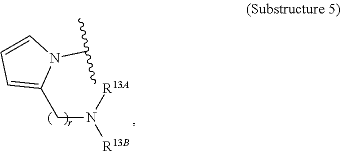

wherein

and each R^{13A} and R^{13B} is independently unsubstituted C₁₋₆ alkyl, or R^{13A} and R^{13B} together with the nitrogen atom to which they are attached form a N-methyl 3- to 8-membered heterocycloalkyl ring.

In embodiments, R¹ is

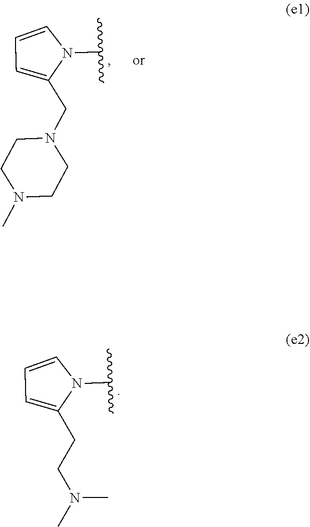 [SMILES: *n1cccc1CN1CCN(C)CC1 ; *n1cccc1CCN(C)C]

In embodiments, R¹ has a structure according to Substructure 6,

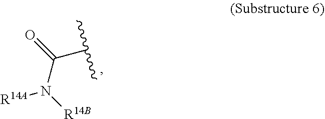

wherein

each R^{14A} and R^{14B} is independently H, unsubstituted C₁₋₆ alkyl, or 5- to 6-membered cycloalkyl ring substituted with CN.

In embodiments, R¹ is

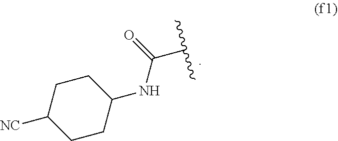 [SMILES: *C(=O)NC1CCC(C#N)CC1]

In embodiments, R¹ has a structure according to Substructure 7,

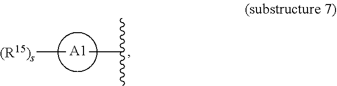

wherein

s is 0, 1, 2, or 3;

v is 0, 1, 2, or 3;

A1 is phenyl, 5- to 6-membered heteroarylene or 5- to 6-membered heterocycloalkyl;

R¹⁵ is independently
halogen
unsubstituted C₁₋₆ alkyl;
C₃₋₆ cycloalkyl;
C₁₋₆ alkyl substituted by OH or OMe;
C₁₋₆ alkyl substituted by halo, amino, monoalkylamino, or dialkylamino;
C₁₋₆ alkoxyl substituted by halo, amino, monoalkylamino, or dialkylamino;
8- to 9-membered heterocycloalkyl;
—(CH₂)ᵥ-(5- to 6-membered heterocycloalkyl);
—(CH₂)ᵥ-(5- to 6-membered heteroaryl);
—(CO)-(5- to 6-membered heterocycloalkyl);
—(CO)-(5- to 6-membered heteroaryl);
—O-(5- to 6-membered heterocycloalkyl);
—O-(5- to 6-membered heteroaryl);
—(CH₂)ᵥ—NH—(C₁₋₆ alkyl substituted by halo, OH, OMe, amino, monoalkylamino, or dialkylamino);
—(CH₂)ᵥ—NMe-(C₁₋₆ alkyl substituted by halo, OH, OMe, amino, monoalkylamino, or dialkylamino).

In embodiments, A1 is furan, pyrazole, pyrrole, thiazole, oxazole, phenyl, pyridyl, or a bicyclic nitrogen-containing 8- to 9-membered heterocycloalkyl.

In embodiments, substructure 7 is

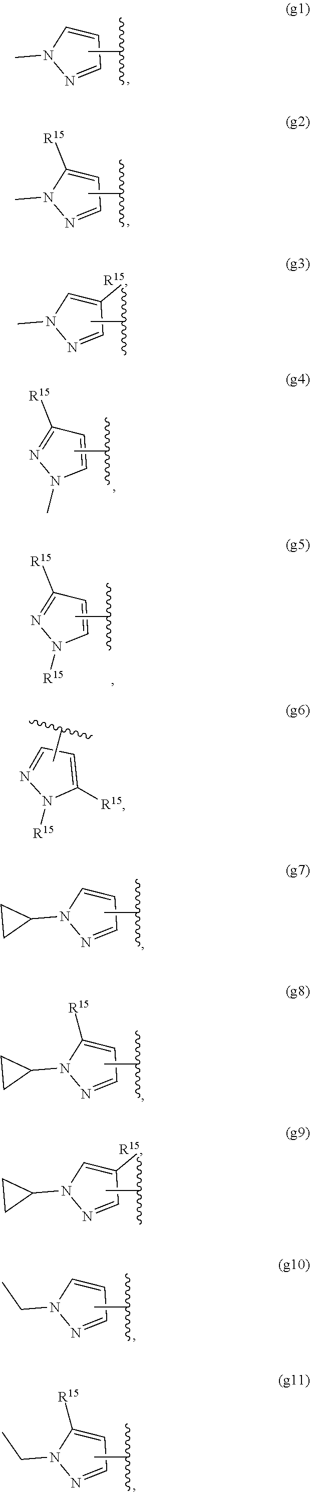

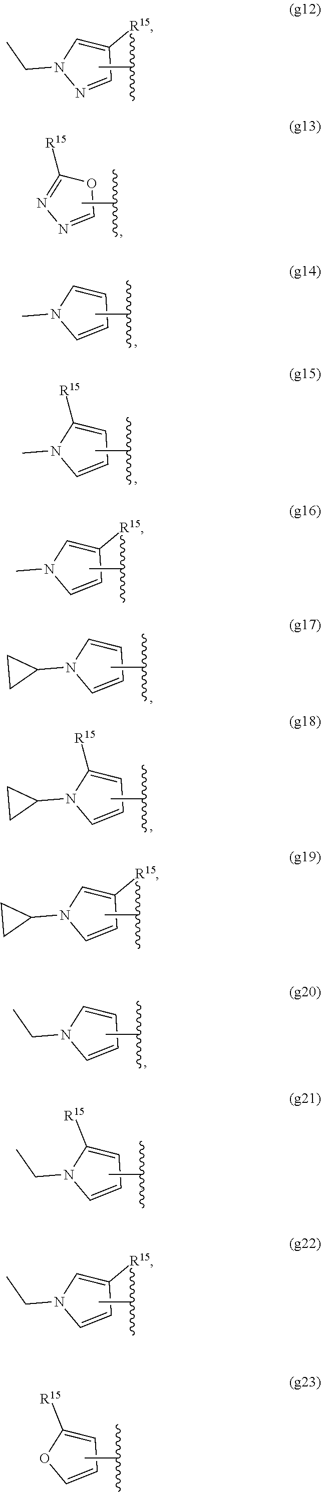

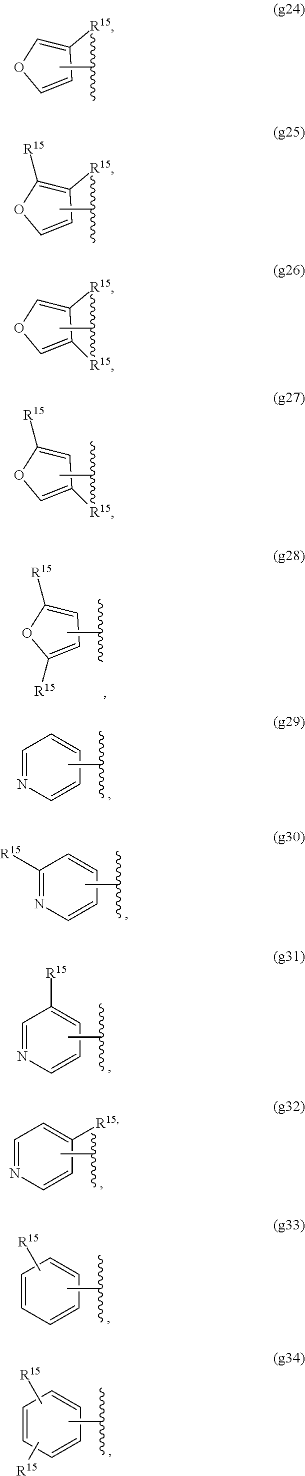

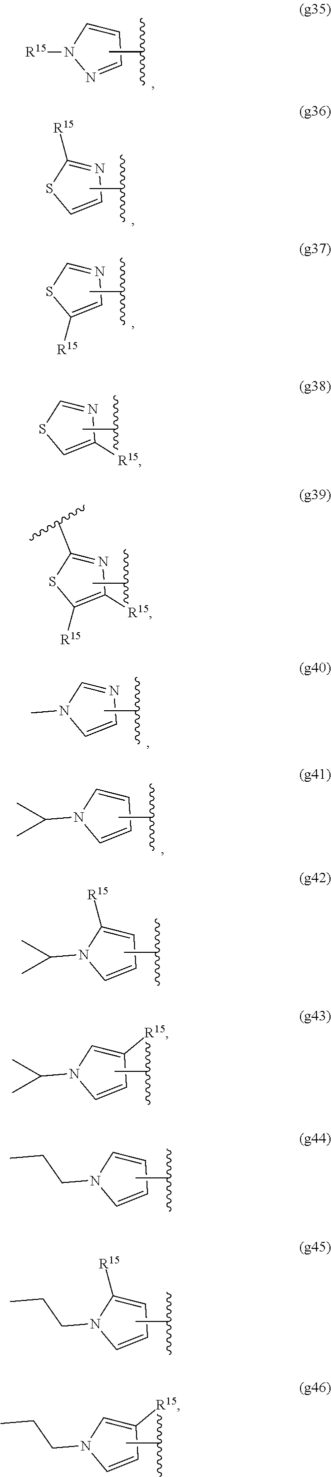

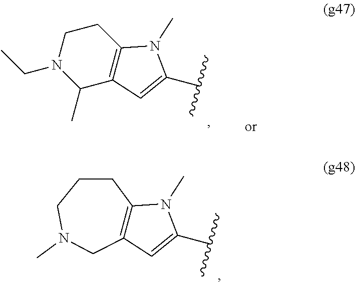 [SMILES: *c1cc2c(n1C)CCN(CC)C2C ; *c1cc2c(n1C)CCCN(C)C2]

In embodiments, each R¹⁵ is independently —F, —Cl, —CH₃, —CH₂CH₃, —CH(CH₃)₂,

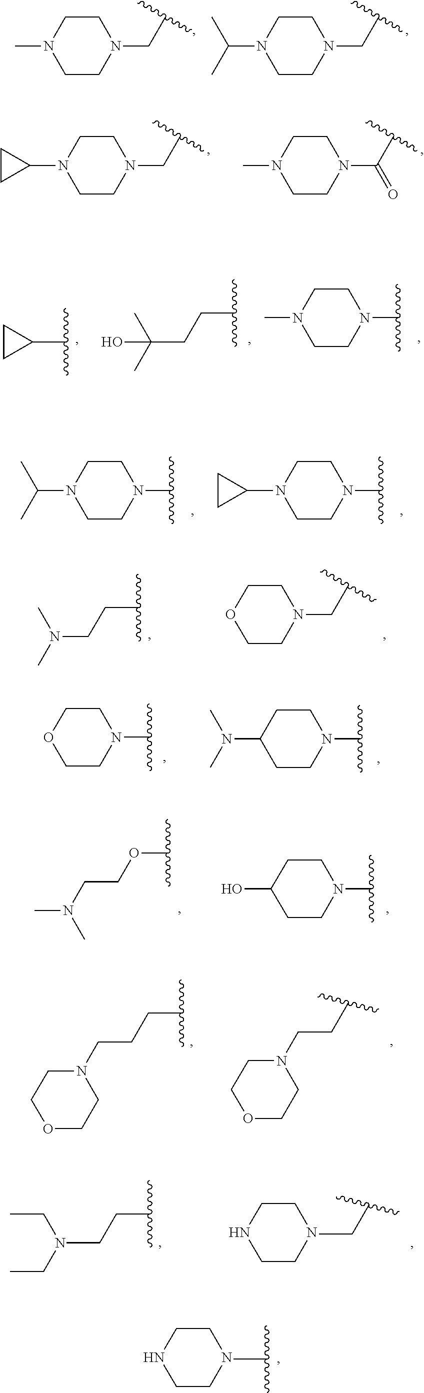

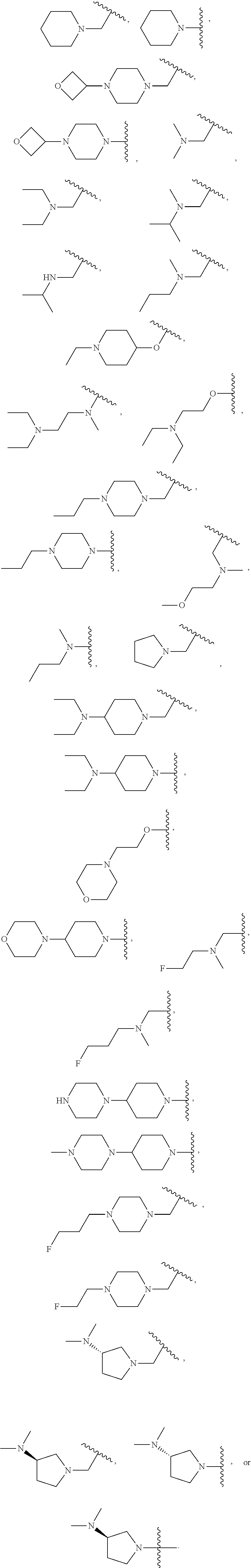

In embodiments, a compound is selected from the group consisting of any one of Compounds (1)-(58), (61)-(71), (73)-(80), and (82)-(193), or a pharmaceutically acceptable salt thereof.

In another aspect, the invention features a pharmaceutical composition comprising any compound described herein, or a pharmaceutically acceptable salt thereof.

In another aspect, the invention features a method of treating cancer comprising administering to a human in need thereof an effective amount of any compound described herein, or a pharmaceutically acceptable salt thereof, in a pharmaceutical composition.

In embodiments, a cancer is a lung cancer.

In embodiments, a cancer is non-small cell lung cancer.

In embodiments, a cancer (e.g., a lung cancer such as non-small cell lung cancer) is an EGFR-driven cancer.

In embodiments, a cancer (e.g., a lung cancer such as non-small cell lung cancer) is characterized by an EGFR mutation.

### DETAILED DESCRIPTION OF THE INVENTION

### Definitions

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Animal: As used herein, the term “animal” refers to any member of the animal kingdom. In some embodiments, “animal” refers to humans, at any stage of development. In some embodiments, “animal” refers to non-human animals, at any stage of development. In certain embodiments, the non-human animal is a mammal (e.g., a rodent, a mouse, a rat, a rabbit, a monkey, a dog, a cat, a sheep, a bovine, a primate, and/or a pig). In some embodiments, animals include, but are not limited to, mammals, birds, reptiles, amphibians, fish, insects, and/or worms. In some embodiments, an animal may be a transgenic animal, genetically-engineered animal, and/or a clone.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used in the description and the appended claims, the singular forms “a,” “an,” and “the” include plural referents unless the context clearly dictates otherwise. Thus, for example, reference to “a composition” includes mixtures of two or more such compositions.

Throughout the description and claims of this specification the word “comprise” and other forms of the word, such as “comprising” and “comprises,” means including but not limited to, and is not intended to exclude, for example, other additives, components, integers, or steps.

“Optional” or “optionally” means that the subsequently described event or circumstance may or may not occur, and that the description includes instances where the event or circumstance occurs and instances where it does not.

Improve, increase, or reduce: As used herein, the terms “improve,” “increase,” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Patient: As used herein, the term “patient” or “subject” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable,” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio. Accordingly, pharmaceutically acceptable relates to substances that are not biologically or otherwise undesirable, i.e., the material can be administered to an individual along with the relevant active compound without causing clinically unacceptable biological effects or interacting in a deleterious manner with any of the other components of the pharmaceutical composition in which it is contained.

Pharmaceutically acceptable salt: Pharmaceutically acceptable salts are well known in the art. For example, S. M. Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid, or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄-alkyl)₄ salts. Representative alkali or alkaline earth metal salts include sodium, lithium, potassium, calcium, magnesium, and the like. Further pharmaceutically acceptable salts include, when appropriate, nontoxic ammonium. quaternary ammonium, and amine cations formed using counterions such as halide, hydroxide, carboxylate, sulfate, phosphate, nitrate, sulfonate, and aryl sulfonate. Further pharmaceutically acceptable salts include salts formed from the quarternization of an amine using an appropriate electrophile, e.g., an alkyl halide, to form a quarternized alkylated amino salt.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Whenever a term (e.g., alkyl or aryl) or either of their prefix roots (e.g., alk- or ar-) appear in a name of a substituent the name is to be interpreted as including those limitations provided herein. For example, affixing the suffix “-ene” to a group indicates the group is a divalent moiety, e.g., arylene is the divalent moiety of aryl, heteroarylene is the divalent moiety of heteroaryl, and heterocycloalkylene is the divalent moiety of heterocycloalkyl. Similarly, affixing the suffix “-oxy” to a group indicates the group is attached to the parent molecular structure through an oxygen atom (—O—).

Alkyl: As used herein, the term “alkyl” means acyclic linear and branched hydrocarbon groups, e.g. “C₁-C₂₀ alkyl” refers to alkyl groups having 1-20 carbons and “C₁-C₄ alkyl” refers to alkyl groups having 1-4 carbons. Alkyl groups include C₁-C₂₀ alkyl, C₁-C₁₅ alkyl, C₁-C₁₀ alkyl, C₁-C₄ alkyl, and C₁-C₃ alkyl). In embodiments, an alkyl group is C₁-C₄ alkyl. An alkyl group may be linear or branched. Examples of alkyl groups include, but are not limited to, methyl, ethyl, n-propyl, isopropyl, butyl, isobutyl, sec-butyl, tert-butyl, pentyl, isopentyl tert-pentylhexyl, isohexyl, etc. The term “lower alkyl” means an alkyl group straight chain or branched alkyl having 1 to 6 carbon atoms. Other alkyl groups will be readily apparent to those of skill in the art given the benefit of the present disclosure. An alkyl group may be unsubstituted or substituted with one or more substituent groups as described herein. For example, an alkyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, C₁-C₄ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkyl is substituted (e.g., with 1, 2, 3, 4, 5, or 6 substituent groups as described herein). In some embodiments, an alkyl group is substituted with a-OH group and may also be referred to herein as a “hydroxyalkyl” group, where the prefix denotes the —OH group and “alkyl” is as described herein. In some embodiments, an alkyl group is substituted with a-OR′ group.

Alkylene: The term “alkylene,” as used herein, represents a saturated divalent straight or branched chain hydrocarbon group and is exemplified by methylene, ethylene, isopropylene and the like. Likewise, the term “alkenylene” as used herein represents an unsaturated divalent straight or branched chain hydrocarbon group having one or more unsaturated carbon-carbon double bonds that may occur in any stable point along the chain, and the term “alkynylene” herein represents an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl).

In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms.

Alkenyl: As used herein, “alkenyl” means any linear or branched hydrocarbon chains having one or more unsaturated carbon-carbon double bonds that may occur in any stable point along the chain, e.g. “C₂-C₂₀ alkenyl” refers to an alkenyl group having 2-20 carbons. For example, an alkenyl group includes prop-2-enyl, but-2-enyl, but-3-enyl, 2-methylprop-2-enyl, hex-2-enyl, hex-5-enyl, 2,3-dimethylbut-2-enyl, and the like. In some embodiments, the alkenyl comprises 1, 2, or 3 carbon-carbon double bond. In some embodiments, the alkenyl comprises a single carbon-carbon double bond. In some embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkenyl is unsubstituted. In some embodiments, the alkenyl is substituted (e.g., with 1, 2, 3, 4, 5, or 6 substituent groups as described herein). In some embodiments, an alkenyl group is substituted with a-OH group and may also be referred to herein as a “hydroxyalkenyl” group, where the prefix denotes the —OH group and “alkenyl” is as described herein.

Alkynyl: As used herein, “alkynyl” means any hydrocarbon chain of either linear or branched configuration, having one or more carbon-carbon triple bonds occurring in any stable point along the chain, e.g. “C₂-C₂₀ alkynyl” refers to an alkynyl group having 2-20 carbons. Examples of an alkynyl group include prop-2-ynyl, but-2-ynyl, but-3-ynyl, pent-2-ynyl, 3-methylpent-4-ynyl, hex-2-ynyl, hex-5-ynyl, etc. In some embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR′, —CO₂H, —CO₂R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In some embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In some embodiments, R′ independently is unsubstituted C₁-C₃ alkyl. In some embodiments, the alkynyl is unsubstituted. In some embodiments, the alkynyl is substituted (e.g., with 1, 2, 3, 4, 5, or 6 substituent groups as described herein).

Alkoxy: The term “alkoxy” refers to the group —O-alkyl, including from 1 to 10 carbon atoms of a straight, branched, saturated cyclic configuration and combinations thereof, attached to the parent molecular structure through an oxygen. Examples include methoxy, ethoxy, propoxy, isopropoxy, butoxy, t-butoxy, pentoxy, cyclopropyloxy, cyclohexyloxy and the like. “Lower alkoxy” refers to alkoxy groups containing one to six carbons. In some embodiments, C₁₋₄ alkoxy is an alkoxy group which encompasses both straight and branched chain alkyls of from 1 to 4 carbon atoms. Unless stated otherwise in the specification, an alkoxy group can be optionally substituted by one or more substituents (e.g., as described herein for alkyl). The terms “alkenoxy” and “alkynoxy” mirror the above description of “alkoxy” wherein the prefix “alk” is replaced with “alken” or “alkyn” respectively, and the parent “alkenyl” or “alkynyl” terms are as described herein.

Amide: The term “amide” or “amido” refers to a chemical moiety with formula —C(O)N(R′)₂, —C(O)N(R′)—, —NR′C(O)R′, or —NR′C(O)—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Amino: The term “amino” or “amine” refers to a —N(R′)₂ group, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated otherwise in the specification, each of which moiety can itself be optionally substituted as described herein, or two R′ can combine with the nitrogen atom to form a 3-, 4-, 5-, 6-, or 7-membered ring. In embodiments, an amino group is —NHR′, where R′ is aryl (“arylamino”), heteroaryl (“heteroarylamino”), or alkyl (“alkylamino”).

Aryl: The term “aryl” used alone or as part of a larger moiety as in “aralkyl,” refers to a monocyclic, bicyclic, or tricyclic carbocyclic ring system having a total of six to fourteen ring members, wherein said ring system has a single point of attachment to the rest of the molecule, wherein at least one ring in the system is aromatic, and wherein each ring in the system contains 4 to 7 ring members. In some embodiments, an aryl group has 6 ring carbon atoms (“C₆ aryl,” e.g., phenyl). In some embodiments, an aryl group has 10 ring carbon atoms (“C₁₀ aryl,” e.g., naphthyl such as 1-naphthyl and 2-naphthyl). In some embodiments, an aryl group has 14 ring carbon atoms (“C₁₋₄ aryl,” e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Exemplary aryls include phenyl, naphthyl, and anthracene.

Arylalkyl: The term “arylalkyl” refers to an -(alkylene)-aryl radical where aryl and alkylene are as disclosed herein and which are optionally substituted by one or more of the exemplary substituent groups described herein. The “arylalkyl” group is bonded to the parent molecular structure through the alkylene moiety. The term “arylalkoxy” refers to an —O-[arylalkyl] radical (—O-[(alkylene)-aryl]), which is attached to the parent molecular structure through the oxygen.

Arylene: The term “arylene” as used herein refers to an aryl group that is divalent (that is, having two points of attachment to the molecule). Exemplary arylenes include phenylene (e.g., unsubstituted phenylene or substituted phenylene).

Cyclic: The term “cyclic” as used herein, refers to any covalently closed structure. Cyclic moieties include, for example, carbocycles (e.g., aryls and cycloalkyls), heterocycles (e.g., heteroaryls and heterocycloalkyls), aromatics (e.g. aryls and heteroaryls), and non-aromatics (e.g., cycloalkyls and heterocycloalkyls). In some embodiments, cyclic moieties are optionally substituted. In some embodiments, cyclic moieties form part of a ring system.

Cycloaliphatic: The term “cycloaliphatic” refers to a monocyclic or polycyclic radical that contains only carbon and hydrogen, and can be saturated or partially unsaturated. Fully saturated cycloaliphatics can be termed “cycloalkyl”. Partially unsaturated cycloalkyl groups can be termed “cycloalkenyl” if the carbocycle contains at least one double bond, or “cycloalkynyl” if the carbocycle contains at least one triple bond. Cycloaliphatic groups include groups having from 3 to 13 ring atoms (e.g., C₃₋₁₃ cycloalkyl). Whenever it appears herein, a numerical range such as “3 to 10” refers to each integer in the given range; e.g., “3 to 10 carbon atoms” means that the cycloaliphatic group (e.g., cycloalkyl) can consist of 3 carbon atoms, 4 carbon atoms, 5 carbon atoms, etc., up to and including 10 carbon atoms. The term “cycloaliphatic” also includes bridged and spiro-fused cyclic structures containing no heteroatoms. The term also includes monocyclic or fused-ring polycyclic (i.e., rings which share adjacent pairs of ring atoms) groups. Polycyclic cycloaliphatic groups include bicycles, tricycles, tetracycles, and the like. In some embodiments, “cycloalkyl” can be a C₃₋₈ cycloalkyl group. In some embodiments, “cycloalkyl” can be a C₃₋₅ cycloalkyl group. Illustrative examples of cycloaliphatic groups include, but are not limited to the following moieties: C₃₋₆ cycloaliphatic groups include, without limitation, cyclopropyl (C₃), cyclobutyl (C₄), cyclopentyl (C₅), cyclopentenyl (C₅), cyclohexyl (C₆), cyclohexenyl (C₆), cyclohexadienyl (C₆) and the like. Examples of C₃₋₇ cycloaliphatic groups include norbornyl (C₇). Examples of C₃₋₈ cycloaliphatic groups include the aforementioned C₃₋₇ carbocyclyl groups as well as cycloheptyl(C₇), cycloheptadienyl (C₇), cyclohept-atrienyl (C₇), cyclooctyl (C₈), bicyclo[2.2.1]heptanyl, bicyclo[2.2.2]octanyl, and the like. Examples of C₃₋₁₃ cycloaliphatic groups include the aforementioned C₃₋₈ carbocyclyl groups as well as octahydro-1H indenyl, decahydronaphthalenyl, spiro[4.5]decanyl, and the like.

Cyano: The term “cyano” refers to a —CN group.

Deuterium: The term “deuterium” is also called heavy hydrogen. Deuterium is isotope of hydrogen with a nucleus consisting of one proton and one neutron, which is double the mass of the nucleus of ordinary hydrogen (one proton). In embodiments, deuterium can also be identified as ²H.

Ester: The term “ester” refers to a group of formula —C(O)OR′ or —R′OC(O)—, where R′ is selected from alkyl, alkenyl, alkynyl, heteroalkyl (bonded through a chain carbon), cycloalkyl, aryl, arylalkyl, heteroaryl, heteroarylalkyl, or heterocycloalkyl as described herein.

Halogen or Halo: As used herein, the term “halogen” or “halo” means fluorine, chlorine, bromine, or iodine.

Heteroalkyl: The term “heteroalkyl” is meant a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl. Accordingly, the term “heteroalkoxy” refers to the group —O— heteroalkyl, where the group is attached to the parent molecular structure via the oxygen.

Heteroalkylene: The term “heteroalkylene,” as used herein, represents a divalent form of a heteroalkyl group as described herein.

Heteroaryl: The term “heteroaryl,” as used herein, refers to a monocyclic, bicyclic, or tricyclic carbocyclic ring system having a total of six to fourteen ring members, wherein said ring system has a single point of attachment to the rest of the molecule, wherein at least one ring in the system is aromatic, wherein each ring in the system contains 4 to 7 ring members, and wherein at least one ring atom is a heteroatom such as, but not limited to, nitrogen and oxygen. Examples of heteroaryl groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. Accordingly, the term “heteroaryloxy” refers to the group —O-heteroaryl, where the group is attached to the parent molecular structure via the oxygen.

Heteroarylalkyl: The term “heteroarylalkyl” refers to an -(alkylene)-heteroaryl radical where heteroaryl and alkylene are as disclosed herein and which are optionally substituted by one or more of the exemplary substituent groups described herein. The “heteroarylalkyl” group is bonded to the parent molecular structure through the alkylene moiety. The term “heteroarylalkoxy” refers to an —O-[heteroarylalkyl] radical (—O-[(alkylene)-heteroaryl]), which is attached to the parent molecular structure through the oxygen.

Heterocycloalkyl: The term “heterocycloalkyl,” as used herein, is a non-aromatic ring wherein at least one atom is a heteroatom such as, but not limited to, nitrogen, oxygen, sulfur, or phosphorus, and the remaining atoms are carbon. Examples of heterocycloalkyl groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. The heterocycloalkyl group can be substituted or unsubstituted.

Heterocycle: The term “heterocycle” refers to heteroaryl and heterocycloalkyl as used herein, refers to groups containing one to four heteroatoms each selected from O, S and N, wherein each heterocycle group has from 4 to 10 atoms in its ring system, and with the proviso that the ring of said group does not contain two adjacent O or S atoms. Herein, whenever the number of carbon atoms in a heterocycle is indicated (e.g., C₁-C₆-heterocycle), at least one other atom (the heteroatom) must be present in the ring. Designations such as “C₁-C₆-heterocycle” refer only to the number of carbon atoms in the ring and do not refer to the total number of atoms in the ring. In some embodiments, it is understood that the heterocycle ring has additional heteroatoms in the ring. Designations such as “4-6-membered heterocycle” refer to the total number of atoms that are contained in the ring (i.e., a four, five, or six membered ring, in which at least one atom is a carbon atom, at least one atom is a heteroatom and the remaining two to four atoms are either carbon atoms or heteroatoms). In some embodiments, in heterocycles that have two or more heteroatoms, those two or more heteroatoms are the same or different from one another. In some embodiments, heterocycles are optionally substituted. In some embodiments, binding to a heterocycle is at a heteroatom or via a carbon atom. Heterocycloalkyl groups include groups having only 4 atoms in their ring system, but heteroaryl groups must have at least 5 atoms in their ring system. The heterocycle groups include benzo-fused ring systems. An example of a 4-membered heterocycle group is azetidinyl (derived from azetidine). An example of a 5-membered heterocycle group is thiazolyl. An example of a 6-membered heterocycle group is pyridyl, and an example of a 10-membered heterocycle group is quinolinyl. In some embodiments, the foregoing groups, as derived from the groups listed above, are C-attached or N-attached where such is possible. For instance, in some embodiments, a group derived from pyrrole is pyrrol-1-yl (N-attached) or pyrrol-3-yl (C-attached). Further, in some embodiments, a group derived from imidazole is imidazol-1-yl or imidazol-3-yl (both N-attached) or imidazol-2-yl, imidazol-4-yl or imidazol-5-yl (all C-attached). The heterocycle groups include benzo-fused ring systems and ring systems substituted with one or two oxo (═O) moieties such as pyrrolidin-2-one. In some embodiments, depending on the structure, a heterocycle group is a monoradical or a diradical (i.e., a heterocyclene group). The heterocycles described herein are substituted with 0, 1, 2, 3, or 4 substituents independently selected from alkenyl, alkoxy, alkoxyalkyl, alkoxycarbonyl, alkyl, alkylcarbonyl, alkylcarbonyloxy, alkylthio, alkylthioalkyl, alynyl, carboxy, cyano, formyl, haloalkoxy, haloalkyl, halogen, hydroxyl, hydroxyalkylene, mercapto, nitro, amino, and amido moities.

Isotope: The term “isotope” refers to a variant of a particular chemical element which differs in neutron number, and consequently in nucleon number. All isotopes of a given element have the same number of protons but different numbers of neutrons in each atom.

Nitro: The term “nitro” refers to a —NO₂ group.

Sulfonamide: The term “sulfonamide” or sulfonamido” refers to the following groups: —S(═O)₂—(R′)₂, —N(R′)—S(═O)₂—R′, —S(═O)₂—N(R′)—, or —N(R′)—S(═O)₂—, where each R′ is independently selected from hydrogen, alkyl, alkenyl, alkynyl, heteroalkyl (bonded through a chain carbon), cycloalkyl, aryl, arylalkyl, heteroaryl (bonded through a ring carbon), heteroarylalkyl, or heterocycloalkyl (bonded through a ring carbon), unless stated other-wise in the specification, each of which moiety can itself be optionally substituted as described herein, or two R′ can combine with the nitrogen atom to form a 3-, 4-, 5-, 6-, or 7-membered ring.

Moiety: The term “moiety” refers to a specific segment or functional group of a molecule. Chemical moieties are often recognized chemical entities embedded in or appended to a molecule.

Molecular groups herein may be substituted or unsubstituted (e.g., as described herein). The term “substituted” means that the specified group or moiety bears one or more substituents: at least one hydrogen present on a group atom (e.g., a carbon or nitrogen atom) is replaced with a permissible substituent, e.g., a substituent which upon substitution for the hydrogen results in a stable compound, e.g., a compound which does not spontaneously undergo transformation such as by rearrangement, cyclization, elimination, or other reaction. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In embodiments, a group described herein is substituted. In embodiments, a group described herein is unsubstituted. In cases where a specified moiety or group is not expressly noted as being optionally substituted or substituted with any specified substituent, it is understood that such a moiety or group is intended to be unsubstituted.

A wide variety of substituents are well known, and methods for their formation and introduction into a variety of parent groups are also well known. Representative substituents include but are not limited to alkyl, cycloalkyl, alkenyl, cycloalkenyl, alkynyl, arylalkyl, alkylaryl, aryl, heteroaryl, heterocycloalkyl, hydroxyalkyl, arylalkyl, aminoalkyl, haloalkyl, thioalkyl, alkylthioalkyl, carboxyalkyl, imidazolylalkyl, indolylalkyl, mono-, di- and trihaloalkyl, mono-, di- and trihaloalkoxy, amino, alkylamino, dialkylamino, alkoxy, hydroxy, halo (e.g., Cl and Br), nitro, oximino, COOR⁵⁰, COR⁵⁰, SO₀₋₂R⁵⁰, SO₂NR⁵⁰R⁵¹, NR⁵²SO₂R⁵⁰, ═C(R⁵⁰R⁵¹), ═N—OR⁵⁰, ═N—CN, ═C(halo)₂, ═S, ═O, CON(R⁵⁰R⁵¹), OCOR⁵⁰, OCON(R⁵⁰R⁵¹), N(R⁵²)CO(R⁵⁰), N(R⁵²)COOR⁵⁰, N(R⁵²)CON(R⁵⁰(R⁵¹), P(OR⁵⁰)₂, P(O)R⁵⁰R⁵¹, and P(O)OR⁵⁰OR⁵¹, wherein R⁵⁰, R⁵¹ and R⁵² may be independently selected from the following: a hydrogen atom and a branched or straight-chain, C₁₋₆-alkyl, C₃₋₆-cycloalkyl, C₄₋₆-heterocycloalkyl, heteroaryl and aryl group, with or without substituents. When permissible, R⁵⁰ and R⁵¹ can be joined together to form a carbocyclic or heterocyclic ring system.

In preferred embodiments, the substituent is selected from halogen, —COR′, —CO₂H, —CO₂R′, —CN, —OH, —OR′, —OCOR′, —OCO₂R′, —NH₂, —NHR′, —N(R′)₂, —SR′, and —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In certain embodiments thereof, R′ independently is an unsubstituted alkyl (e.g., unsubstituted C₁-C₂₀ alkyl, C₁-C₁₅ alkyl, C₁-C₁₀ alkyl, or C₁-C₃ alkyl). Preferably, R′ independently is unsubstituted C₁-C₃ alkyl.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of the general formula, and mixtures thereof, are considered within the scope of the formula. Thus, any formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any formula given herein is intended to embrace hydrates, solvates, and polymorphs of such compounds, and mixtures thereof.

### Compounds of Formula (I) and Formula (I′)

Exemplary compounds are described herein.

Any structural feature described herein (e.g., for any exemplary formula described herein) can be used in combination with any other structural feature(s) described for any exemplary formula described herein.

In one embodiment, the invention features a compound having a structure according to Formula (I),

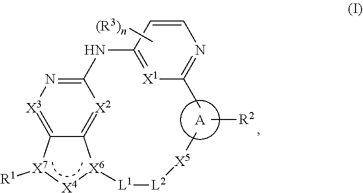

or a pharmaceutically acceptable salt thereof,

wherein:

A is C₆₋₁₀ arylene, 5-12-membered heteroarylene, or 5-12-membered heterocycloalkylene;

X¹ is N or CR^{X};

X² is N or CR^{X};

X³ is N or CR^{X};

X⁴ is N or CR^{X};

X⁶ is N or CR^{X′};

X⁷ is N or CR^{X′};

[symbol] represents an optional double bond between X⁷ and X⁴ or X⁴ and X⁶, wherein one and only one double bond is present;

X⁵ is a covalent bond, CH₂, O, NR⁴, C(O)NR⁴, or NR⁴C(O);

L¹ is a covalent bond or C(R⁵)₂, and L² is C₁₋₄ alkylene, or L¹ and L² combine to form a C₃₋₆ cycloalkyl or a 4- to 6-membered heterocycloalkyl;

R¹ is halogen, C₁₋₆ alkyl, C₃₋₇ cycloalkyl, C₆₋₁₀ aryl, 5- to 10-membered heteroaryl, 3- to 10-membered heterocycloalkyl, CN, NR⁶R⁷, NR⁶C(O)R⁷, NR⁶C(O)NH₂, OR⁸, or C(O)NR⁶R⁷;

R² is absent, H, C₁₋₆ alkyl, halogen, CN, or C₁₋₆ alkoxy;

each R³, when present, is independently OH, CN, halogen, C₁₋₆ alkyl, or C₁₋₆ alkoxy;

n is 0, 1, or 2;

each R^{X} is independently H, OR^{X1}, CN, halogen, or C₁₋₆ alkyl, wherein R^{X1} is H or C₁₋₆ alkyl;

each R^{X′} is independently H, OR^{X1}, CN, halogen, or C₁₋₆ alkyl, wherein R^{X1} is H or C₁₋₆ alkyl, or R^{X′} is absent if the carbon to which it is attached is part of a double bond;

each R⁴ and R⁵ is independently H or C₁₋₆ alkyl;

each R⁶ and R⁷ is independently H, C₁₋₆ alkyl, C₃₋₇ cycloalkyl, or 3- to 10-membered heterocycloalkyl; or R⁶ and R⁷ together with the nitrogen atom to which they are attached form a 3- to 8-membered heterocycloalkyl ring; and

R⁸ is independently H, C₁₋₆ alkyl, or 4- to 6-membered heterocycloalkyl.

Certain exemplary structure features are described herein. Exemplary structural formulae and compounds can feature any combination of features as described herein.

In embodiments, [symbol] represents a double bond between X⁷ and X⁴, and there is a single bond between X⁶ and X⁴. In embodiments, X⁷ is N and X⁴ is CR^{X}. In embodiments, X⁷ is C and X⁴ is CR^{X}. In embodiments, X⁷ is N and X⁴ is N. In embodiments, X⁶ is N. In embodiments, X⁶ is CR^{X} (e.g., C—H).

In embodiments, [symbol] represents a double bond between X⁶ and X⁴, and there is a single bond between X⁷ and X⁴. In embodiments, X⁶ is C, and X⁴ is CR^{X}. In embodiments, X⁶ is C, and X⁴ is N. In embodiments, X⁷ is N. In embodiments, X⁷ is CR^{X} (e.g., C—H).

In embodiments, A is C₆₋₁₀ arylene. In embodiments, A is unsubstituted C₆_10 arylene. In embodiments, A is substituted C₆₋₁₀ arylene (e.g., comprising 1, 2, 3, or 4 substituents as described herein).

In embodiments, A is 5-12-membered heteroarylene. In embodiments, A is unsubstituted 5-12-membered heteroarylene. In embodiments, A is substituted 5-12-membered heteroarylene (e.g., comprising 1, 2, 3, or 4 substituents as described herein).

In embodiments, A is 5-12-membered heterocycloalkylene. In embodiments, A is unsubstituted 5-12-membered heterocycloalkylene. In embodiments, A is substituted 5-12-membered heterocycloalkylene (e.g., comprising 1, 2, 3, or 4 substituents as described herein).

In embodiments, X¹ is N. In embodiments, X¹ is CR^{X} (e.g., C—H or C—CH₃).

In embodiments, X² is N. In embodiments, X² is CR^{X} (e.g., C—H or C—CH₃).

In embodiments, X³ is N. In embodiments, X³ is CR^{X} (e.g., C—H or C—CH₃).

In embodiments, X⁴ is N. In embodiments, X⁴ is CR^{X} (e.g., C—H or C—CH₃).

In embodiments, X⁶ is N. In embodiments, X⁶ is CR^{X′} (e.g., C, C—H, or C—CH₃).

In embodiments, X⁷ is N. In embodiments, X⁷ is CR^{X′} (e.g., C, C—H, or C—CH₃).

In embodiments, X⁵ is a covalent bond. In embodiments, X⁵ is CH₂. In embodiments, X⁵ is O. In embodiments, X⁵ is NR⁴ (e.g., NH or NCH₃). In embodiments, X⁵ is C(O)NR⁴ (e.g., C(O)NH or C(O)CH₃). In embodiments, X⁵ is NR⁴C(O) (e.g., NHC(O) or NCH₃C(O)).

In embodiments, the compound of Formula I has a structure according to Formula I′:

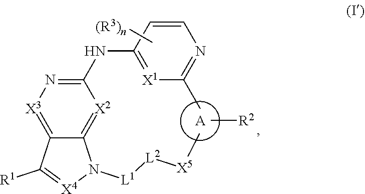

or a pharmaceutically acceptable salt thereof.

In embodiments, L¹ is a covalent bond or C(R⁵)₂, and L² is C₁₋₄ alkylene. In embodiments, L¹ is a covalent bond. In embodiments, L¹ is C(R⁵)₂ (e.g., CH₂, CHCH₃, CH(CH₂CH₃), or C(CH₃)₂). In embodiments, L² is unsubstituted C₁₋₄ alkylene (e.g. CH₂, (CH₂)₂, (CH₂)₃, or (CH₂)₄). In embodiments, L² is substituted C₁₋₄ alkylene (e.g., C₁₋₄ alkylene substituted by OH, oxo (═O), or unsubstituted C₁₋₃ alkyl)).

In embodiments, L¹ and L² combine to form a C₃₋₆ cycloalkyl or a 4- to 6-membered heterocycloalkyl. In embodiments, L¹ and L² combine to form a C₃₋₆ cycloalkyl. In embodiments, L¹ and L² combine to form cyclopropyl. In embodiments, L¹ and L² combine to form cyclobutyl. In embodiments, L¹ and L² combine to form cyclopentyl. In embodiments, L¹ and L² combine to form cyclohexyl. In embodiments, L¹ and L² combine to form an unsubstituted C₄₋₆ cycloalkyl. In embodiments, L¹ and L² combine to form a substituted C₄_6 cycloalkyl (e.g., comprising 1, 2, or 3 substituents as described herein). In embodiments, L¹ and L² combine to form a 4- to 6-membered heterocycloalkyl. In embodiments, L¹ and L² combine to form tetrahydropyranyl. In embodiments, L¹ and L² combine to form an unsubstituted 4- to 6-membered heterocycloalkyl. In embodiments, L¹ and L² combine to form a substituted 4- to 6-membered heterocycloalkyl (e.g., comprising 1, 2, or 3 substituents as described herein).

In embodiments, R¹ is halogen. In embodiments, R¹ is C₁₋₆ alkyl. In embodiments, R¹ is C₃₋₇ cycloalkyl. In embodiments, R¹ is C₆₋₁₀ aryl. In embodiments, R¹ is 5- to 10-membered heteroaryl (e.g., monocyclic or bicyclic heteroaryl). In embodiments, R¹ is 3- to 10-membered heterocycloalkyl (e.g., monocyclic or bicyclic heterocycloalkyl). In embodiments, R¹ is CN. In embodiments, R¹ is NR⁶R⁷. In embodiments, R¹ is NR⁶C(O)R⁷. In embodiments, R¹ is NR⁶C(O)NH₂. In embodiments, R¹ is OR⁸. In embodiments, R¹ is C(O)NR⁶R⁷.

In embodiments, R¹ is unsubstituted C₁₋₆ alkyl. In embodiments, R¹ is unsubstituted C₃₋₇ cycloalkyl. In embodiments, R¹ is unsubstituted C₆₋₁₀ aryl. In embodiments, R¹ is unsubstituted 5- to 10-membered heteroaryl (e.g., unsubstituted monocyclic or bicyclic heteroaryl). In embodiments, R¹ is unsubstituted 3- to 10-membered heterocycloalkyl (e.g., unsubstituted monocyclic or bicyclic heterocycloalkyl).

In embodiments, R¹ is substituted C₁₋₆ alkyl. In embodiments, R¹ is substituted C₃₋₇ cycloalkyl. In embodiments, R¹ is substituted C₆₋₁₀ aryl. In embodiments, R¹ is substituted 5- to 10-membered heteroaryl (e.g., substituted monocyclic or bicyclic heteroaryl). In embodiments, R¹ is substituted 3- to 10-membered heterocycloalkyl (e.g., substituted monocyclic or bicyclic heterocycloalkyl). In embodiments, a substituted group comprises 1, 2, or 3 substituent groups as described herein.

In embodiments, R¹ is a substituted or unsubstituted 5- or 6-membered heteroarylene; a substituted or unsubstituted 5- or 6-membered heterocycloalkyl, C₁₋₆ alkyl substituted by a 5- or 6-membered heteroarylene that is substituted or unsubstituted; or C₁₋₆ alkyl substituted by a 5- or 6-membered heterocycloalkyl that is substituted or unsubstituted, or substituted phenyl.

In embodiments, R² is absent. In embodiments, R² is H. In embodiments, R² is C₁₋₆ alkyl. In embodiments, R² is halogen. In embodiments, R² is CN. In embodiments, R² is C₁₋₆ alkoxy. In embodiments, R² is unsubstituted C₁₋₆ alkyl. In embodiments, R² is substituted C₁₋₆ alkyl (e.g., comprising 1, 2, or 3 substituent groups as described herein). In embodiments, R² is unsubstituted C₁₋₆ alkoxy. In embodiments, R² is substituted C₁₋₆ alkoxy (e.g., comprising 1, 2, or 3 substituent groups as described herein).

In embodiments, R³ is not present. In embodiments, R³ is present. In embodiments, R³ is OH. In embodiments, R³ is CN. In embodiments, R³ is halogen. In embodiments, R³ is C₁₋₆ alkyl. In embodiments, R³ is C₁₋₆ alkoxy. In embodiments, R³ is unsubstituted C₁₋₆ alkyl. In embodiments, R³ is substituted C₁₋₆ alkyl (e.g., comprising 1, 2, or 3 substituent groups as described herein). In embodiments, R³ is unsubstituted C₁₋₆ alkoxy. In embodiments, R³ is substituted C₁₋₆ alkoxy (e.g., comprising 1, 2, or 3 substituent groups as described herein).

In embodiments, n is 0. In embodiments, n is 1. In embodiments, n is 2.

In embodiments, R^{X} is H. In embodiments, R^{X} is OR^{X1}. In embodiments, R^{X} is CN. In embodiments, R^{X} is halogen. In embodiments, R^{X} is C₁₋₆ alkyl. In embodiments, R^{X} is unsubstituted C₁₋₆ alkyl. In embodiments, R^{X} is substituted C₁₋₆ alkyl (e.g., comprising 1, 2, or 3 substituent groups as described herein).

In embodiments, R^{X′} is H. In embodiments, R^{X′} is OR^{X1}. In embodiments, R^{X′} is CN. In embodiments, R^{X′} is halogen. In embodiments, R^{X′} is C₁₋₆ alkyl. In embodiments, R^{X′} is absent if the carbon to which it is attached is part of a double bond.

In embodiments, R^{X1} is H. In embodiments, R^{X1} is C₁₋₆ alkyl. In embodiments, R^{X1} is unsubstituted C₁₋₆ alkyl. In embodiments, R^{X1} is substituted C₁₋₆ alkyl (e.g., comprising 1, 2, or 3 substituent groups as described herein).

In embodiments, R⁴ is H. embodiments, R⁴ is C₁₋₆ alkyl. In embodiments, R⁴ is unsubstituted C₁₋₆ alkyl. In embodiments, R⁴ is substituted C₁₋₆ alkyl (e.g., comprising 1, 2, or 3 substituent groups as described herein).

In embodiments, R⁵ is H. embodiments, R⁵ is C₁₋₆ alkyl. In embodiments, R⁵ is unsubstituted C₁₋₆ alkyl. In embodiments, R⁵ is substituted C₁₋₆ alkyl (e.g., comprising 1, 2, or 3 substituent groups as described herein).

In embodiments, R⁶ is H. In embodiments, R⁶ is C₁₋₆ alkyl. In embodiments, R⁶ is C₃₋₇ cycloalkyl. In embodiments, R⁶ is 3- to 10-membered heterocycloalkyl (e.g. monocyclic or bicyclic heterocycloalkyl). In embodiments, R⁶ is unsubstituted C₁₋₆ alkyl. In embodiments, R⁶ is unsubstituted C₃₋₇ cycloalkyl. In embodiments, R⁶ is unsubstituted 3- to 10-membered heterocycloalkyl (e.g. monocyclic or bicyclic heterocycloalkyl). In embodiments, R⁶ is substituted C₁₋₆ alkyl (e.g., comprising 1, 2, or 3 substituent groups as described herein). In embodiments, R⁶ is substituted C₃₋₇ cycloalkyl (e.g., comprising 1, 2, or 3 substituent groups as described herein). In embodiments, R⁶ is substituted 3- to 10-membered heterocycloalkyl (e.g. a monocyclic or bicyclic heterocycloalkyl comprising 1, 2, or 3 substituent groups as described herein).

In embodiments, R⁷ is H. In embodiments, R⁷ is C₁₋₆ alkyl. In embodiments, R⁷ is C₃₋₇ cycloalkyl. In embodiments, R⁷ is 3- to 10-membered heterocycloalkyl (e.g. monocyclic or bicyclic heterocycloalkyl). In embodiments, R⁷ is unsubstituted C₁₋₆ alkyl. In embodiments, R⁷ is unsubstituted C₃₋₇ cycloalkyl. In embodiments, R⁷ is unsubstituted 3- to 10-membered heterocycloalkyl (e.g. monocyclic or bicyclic heterocycloalkyl). In embodiments, R⁷ is substituted C₁₋₆ alkyl (e.g., comprising 1, 2, or 3 substituent groups as described herein). In embodiments, R⁷ is substituted C₃₋₇ cycloalkyl (e.g., comprising 1, 2, or 3 substituent groups as described herein). In embodiments, R⁷ is substituted 3- to 10-membered heterocycloalkyl (e.g. a monocyclic or bicyclic heterocycloalkyl comprising 1, 2, or 3 substituent groups as described herein).

In embodiments, R⁶ and R⁷ together with the nitrogen atom to which they are attached form a 3- to 8-membered heterocycloalkyl ring (e.g., monocyclic or bicyclic heterocycloalkyl).

In embodiments, R⁸ is H. In embodiments, R⁸ is C₁₋₆ alkyl. In embodiments, R⁸ is 4- to 6-membered heterocycloalkyl. In embodiments, R⁸ is unsubstituted C₁₋₆ alkyl. In embodiments, R⁸ is substituted C₁₋₆ alkyl (e.g., comprising 1, 2, or 3 substituent groups as described herein). In embodiments, R⁸ is a substituted C₁₋₆ alkyl that is piperidinyl substituted C₁₋₆ alkyl (e.g., —CH₂CH₂(piperidinyl)). In embodiments, R⁸ is unsubstituted 4- to 6-membered heterocycloalkyl. In embodiments, R⁸ is substituted 4- to 6-membered heterocycloalkyl (e.g., comprising 1, 2, or 3 substituent groups as described herein).

In embodiments, n is 0.

In embodiments, X³ is CH.

In embodiments, X² is N. In embodiments, X² is CH.

In embodiments, X¹ is N. In embodiments, X¹ is CH.

In embodiments, one of X¹ and X² is N and the other is CH.

In embodiments, X⁴ is N or CH.

In embodiments, L¹ is CHR⁵, and R⁵ is H, CH₃, or CH₂CH₃.

In embodiments, L¹ is C(CH₃)₂.

In embodiments, L¹ is CHCH₃.

In embodiments, L² is unsubstituted C₁₋₄ alkylene, or C₁₋₄ alkylene substituted by unsubstituted C₁₋₃ alkyl.

In embodiments, L² is (CH₂)₂, (CH₂)₃, CH(CH₃)CH₂, or CH₂CH(CH₃).

In embodiments, L¹ and L² combine to form cyclopropyl, cyclobutyl, cyclopentyl, or cyclohexyl.

In embodiments, X⁵ is O or NR⁴.

In embodiments, X⁵ is O, NH, or NCH₃.

In embodiments, A is C₆₋₁₀ arylene or 5-12-membered heteroarylene.

In embodiments, A is 5-12-membered heteroarylene or 5-12-membered heterocycloalkylene.

In embodiments, A is pyridyl, pyrazolyl, thiazolyl, oxazolyl, imidazyolyl,

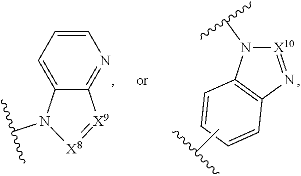

wherein each X⁸, X⁹, and X¹⁰ is CH or N.

In embodiments, A is pyrazolyl optionally substituted by methyl.

In embodiments, A is pyrazolyl substituted by methyl.

In embodiments, A is 1-methylpyrazolyl.

In embodiments, A is phenyl.

In embodiments, R² is absent, H, unsubstituted C₁₋₃ alkyl, or C₁₋₃ alkyl substituted by unsubstituted C₃₋₆ cycloalkyl.

In embodiments, R¹ is F, CN, NH₂, O-(oxetan-3-yl), NH-(oxetan-3-yl), O-(tetrahydrofuran-3-yl), O-(1-N,N-dimethylaminocyclohexan-4-yl), NH-(tetrahydrofuran-3-yl), NH(C₁₋₆ alkyl), NCH₃(C₁₋₆ alkyl), and wherein said C₁₋₆ alkyl comprises one or two substituents selected from OH, NH₂, piperidinyl, and CONH₂.

In embodiments, R¹ is an N-linked group that is azetidine, pyrrolidine, pyrrolyl, or piperazinyl, and wherein said N-linked group is unsubstituted or substituted with a substituent that is OH, CN, oxo, C₁₋₄ alkyl, NR^{1A}R^{1B}, or C(O)NR^{1A}R^{1B}, wherein
said C₁₋₄ alkyl is unsubstituted or substituted with at least one group that is OH, CN, NH₂, NHCH₃, N(CH₃)₂, N-methylpiperazinyl, C(O)NH₂, C(O)NHCH₃, C(O)N(CH₃)₂,
each R^{1A} and R^{1B} is independently H, C₁₋₆ alkyl, C₃₋₇ cycloalkyl, or 3- to 10-membered heterocycloalkyl; or R^{1A} and R^{1B} together with the nitrogen atom to which they are attached form a 3- to 8-membered heterocycloalkyl ring, wherein said C₁₋₆ alkyl is unsubstituted or substituted with a group that is alkoxy.

In embodiments, R¹ is C(O)NHR⁷, and R⁷ is a cyclic group that is cyclopentyl, cyclohexyl,
wherein said cyclic group is unsubstituted or substituted by a group that is CN, OH, oxo, C₁₋₄ alkyl, —NR^{1A}R^{1B} or —C(O)NR^{1A}R^{1B}, wherein
said C₁₋₄ alkyl is unsubstituted or substituted with a group that is OH, NH₂, NHCH₃, N(CH₃)₂, N-methylpiperazinyl, C(O)NH₂, C(O)NHCH₃, C(O)N(CH₃)₂,
each R^{1A} and R^{1B} is independently H, C₁₋₆ alkyl, C₃₋₇ cycloalkyl, or 3- to 10-membered heterocycloalkyl; or R^{1A} and R^{1B} together with the nitrogen atom to which they are attached form a 3- to 8-membered heterocycloalkyl ring.

In embodiments, R¹ is NR⁶R⁷, wherein
R⁶ is independently H or unsubstituted C₁₋₃ alkyl; and
R⁷ is independently C₁₋₆ alkyl, wherein said C₁₋₆ alkyl is unsubstituted or comprises one or two substituent groups selected from —OH and —C(O)NH₂.

In embodiments, R¹ is a substituted or unsubstituted 5- or 6-membered heteroarylene; a substituted or unsubstituted 5- or 6-membered heterocycloalkyl, C₁₋₆ alkyl substituted by a 5- or 6-membered heteroarylene that is substituted or unsubstituted; or C₁₋₆ alkyl substituted by a 5- or 6-membered heterocycloalkyl that is substituted or unsubstituted, or substituted phenyl.

Compounds of Formula (I-A)

In embodiments, a compound of Formula (I) has a structure according to Formula (I-A),

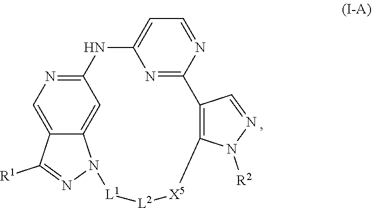

or a pharmaceutically acceptable salt thereof.

In embodiments, R¹, L¹, L², X⁵, and R² are according to any embodiment described herein.

In embodiments, R² is unsubstituted C₁₋₆ alkyl or C₁₋₆ alkyl substituted by a group that is unsubstituted C₃₋₆ cycloalkyl.

In embodiments, L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄.

In embodiments, L¹-L²-X⁵ is

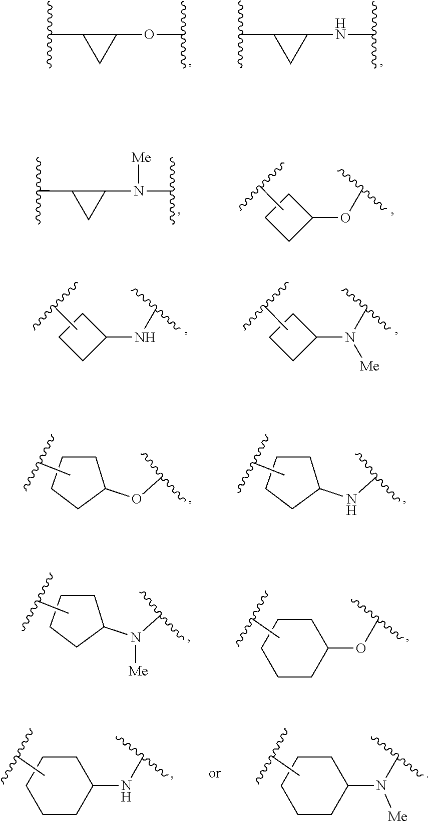

In embodiments, R² is CH₃ or C₁₋₃ alkyl substituted by unsubstituted C₃₋₆ cycloalkyl.

In embodiments, R² is CH₃.

In embodiments, L¹-L²-X⁵ is CH(CH₃)—(CH₂)₂—O, CH(CH₃)(CH₂)₃—O, or (CH₂)₃—O.

In embodiments, a compound has a structure according to Formula (I-A-1),

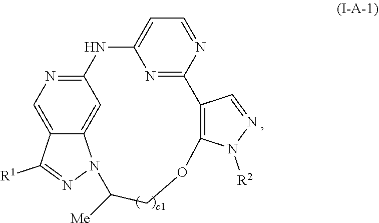

or a pharmaceutically acceptable salt thereof.

In embodiments, R¹ is according to any embodiment described herein.

In embodiments, c1 is 2 or 3. In embodiments, c1 is 2. In embodiments, c1 is 3.

In embodiments, a compound has a structure according to Formula (I-A-1′),

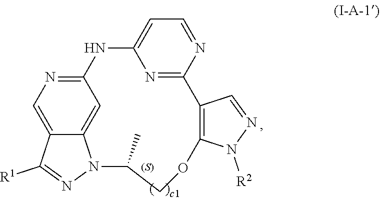

or a pharmaceutically acceptable salt thereof.

In embodiments, R¹ is according to any embodiment described herein.

In embodiments, c1 is 2 or 3. In embodiments, c1 is 2. In embodiments, c1 is 3.

In embodiments, a compound has a structure according to Formula (I-A-1″),

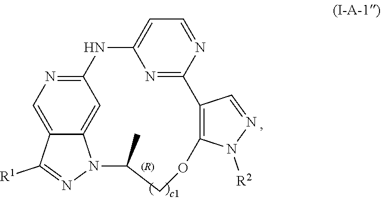

or a pharmaceutically acceptable salt thereof.

In embodiments, R¹ is according to any embodiment described herein.

In embodiments, c1 is 2 or 3. In embodiments, c1 is 2. In embodiments, c1 is 3.

In embodiments, a compound has a structure according to Formula (I-A-2),

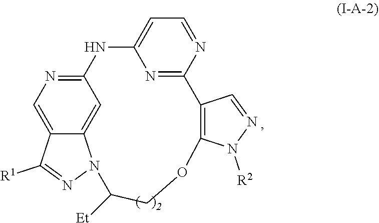

or a pharmaceutically acceptable salt thereof.

In embodiments, R¹ is according to any embodiment described herein.

In embodiments, the sp³ carbon substituted by Et has the (R)-configuration.

In embodiments, the sp³ carbon substituted by Et has the (S)-configuration.

In embodiments, a compound has a structure according to Formula (I-A-3),

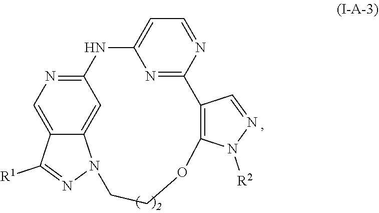

or a pharmaceutically acceptable salt thereof.

In embodiments, R¹ is according to any embodiment described herein.

### Compounds of Formula (I-B)

In embodiments, a compound has a structure according to Formula (I-B),

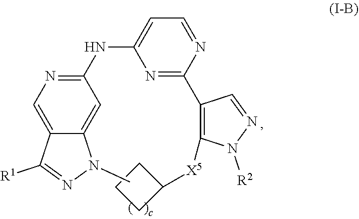

or a pharmaceutically acceptable salt thereof, wherein R² is unsubstituted C₁₋₆ alkyl or C₁₋₆ alkyl substituted by a group that is unsubstituted C₃₋₆ cycloalkyl; X⁵ is O; and c is 0, 1, 2, or 3.

In embodiments, each of R¹, c, X⁵, and R² is according to any embodiment described herein.

In embodiments, R² is CH₃.

In embodiments, a compound has a structure according to Formula (I-B-1),

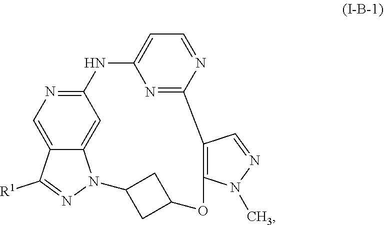

or a pharmaceutically acceptable salt thereof.

In embodiments, R¹ is according to any embodiment described herein.

In embodiments, a compound has a structure according to Formula (I-B-2),

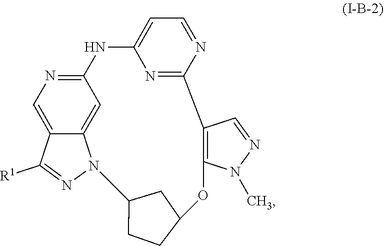

or a pharmaceutically acceptable salt thereof.

In embodiments, R¹ is according to any embodiment described herein.

In embodiments, a compound has a structure according to Formula (I-B-3),

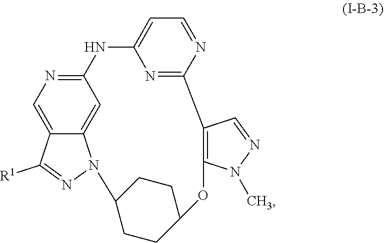

or a pharmaceutically acceptable salt thereof.

In embodiments, R¹ is according to any embodiment described herein.

### Compounds of Formula (I-C)

In embodiments, a compound has a structure according to Formula (I-C),

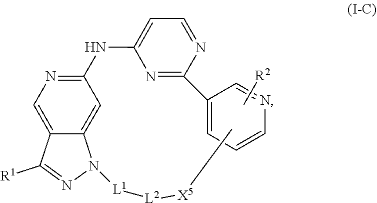

or a pharmaceutically acceptable salt thereof.

In embodiments, each of R¹, L¹, L², X⁵, and R² is according to any embodiment described herein.

In embodiments, R² is H, unsubstituted C₁₋₆ alkyl or C₁₋₆ alkyl substituted by a group that is unsubstituted C₃₋₆ cycloalkyl.

In embodiments, L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄.

In embodiments, L¹-L²-X⁵ is

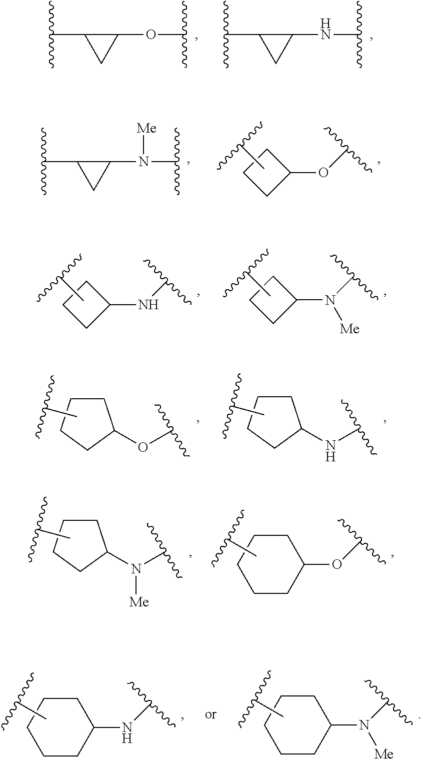

In embodiments, R² is H or CH₃.

In embodiments, L¹-L²-X⁵ is CH(CH₃)—(CH₂)₂—O, CH(CH₃)(CH₂)₃—O, (CH₂)₃—O, CH(CH₃)—(CH₂)₂—NHC(O), CH(CH₃)—(CH₂)₂—NCH₃C(O), CH(CH₃)—(CH₂)₃—NHC(O), CH(CH₃)—(CH₂)₃—NCH₃C(O), CH(CH₃)—(CH₂)₂—C(O)NH, CH(CH₃)—(CH₂)₂—C(O)NCH₃, CH(CH₃)—(CH₂)₃—C(O)NH, or CH(CH₃)—(CH₂)₃—C(O)NCH₃.

In embodiments, a compound has a structure according to Formula (I-C-1),

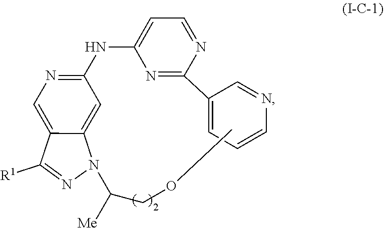

or a pharmaceutically acceptable salt thereof.

In embodiments, R¹ is according to any embodiment described herein.

In embodiments, the sp³ carbon substituted by Me has the (R)-configuration.

In embodiments, the sp³ carbon substituted by Me has the (S)-configuration.

In embodiments, a compound has a structure according to Formula (I-C-2),

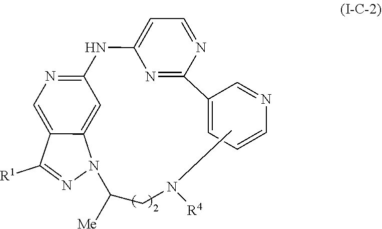

or a pharmaceutically acceptable salt thereof, wherein R⁴ is H or CH₃.

In embodiments, R¹ is according to any embodiment described herein.

In embodiments, the sp³ carbon substituted by Me has the (R)-configuration.

In embodiments, the sp³ carbon substituted by Me has the (S)-configuration.

In embodiments, a compound has a structure according to Formula (I-C-3),

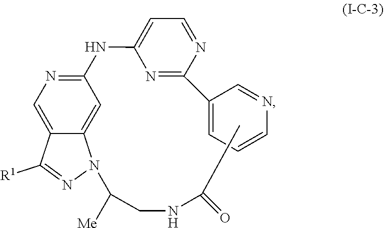

or a pharmaceutically acceptable salt thereof.

In embodiments, R¹ is according to any embodiment described herein.

In embodiments, the sp³ carbon substituted by Me has the (R)-configuration.

In embodiments, the sp³ carbon substituted by Me has the (S)-configuration.

In embodiments, a compound has a structure according to Formula (I-C-4),

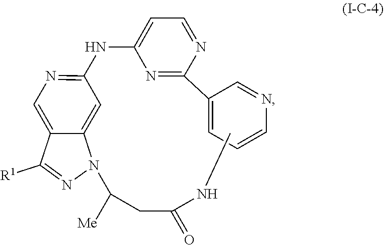

or a pharmaceutically acceptable salt thereof.

In embodiments, R¹ is according to any embodiment described herein.

In embodiments, the sp³ carbon substituted by Me has the (R)-configuration.

In embodiments, the sp³ carbon substituted by Me has the (S)-configuration.

### Compounds of Formula (I-D)

In embodiments, a compound has a structure according to Formula (I-D),

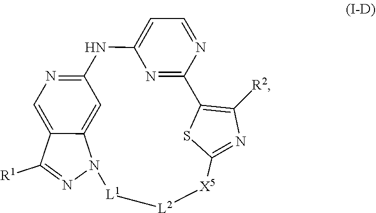

or a pharmaceutically acceptable salt thereof.

In embodiments, each of R¹, L¹, L², X⁵, and R² is according to any embodiment described herein.

In embodiments R² is H or unsubstituted C₁₋₆ alkyl.

In embodiments, L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄.

In embodiments, L¹-L²-X⁵ is

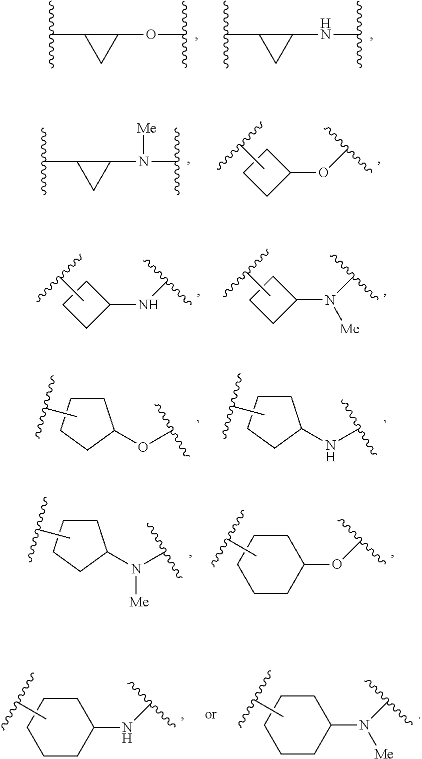

In embodiments, R² is H or CH₃.

In embodiments, L¹-L²-X⁵ is CH(CH₃)—(CH₂)₂—NH, CH(CH₃)—(CH₂)₂—NCH₃, CH(CH₃)—(CH₂)₃—NH, or CH(CH₃)—(CH₂)₃—NCH₃.

In embodiments, a compound has a structure according to Formula (I-D-1),

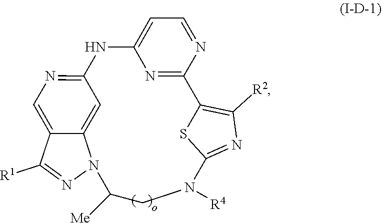

or a pharmaceutically acceptable salt thereof, wherein R² is H or CH₃; R⁴ is H or CH₃; and o is 1 or 2.

In embodiments, each of R¹ is according to any embodiment described herein.

In embodiments, the sp³ carbon substituted by Me has the (R)-configuration.

In embodiments, the sp³ carbon substituted by Me has the (S)-configuration.

### Compounds of Formula (I-E)

In embodiments, a compound has a structure according to Formula (I-E),

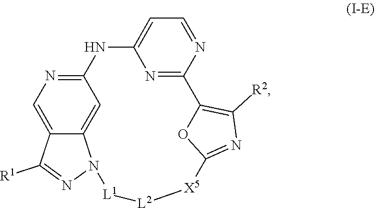

or a pharmaceutically acceptable salt thereof.

In embodiments, each of R¹, L¹, L², X⁵, and R² is according to any embodiment described herein.

In embodiments, R² is H or unsubstituted C₁₋₆ alkyl.

In embodiments, L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄.

In embodiments, L¹-L²-X⁵ is

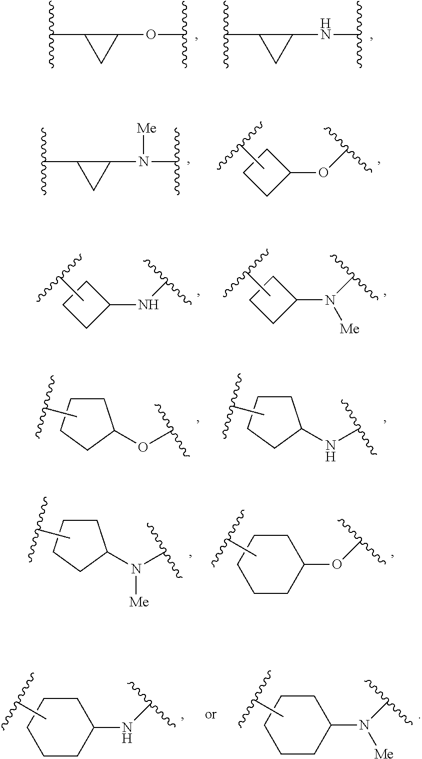

In embodiments, R² is H or CH₃.

In embodiments, L¹-L²-X⁵ is CH(CH₃)—(CH₂)₃, or CH(CH₃)—(CH₂)₄.

In embodiments, a compound has a structure according to Formula (I-E-1),

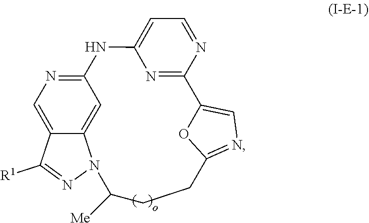

or a pharmaceutically acceptable salt thereof, wherein

o is 2 or 3.

In embodiments, R¹ is according to any embodiment described herein.

In embodiments, the sp³ carbon substituted by Me has the (R)-configuration.

In embodiments, the sp³ carbon substituted by Me has the (S)-configuration.

### Compounds of Formula (I-F)

In embodiments, a compound has a structure according to Formula (I-F),

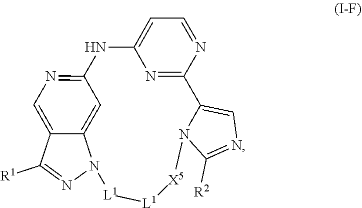

or a pharmaceutically acceptable salt thereof.

In embodiments, each of R¹, L¹, L², X⁵, and R² is according to any embodiment described herein.

In embodiments, R² is H or unsubstituted C₁₋₆ alkyl.

In embodiments, L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄.

In embodiments, L¹-L²-X⁵ is

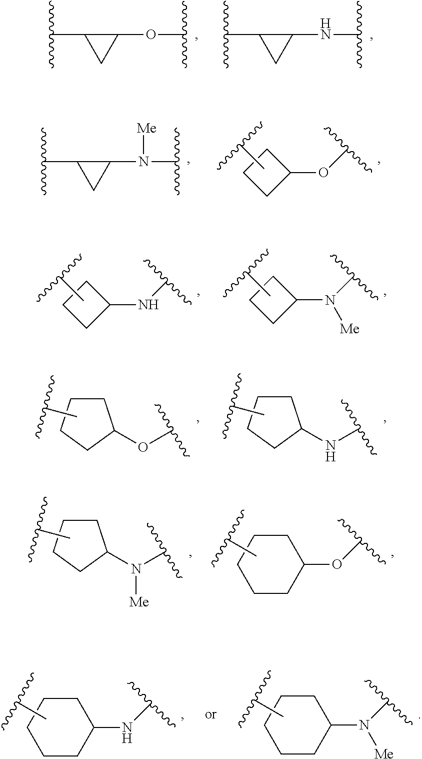

In embodiments, R² is H or CH₃.

In embodiments, L¹-L²-X⁵ is CH(CH₃)—(CH₂)₃, or CH(CH₃)—(CH₂)₄.

In embodiments, a compound has a structure according to Formula (I-F-1),

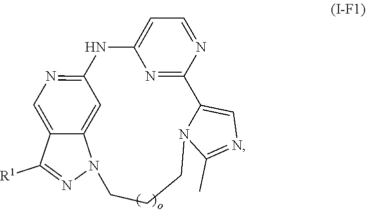

or a pharmaceutically acceptable salt thereof, wherein o is 1 or 2.

In embodiments, R¹ is according to any embodiment described herein.

### Compounds of Formula (I-G)

In embodiments, a compound has a structure according to Formula (I-G),

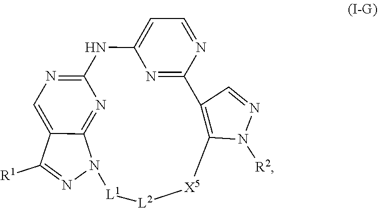

or a pharmaceutically acceptable salt thereof.

In embodiments, each of R¹, L¹, L², X⁵, and R² is according to any embodiment described herein.

In embodiments, R² is unsubstituted C₁₋₆ alkyl or C₁₋₆ alkyl substituted by a group that is unsubstituted C₃₋₆ cycloalkyl.

In embodiments, L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄.

In embodiments, L¹-L²-X⁵ is

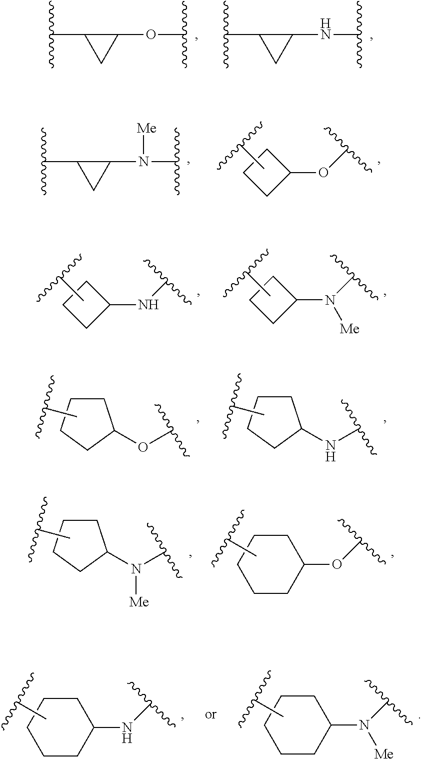

In embodiments, R² is H or CH₃.

In embodiments, L¹-L²-X⁵ is CH(CH₃)—(CH₂)₂—O, CH(CH₃)(CH₂)₃—O, or (CH₂)₃—O.

In embodiments, a compound has a structure according to Formula (I-G-1),

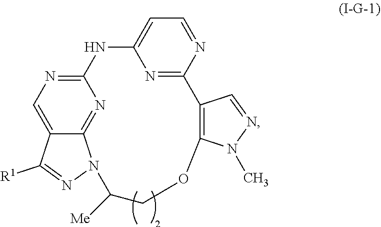

or a pharmaceutically acceptable salt thereof.

In embodiments, R¹ is according to any embodiment described herein.

In embodiments, the sp³ carbon substituted by Me has the (R)-configuration.

In embodiments, the sp³ carbon substituted by Me has the (S)-configuration.

### Compounds of Formula (I-H)

In embodiments, a compound has a structure according to Formula (I-H),

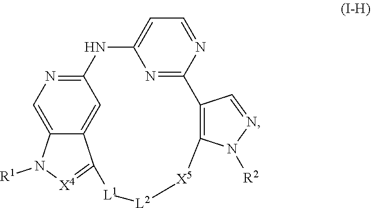

or a pharmaceutically acceptable salt thereof.

In embodiments, each of R¹, L¹, L², X⁵, and R² is according to any embodiment described herein.

In embodiments, X⁴ is CH or N.

In embodiments, R² is unsubstituted C₁₋₆ alkyl or C₁₋₆ alkyl substituted by a group that is unsubstituted C₃₋₆ cycloalkyl.

In embodiments, L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄.

In embodiments, L¹-L²-X⁵ is

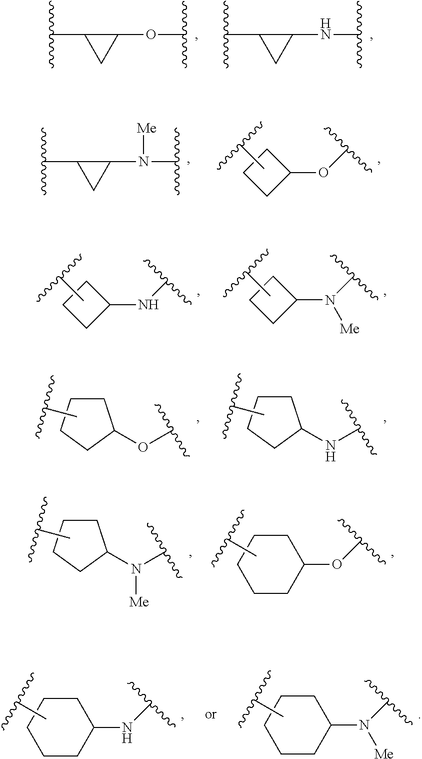

In embodiments, R² is H or CH₃.

In embodiments, L¹-L²-X⁵ is CH(CH₃)—(CH₂)₂—O, CH(CH₃)(CH₂)₃—O, or (CH₂)₃—O.

In embodiments, a compound has a structure according to Formula (I-H-1),

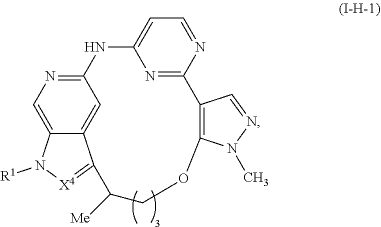

or a pharmaceutically acceptable salt thereof.

In embodiments, R¹ is according to any embodiment described herein.

In embodiments, the sp³ carbon substituted by Me has the (R)-configuration.

In embodiments, the sp³ carbon substituted by Me has the (S)-configuration.

### Compounds of Formula (I-I)

In embodiments, a compound has a structure according to Formula (I-I),

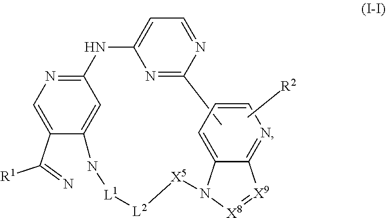

or a pharmaceutically acceptable salt thereof.

In embodiments, each of R¹, L¹, L², X⁵, X⁸, X⁹, and R² is according to any embodiment described herein.

In embodiments, R² is H or unsubstituted C₁₋₆ alkyl.

In embodiments, each X⁸ and X⁹ is CH or N.

In embodiments, L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄.

In embodiments, L¹-L²-X⁵ is

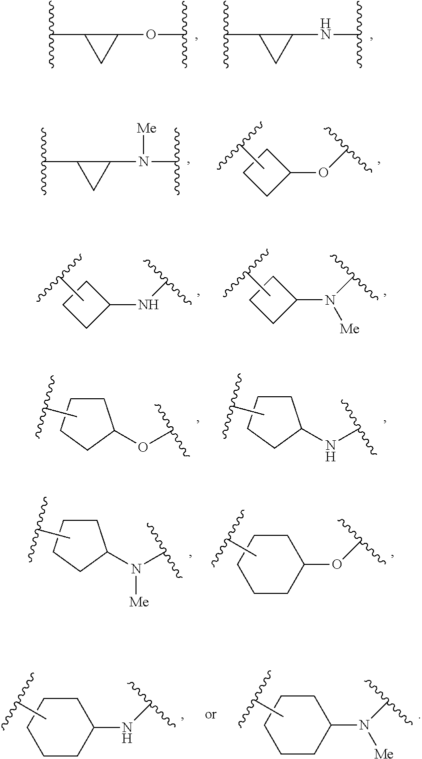

In embodiments, R² is H or CH₃.

In embodiments, L¹-L²-X⁵ is CH₂CH₂, CH(CH₃)—(CH₂)₃, or CH(CH₃)—(CH₂)₄.

In embodiments, a compound has a structure according to Formula (I-I-1).

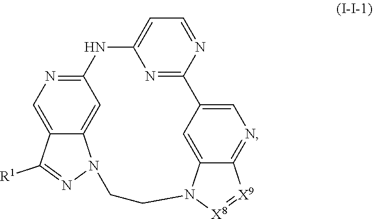

or a pharmaceutically acceptable salt thereof, wherein each X⁸ and X⁹ is CH or N.

In embodiments, R¹ is according to any embodiment described herein.

### Compounds of Formula (I-J)

In embodiments, a compound has a structure according to Formula (I-J),

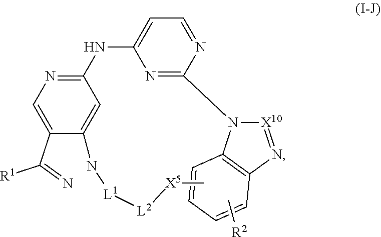

or a pharmaceutically acceptable salt thereof.

In embodiments, each of R¹, L¹, L², X⁵, R², and X¹⁰ is according to any embodiment described herein.

In embodiments, R² is H or unsubstituted C₁₋₆ alkyl.

In embodiments, X¹⁰ is CH or N.

In embodiments, L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄.

In embodiments, L¹-L²-X⁵ is

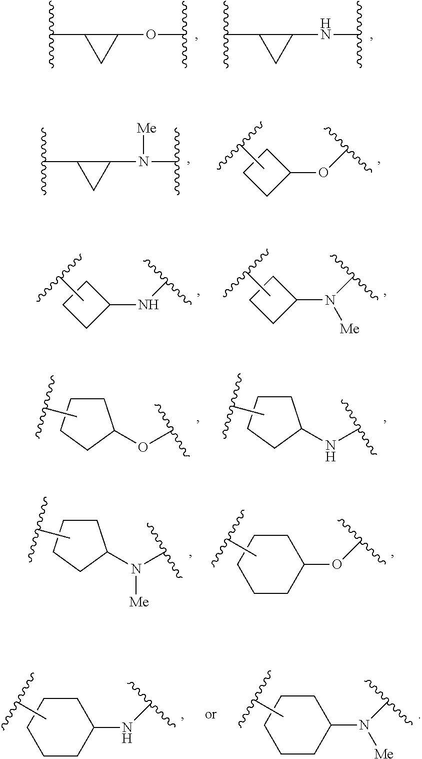

In embodiments, R² is H or CH₃.

In embodiments, L¹-L²-X⁵ is CH₂CH₂, CH(CH₃)—(CH₂)₃, or CH(CH₃)—(CH₂)₄.

In embodiments, a compound has a structure according to Formula (I-J-1),

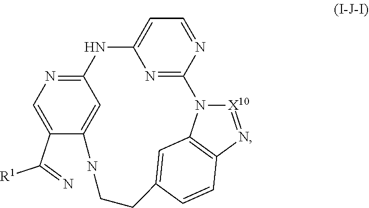

or a pharmaceutically acceptable salt thereof, wherein X¹⁰ is CH or N.

In embodiments, R¹ is according to any embodiment described herein.

### Exemplary R¹ Groups

Still further exemplary R¹ groups are described herein. That is, embodiments of compounds of Formula (I) (e.g., any compound according to Formula (I-A), (I-B), (I-C), (I-D), (I-E), (I-F), (I-H), (I-I) and/or (I-J) and subformulas thereof) can feature any R¹ group described herein.

In embodiments, R¹ is a substituted or unsubstituted 5- or 6-membered heteroarylene.

In embodiments, R¹ is a substituted or unsubstituted 5- or 6-membered heterocycloalkyl.

In embodiments, R¹ is C₁₋₆ alkyl substituted by a 5- or 6-membered heteroarylene that is substituted or unsubstituted.

In embodiments, R¹ is C₁₋₆ alkyl substituted by a 5- or 6-membered heterocycloalkyl that is substituted or unsubstituted.

In embodiments, R¹ is substituted phenyl.

In embodiments of any formula described herein, R¹ is F, CN, or NH₂.

### Substructure 1

In embodiments, R¹ has a structure according to Substructure 1,

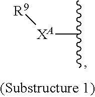

wherein
X^{A} is NH, NCH₃, or O; and R⁹ is a 3- to 6-membered oxygen-containing or nitrogen-containing heterocycloalkyl, C₃₋₇ cycloalkyl, or C₁₋₆ alkyl, and wherein said C₃₋₇ cycloalkyl or C₁₋₆ alkyl optionally comprises one or two substituents selected from OH, NH₂, NMe₂, piperidinyl, and CONH₂.

In embodiments, X^{A} is NH. In embodiments, X^{A} is NCH₃. In embodiments, X^{A} is O.

In embodiments, R⁹ is a 3- to 6-membered oxygen-containing heterocycloalkyl.

In embodiments, R⁹ is a 3- to 6-membered nitrogen-containing heterocycloalkyl.

In embodiments, R⁹ is C₃₋₇ cycloalkyl, optionally comprising one or two substituents selected from OH, NH₂, NMe₂, piperidinyl, and CONH₂.

In embodiments, R⁹ is C₁₋₆ alkyl, optionally comprising one or two substituents selected from OH, NH₂, NMe₂, piperidinyl, and CONH₂.

In embodiments, R¹ is any one of substructures (a1)-(a10):

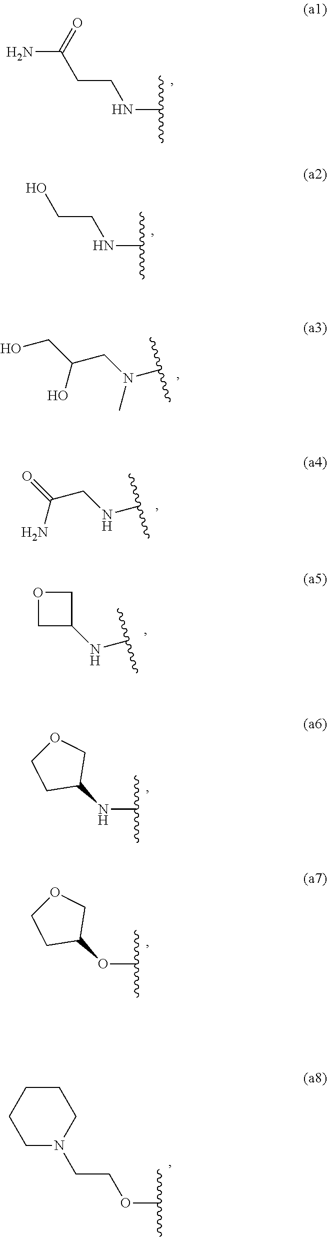

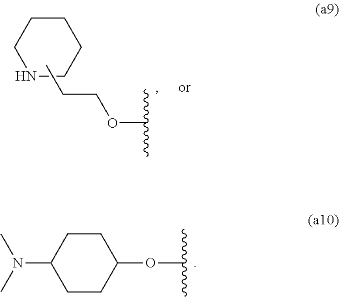

### Substructure 2

In embodiments of any formula described herein, R¹ has a structure according to Substructure 2,

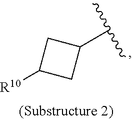

wherein

R¹⁰ is H, OH, C₁₋₆ alkyl, or CONR^{10A}R^{10B} and wherein said C₁₋₆ alkyl optionally comprises one or two substituents selected from OH and CN;

each R^{10A} and R^{10B} is independently H, unsubstituted C₁₋₆ alkyl, C₁₋₆ alkyl substituted by alkoxy, or R^{10A} and R^{10B} together with the nitrogen atom to which they are attached form an unsubstituted 3- to 8-membered heterocycloalkyl ring.

In embodiments, R¹⁰ is H or OH.

In embodiments, R¹⁰ is C₁₋₆ alkyl, optionally comprising one or two substituents selected from OH and CN;

In embodiments, R¹⁰ is CONR^{10A}R^{10B}

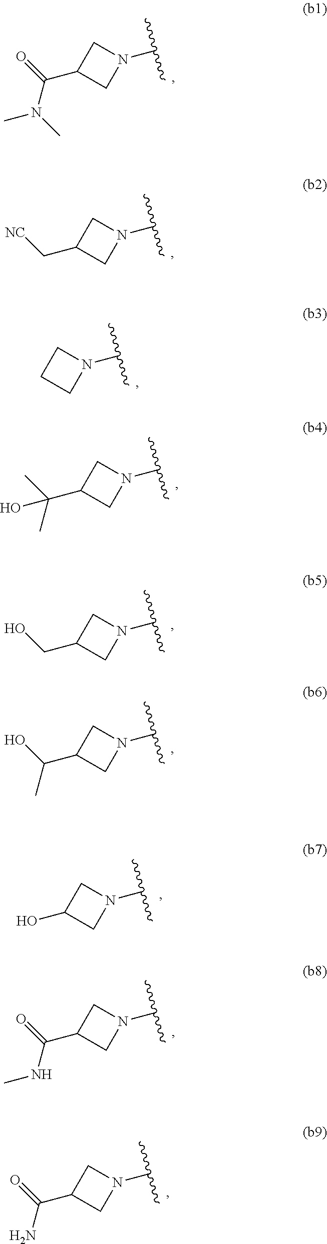

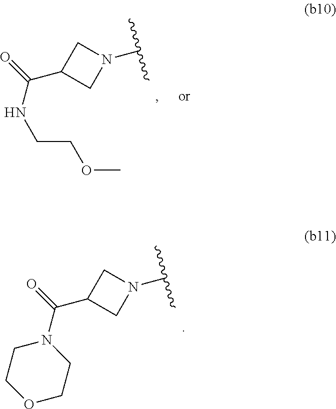 [SMILES: *N1CC(C(=O)NCCOC)C1 ; *N1CC(C(=O)N2CCOCC2)C1]

In embodiments, R¹ is any one of substructures (b1)-(b11):

### Substructure 3

In embodiments of any formula described herein, R¹ has a structure according to Substructure 3,

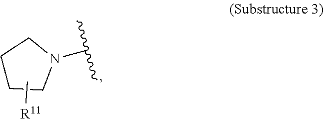

wherein

R¹¹ is H, OH, amino, mono(C₁₋₆ alkyl)amino, di(C₁₋₆ alkyl)amino, —CH₂-[di(C₁₋₆ alkyl)amino], CN, C₁₋₆ alkyl, CONH₂, CONHMe, COOH, CO₂Me, or CONR^{11A}R^{11B}; and wherein said C₁₋₆ alkyl optionally comprises one or two substituents selected from OH, F, and NR^{11A}R^{11B}.

each R^{11A} and R^{11B} is independently unsubstituted C₁₋₆ alkyl, or R^{11A} and R^{11B} together with the nitrogen atom to which they are attached form a methyl or isopropyl substituted 3- to 8-membered heterocycloalkyl ring.

In embodiments, R¹¹ is H, CN, or OH.

In embodiments, R¹¹ is amino, mono(C₁₋₆ alkyl)amino, di(C₁₋₆ alkyl)amino, or —CH₂-[di(C₁₋₆ alkyl)amino], and wherein said C₁₋₆ alkyl optionally comprises one or two substituents selected from OH, F, and NR^{11A}R^{11B}.

In embodiments, R¹¹ is C₁₋₆ alkyl, and wherein said C₁₋₆ alkyl optionally comprises one or two substituents selected from OH, F, and NR^{11A}R^{11B}.

In embodiments, R¹¹ is CONH₂, CONHMe, or CONR^{11A}R^{11B}.

In embodiments, R¹¹ is COOH or CO₂Me.

In embodiments, R¹ is any one of substructures (c1)-(c28):

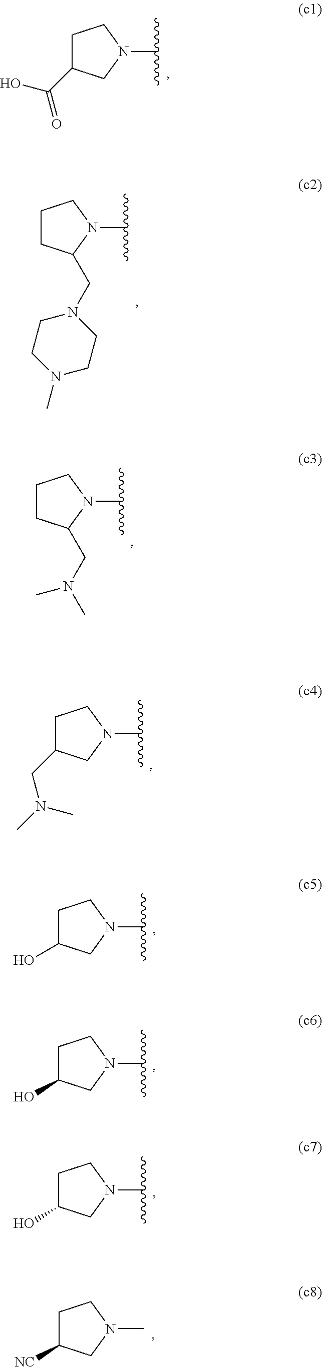

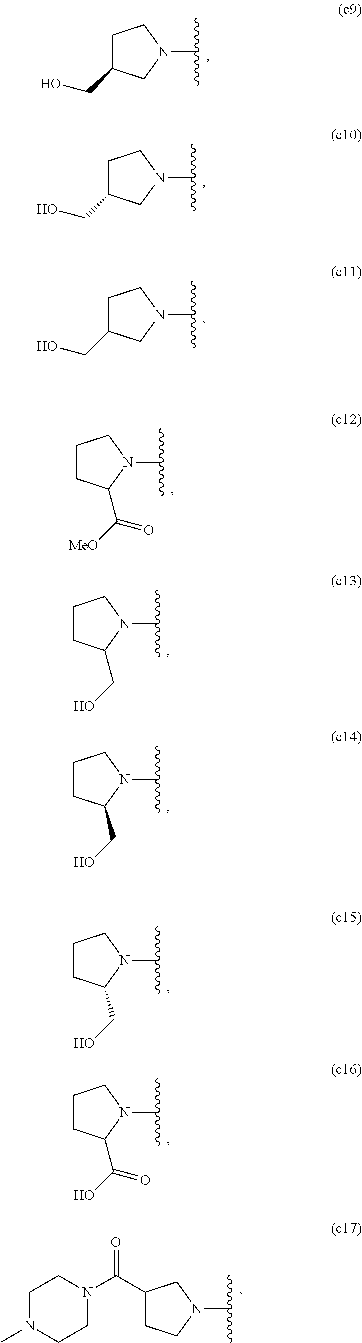

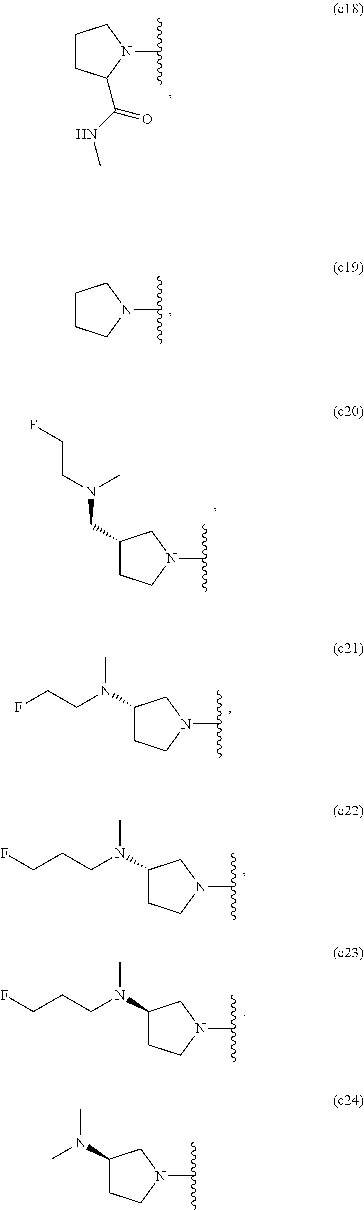

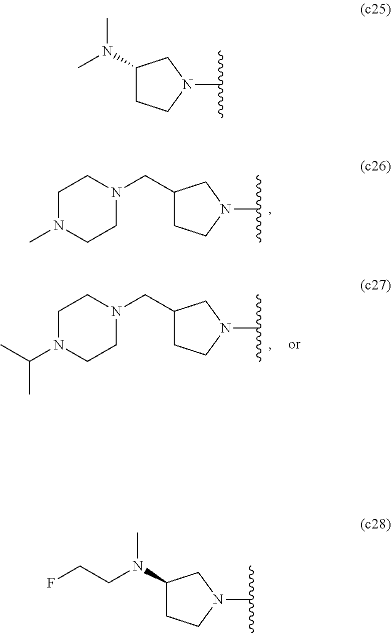

### Substructure 4

In embodiments of any formula described herein, R¹ has a structure according to Substructure 4,

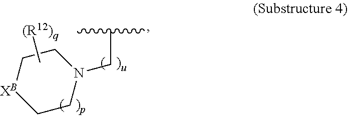

wherein

X^{B} is N, O, S, SO, or SO₂; each R¹², when present, is oxo, methyl, or cyclopropyl; p is 0 or 1; q is 0, 1, or 2, and u is 0 or 1.

In embodiments, X^{B} is N. In embodiments, p is 0. In embodiments, p is 1. In embodiments, q is 0. In embodiments, q is 1, and R¹² is oxo.

In embodiments, X^{B} is O. In embodiments, p is 0. In embodiments, p is 1. In embodiments, q is 0. In embodiments, q is 1, and R¹² is oxo. In embodiments, q is 1, and R¹² is methyl. In embodiments, q is 1, and R¹² is cyclopropyl.

In embodiments, X^{B} is SO₂.

In embodiments, R¹ is any one of substructures (d1)-(d6):

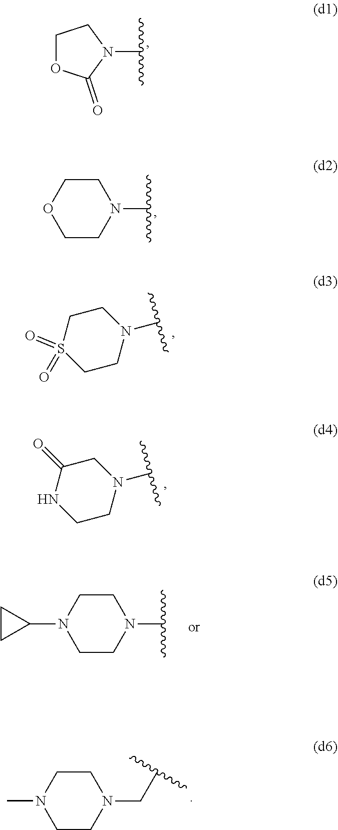

### Substructure 5

In embodiments of any formula described herein, R¹ has a structure according to Substructure 5,

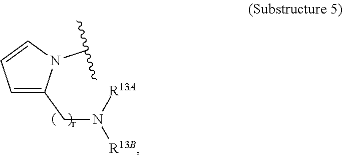

wherein

r is 1 or 2; and each R^{13A} and R^{13B} is independently unsubstituted C₁₋₆ alkyl, or R^{13A} and R^{13B} together with the nitrogen atom to which they are attached form a 3- to 8-membered heterocycloalkyl ring optionally substituted with methyl (e.g., a N-methyl 3- to 8-membered heterocycloalkyl ring).

In embodiments, r is 1. In embodiments, r is 2.

In embodiments, R^{13A} and R^{13B} are both unsubstituted C₁₋₆ alkyl.

In embodiments, R^{13A} and R^{13B} together with the nitrogen atom to which they are attached form a 3- to 8-membered heterocycloalkyl ring optionally substituted with methyl. In embodiments, R^{13A} and R^{13B} together with the nitrogen atom to which they are attached form a N-methyl 3- to 8-membered heterocycloalkyl ring.

In embodiments, R¹ is substructure (e1) or (e2):

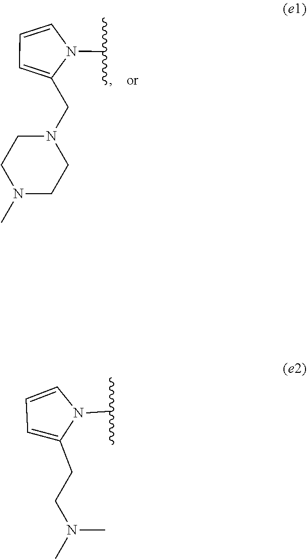 [SMILES: *n1cccc1CN1CCN(C)CC1 ; *n1cccc1CCN(C)C]

### Substructure 6

In embodiments of any formula described herein, R¹ has a structure according to Substructure 6,

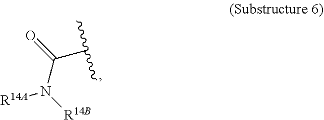

wherein

each R^{14A} and R^{14B} is independently H, unsubstituted C₁₋₆ alkyl, or 5- to 6-membered cycloalkyl ring optionally substituted with CN.

In embodiments, R^{14A} is H. In embodiments, R^{14B} is unsubstituted C₁₋₆ alkyl or 5- to 6-membered cycloalkyl ring optionally substituted with CN.

In embodiments, R¹ is

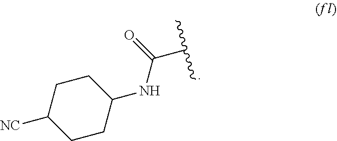 [SMILES: *C(=O)NC1CCC(C#N)CC1]

### Substructures 7 and 8

In embodiments of any formula described herein, R¹ has a structure according to Substructure 7,

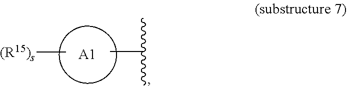

or

Substructure 8,

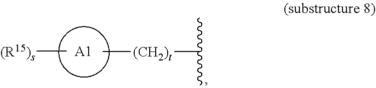

wherein

s is 0, 1, 2, or 3;

t is an integer of 1-6;

v is 0, 1, 2, or 3;

A1 is phenyl, 5- to 6-membered heteroarylene or 5- to 6-membered heterocycloalkyl;

R¹⁵ is independently
halogen
unsubstituted C₁₋₆ alkyl;
C₃₋₆ cycloalkyl;
C₁₋₆ alkyl substituted by OH or OMe;
C₁₋₆ alkyl substituted by halo, amino, monoalkylamino, or dialkylamino;
C₁₋₆ alkoxyl substituted by halo, amino, monoalkylamino, or dialkylamino;
8- to 9-membered heterocycloalkyl;
—(CH₂)ᵥ-(5- to 6-membered heterocycloalkyl);
—(CH₂)ᵥ-(5- to 6-membered heteroaryl);
—(CO)-(5- to 6-membered heterocycloalkyl);
—(CO)-(5- to 6-membered heteroaryl);
—O-(5- to 6-membered heterocycloalkyl);
—O-(5- to 6-membered heteroaryl);
—(CH₂)ᵥ—NH—(C₁₋₆ alkyl substituted by halo, OH, OMe, amino, monoalkylamino, or dialkylamino);
—(CH₂)ᵥ—NMe-(C₁₋₆ alkyl substituted by halo, OH, OMe, amino, monoalkylamino, or dialkylamino).

In embodiments, t is 1 or 2.

In embodiments, R¹ is according to Substructure 7.

In embodiments, R¹ is according to Substructure 8.

In embodiments, s is 1. In embodiments, s is 2.

In embodiments, R¹⁵ is halogen.

In embodiments, R¹⁵ is unsubstituted C₁₋₆ alkyl.

In embodiments, R¹⁵ is C₃₋₆ cycloalkyl.

In embodiments, R¹⁵ is C₁₋₆ alkyl substituted by OH or OMe.

In embodiments, R¹⁵ is C₁₋₆ alkyl substituted by halo, amino, monoalkylamino (e.g., NHMe or NHEt), or dialkylamino (e.g., NMe₂, NMeEt, or NEt₂).

In embodiments, R¹⁵ is C₁₋₆ alkoxyl substituted by halo, amino, monoalkylamino (e.g., NHMe or NHEt), or dialkylamino (e.g., NMe₂, NMeEt, or NEt₂).

In embodiments, R¹⁵ is 8- to 9-membered heterocycloalkyl.

In embodiments, R¹⁵ is —(CH₂)ᵥ-(5- to 6-membered heterocycloalkyl). In embodiments, v is 0. In embodiments, v is 1. In embodiments, v is 2.

In embodiments, R¹⁵ is —(CH₂)ᵥ-(5- to 6-membered heteroaryl). In embodiments, v is 0. In embodiments, v is 1. In embodiments, v is 2.

In embodiments, R¹⁵ is —(CO)-(5- to 6-membered heterocycloalkyl).

In embodiments, R¹⁵ is —(CO)-(5- to 6-membered heteroaryl).

In embodiments, R¹⁵ is —O-(5- to 6-membered heterocycloalkyl).

In embodiments, R¹⁵ is —O-(5- to 6-membered heteroaryl).

In embodiments, R¹⁵ is —(CH₂)ᵥ—NH—(C₁₋₆ alkyl substituted by halo, OH, OMe, amino, monoalkylamino (e.g., NHMe or NHEt), or dialkylamino (e.g., NMe₂, NMeEt, or NEt₂)).

In embodiments, v is 0. In embodiments, v is 1. In embodiments, v is 2.

In embodiments, R¹⁵ is —(CH₂)ᵥ—NMe-(C₁₋₆ alkyl substituted by halo, OH, OMe, amino, monoalkylamino (e.g., NHMe or NHEt), or dialkylamino (e.g., NMe₂, NMeEt, or NEt₂)).

In embodiments, v is 0. In embodiments, v is 1. In embodiments, v is 2.

In embodiments, A1 is furan, pyrazole, pyrrole, thiazole, oxazole, phenyl, pyridyl, or a bicyclic nitrogen-containing 8- to 9-membered heterocycloalkyl.

In embodiments, R¹ is any one of substructures (g1)-(g48):

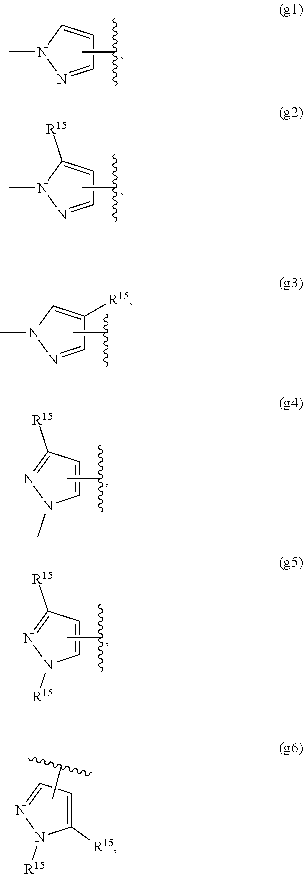

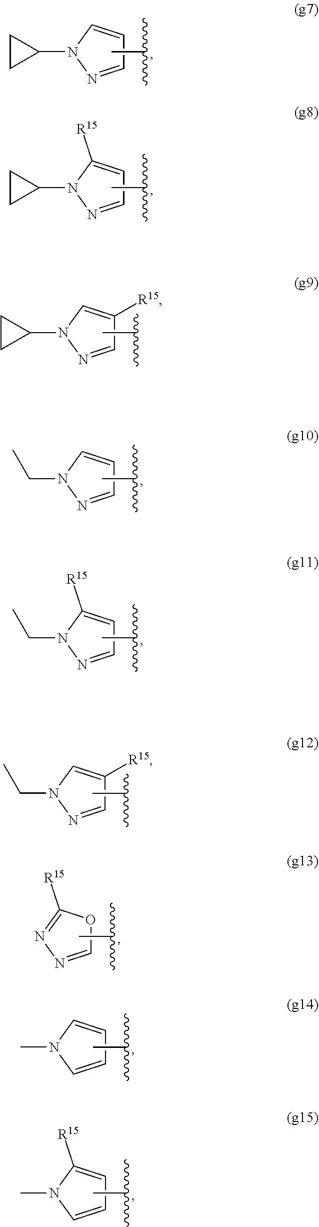

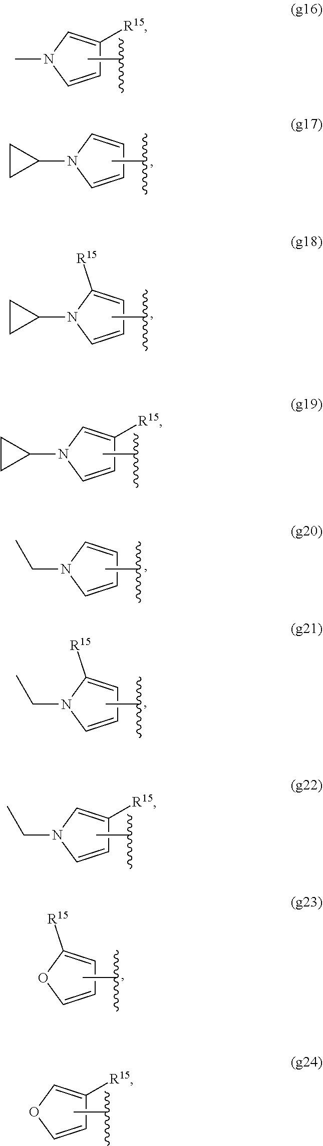

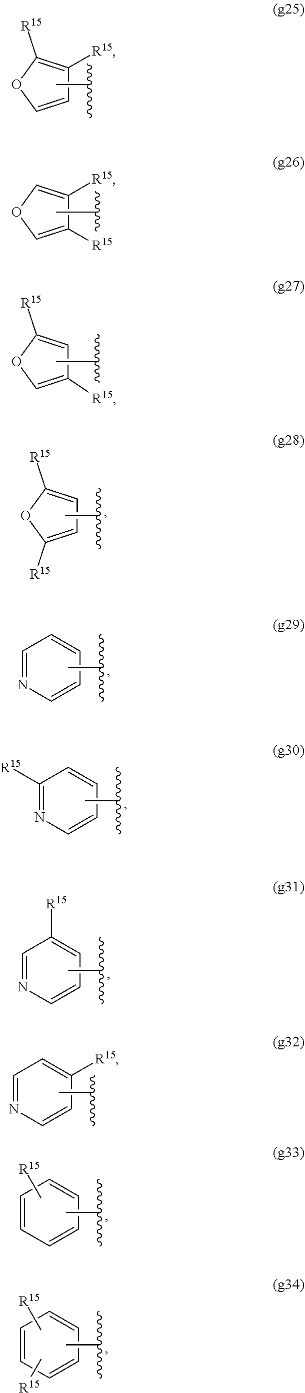

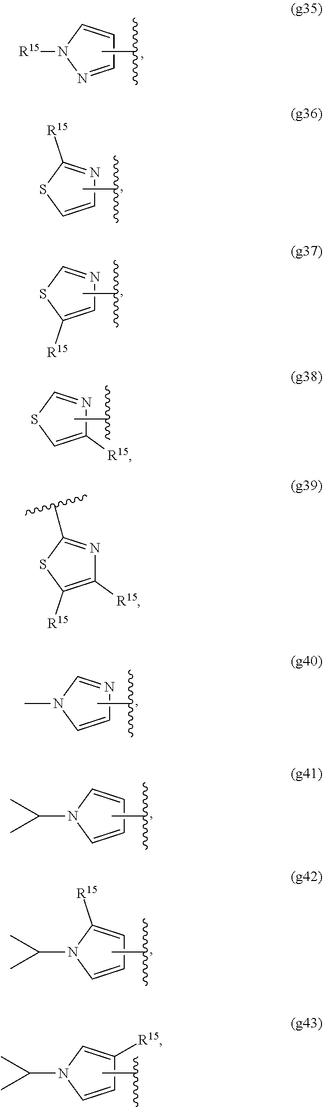

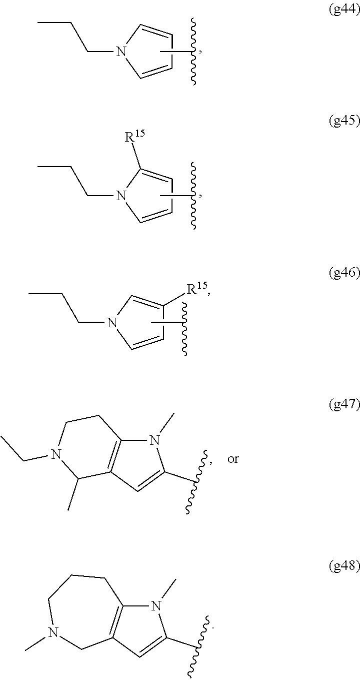

In embodiments, each R¹⁵ is independently

—F, —Cl, —CH₃, —CH₂CH₃, —CH(CH₃)₂,

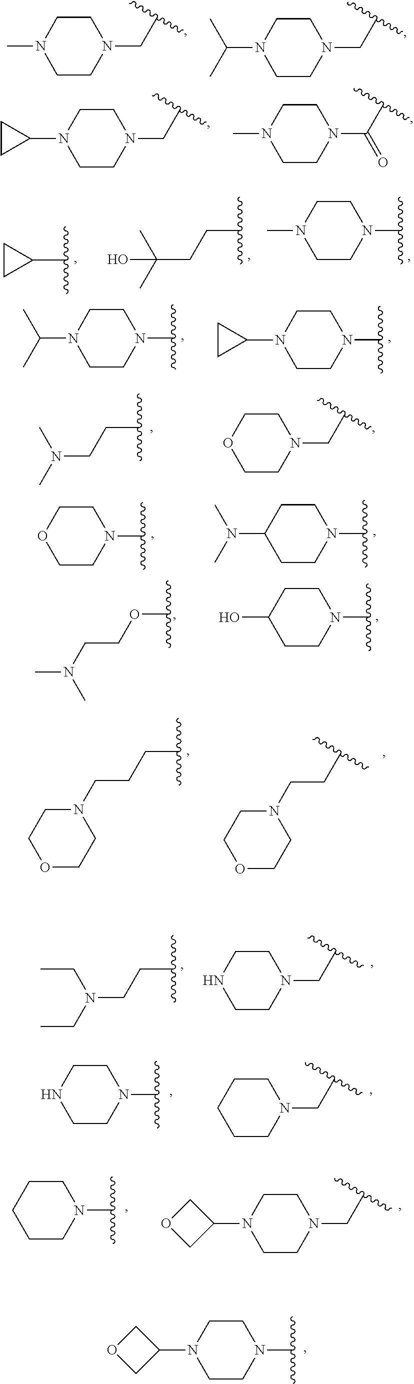

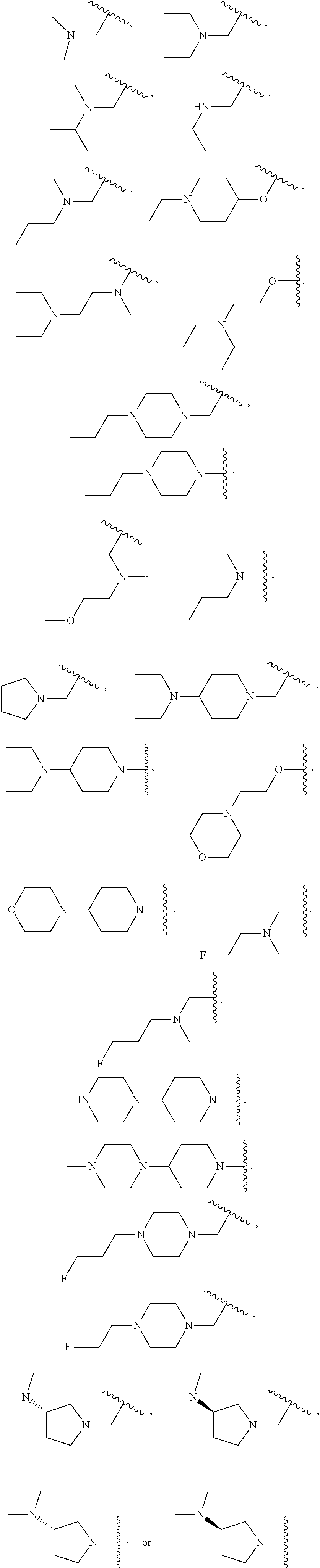

### Exemplary Compounds

Exemplary compounds (e.g., according to Formula I or any other formula described herein) include any one of the following compounds. Accordingly, exemplary compounds include any of Compounds (1)-(58), (61)-(71), (73)-(80), and (82)-(193), or a pharmaceutically acceptable salt thereof.

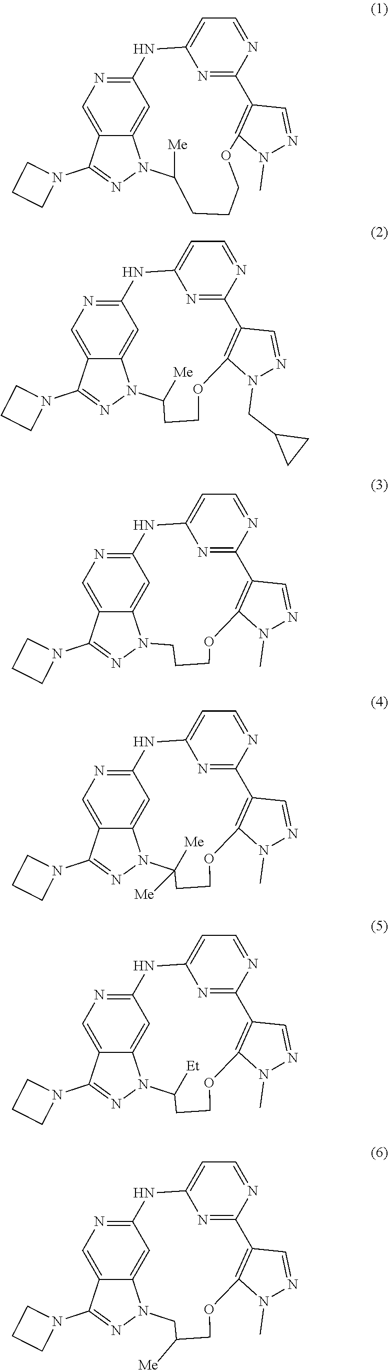

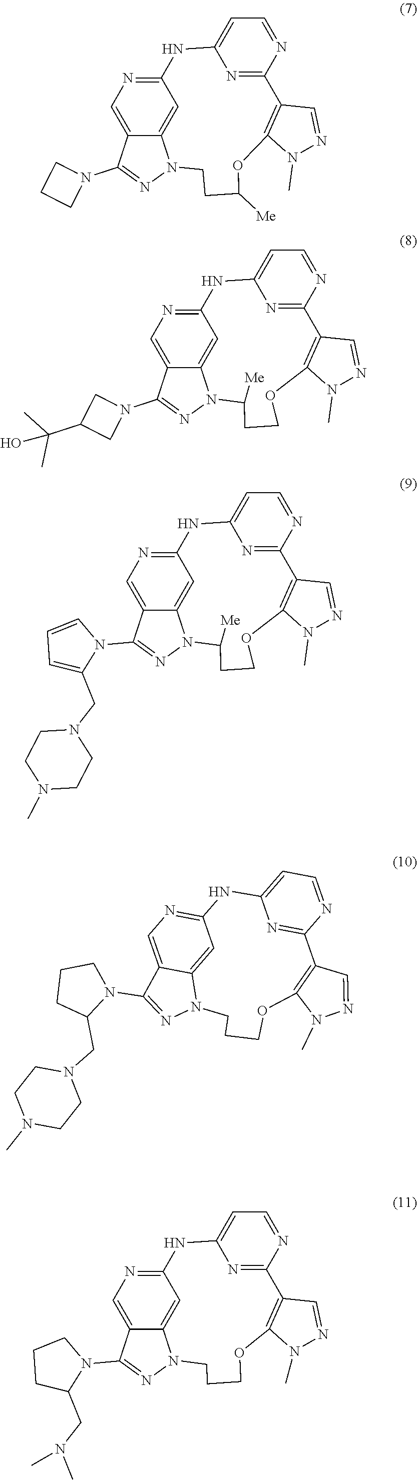

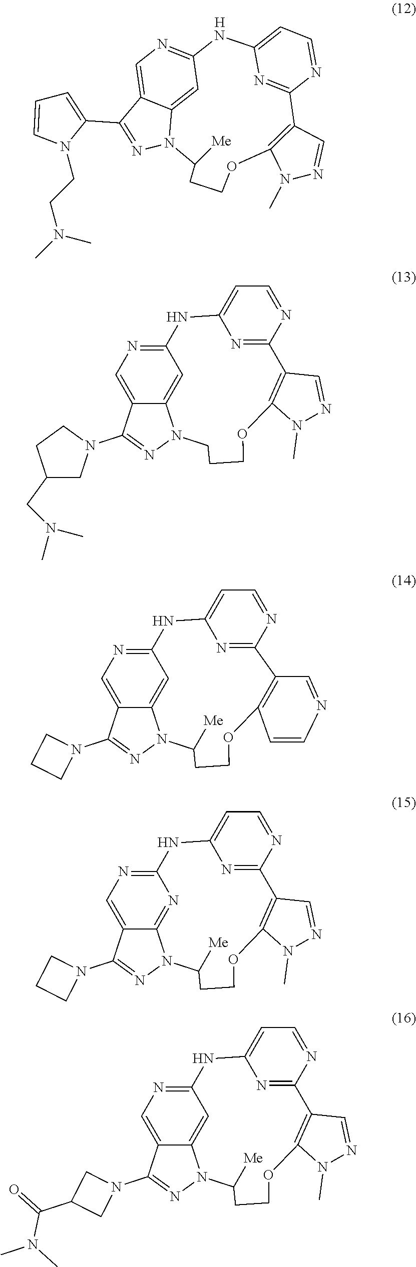

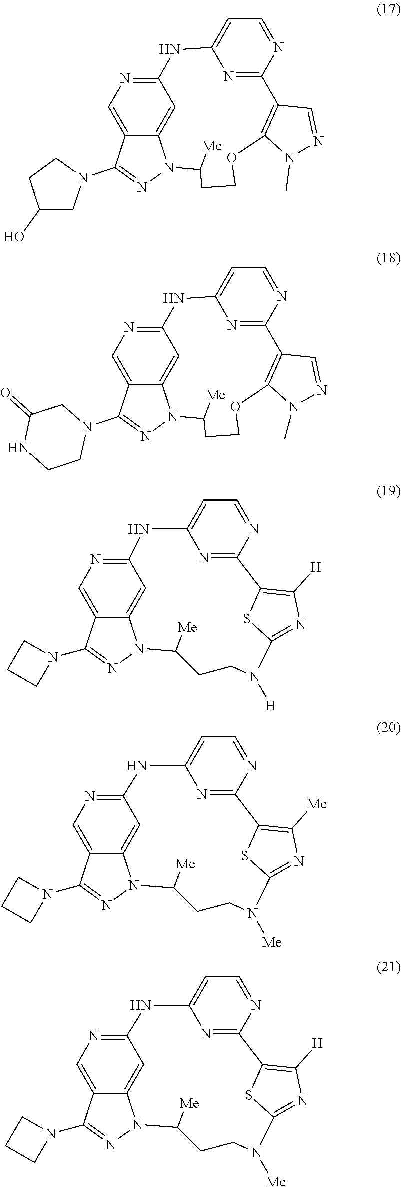

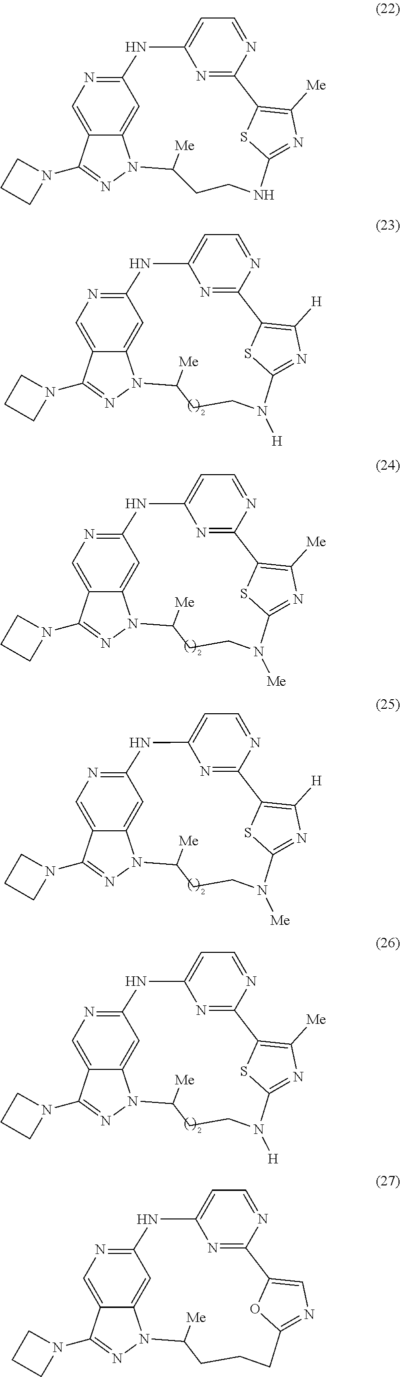

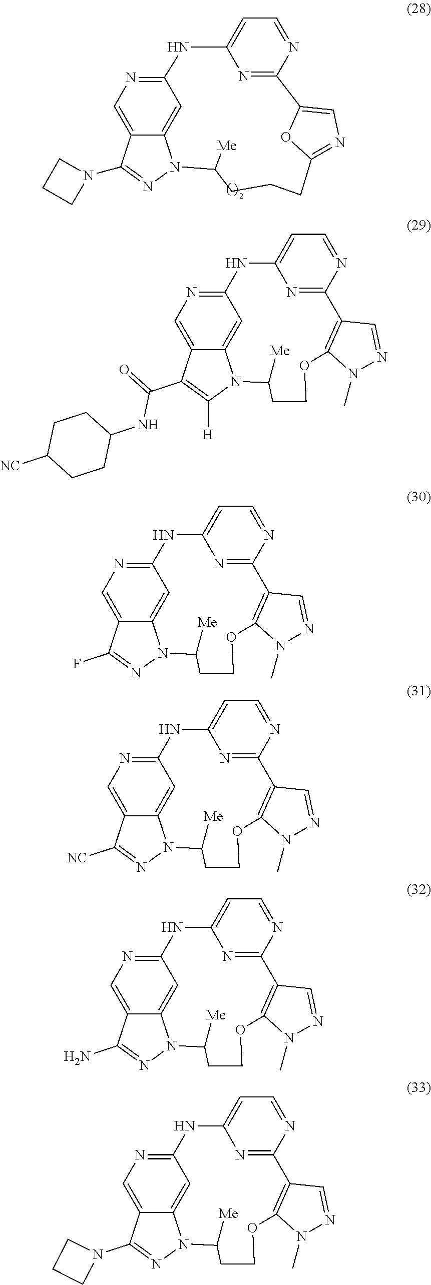

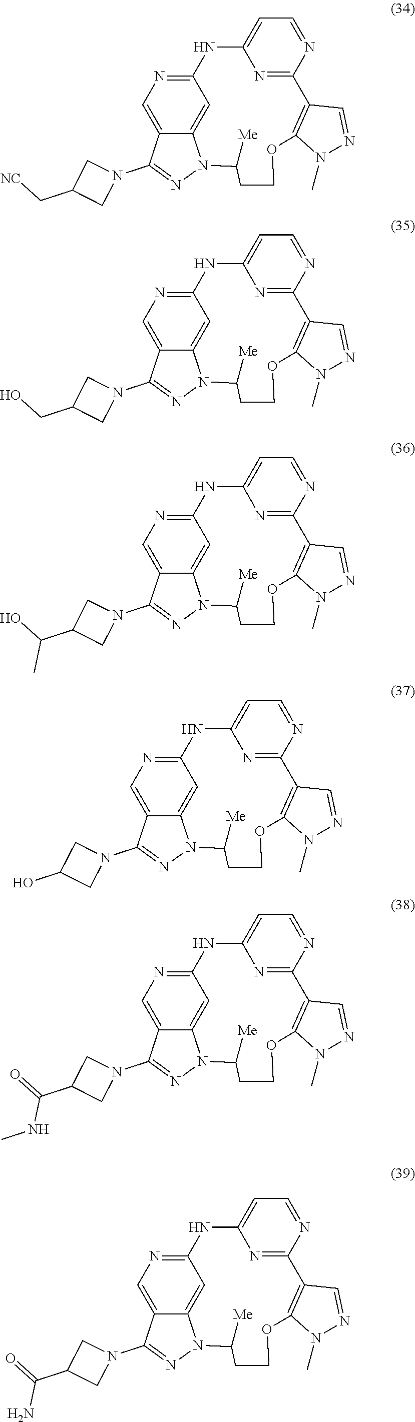

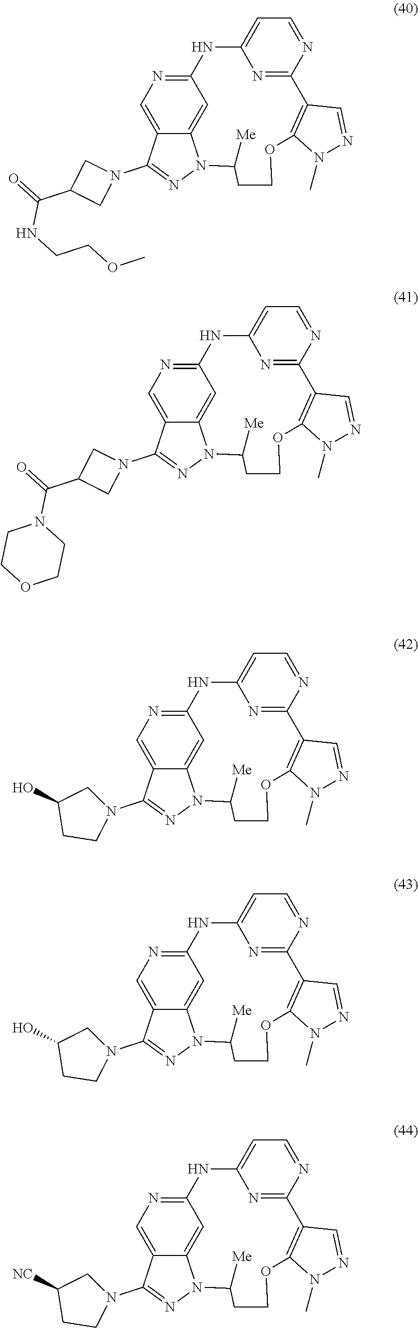 [SMILES: COCCNC(=O)C1CN(c2nn3c4cc(ncc24)Nc2ccnc(n2)-c2cnn(C)c2OCCC3C)C1 ; CC1CCOc2c(cnn2C)-c2nccc(n2)Nc2cc3c(cn2)c(N2CC(C(=O)N4CCOCC4)C2)nn31 ; CC1CCOc2c(cnn2C)-c2nccc(n2)Nc2cc3c(cn2)c(N2CC[C@@H](O)C2)nn31 ; CC1CCOc2c(cnn2C)-c2nccc(n2)Nc2cc3c(cn2)c(N2CC[C@H](O)C2)nn31 ; CC1CCOc2c(cnn2C)-c2nccc(n2)Nc2cc3c(cn2)c(N2CC[C@@H](C#N)C2)nn31]

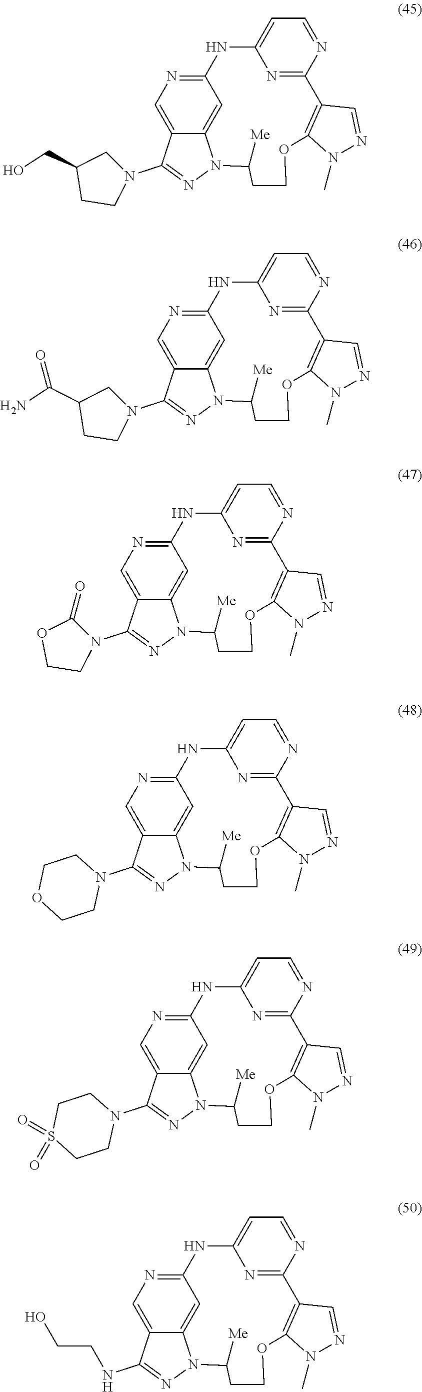 [SMILES: CC1CCOc2c(cnn2C)-c2nccc(n2)Nc2cc3c(cn2)c(N2CC[C@@H](CO)C2)nn31 ; CC1CCOc2c(cnn2C)-c2nccc(n2)Nc2cc3c(cn2)c(N2CCC(C(N)=O)C2)nn31 ; CC1CCOc2c(cnn2C)-c2nccc(n2)Nc2cc3c(cn2)c(N2CCOC2=O)nn31 ; CC1CCOc2c(cnn2C)-c2nccc(n2)Nc2cc3c(cn2)c(N2CCOCC2)nn31 ; CC1CCOc2c(cnn2C)-c2nccc(n2)Nc2cc3c(cn2)c(N2CCS(=O)(=O)CC2)nn31 ; CC1CCOc2c(cnn2C)-c2nccc(n2)Nc2cc3c(cn2)c(NCCO)nn31]

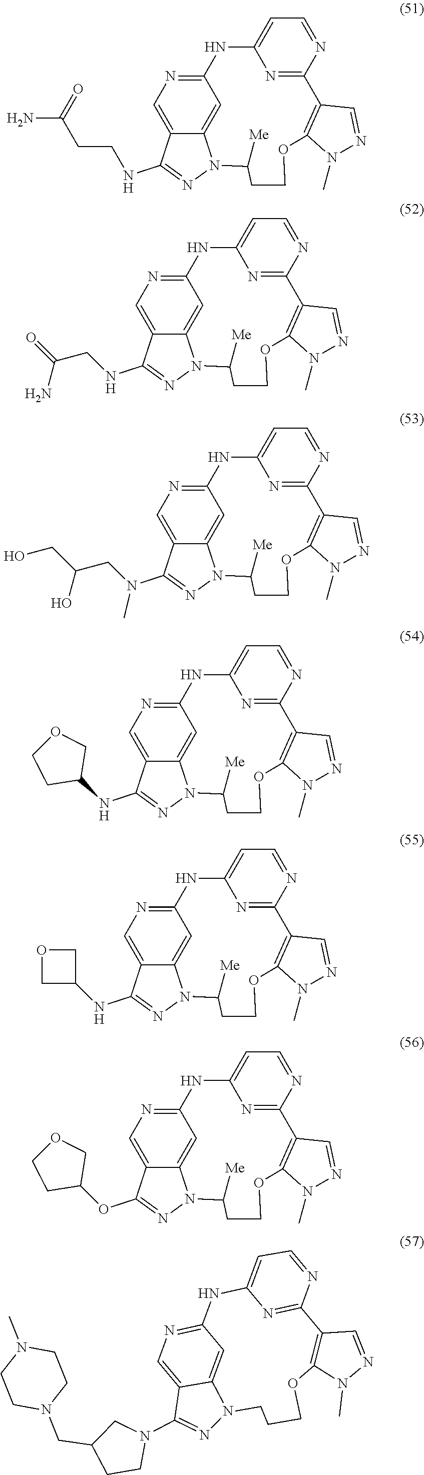

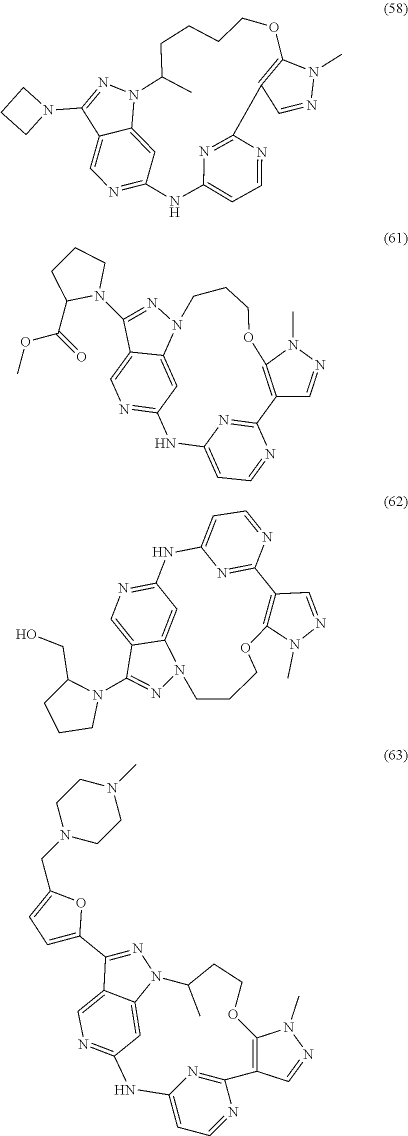

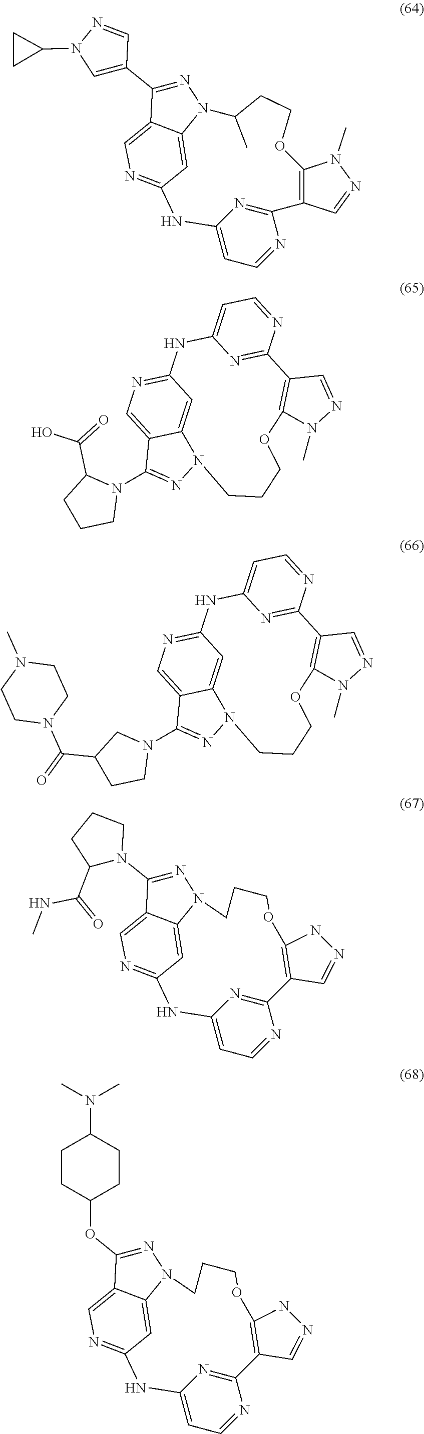

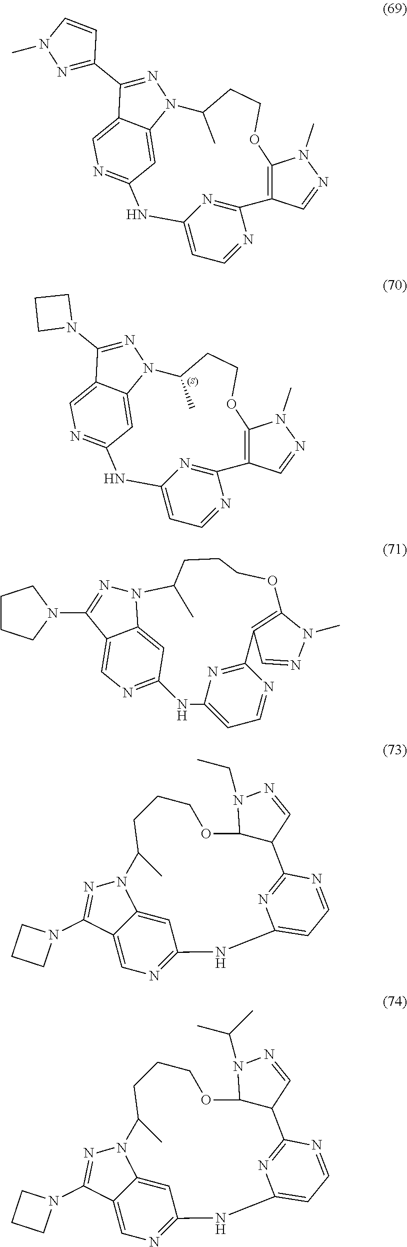

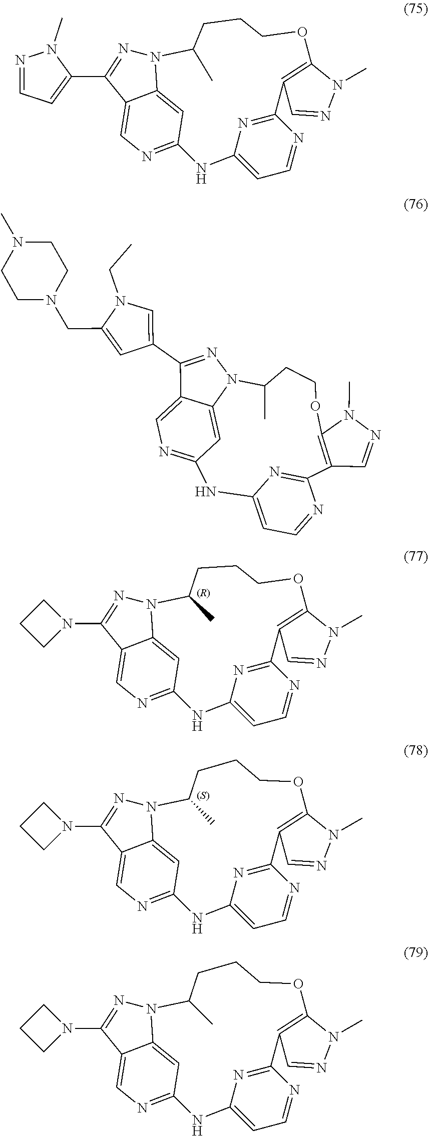

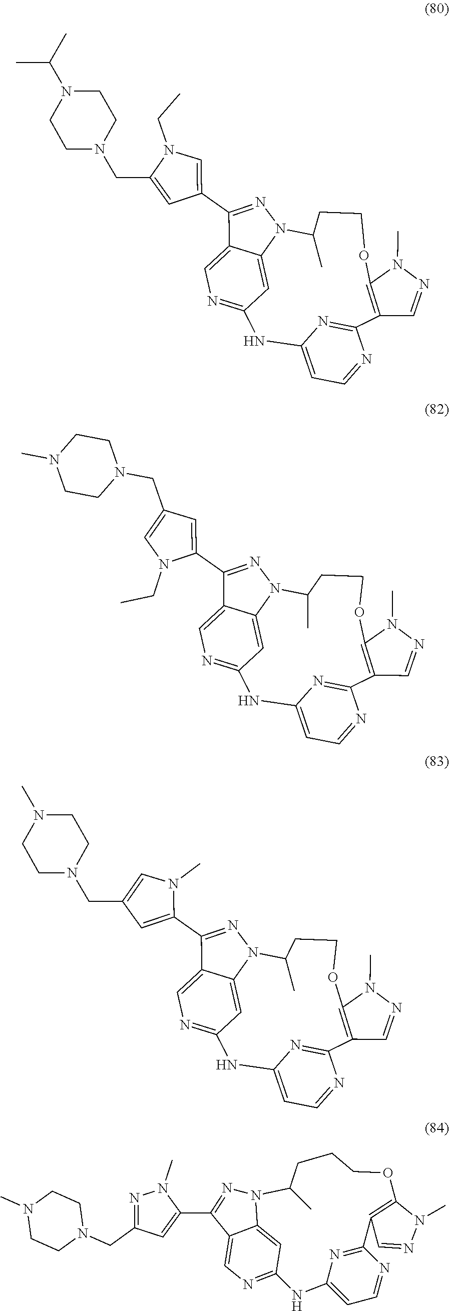

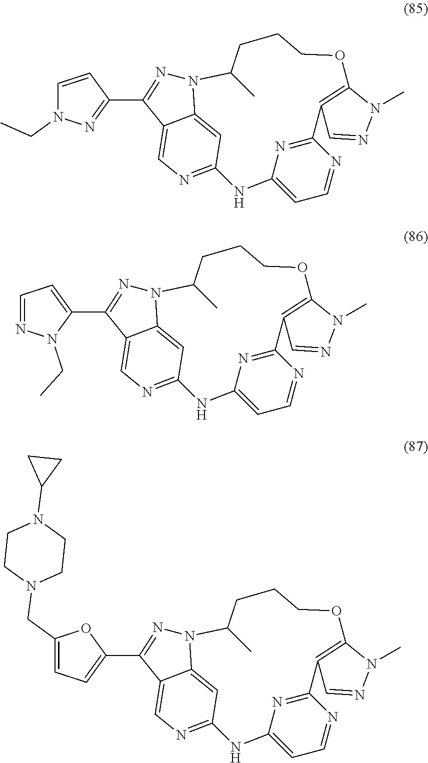 [SMILES: CCn1ccc(-c2nn3c4cc(ncc24)Nc2ccnc(n2)-c2cnn(C)c2OCCCC3C)n1 ; CCn1nccc1-c1nn2c3cc(ncc13)Nc1ccnc(n1)-c1cnn(C)c1OCCCC2C ; CC1CCCOc2c(cnn2C)-c2nccc(n2)Nc2cc3c(cn2)c(-c2ccc(CN4CCN(C5CC5)CC4)o2)nn31]

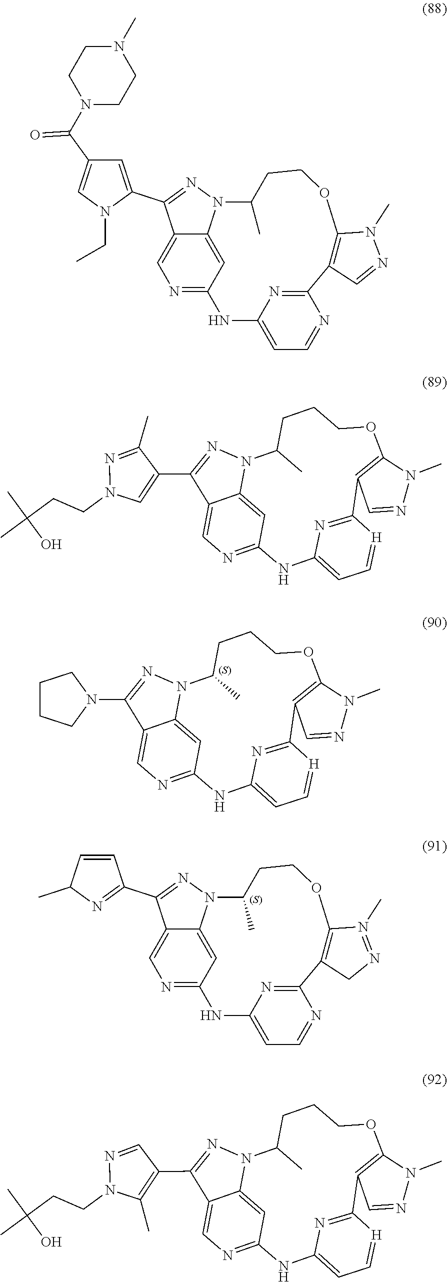

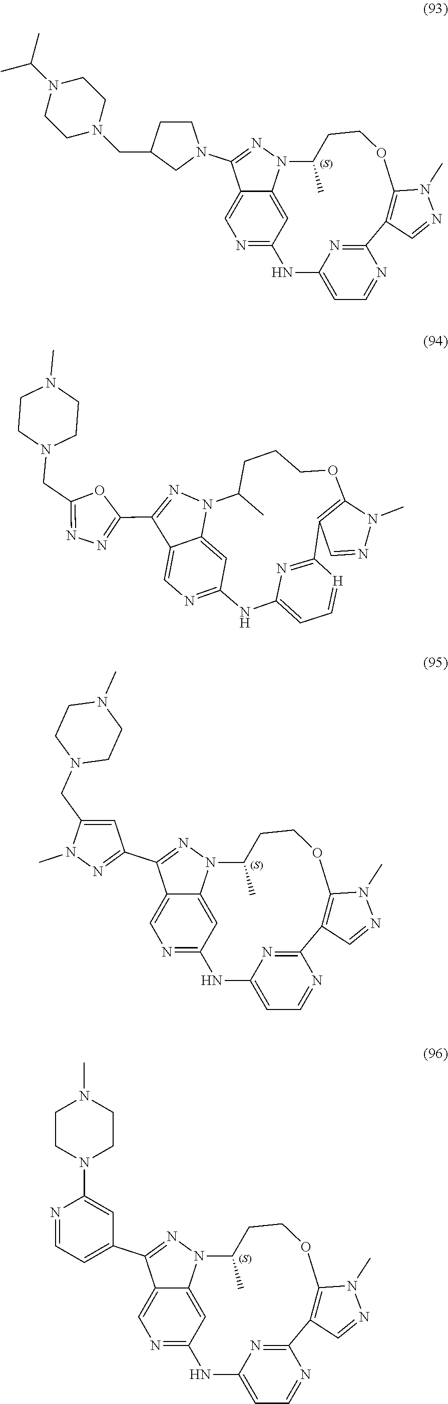

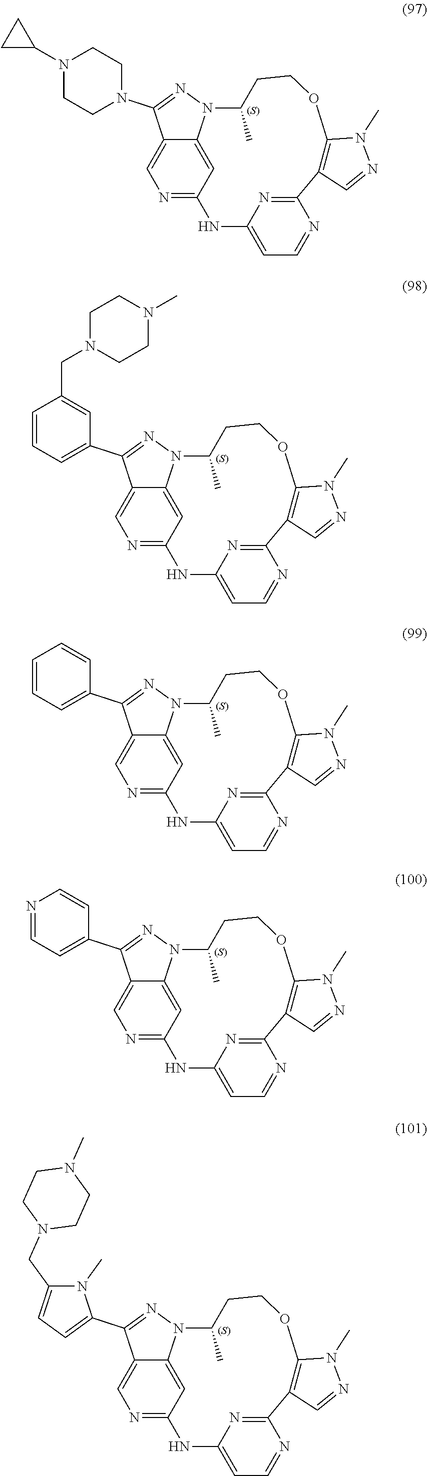

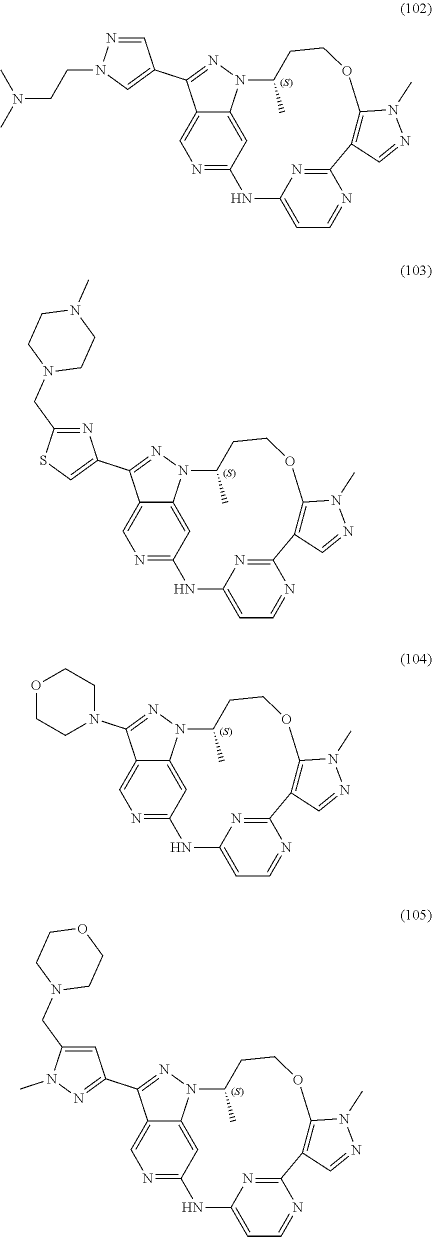

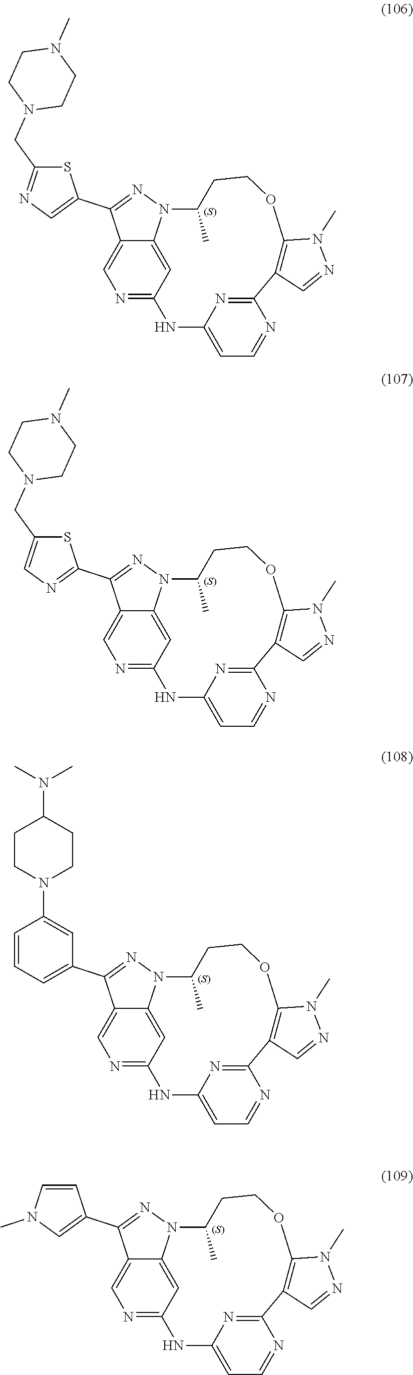

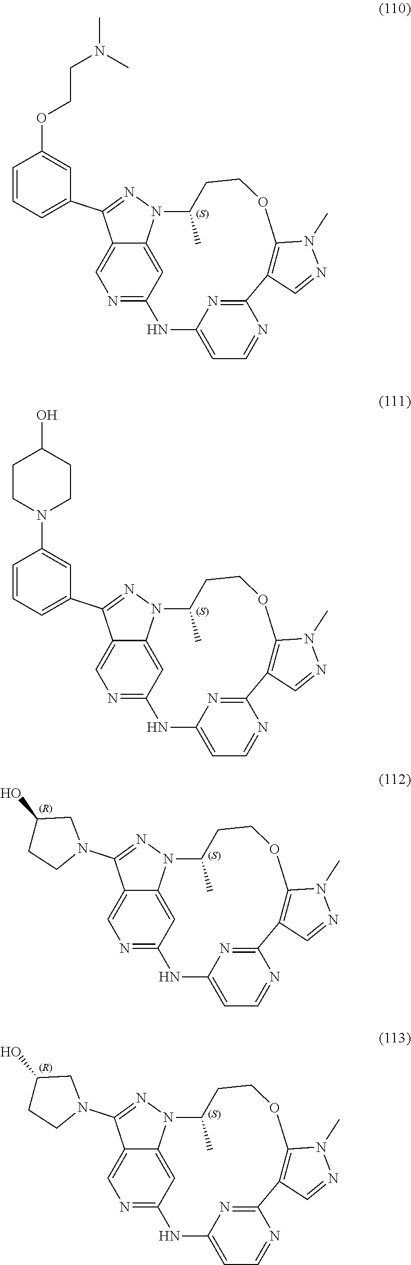

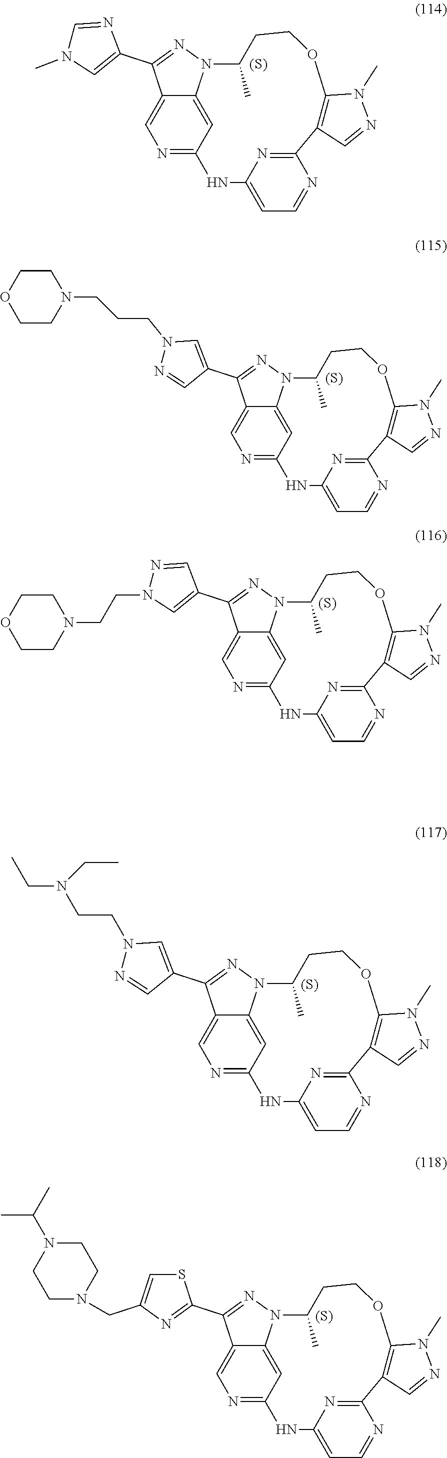

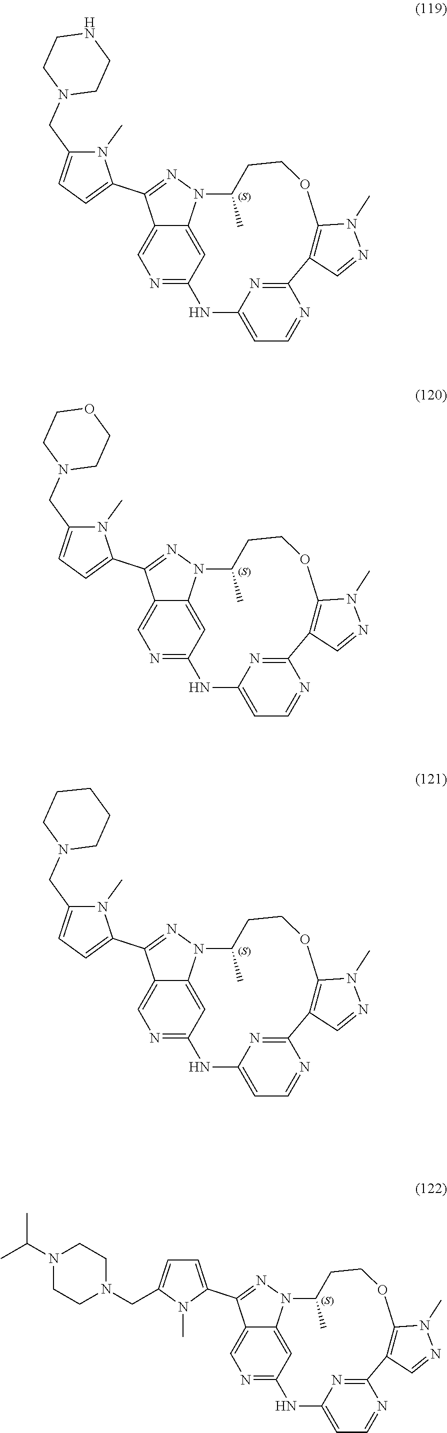

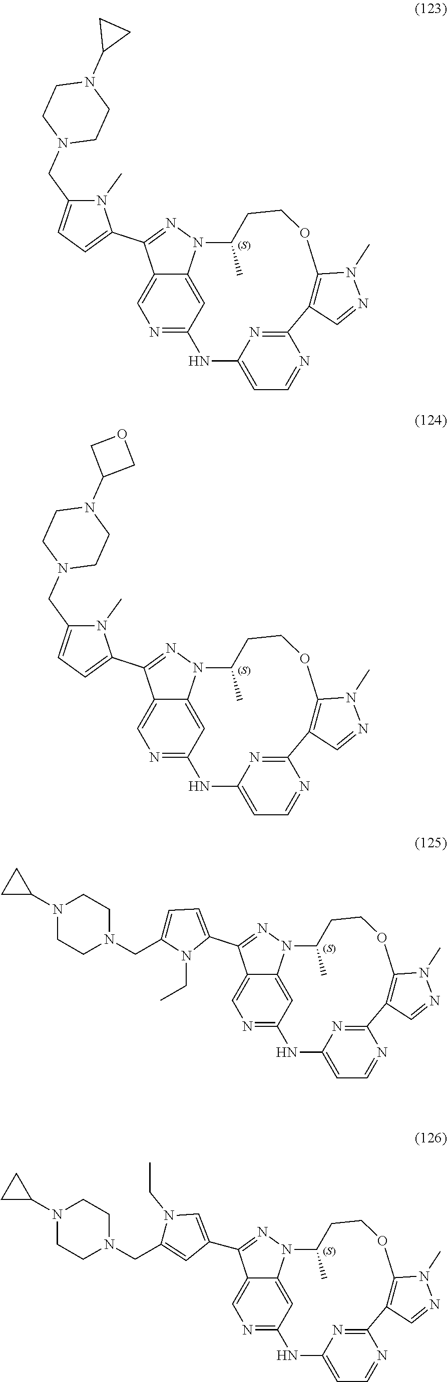

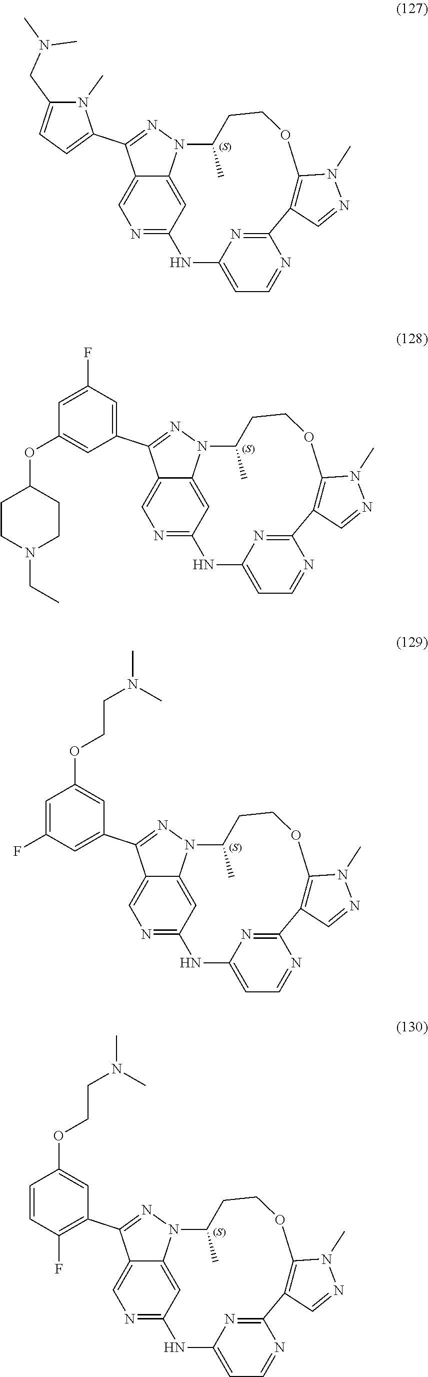

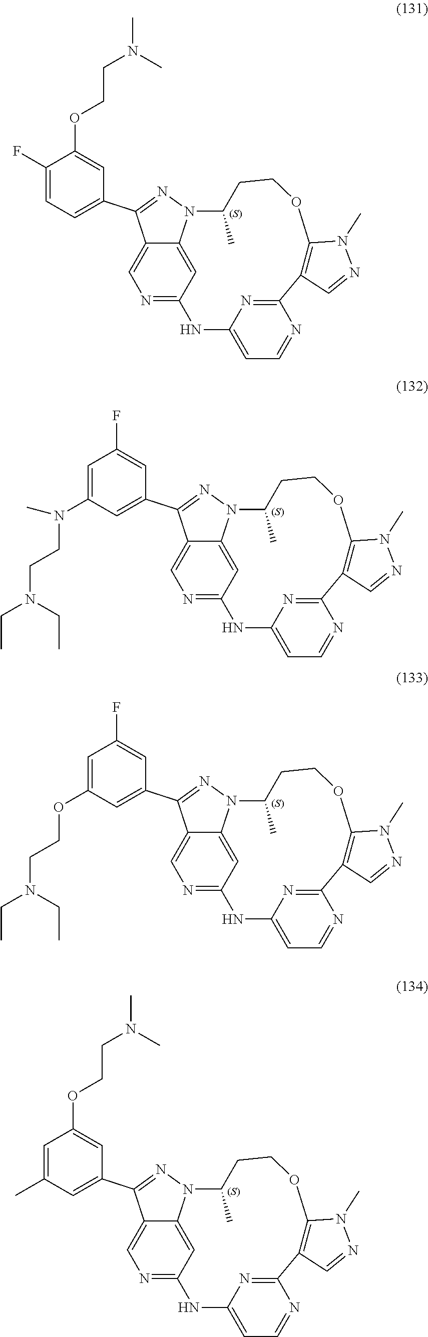

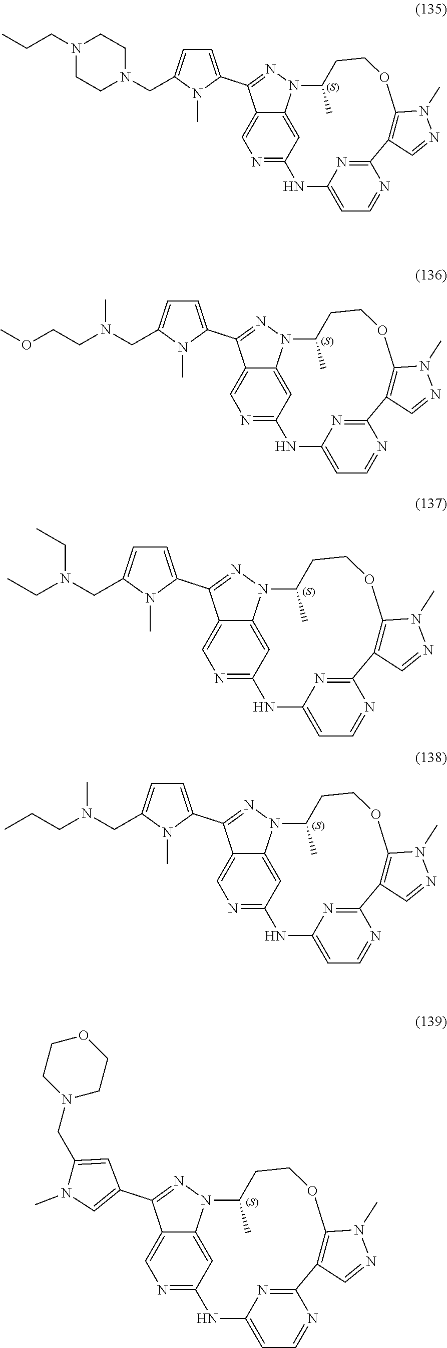

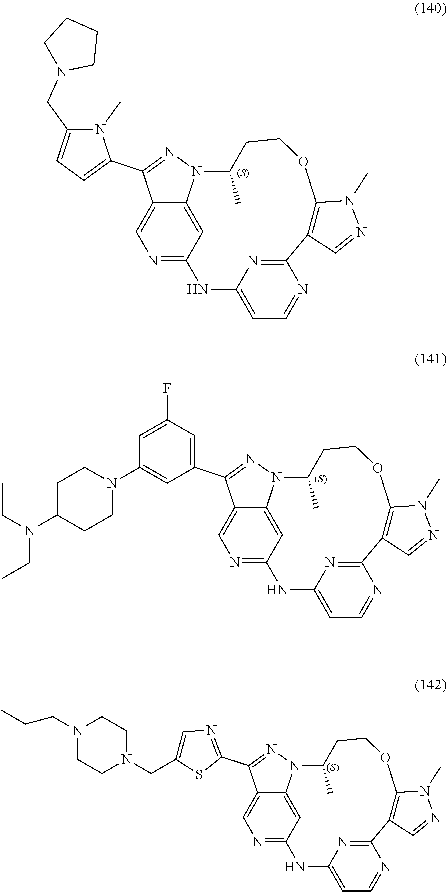

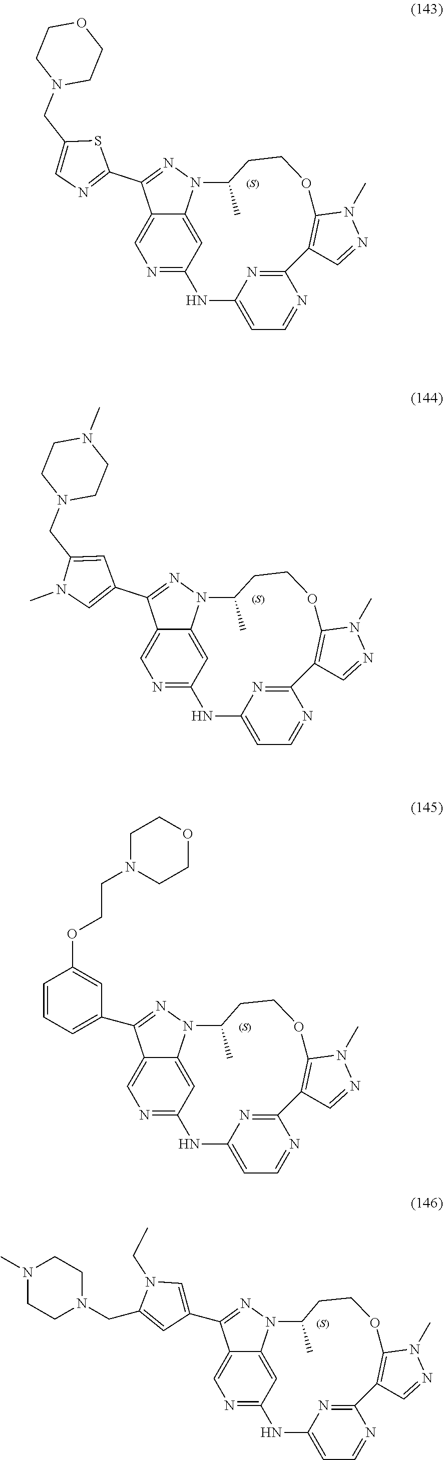

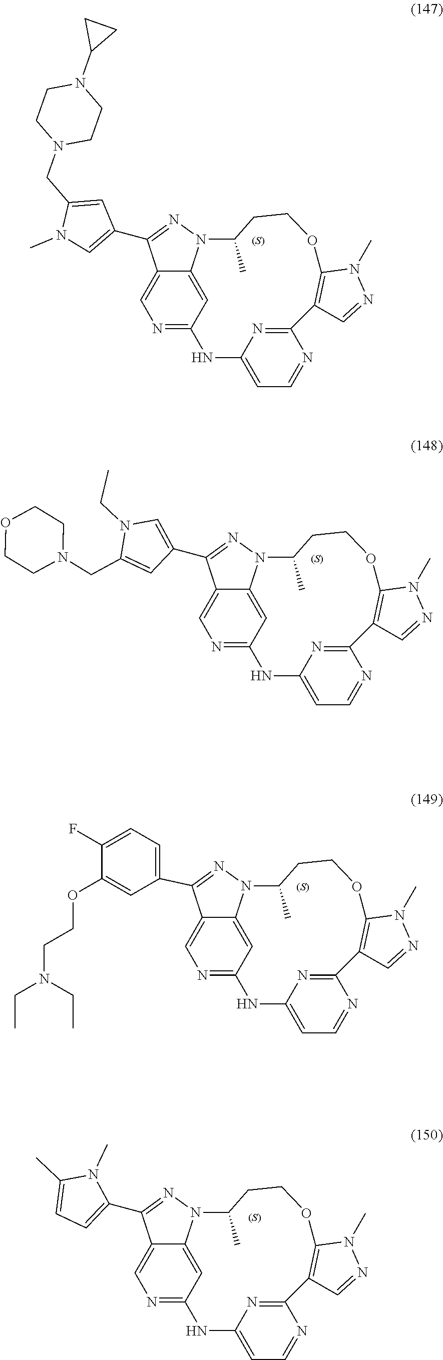

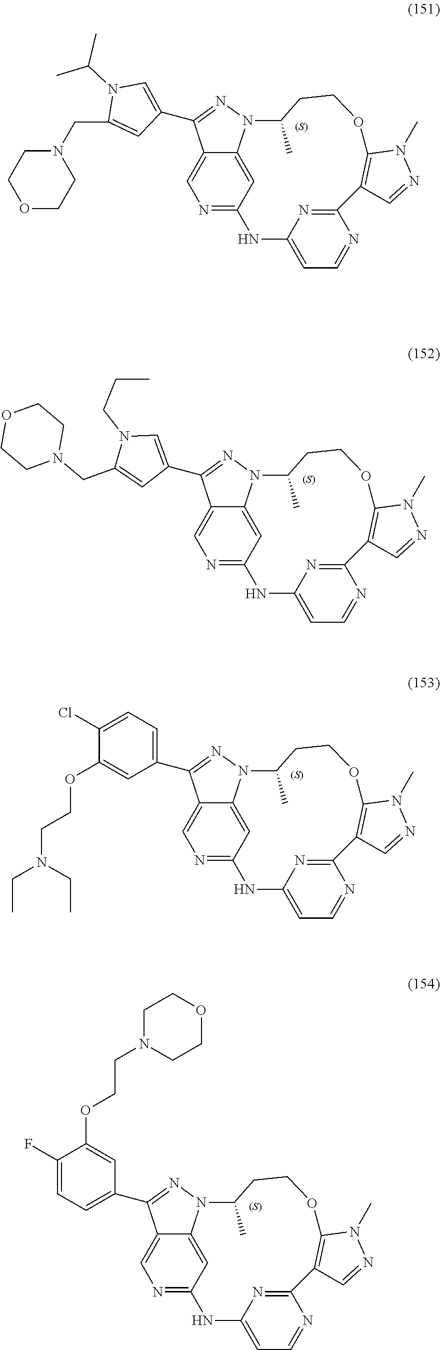

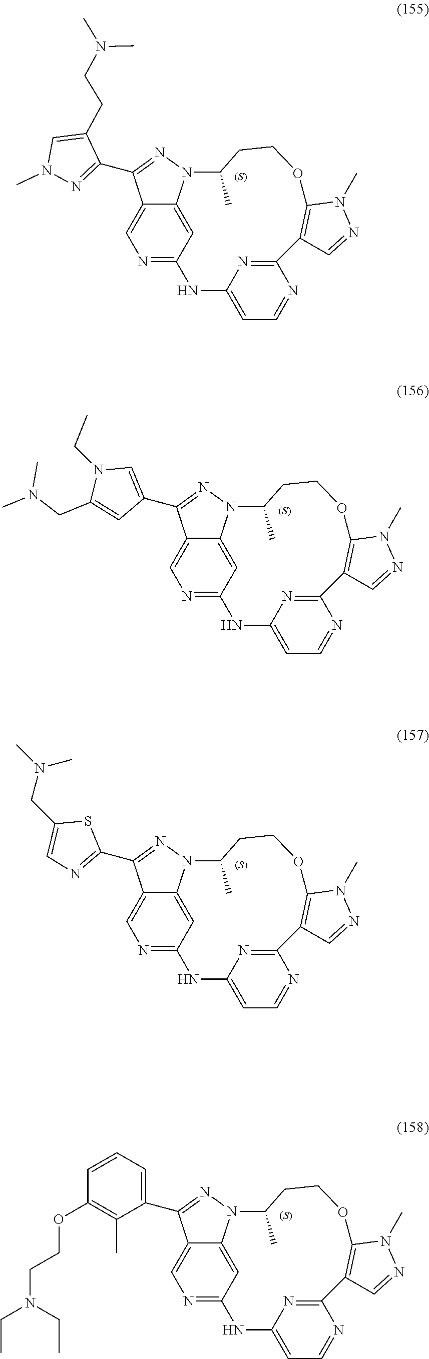

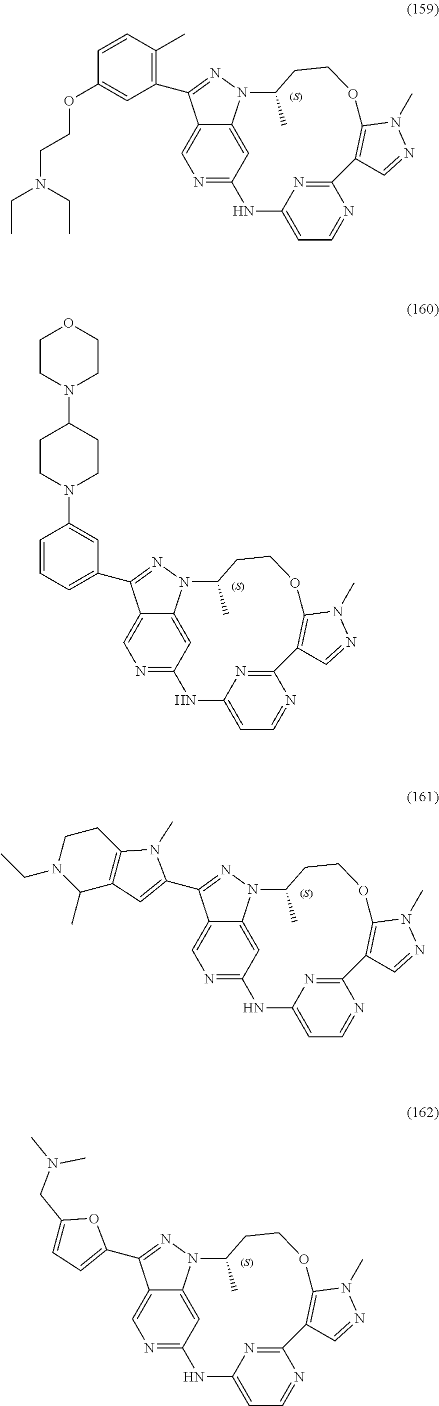

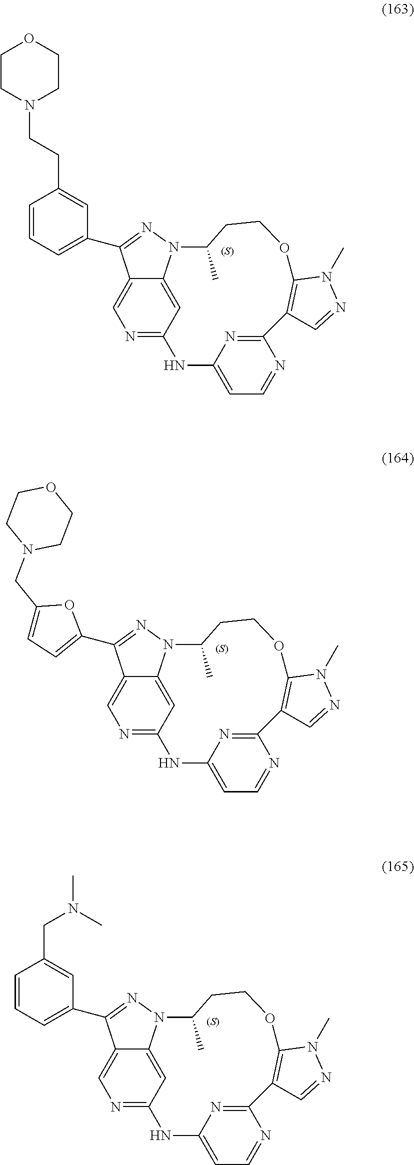

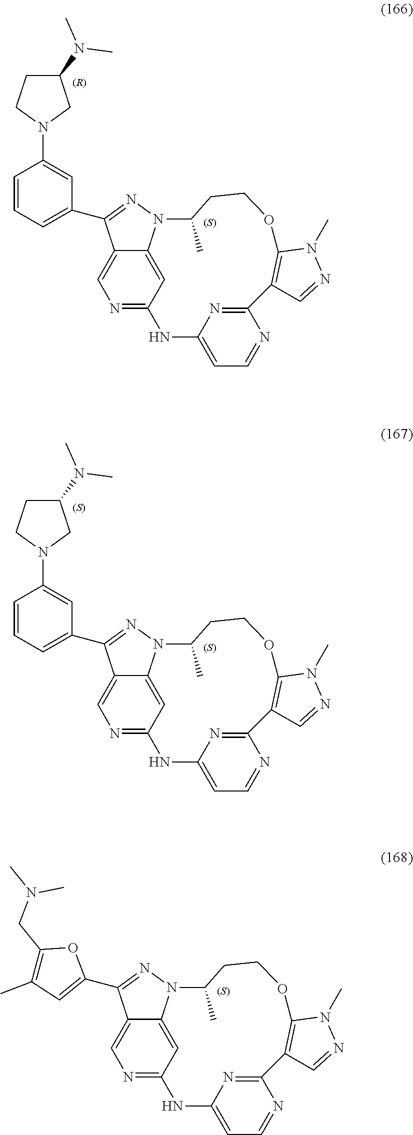

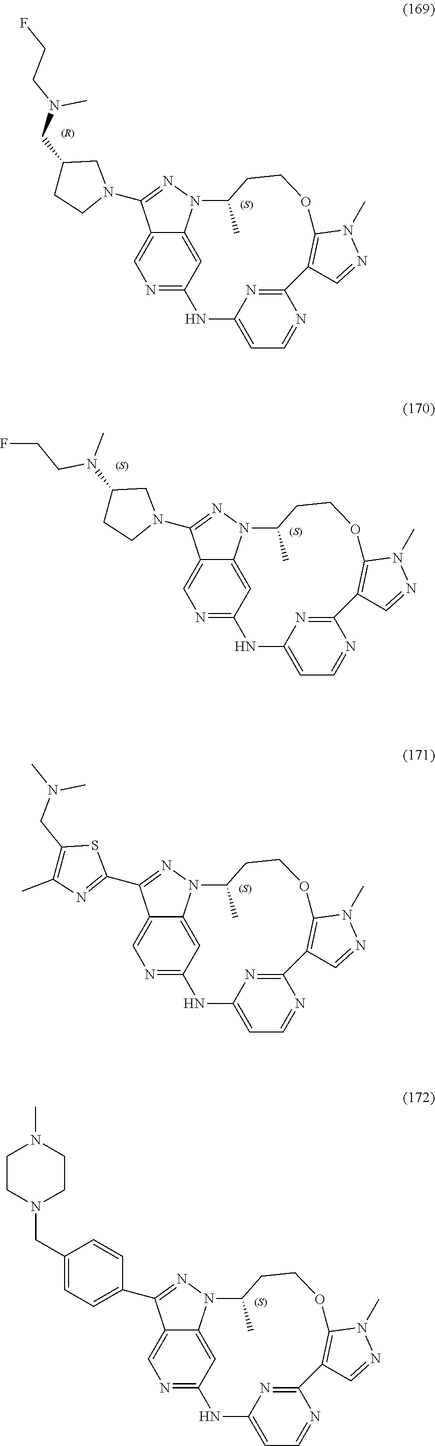

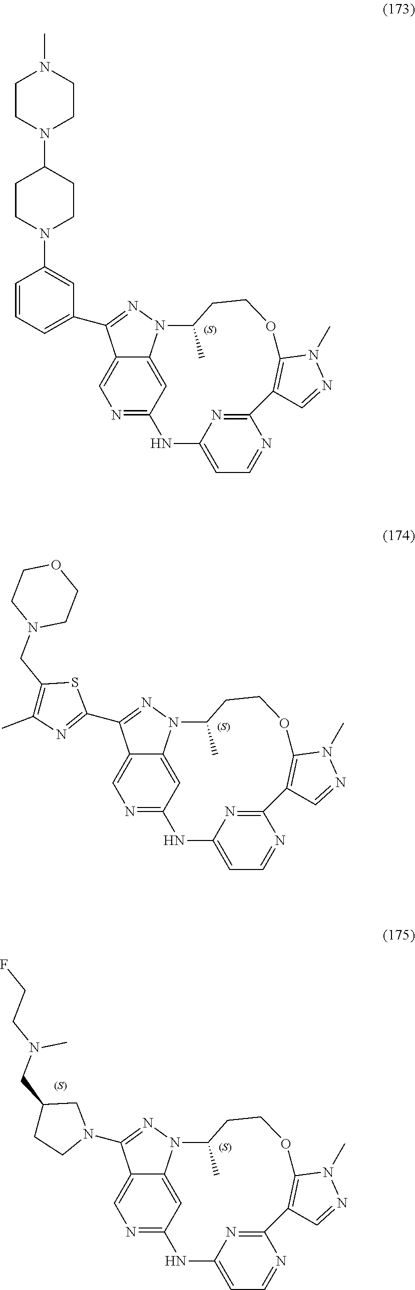

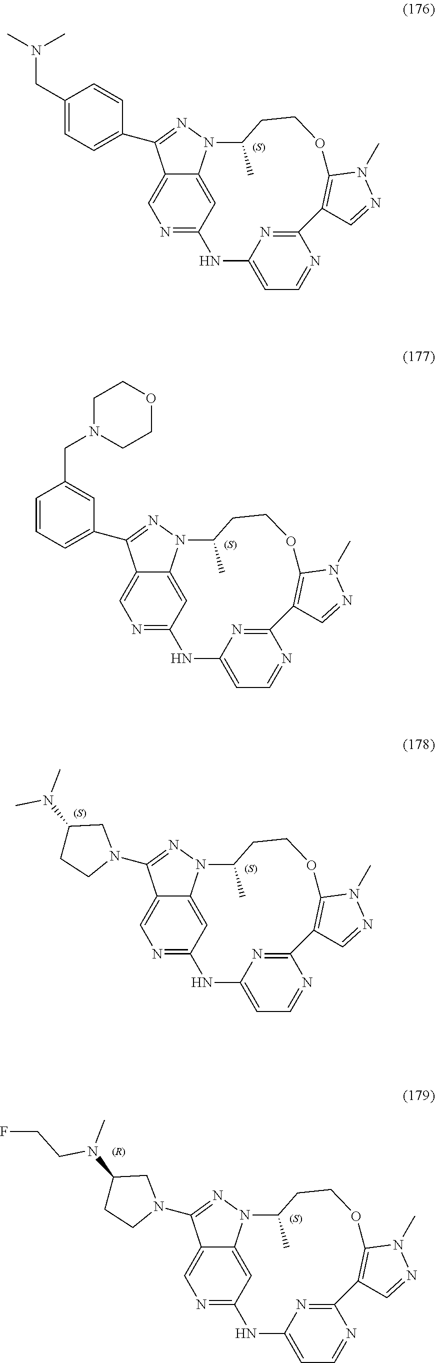

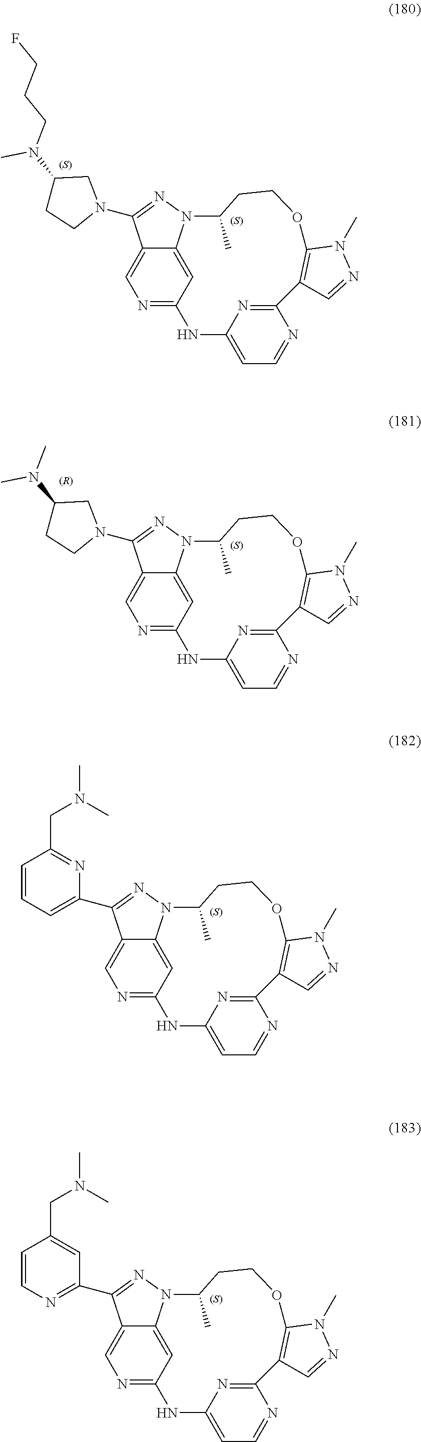

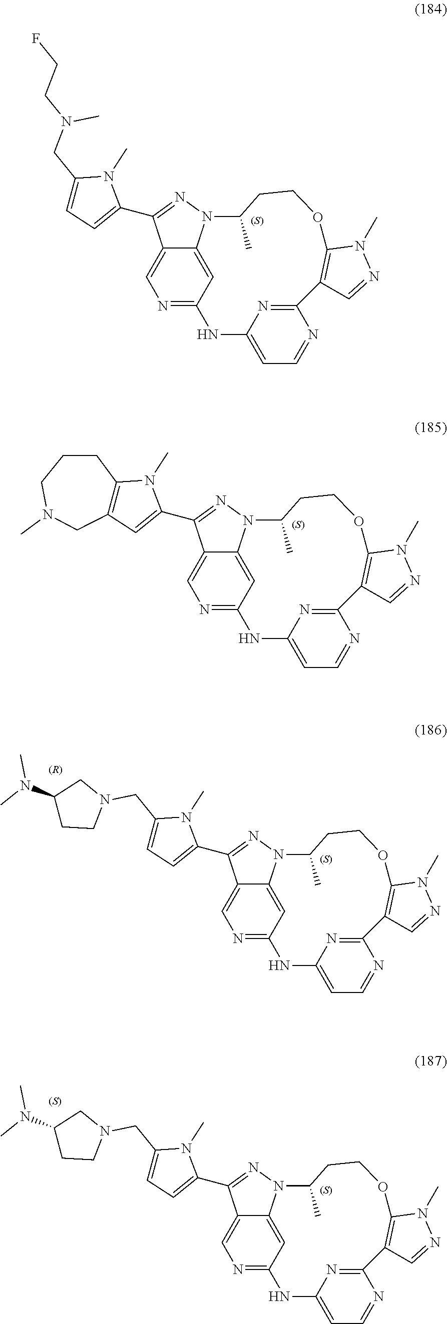

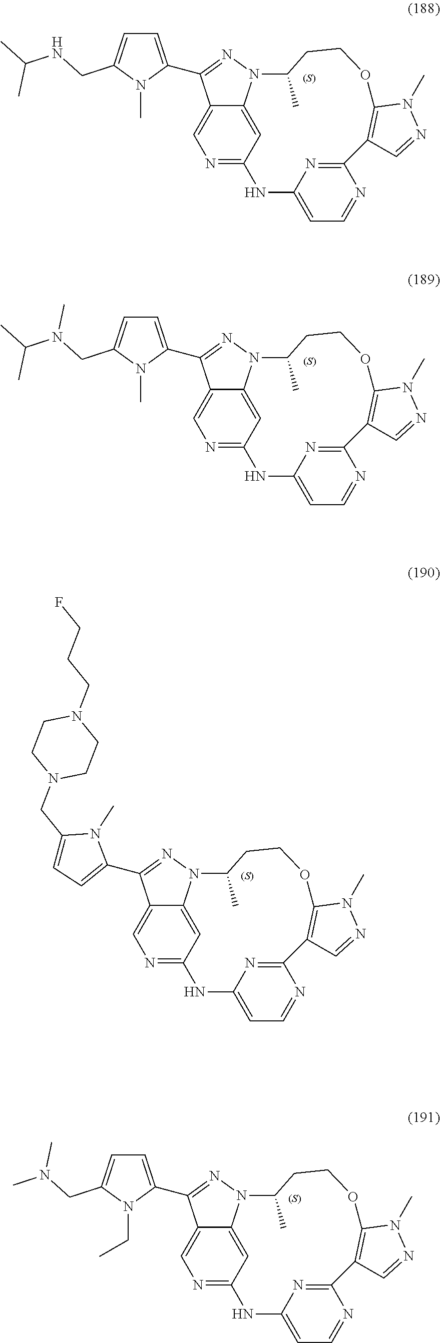

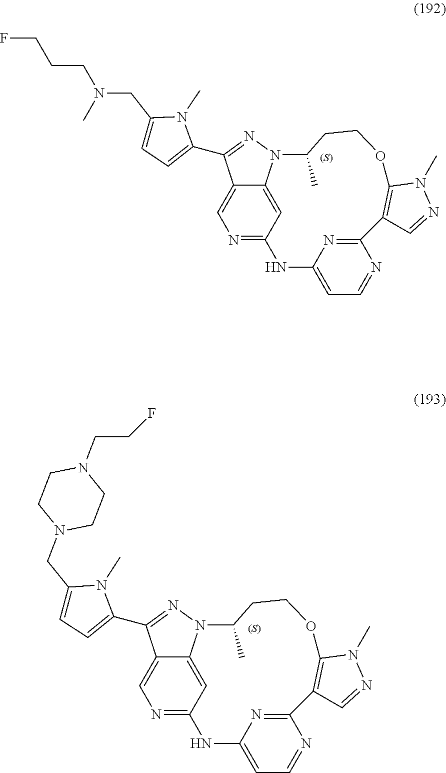

or a pharmaceutically acceptable salt thereof.

### Deuterated Compounds

Compounds described herein can comprise atoms that exhibit their natural isotopic abundances, or one or more of the atoms may be artificially enriched in a particular isotope having the same atomic number, but an atomic mass or mass number different from the atomic mass or mass number predominately found in nature. The term “isotopologue” refers to a species that has the same chemical structure and formula as a specific compound provided herein, with the exception of the positions of isotopic substitution and/or level of isotopic enrichment at one or more positions, e.g., hydrogen vs. deuterium. The present invention is meant to include all suitable isotopic variations of the compounds of the compounds described herein. For example, different isotopic forms of hydrogen (H) include protium (¹H), deuterium (²H), and tritium (³H), as well as compositions enriched in isotopologues of any compound described herein.

In embodiments, one or more of the hydrogens of the compounds described herein is replaced by a deuterium. When a position is designated as “H” or “hydrogen”, the position is understood to have hydrogen at its natural abundance isotopic composition. When a position is designated as “²H” or “deuterium”, the position is understood to have deuterium at an abundance that is at least 3340 times greater than the natural abundance of deuterium, which is 0.015% (i.e., the term “2H” or “deuterium” indicates at least 50.1% incorporation of deuterium). Accordingly, the invention also features compositions enriched in deuterated compounds.

In embodiments, compositions of any compound provided herein may have an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### Synthetic Methods

Compounds described herein can be prepared according to methods known in the art. For example, the exemplary synthetic methods described in the instant Examples can be used to prepare still other compounds of the invention.

Accordingly, disclosed compounds can generally be synthesized by an appropriate combination of generally well known synthetic methods. Techniques useful in synthesizing these chemical entities are both readily apparent and accessible to those of skill in the relevant art, based on the instant disclosure. Many of the optionally substituted starting compounds and other reactants are commercially available, e.g., from Aldrich Chemical Company (Milwaukee, Wis.) or can be readily prepared by those skilled in the art using commonly employed synthetic methodology.

An exemplary synthetic scheme for preparing certain compounds according to the invention is provided in Scheme 1.

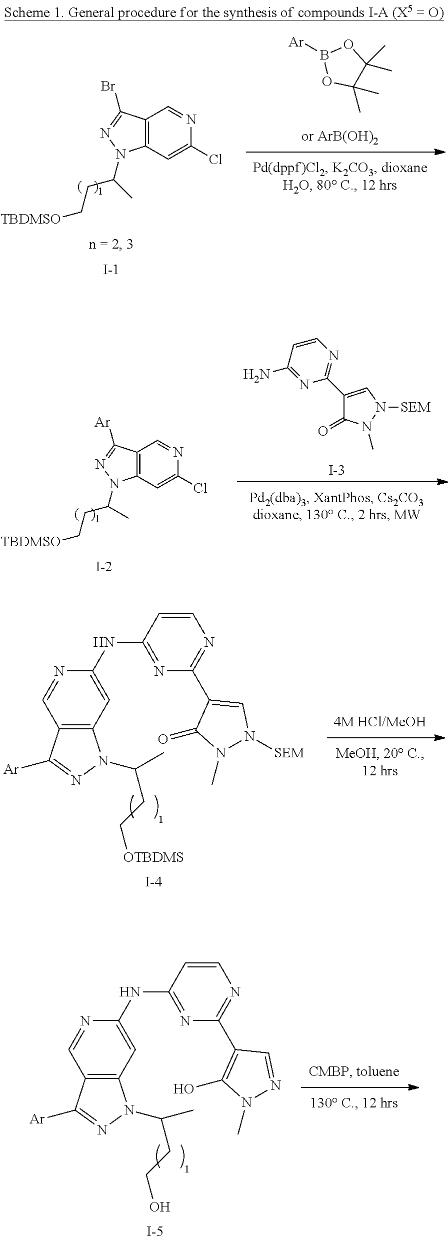

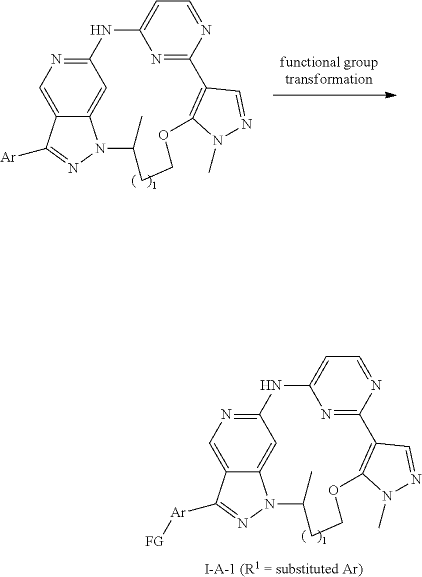

Table A below summarizes exemplary synthetic procedures that were used to prepare certain compounds described herein.

TABLE A
Summary of Synthetic Procedures

| | example # followed | ESI-MS |
|---|---|---|
| cmpnd | for compound | m/z |
| # | synthesis | [M + H]⁺ |
| (1) | 1 | 432.0 |
| (2) | 1 | 458.1 |
| (3) | 1 | 404.1 |
| (4) | 2 | 432.1 |
| (5) | 1 | 432.0 |
| (6) | 1 | 418.0 |
| (8) | 1 | 476.1 |
| (10) | 1 | 530.1 |
| (11) | 1 | 475.2 |
| (12) | 3 | 499.1 |
| (13) | 1 | 475.1 |
| (14) | 4 | 415.1 |
| (15) | 1 | 418.0 |
| (16) | 1 | 489.1 |
| (33) | 1 | 418.1 |
| (37) | 1 | 448.1 |
| (57) | 1 | 530.3 |
| (58) | 1 | 446.0 |
| (61) | 1 | 476.0 |
| (62) | 1 | 448.0 |
| (63) | 6 | 541.1 |
| (64) | 5 | 469.1 |
| (65) | 1 | 462.1 |
| (66) | 1 | 544.1 |
| (67) | 1 | 475.1 |
| (68) | 1 | 490.1 |
| (69) | 5 | 443.0 |
| (70) | 1 | 418.0 |
| (71) | 1 | 446.2 |
| (73) | 1 | 446.1 |
| (74) | 1 | 460.1 |
| (75) | 5 | 457.1 |
| (76) | 6 | 568.1 |
| (77) | 1 | 432.0 |
| (78) | 1 | 432.0 |
| (79) | 5 | 457.1 |
| (80) | 6 | 596.3 |
| (82) | 6 | 568.2 |
| (83) | 6 | 554.1 |
| (84) | 7 | 569.2 |
| (85) | 5 | 471.1 |
| (86) | 5 | 471.1 |
| (87) | 6 | 581.2 |
| (88) | 7 | 582.1 |
| (89) | 5 | 543.1 |
| (90) | 1 | 446.1 |
| (91) | 5 | 443.1 |
| (92) | 5 | 543.2 |
| (93) | 1 | 572.2 |
| (94) | 6 | 557.1 |
| (95) | 7 | 555.1 |
| (96) | 5 | 538.1 |
| (97) | 1 | 487.1 |
| (98) | 5 | 551.1 |
| (99) | 5 | 440.0 |
| (100) | 5 | 440.1 |
| (101) | 7 | 554.1 |
| (102) | 5 | 500.1 |
| (103) | 9 | 558.1 |
| (104) | 1 | 448.0 |
| (105) | 7 | 542.1 |
| (106) | 9 | 558.1 |
| (107) | 9 | 558.1 |
| (108) | 5 | 565.1 |
| (109) | 5 | 442.1 |
| (110) | 5 | 526.1 |
| (111) | 5 | 538.1 |
| (112) | 1 | 448.0 |
| (113) | 1 | 448.1 |
| (114) | 5 | 443.1 |
| (115) | 5 | 556.1 |
| (116) | 5 | 542.1 |
| (117) | 5 | 528.1 |
| (118) | 9 | 586.2 |
| (119) | 7 | 540.2 |
| (120) | 7 | 541.1 |
| (121) | 7 | 539.3 |
| (122) | 7 | 582.3 |
| (123) | 7 | 580.2 |
| (124) | 7 | 596.4 |
| (125) | 7 | 594.3 |
| (126) | 6 | 594.3 |
| (127) | 7 | 499.2 |
| (128) | 5 | 584.3 |
| (129) | 5 | 544.1 |
| (130) | 5 | 544.1 |
| (131) | 5 | 544.1 |
| (132) | 5 | 585.2 |
| (133) | 5 | 572.3 |
| (134) | 5 | 540.2 |
| (135) | 7 | 582.2 |
| (136) | 7 | 543.1 |
| (137) | 7 | 527.1 |
| (138) | 7 | 527.1 |
| (139) | 6 | 541.2 |
| (140) | 7 | 525.1 |
| (141) | 5 | 611.2 |
| (142) | 9 | 586.2 |
| (143) | 9 | 545.2 |
| (144) | 6 | 554.2 |
| (145) | 5 | 568.1 |
| (146) | 6 | 568.3 |
| (147) | 6 | 580.2 |
| (148) | 6 | 555.2 |
| (149) | 5 | 572.1 |
| (150) | 5 | 456.1 |
| (151) | 6 | 569.2 |
| (152) | 6 | 569.2 |
| (153) | 5 | 588.2 |
| (154) | 5 | 586.1 |
| (155) | 5 | 514.2 |
| (156) | 6 | 513.1 |
| (157) | 9 | 503.1 |
| (158) | 5 | 568.3 |
| (159) | 5 | 568.3 |
| (160) | 5 | 607.3 |
| (161) | 5 | 539.2 |
| (162) | 6 | 486.1 |
| (163) | 5 | 552.2 |
| (164) | 6 | 528.1 |
| (165) | 5 | 496.1 |
| (166) | 5 | 551.2 |
| (167) | 5 | 551.2 |
| (168) | 6 | 500.1 |
| (169) | 1 | 521.2 |
| (170) | 1 | 507.3 |
| (171) | 9 | 517.1 |
| (172) | 5 | 551.1 |
| (173) | 5 | 620.3 |
| (174) | 9 | 559.1 |
| (175) | 1 | 521.2 |
| (176) | 5 | 496.2 |
| (177) | 5 | 538.1 |
| (178) | 1 | 475.0 |
| (179) | 1 | 507.0 |
| (180) | 1 | 521.0 |
| (181) | 1 | 475.1 |
| (182) | 5 | 497.1 |
| (183) | 5 | 497.1 |
| (184) | 8 | 531.0 |
| (185) | 5 | 525.1 |
| (186) | 7 | 568.1 |
| (187) | 7 | 568.1 |
| (188) | 8 | 513.3 |
| (189) | 8 | 527.3 |
| (190) | 8 | 600.1 |
| (191) | 7 | 513.1 |
| (192) | 8 | 545.1 |
| (193) | 8 | 586.1 |

### Pharmaceutical Compositions

In another exemplary aspect, the invention features pharmaceutical compositions comprising any compound herein, or a pharmaceutically acceptable form thereof. In embodiments, a pharmaceutical composition comprises a therapeutically effective amount of any compound described herein, or any pharmaceutically acceptable form thereof.

In embodiments, a pharmaceutically acceptable form of a compound includes any pharmaceutically acceptable salts, hydrates, solvates, isomers, prodrugs, and isotopically labeled derivatives thereof.

In embodiments, a pharmaceutical composition comprises any compound described herein, or a pharmaceutically acceptable salt thereof.

In embodiments, a pharmaceutical composition comprises a pharmaceutically acceptable excipient.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

Accordingly, in some embodiments, provided herein are pharmaceutical compositions comprising one or more compounds as disclosed herein, or a pharmaceutically acceptable form thereof (e.g., pharmaceutically acceptable salts, hydrates, solvates, isomers, prodrugs, and isotopically labeled derivatives), and one or more pharmaceutically acceptable excipients, carriers, including inert solid diluents and fillers, diluents, including sterile aqueous solution and various organic solvents, permeation enhancers, solubilizers and adjuvants. In some embodiments, a pharmaceutical composition described herein includes a second active agent such as an additional therapeutic agent, (e.g., a chemotherapeutic).

Accordingly, the present teachings also provide pharmaceutical compositions that include at least one compound described herein, or any pharmaceutically salt thereof, and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the composition and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known 5-hydroxytryptamine receptor 7 activity modulators. Pharmaceutical compositions in the form of oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations described herein can utilize standard delay or time-release formulations to alter the absorption of the compound(s). An oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

In embodiments, a pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.]

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

### Kits

In some embodiments, provided herein are kits. The kits can include a compound or pharmaceutically acceptable form thereof, or pharmaceutical composition as described herein, in suitable packaging, and written material that can include instructions for use, discussion of clinical studies, listing of side effects, and the like. Kits are well suited for the delivery of solid oral dosage forms such as tablets or capsules. Such kits can also include information, such as scientific literature references, package insert materials, clinical trial results, and/or summaries of these and the like, which indicate or establish the activities and/or advantages of the pharmaceutical composition, and/or which describe dosing, administration, side effects, drug interactions, or other information useful to the health care provider. Such information can be based on the results of various studies, for example, studies using experimental animals involving in vivo models and studies based on human clinical trials.

### Therapeutic Methods

Compounds of the present teachings can be useful for the treatment or inhibition of a pathological condition or disorder in a mammal, for example, a human subject. The present teachings accordingly provide methods of treating or inhibiting a pathological condition or disorder by providing to a mammal a compound of the present teachings (including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder.

Accordingly, compounds described herein can be particularly useful in treating diseases or disorders associated with defects in various components of signal transduction pathways and which are responsive to modulation (e.g., inhibition) of protein kinases. In embodiments, a compound described herein modulates (e.g., inhibitors) a protein kinase that is abl, Akt, bcr-abl, Blk, Brk, c-kit, c-met, c-src, CDK1, CDK2, CDK3, CDK4, CDK5, CDK6, CDK7, CDK8, CDK9, CDK10, cRaf1, CSK, EGFR, ErbB2, ErbB3, ErbB4, Erk, Pak, fes, FGFR1, FGFR2, FGFR3, FGFR4, FGFR5, Fgr, fit-1, Fps, Frk, Fyn, Hck, IGF-1R, INS-R, Jak, KDR, Lck, Lyn, MEK, p38, PDGFR, PIK, PKC, PYK2, ros, tie, tie2, TRK or Zap70. In embodiments, a compound described herein modulates (e.g., inhibits) a wild-type form of a kinase (e.g., EGFR). In embodiments, a compound described herein modulates (e.g., inhibits) a mutant form of a kinase (e.g., EGFR).

In embodiments, a compound described herein, or any pharmaceutically acceptable form thereof such as a pharmaceutically acceptable salt thereof, modulates (e.g., inhibits) a kinase that is a tyrosine kinase (e.g., KIT, erb2, PDGFR, EGFR, VEGFR, src, or abl).

In embodiments, a compound described herein, or any pharmaceutically acceptable form thereof such as a pharmaceutically acceptable salt thereof, modulates (e.g., inhibits) a kinase that is a serine/threonine kinase (e.g., mTorC1, mTorC2, ATM, ATR, DNA-PK, or Akt).

In embodiments, a compound described herein, or any pharmaceutically acceptable form thereof such as a pharmaceutically acceptable salt thereof, can be used to treat or prevent a disease or disorder that is responsive to modulation (e.g., inhibition) of a protein kinase (e.g., abl, Akt, bcr-abl, Blk, Brk, c-kit, c-met, c-src, CDK1, CDK2, CDK3, CDK4, CDK5, CDK6, CDK7, CDK8, CDK9, CDK10, cRaf1, CSK, EGFR, ErbB2, ErbB3, ErbB4, Erk, Pak, fes, FGFR1, FGFR2, FGFR3, FGFR4, FGFR5, Fgr, fit-1, Fps, Frk, Fyn, Hck, IGF-1R, INS-R, Jak, KDR, Lck, Lyn, MEK, p38, PDGFR, PIK, PKC, PYK2, ros, tie, tie2, TRK or Zap70).

In embodiments, a compound described herein, or any pharmaceutically acceptable form thereof such as a pharmaceutically acceptable salt thereof, can be used to treat or prevent a disease or disorder that is responsive to modulation (e.g., inhibition) of a tyrosine kinase (e.g., KIT, erb2, PDGFR, EGFR, VEGFR, src, or abl).

In embodiments, a compound described herein, or any pharmaceutically acceptable form thereof such as a pharmaceutically acceptable salt thereof, can be used to treat or prevent a disease or disorder that is responsive to modulation (e.g., inhibition) of a serine/threonine kinase (e.g., mTorC1, mTorC2, ATM, ATR, DNA-PK, or Akt).

In embodiments, a compound described herein modulates (e.g., inhibits) a wild-type form of a kinase (e.g., EGFR). In embodiments, a compound described herein modulates (e.g., inhibits) a mutant form of a kinase (e.g., EGFR).

### Selective Inhibition of Kinases

The term “selective inhibition” or “selectively inhibit” as applied to a biologically active agent refers to the agent's ability to selectively reduce the target signaling activity as compared to off-target signaling activity, via direct or interact interaction with the target.

In some embodiments, a compound described herein, or any pharmaceutically acceptable salt thereof, selectively inhibits a kinase or kinase form over other kinases or other kinase forms. In embodiments, a compound selectively inhibits a mutant kinase form over the wild-type of the same kinase.

In embodiments, a compound described herein, or any pharmaceutically acceptable salt thereof, selectively inhibits a kinase (e.g., EGFR) over other kinases.

In embodiments, a compound described herein, or any pharmaceutically acceptable salt thereof, selectively inhibits a kinase form (e.g., mutant EGFR) over other kinase forms (e.g., wild-type EGFR).

By way of non-limiting example, the ratio of selectivity can be greater than a factor of about 10, greater than a factor of about 20, greater than a factor of about 30, greater than a factor of about 40, greater than a factor of about 50, greater than a factor of about 60, greater than a factor of about 70, greater than a factor of about 80, greater than a factor of about 100, greater than a factor of about 120, or greater than a factor of about 150, where selectivity can be measured by in vitro assays known in the art. Non-limiting examples of assays to measure selectivity include enzymatic assays, cellular proliferation assays, and EGFR phosphorylation assays. In one embodiment, selectivity can be determined by cellular proliferation assays. In another embodiment, selectivity can be determined by EGFR phosphorylation assays. In some embodiments, the mutant EGFR inhibitory activity of a compound as disclosed herein can be less than about 1000 nM, less than about 100 nM, less than about 50 nM, less than about 30 nM, or less than about 10 nM.

In embodiments, the IC₅₀ of a kinase inhibitor compound can be less than about 100 nM, less than about 50 nM, less than about 10 nM, less than about 1 nM, less than about 0.5 nM, or less than about 1 pM.

Determination of IC₅₀ values can be performed according to methods known in the art.

In embodiments, a compound described herein, or any pharmaceutically acceptable form thereof such as a pharmaceutically acceptable salt thereof, can be used to treat or prevent a disease or disorder that is cancer, an inflammatory disorder, a metabolic disorder, vascular disease, or neuronal disease.

Compounds described herein, or any pharmaceutically acceptable form thereof, or any pharmaceutical composition thereof, can be useful for treating diseases and disorders associated with abnormal cell proliferation.

In embodiments, a compound described herein, or a pharmaceutically acceptable form thereof (e.g., a pharmaceutically acceptable salt thereof), or a pharmaceutical composition thereof, can be used to treat cancer.

### Cancer

The compositions and methods provided herein can potentially be useful for the treatment of cancer including tumors such as astrocytic, breast, cervical, colorectal, endometrial, esophageal, gastric, head and neck, hepatocellular, laryngeal, lung, oral, ovarian, prostate and thyroid carcinomas and sarcomas.

In embodiments, a cancer is a cardiac cancer such as sarcoma (angiosarcoma, fibrosarcoma, rhabdomyosarcoma, liposarcoma), myxoma, rhabdomyoma, fibroma, lipoma or teratoma.

In embodiments, a cancer is a lung cancer such as bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, or mesothelioma.

In embodiments, a cancer is a gastrointestinal cancer such as: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma).

In embodiments, a cancer is a cancer of the genitourinary tract such as: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma).

In embodiments a cancer is a liver cancer such as hepatoma (hepatocellular carcinoma), cholangiocarcinoma, hepatoblastoma, angiosarcoma, hepatocellular adenoma, hemangioma.

In embodiments, a cancer is a bone cancer such as: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors.

In embodiments a cancer is a cancer of the central nervous system (CNS) such as: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma).

In embodiments, a cancer is a gynecological cancer such as: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma).

In embodiments, a cancer is a hematological cancer such as: blood (myeloid leukemia (acute and chronic), acute lymphoblastic leukemia, chronic lymphocytic leukemia, myeloproliferative diseases, multiple myeloma, myelodysplasia syndrome), Hodgkin's disease, non-Hodgkin's lymphoma (malignant lymphoma).

In embodiments, a cancer is a skin cancer such as: malignant melanoma, basal cell carcinoma, squamous cell carcinoma, Kaposi's sarcoma, moles dysplastic nevi, lipoma, angioma, dermatofibroma, keloids, psoriasis.

In embodiments, a cancer is a cancer of the adrenal glands such as neuroblastoma.

Thus, the term “cancerous cell” as provided herein, includes a cell afflicted by any one of or related to the above identified conditions.

In embodiments, a cancer is an EGFR-driven cancer (e.g., as described herein). In embodiments, an EGFR-driven cancer is non-small cell lung cancer (NSCLC), squamous cell carcinoma, adenocarcinoma, adenocarcinoma, bronchioloalveolar carcinoma (BAC), BAC with focal invasion, adenocarcinoma with BAC features, and large cell carcinoma; neural tumors, such as glioblastomas; pancreatic cancer; head and neck cancers (e.g., squamous cell carcinoma); breast cancer; colorectal cancer; epithelial cancer, including squamous cell carcinoma; ovarian cancer; prostate cancer; or adenocarcinomas.

In embodiments, a cancer is an EGFR mutant cancer (e.g., as described herein). In embodiments, an EGFR mutant cancer is non-small cell lung cancer (NSCLC), squamous cell carcinoma, adenocarcinoma, adenocarcinoma, bronchioloalveolar carcinoma (BAC), BAC with focal invasion, adenocarcinoma with BAC features, and large cell carcinoma; neural tumors, such as glioblastomas; pancreatic cancer; head and neck cancers (e.g., squamous cell carcinoma); breast cancer; colorectal cancer; epithelial cancer, including squamous cell carcinoma; ovarian cancer; prostate cancer; or adenocarcinomas.

In one embodiment, the compositions and methods provided herein are useful for the treatment of lung cancer and pancreatic cancer, most specifically, non-small cell lung cancer (NSCLC).

In embodiments, a cancer is refractory to TKI therapies (e.g., erlotinib, gefitinib, dacomitinib, afatinib, osimertinib).

### Lung Cancer

In embodiments, a cancer is a lung cancer.

Lung cancer is the most common cause of cancer mortality globally and the second most common cancer in both men and women. About 14% of all new cancers are lung cancers. In the United States (US), there are projected to be 222,500 new cases of lung cancer (116,990 in men and 105,510 in women) and 155,870 deaths from lung cancer (84,590 in men and 71,280 in women) in 2017.

The two major forms of lung cancer are non-small cell lung cancer (NSCLC) and small cell lung cancer. NSCLC is a heterogeneous disease that consists of adenocarcinoma, large-cell carcinoma, and squamous cell carcinoma (sqNSCLC), and comprises approximately 80% to 85% of all lung cancers. Squamous cell carcinoma of the lung accounts for 20% to 30% of NSCLC. Despite advances in early detection and standard treatment, NSCLC is often diagnosed at an advanced stage, has poor prognosis, and is the leading cause of cancer deaths worldwide.

Platinum-based doublet therapy, maintenance chemotherapy, and anti-angiogenic agents in combination with chemotherapy have contributed to improved patient outcomes in advanced NSCLC.

In embodiments, an advanced lung cancer is stage III cancer or stage IV cancer.

In embodiments, an advanced lung cancer is stage III cancer. In embodiments, an advanced lung cancer is stage IV cancer. In embodiments, an advanced lung cancer is locally advanced. In embodiments, an advanced lung cancer is metastatic.

In embodiments, a lung cancer is small cell lung cancer (SCLC).

In embodiments, a lung cancer is non-small cell lung cancer (NSCLC) such as adenocarcinoma, large-cell carcinoma, or squamous cell carcinoma (sqNSCLC). In embodiments, a NSCLC is lung adenocarcinoma. In embodiments, a NSCLC is large cell carcinoma of the lung. In embodiments, a NSCLC is squamous cell carcinoma of the lung (sqNSCLC).

In embodiments, a lung cancer (e.g., NSCLC) is an EGFR-mutant lung cancer (e.g., EGFR-mutant NSCLC). In embodiments, a cancer is NSCLC (e.g., advanced NSCLC) with an identified EGFR mutation.

### EGFR Driven and EGFR Mutant Cancers

The invention features compounds which can be useful for treating patients who have an EGFR-driven cancer, including cancers which are, or have become, refractory to erlotinib, gefitinib, dacomitinib, afatinib, osimertinib, or cancers which bear an EGFR mutation identified herein, by administering a compound of formula (I) to a subject.

EGFR-driven cancers which can be treated using the compositions and method of the invention include, for example, EGFR mutants including one or more deletions, substitutions, or additions in the amino acid or nucleotide sequences of EGFR, or fragments thereof.

An EGFR-driven cancer may result from an EGFR fusion. For example, the N-terminal of EGFR can be linked to various fusion partners such as RAD51. Cancers (e.g., lung cancers) characterized by an EGFR-fusion (e.g., an EGFR-RAD51 fusion) may be particularly suitable for therapy using any compound described herein, or any pharmaceutically acceptable form (e.g., a pharmaceutically acceptable salt) thereof.

Mutations in EGFR can occur in any part of the EGFR sequence. Generally, EGFR mutants arise from mutations in the kinase domain (i.e., exons 18-24 in the EGFR sequence) or in the extracellular domain (i.e., exons 2-16 in the EGFR sequence).

A mutation in EGFR can be an activating mutation, which lead to a ligand-independent activation of TK activity. A mutation in EGFR can also be a resistance mutation, which can confer resistance to TKI therapies such as resistance to one or more of erlotinib, gefitinib, dacomitinib, afatinib, or osimertinib.

For example, mutations typically occur in the kinase domain, including one or more of a point mutation in exon 18 (e.g., L688P, V689M, P694L/S, N700D, L703V, E709K/Q/A/G/V, I715S, L718P, G719C/A/S/R, or S720P/F), a deletion in exon 19 that may or may not include an insertion (e.g., delG719, delE746_E749, delE746_A750, delE746_A750insRP, delE746_A750insQP, delE746_T751, delE746_T751insA/I/V, delE746_T751insVA, delE746_S752, delE746_S752insA/V/D, delE746_P53insLS, delL747_E749, delL747_A750, delL747_A750insP, delL747_T751, delL747_T751insP/S/Q, delL747_T751insPI, delL747_S752, delL747_S752insQ, delL747_P753, delL747_P753insS/Q, delL747_L754insSR, delE749_A750, delE749_A750insRP, delE749_T751, delT751_I759, delT751_I759insS/N, or delS752_I759), a duplication in exon 19 (e.g., K739_I44dupKIPVAI), a point mutation in exon 19 (e.g., L730F, W731Stop, P733L, G735S, V742A, E746V/K, A750P, T751I, S752Y, P753S, A754P, or D761Y), an in-frame insertion in exon 20 (e.g., D761_E762insEAFQ, A767_S768insTLA, V769_D770insY, V769_D770insCV, V769_D770insASV, D770_N771insD/G, D770_N771insNPG, D770_N771insSVQ, P772_H773insN/V, P772_H773insYNP, or V774_C775insHV), a deletion in exon 20 that may or may not include an insertion (e.g., delM766_A767, delM766_A767insAI, delA767_V769, delD770, or delP772_H773insNP), a duplication in exon 20 (e.g., S768_D770dupSVD, A767_V769dupASV, or H773dupH), a point mutation in exon 20 (e.g., D761N, A763V, V765A/M, S768I, V769L/M, S768I, P772R, N771T, H773R/Y/L, V774M, R776G/H/C, G779S/F, T783A, T784F, L792P, L798H/F, T790M, R803W, K806E, or L814P), or a point mutation in exon 21 (e.g., G810S, N826S, L833V, H835L, L838V, A839T, K846R, T847I, H850N, V851I/A, I853T, L858M/R, A859T, L861Q/R, G863D, A864T, E866K, or G873E).

In lung cancer, activation mutants are typical.

In embodiments, a mutation is a resistance mutation. In particular, drug resistance in 50% of lung cancers arises from the T790M point mutation. Other exemplary resistance mutation include point mutations such as: C797X (e.g., C797S, C797G, or C797N); G796X (e.g., G796R, G796S, or G796D); L792X (e.g. L792H, L792F, L792R, or L792Y); G724S; L718X (e.g., L718P, L718Q, or L718V); S768I; or G719A.

In glioblastoma, mutations typically, but not exclusively, occur in the extracellular domain, including EGFR variant I (EGFRvI) lacking the extracellular domain and resembling the v-erbB oncoprotein; EGFRvII lacking 83 amino acids from domain IV; and EGFRvIII lacking amino acids 30-297 from domains I and II, which is the most common amplification and is reported in 30-50% of glioblastomas and 5% of squamous cell carcinoma. Other mutations for glioblastoma include one or more of point mutations in exon 2 (e.g., D46N or G63R), exon 3 (e.g., R108K in domain I), exon 7 (e.g., T263P or A289D/T/V in domain II), exon 8 (e.g., R324L or E330K), exon 15 (e.g., P596L or G598V in domain IV), or exon 21 (L861Q in the kinase domain).

EGFR mutants also include those with a combination of two or more mutations, as described herein. Exemplary combinations include S768I and G719A; S768I and V769L; H773R and W731Stop; R776G and L858R; R776H and L861Q; T790M and L858R; T790M and delE746_A750; R803W and delE746_T751insVA; delL747_E749 and A750P; delL747_S752 and E746V; delL747_S752 and P753S; P772_H773insYNP and H773Y; P772_H773insNP and H773Y; and D770_N771insG and N771T. Other exemplary combinations include any including T790M (e.g., T790M and L858R or T790M and delE746_A750.

EGFR mutants can be either activation mutants or resistant mutants. Activation mutants include those with substitutions that increase drug sensitivity (e.g., G719C/S/A, delE746_A750, or L858R). Resistant mutants include those with substitutions that increase drug resistance (e.g., T790M or any combination including T790M).

In embodiments, an EGFR mutation is a deletion in exon19 (del19). In embodiments, an EGFR mutation is a T790M mutation. In embodiments, an EGFR mutation is a L858R mutation. In embodiments, an EGFR mutation is a C797S mutation. In embodiments, an EGFR-driven cancer (e.g., non-small cell lung cancer) is characterized by at least one of these mutations. In embodiments, an EGFR-driven cancer (e.g., non-small cell lung cancer) is characterized by at least two of these mutations. In embodiments, an EGFR-driven cancer (e.g., non-small cell lung cancer) is characterized by at least three of these mutations.

EGFR-driven cancers include those having any mutant described herein. For example, EGFRvIII is commonly found in glioblastoma and has also been reported in breast, ovarian, prostate, and lung carcinomas. Exemplary EGFR-driven cancers: glioblastoma, lung cancer (e.g., squamous cell carcinoma, non-small cell lung cancer, adenocarcinoma, bronchioloalveolar carcinoma (BAC), BAC with focal invasion, adenocarcinoma with BAC features, and large cell carcinoma), pancreatic cancer, head and neck cancers (e.g., squamous cell carcinoma), breast cancer, colorectal cancer, epithelial cancer (e.g., squamous cell carcinoma), ovarian cancer, and prostate cancer.

In particular, the invention described herein would benefit patient populations having higher risk for TKI-resistant mutations. About 8,000 to 16,000 new cases per year can be estimated based on: incidence of non-small cell lung cancer (about 160,000 new cases in the U.S.), the response to erlotinib in the general population (about 10%, resulting in a sensitive population of 16,000), the presence of activation mutations (10-20% in white and 30-40% in Asian population, resulting in a sensitive population of 16,000-32,000), acquisition of secondary resistance (most if not all patients, resulting in a sensitive population of 16,000-32,000), and percentage of patients carrying the T790M point mutations (about 50%, resulting in a sensitive population of 8,000-16,000). Patients having TKI-resistant mutations include those patients having cancers resistant to one or more of erlotinib, gefitinib, dacomitinib, afatinib, osimertinib, CL-387,785, BIBW 2992 (CAS Reg. No. 439081-18-2), CI-1033, neratinib (HKI-272), MP-412 (AV-412), PF-299804, AEE78, and XL64.

In particular, the inventions relate to treatment of EGFR-driven cancers having the T790M point mutation. Generally, irreversible inhibitors (e.g., CI-1033, neratinib (HKI-272), and PF-299804) are less potent in cell lines having the T790M mutation and do not inhibit T790M at clinically achievable concentrations. Since the ATP Km of T790M and WT are similar, concentrations that inhibit the mutant will inhibit the WT and result in gastrointestinal and cutaneous events.

An EGFR mutant also includes other amino acid and nucleotide sequences of EGFR with one or more deletions, substitutions, or additions, such as point mutations, that retain or increase tyrosine kinase or phosphorylation activity. Where the mutant is a protein or polypeptide, preferable substitutions are conservative substitutions, which are substitutions between amino acids similar in properties such as structural, electric, polar, or hydrophobic properties. For example, the substitution can be conducted between basic amino acids (e.g., Lys, Arg, and His), or between acidic amino acids (e.g., Asp and Glu), or between amino acids having non-charged polar side chains (e.g., Gly, Asn, Gln, Ser, Thr, Tyr, and Cys), or between amino acids having hydrophobic side chains (e.g., Ala, Val, Leu, Ile, Pro, Phe, and Met), or between amino acids having branched side chains (e.g., Thr, Val, Leu, and Ile), or between amino acids having aromatic side chains (e.g., Tyr, Trp, Phe, and His).

Where the mutant is a nucleic acid, the DNA encoding an EGFR mutant protein may comprise a nucleotide sequence capable of hybridizing to a complement sequence of the nucleotide sequence encoding an EGFR mutant, as defined herein, under stringent conditions. As used herein, the stringent conditions include low, medium or high stringent conditions. An example of the stringent conditions includes hybridization at approximately 42-55° C. in approximately 2-6×SSC, followed by wash at approximately 50-65° C. in approximately 0.1-1×SSC containing approximately 0.1-0.2% SDS, where 1×SSC is a solution containing 0.15 M NaCl and 0.015 M Na citrate, pH 7.0. Wash can be performed once or more. In general, stringent conditions may be set at a temperature approximately 5° C. lower than a melting temperature (Tm) of a specific nucleotide sequence at defined ionic strength and pH.

The amino acid and nucleotide sequences of EGFR and DNAs encoding them are available from known databases such as NCBI GenBank (USA), EMBL (Europe), etc. For example, GenBank accession numbers for EGFR [Homo sapiens] include MIM131550, AAI28420, NM_005228, NP_005219.2, and GeneID: 1956.

### EGFR-Selective Inhibition

In some embodiments, a compound described herein, or any pharmaceutically acceptable salt thereof, selectively inhibits EGFR (including any mutant EGFR described herein) over other kinases.

In some embodiments, a compound described herein, or any pharmaceutically acceptable salt thereof, selectively inhibits mutant EGFR (e.g., any mutant EGFR described herein) over wild-type EGFR. In embodiments, a compound described herein selectively inhibits EGFR characterized by a mutation that is: a deletion in exon19 (del19), a T790M mutation, a L858R mutation, and/or a C797S mutation, or any combination thereof. Such inhibitors can be effective in ameliorating diseases and disorders associated with mutant EGFR activity.

By way of non-limiting example, the ratio of selectivity can be greater than a factor of about 10, greater than a factor of about 20, greater than a factor of about 30, greater than a factor of about 40, greater than a factor of about 50, greater than a factor of about 60, greater than a factor of about 70, greater than a factor of about 80, greater than a factor of about 100, greater than a factor of about 120, or greater than a factor of about 150, where selectivity can be measured by in vitro assays known in the art. Non-limiting examples of assays to measure selectivity include enzymatic assays, cellular proliferation assays, and EGFR phosphorylation assays. In one embodiment, selectivity can be determined by cellular proliferation assays. In another embodiment, selectivity can be determined by EGFR phosphorylation assays. In some embodiments, the mutant EGFR inhibitory activity of a compound as disclosed herein can be less than about 1000 nM, less than about 100 nM, less than about 50 nM, less than about 30 nM, or less than about 10 nM.

In embodiments, the IC₅₀ of a subject compound for mutant EGFR inhibition can be less than about 100 nM, less than about 50 nM, less than about 10 nM, less than about 1 nM, less than about 0.5 nM, or less than about 1 pM.

### Characterization of EGFR-Driven Cancers

The compositions and methods of the invention can be used to treat subjects having an EGFR-driven cancer (i.e., cancers characterized by EGFR mutant expression or overexpression). EGFR mutant expression or overexpression can be determined in a diagnostic or prognostic assay by evaluating levels of EGFR mutants in biological sample, or secreted by the cell (e.g., via an immunohistochemistry assay using anti-EGFR antibodies or anti-p-EGFR antibodies; FACS analysis, etc.). Alternatively, or additionally, one can measure levels of EGFR mutant-encoding nucleic acid or mRNA in the cell, e.g., via fluorescent in situ hybridization using a nucleic acid based probe corresponding to an EGFR mutant-encoding nucleic acid or the complement thereof; (FISH; see WO98/45479, published October, 1998), Southern blotting, Northern blotting, or polymerase chain reaction (PCR) techniques, such as real time quantitative PCR (RT-PCR). One can also study EGFR mutant expression by measuring shed antigen in a biological sample, such as serum, e.g., using antibody-based assays (see also, e.g., U.S. Pat. No. 4,933,294, issued Jun. 12, 1990; WO91/05264, published Apr. 18, 1991; U.S. Pat. No. 5,401,638, issued Mar. 28, 1995; and Sias et al., J. Immunol. Methods 132:73 (1990)). Aside from the above assays, various in vivo assays are available to the skilled practitioner. For example, one can expose cells within the body of the mammal to an antibody which is optionally labeled with a detectable label, e.g., a radioactive isotope, and binding of the antibody to cells in the mammal can be evaluated, e.g., by external scanning for radioactivity or by analyzing a biopsy taken from a mammal previously exposed to the antibody.

Examples of biological properties that can be measured in isolated cells include mRNA expression, protein expression, and DNA quantification. Additionally, the DNA of cells isolated by the methods of the invention can be sequenced, or certain sequence characteristics (e.g., polymorphisms and chromosomal abnormalities) can be identified using standard techniques, e.g., FISH or PCR. The chemical components of cells, and other analytes, may also be assayed after isolation. Cells may also be assayed without lysis, e.g., using extracellular or intracellular stains or by other observation, e.g., morphology or growth characteristics in various media.

While any hybridization technique can be used to detect the gene rearrangements, one preferred technique is fluorescent in situ hybridization (FISH). FISH is a cytogenetic technique which can be used to detect and localize the presence or absence of specific DNA or RNA sequences on chromosomes. FISH incorporates the use of fluorescently labeled nucleic acid probes which bind only to those parts of the chromosome with which they show a high degree of sequence similarity. Fluorescence microscopy can be used to find out where the fluorescent probe bound to the chromosome. The basic steps of FISH are outlined below. Exemplary FISH probes include Vysis EGFR SpectrumOrange/CEP SpectrumGreen Probe (Abbott, Downers Grove, Ill.), which hybridizes to band 7p12; and ZytoLight SPEC EGFR/CEN 7 Dual Color Probe (ZytoVision), which hybridizes to the alpha-satellite sequences of the centromere of chromosome 7.

For FISH, a probe is constructed that is long enough to hybridize specifically to its target (and not to similar sequences in the genome), but not too large to impede the hybridization process. Probes are generally labeled with fluorophores, with targets for antibodies, with biotin, or any combination thereof. This can be done in various ways, for example using random priming, nick translation, and PCR using tagged nucleotides.

Generally, a sample or aliquot of a population of cells is used for FISH analysis. For example, in one method of preparation, cells are trypsinized to disperse into single cells, cytospun onto glass slides, and then fixed with paraformaldehyde before storing in 70% ethanol. For preparation of the chromosomes for FISH, the chromosomes are firmly attached to a substrate, usually glass. After preparation, the probe is applied to the chromosome RNA and starts to hybridize. In several wash steps, all unhybridized or partially hybridized probes are washed away. If signal amplification is necessary to exceed the detection threshold of the microscope (which depends on many factors such as probe labeling efficiency, the kind of probe, and the fluorescent dye), fluorescent tagged antibodies or strepavidin are bound to the tag molecules, thus amplifying the fluorescence.

An epifluorescence microscope can be used for observation of the hybridized sequences. The white light of the source lamp is filtered so that only the relevant wavelengths for excitation of the fluorescent molecules arrive onto the sample. Emission of the fluorochromes happens, in general, at larger wavelengths, which allows one to distinguish between excitation and emission light by mean of another optical filter. With a more sophisticated filter set, it is possible to distinguish between several excitation and emission bands, and thus between several fluorochromes, which allows observation of many different probes on the same strand.

Depending on the probes used, FISH can have resolution ranging from huge chromosomes or tiny (˜100 kilobase) sequences. The probes can be quantified simply by counting dots or comparing color.

Allele-specific quantitative real time-PCR may also be used to identify a nucleic acid encoding a mutant EGFR protein (see, for e.g., Diagnostic Innovations DxS BCR-ABL T3151 Mutation Test Kit, and Singer et al., Methods in Molec. Biol. 181:145 (2001)). This technique utilizes Taq DNA polymerase, which is extremely effective at distinguishing between a match and a mismatch at the 3′-end of the primer (when the 3′-base is mismatched, no efficient amplification occurs). Using this technique, the 3′-end of the primer may be designed to specifically hybridize to a nucleic acid sequence that corresponds to a codon that encodes a mutant amino acid in an EGFR mutant, as described herein. In this way, the specific mutated sequences can be selectively amplified in a patient sample. This technique further utilizes a Scorpion probe molecule, which is a bifunctional molecule containing a PCR primer, a fluorophore, and a quencher. The fluorophore in the probe interacts with a quencher, which reduces fluorescence. During a PCR reaction, when the Scorpion probe binds to the amplicon, the fluorophore and quencher in the Scorpion probe become separated, which leads to an increase in fluorescence from the reaction tube. Any of the primers described herein may be used in allele-specific quantitative real time PCR.

A biological sample can be analyzed to detect a mutation in an EGFR gene, or expression levels of an EGFR gene, by methods that are known in the art. For example, methods such as direct nucleic acid sequencing, altered hybridization, aberrant electrophoretic gel migration, binding or cleavage mediated by mismatch binding proteins, single-strand conformational polymorphism (SSCP) analysis, or restriction fragment length polymorphism (RFLP) analysis of PCR products derived from a patient sample can be used to detect a mutation in an EGFR gene; ELISA can be used to measure levels of EGFR polypeptide; and PCR can be used to measure the level of an EGFR nucleic acid molecule.

Any of these techniques may be used to facilitate detection of a mutation in a candidate gene, and each is well known in the art; examples of particular techniques are described, without limitation, in Orita et al. (Proc. Natl. Acad. Sci. USA 86:2766 (1989)) and Sheffield et al. (Proc. Natl. Acad. Sci. USA 86:232 (1989)). Furthermore, expression of the candidate gene in a biological sample (e.g., a biopsy) may be monitored by standard Northern blot analysis or may be aided by PCR (see, e.g., Ausubel et al., Current Protocols in Molecular Biology, John Wiley & Sons, New York, N.Y. (1995); PCR Technology: Principles and Applications for DNA Amplification, H. A. Ehrlich, Ed., Stockton Press, NY; Yap et al., Nucl. Acids. Res. 19:4294 (1991)).

One skilled in the art may identify in a nucleic acid or protein sequence a residue (e.g., amino acid or nucleotide) or codon that corresponds to a residue or codon in wild-type EGFR or EGFR mutants using a number of sequence alignment software programs (e.g., NCBI BLAST website). Such software programs may allow for gaps in the alignment of the compared sequences. Using such software, one skilled in the art may identify a nucleotide, amino acid, or amino acid that corresponding to a specific nucleotide, amino acid, or codon in wild-type EGFR or EGFR mutants.

Levels of EGFR expression (e.g., DNA, mRNA, or protein) in a biological sample can be determined by using any of a number of standard techniques that are well known in the art or described herein. Exemplary biological samples include plasma, blood, sputum, pleural effusion, bronchoalveolar lavage, or biopsy, such as a lung biopsy and lymph node biopsy. For example, EGFR expression in a biological sample (e.g., a blood or tissue sample) from a patient can be monitored by standard northern blot analysis or by quantitative PCR (see, e.g., Ausubel et al., supra; PCR Technology: Principles and Applications for DNA Amplification, H. A. Ehrlich, Ed., Stockton Press, NY; Yap et al., Nucl. Acids. Res. 19:4294 (1991)).

### Combination Therapies

In some embodiments, provided herein are methods for combination therapies in which an agent known to modulate other pathways, or other components of the same pathway, or even overlapping sets of target enzymes are used in combination with a compound as provided herein, or a pharmaceutically acceptable form (e.g., pharmaceutically acceptable salts, hydrates, solvates, isomers, prodrugs, and isotopically labeled derivatives) thereof. In one aspect, such therapy includes, but is not limited to, the combination of the subject compound with chemotherapeutic agents, therapeutic antibodies, and radiation treatment, to provide a synergistic or additive therapeutic effect.

When administered as a combination, the therapeutic agents can be formulated as separate compositions that are administered at the same time or sequentially at different times, or the therapeutic agents can be given as a single composition. The phrase “combination therapy”, in referring to the use of a disclosed compound together with another pharmaceutical agent, means the coadministration of each agent in a substantially simultaneous manner as well as the administration of each agent in a sequential manner, in either case, in a regimen that will provide beneficial effects of the drug combination. Coadministration includes, inter alia, the simultaneous delivery, e.g., in a single tablet, capsule, injection or other dosage form having a fixed ratio of these active agents, as well as the simultaneous delivery in multiple, separate dosage forms for each agent respectively. Thus, the administration of disclosed compounds can be in conjunction with additional therapies known to those skilled in the art in the prevention or treatment of cancer, such as radiation therapy or cytostatic agents, cytotoxic agents, other anti-cancer agents and other drugs to amerliorate symptoms of the cancer or side effects of any of the drugs.

In some embodiments, treatment can be provided in combination with one or more other cancer therapies, include surgery, radiotherapy (e.g., gamma-radiation, neutron beam radiotherapy, electron beam radiotherapy, proton therapy, brachytherapy, and systemic radioactive isotopes, etc.), endocrine therapy, biologic response modifiers (e.g., interferons, interleukins, and tumor necrosis factor (TNF)), hyperthermia, cryotherapy, agents to attenuate any adverse effects (e.g., antiemetics), and other cancer chemotherapeutic drugs. The other agent(s) can be administered using a formulation, route of administration and dosing schedule the same or different from that used with the compounds provided herein.

In embodiments, combination therapy comprises administration of a compound described herein, or any pharmaceutically acceptable form thereof (e.g., any pharmaceutically acceptable salt thereof), or a pharmaceutical composition thereof, in combination with anti-cancer drugs (e.g., antiproliferative agents, anti-angiogenic agents and other chemotherapeutic agents).

In embodiments, combination therapy comprises administration of a compound described herein, or any pharmaceutically acceptable form thereof (e.g., any pharmaceutically acceptable salt thereof), or a pharmaceutical composition thereof, in combination with an amount of an anti-cancer agent (e.g., a chemotherapeutic agent).

### EXAMPLES

### Example 1: Preparation of Compound (33)

Synthesis of 17-(azetidin-2-yl)-20,27-dimethyl-30-oxa-21,22,23,24,25,26,27,28-octazapentacyclopentacosa-2,4(14),5(22),6(23),12(24),13(15),16(18),17(25),19(21)-nonaene (Compound (33))

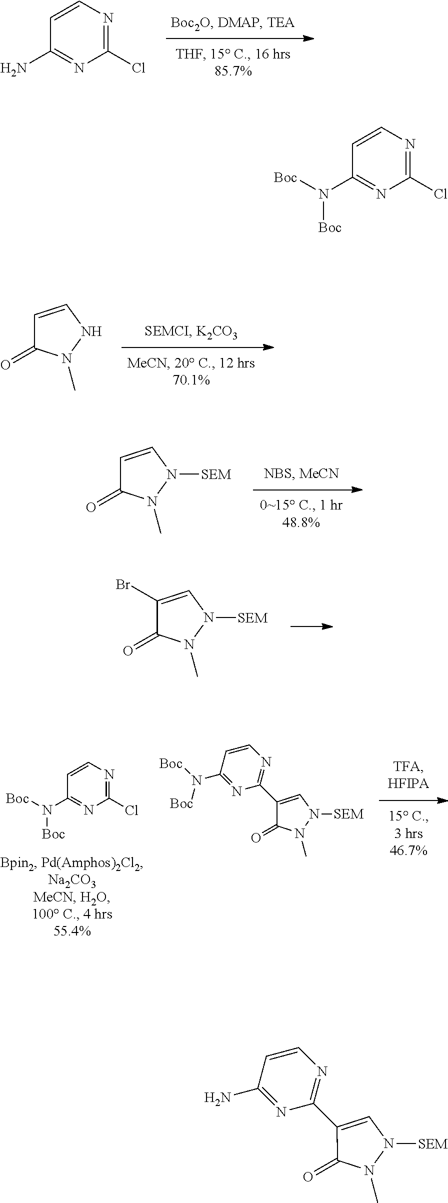

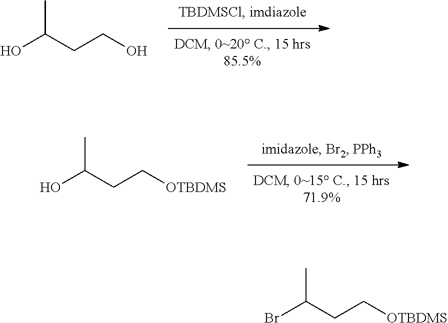

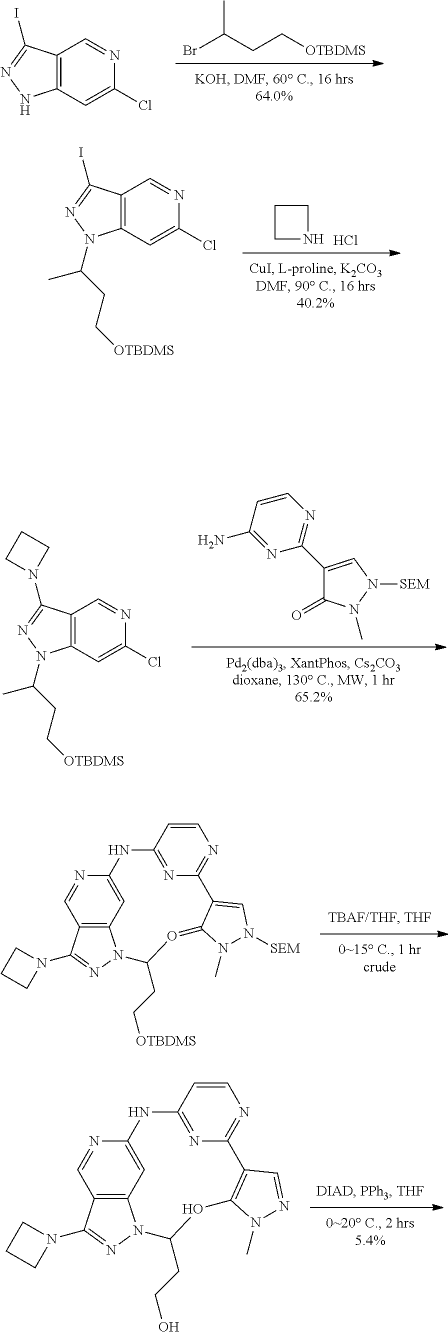

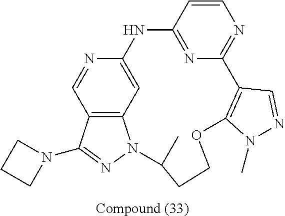

### Step 1: Synthesis of tert-butyl N-tert-butoxycarbonyl-N-(2-chloropyrimidin-4-yl)carbamate

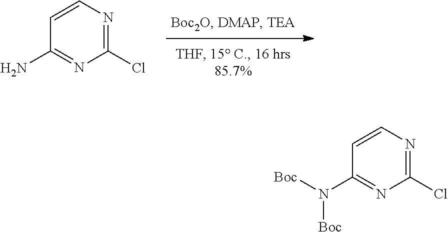 [SMILES: Nc1ccnc(Cl)n1 ; CC(C)(C)OC(=O)N(C(=O)OC(C)(C)C)c1ccnc(Cl)n1]

A mixture of 2-chloropyrimidin-4-amine (2 g, 15.4 mmol, 1 eq) and DMAP (188.6 mg, 1.54 mmol, 0.1 eq) in THF (20 mL) was degassed and purged with nitrogen for 3 times, and TEA (6.25 g, 61.8 mmol, 4 eq) and Boc₂O (10.11 g, 46.3 mmol, 3 eq) were added. The mixture was stirred at 15° C. for 16 hours under nitrogen atmosphere. The reaction mixture was diluted with H₂O (50 mL) and extracted with EtOAc (50 mL*2). The combined organic layers were washed with brine (50 mL), dried over anhydrous Na₂SO₄, filtered and concentrated under reduced pressure. The residue was purified by flash silica gel chromatography (ISCO®; 12 g SepaFlash® Silica Flash Column, petroleum ether/EtOAc with EtOAc from 0-20%, flow rate=20 mL/min) to afford tert-butyl N-tert-butoxycarbonyl-N-(2-chloropyrimidin-4-yl)carbamate (4.5 g, 85.7% yield, 97% purity) as an off white solid.

¹H NMR (400 MHz, DMSO) δ 8.72 (d, J=6.0 Hz, 1H), 7.73 (d, J=6.0 Hz, 1H), 1.52 (s, 18H).

### Step 2: Synthesis of 2-methyl-1-(2-trimethylsilylethoxymethyl)pyrazol-3-one

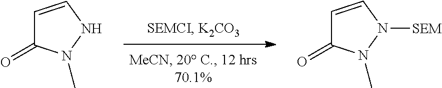

To a solution of 2-methylpyrazol-3-ol (3 g, 30.6 mmol, 1 eq) in MeCN (20 mL) were added SEMCl (11 mL, 62.2 mmol, 2.03 eq) and K₂CO₃ (18.0 g, 0.130 mol, 4.26 eq). The mixture was stirred at 20° C. for 12 hours. The reaction mixture was filtered and concentrated under reduced pressure. The residue was purified by flash silica gel chromatography (ISCO®; 40 g SepaFlash® Silica Flash Column, DCM/MeOH with MeOH from 0-8%, flow rate=40 mL/min) to afford 2-methyl-1-(2-trimethylsilylethoxymethyl)pyrazol-3-one (6.2 g, 70.1% yield, 79% purity) as a white solid.

¹H NMR (400 MHz, chloroform-d) δ 7.30 (d, J=3.6 Hz, 1H), 5.49 (d, J=3.6 Hz, 1H), 4.98 (s, 2H), 3.43-3.47 (m, 5H), 0.87 (t, J=8.0 Hz, 2H), −0.02 (s, 9H).

### Step 3: Synthesis of 4-bromo-2-methyl-1-(2-trimethylsilylethoxymethyl)pyrazol-3-one

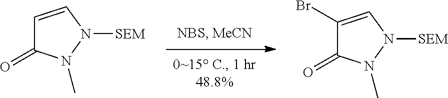 [SMILES: Cn1c(=O)ccn1COCC[Si](C)(C)C ; Cn1c(=O)c(Br)cn1COCC[Si](C)(C)C]

To a mixture of 2-methyl-1-(2-trimethylsilylethoxymethyl)pyrazol-3-one (1.7 g, 7.44 mmol, 1 eq) in MeCN (20 mL) was added NBS (1.99 g, 11.2 mmol, 1.5 eq) at 0° C. under nitrogen and the mixture was stirred at 15° C. for 1 hour under nitrogen atmosphere. The reaction mixture was diluted with saturated Na₂S₂O₃ aqueous solution (50 mL) and extracted with EtOAc (50 mL*2). The combined organic layers were washed with brine (50 mL), dried over anhydrous Na₂SO₄, filtered and concentrated under reduced pressure. The residue was purified by flash silica gel chromatography (ISCO®; 12 g SepaFlash® Silica Flash Column, petroleum ether/EtOAc with EtOAc from 50-100%, flow rate=30 mL/min) to afford 4-bromo-2-methyl-1-(2-trimethylsilylethoxymethyl)pyrazol-3-one (1.2 g, 48.8% yield, 93% purity) as a yellow solid.

¹H NMR (400 MHz, chloroform-d) δ 7.42 (s, 1H), 4.97 (s, 2H), 3.48-3.52 (m, 5H), 0.89 (t, J=8.0 Hz, 2H), −0.01 (s, 9H).

### Step 4: Synthesis of tert-butyl N-tert-butoxycarbonyl-N-[2-[2-methyl-3-oxo-1-(2-trimethylsilylethoxymethyl)pyrazol-4-yl]pyrimidin-4-yl]carbamate

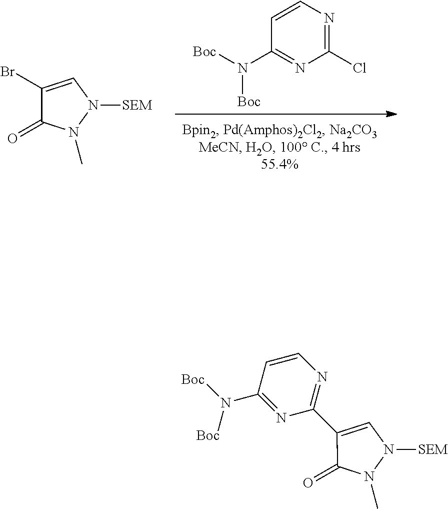 [SMILES: Cn1c(=O)c(Br)cn1COCC[Si](C)(C)C ; CC(C)(C)OC(=O)N(C(=O)OC(C)(C)C)c1ccnc(Cl)n1 ; Cn1c(=O)c(-c2nccc(N(C(=O)OC(C)(C)C)C(=O)OC(C)(C)C)n2)cn1COCC[Si](C)(C)C]

A mixture of tert-butyl N-tert-butoxycarbonyl-N-(2-chloropyrimidin-4-yl)carbamate (2.68 g, 8.14 mmol, 5 eq), 4-bromo-2-methyl-1-(2-trimethylsilylethoxymethyl)pyrazol-3-one (500 mg, 1.63 mmol, 1 eq), 4,4,5,5-tetramethyl-2-(4,4,5,5-tetramethyl-1,3,2-dioxaborolan-2-yl)-1,3,2-dioxaborolane (2.07 g, 8.14 mmol, 5 eq), 4-ditert-butylphosphanyl-N,N-dimethyl-aniline; dichloropalladium (230.4 mg, 0.325 mmol, 0.2 eq) and Na₂CO₃ (862.4 mg, 8.14 mmol, 5 eq) in MeCN (25 mL) and H₂O (2.5 mL) was degassed and purged with nitrogen for 3 times, and then the mixture was stirred at 100° C. for 4 hours under nitrogen atmosphere. The reaction mixture was diluted with H₂O (100 mL) and extracted with EtOAc (150 mL*2). The combined organic layers were washed with brine (200 mL), dried over anhydrous Na₂SO₄, filtered and concentrated under reduced pressure. The residue was purified by flash silica gel chromatography (ISCO®; 40 g SepaFlash® Silica Flash Column, DCM/MeOH with MeOH from 0-10%, flow rate=30 mL/min) to afford tert-butyl N-tert-butoxycarbonyl-N-[2-[2-methyl-3-oxo-1-(2-trimethylsilylethoxymethyl)pyrazol-4-yl]pyrimidin-4-yl]carbamate (2 g, 55.4% yield, 47% purity) as a brown oil.

LCMS [M+H]⁺ m/z: calcd 522.3, found 522.4.

### Step 5: Synthesis of 4-(4-aminopyrimidin-2-yl)-2-methyl-1-(2-trimethylsilylethoxymethyl)pyrazol-3-one

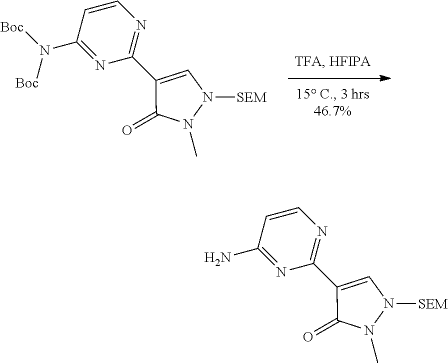 [SMILES: Cn1c(=O)c(-c2nccc(N(C(=O)OC(C)(C)C)C(=O)OC(C)(C)C)n2)cn1COCC[Si](C)(C)C ; Cn1c(=O)c(-c2nccc(N)n2)cn1COCC[Si](C)(C)C]

To a solution of tert-butyl N-tert-butoxycarbonyl-N-[2-[2-methyl-3-oxo-1-(2-trimethylsilylethoxymethyl)pyrazol-4-yl]pyrimidin-4-yl]carbamate (500 mg, 0.958 mmol, 1 eq) in 1,1,1,3,3,3-hexafluoropropan-2-ol (10 mL) was added TFA (1 mL, 13.5 mmol, 14.09 eq). The mixture was stirred at 15° C. for 3 hours. The reaction mixture was diluted with saturated NaHCO₃ aqueous solution (50 mL) and extracted with EtOAc (50 mL*2). The combined organic layers were washed with brine (50 mL), dried over anhydrous Na₂SO₄, filtered and concentrated under reduced pressure. The residue was purified by preparative TLC (silica, DCM/MeOH=10/1, 254 nm) to afford 4-(4-aminopyrimidin-2-yl)-2-methyl-1-(2-trimethylsilylethoxymethyl)pyrazol-3-one (150 mg, 46.7% yield, 96% purity) as a red solid.

¹H NMR (400 MHz, methanol-d₄) δ 8.38 (br s, 1H), 8.02 (br s, 1H), 6.34 (br s, 1H), 5.40 (s, 2H), 3.52-3.62 (m, 5H), 0.92 (t, J=8.0 Hz, 2H), −0.00 (s, 9H).

### Step 6: Synthesis of 4-[tert-butyl(dimethyl)silyl]oxybutan-2-ol TBDMSCl, imidazole

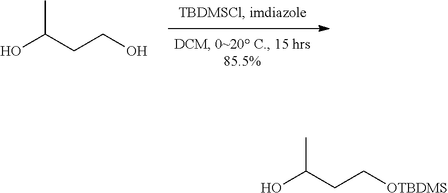

To a solution of butane-1,3-diol (5 g, 55.5 mmol, 1 eq) and imidazole (4.15 g, 61.0 mmol, 1.1 eq) in DCM (80 mL) was added TBDMSCl (8.36 g, 55.5 mmol, 1 eq) at 0° C. The mixture was stirred at 20° C. for 15 hours. The reaction mixture was diluted with H₂O (100 mL) and extracted with DCM (100 mL*2). The combined organic layers were washed with brine (100 mL), dried over anhydrous Na₂SO₄, filtered and concentrated under reduced pressure. The residue was purified by column chromatography (silica, petroleum ether/EtOAc=50/1 to 20/1) to afford 4-[tert-butyl(dimethyl)silyl]oxybutan-2-ol (9.7 g, 85.5% yield) as colorless oil.

¹H NMR (400 MHz, DMSO-d₆) δ 4.30 (d, J=4.4 Hz, 1H), 3.62-3.67 (m, 2H), 1.44-1.55 (m, 2H), 1.50 (d, J=6.4 Hz, 3H), 0.86 (s, 9H), 0.02 (s, 6H).

### Step 7: Synthesis of 3-bromobutoxy-tert-butyl-dimethyl-silane

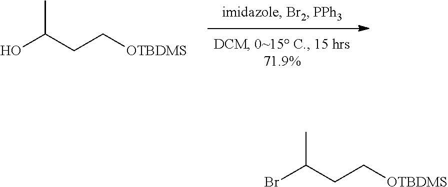

To a mixture of PPh₃ (7.70 g, 29.4 mmol, 1.2 eq) in DCM (80 mL) was added Br₂ (1.5 mL, 29.4 mmol, 1.2 eq) slowly at 0° C. After stirring for 30 minutes, this mixture was added to a mixture of 4-[tert-butyl(dimethyl)silyl]oxybutan-2-ol (5 g, 24.5 mmol, 1 eq) and imidazole (3.33 g, 48.9 mmol, 2 eq) in DCM (50 mL) at 0° C. The mixture was stirred at 15° C. for 15 hours. The reaction mixture was diluted with H₂O (100 mL) and extracted with DCM (100 mL*2). The combined organic layers were washed with brine (150 mL), dried over anhydrous Na₂SO₄, filtered and concentrated under reduced pressure. The residue was purified by column chromatography (silica, petroleum ether/EtOAc=50/1 to 20/1) to afford 3-bromobutoxy-tert-butyl-dimethyl-silane (4.7 g, 71.9% yield) as colorless oil.

¹H NMR (400 MHz, chloroform-d) δ 4.28-4.37 (m, 1H), 3.74-3.80 (m, 2H), 1.99 (q, J=6.4 Hz, 2H), 1.75 (d, J=6.4 Hz, 3H), 0.91 (s, 9H), 0.08 (s, 6H).

### Step 8: Synthesis of tert-butyl-[3-(6-chloro-3-iodo-pyrazolo[4,3-c]pyridin-1-yl)butoxy]-dimethyl-silane

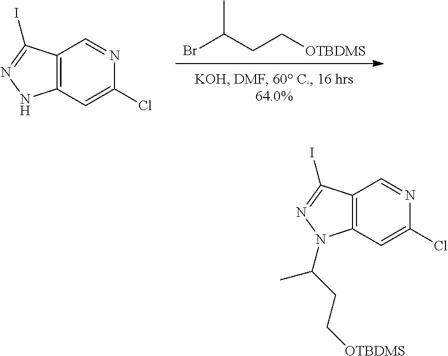 [SMILES: Clc1cc2[nH]nc(I)c2cn1 ; CC(Br)CCO[Si](C)(C)C(C)(C)C ; CC(CCO[Si](C)(C)C(C)(C)C)n1nc(I)c2cnc(Cl)cc21]

A mixture of 6-chloro-3-iodo-1H-pyrazolo[4,3-c]pyridine (2 g, 7.16 mmol, 1 eq), 3-bromobutoxy-tert-butyl-dimethyl-silane (5.74 g, 21.5 mmol, 3 eq) and KOH (1.20 g, 21.5 mmol, 3 eq) in DMF (20 mL) was stirred at 60° C. for 16 hours. The reaction mixture was diluted with H₂O (50 mL) and extracted with EtOAc (100 mL*2). The combined organic layers were washed with brine (100 mL*2), dried over anhydrous Na₂SO₄, filtered and concentrated under reduced pressure. The residue was purified by flash silica gel chromatography (ISCO®; 12 g SepaFlash® Silica Flash Column, petroleum ether/EtOAc with EtOAc from 0-30%, flow rate=20 mL/min) to afford tert-butyl-[3-(6-chloro-3-iodo-pyrazolo[4,3-c]pyridin-1-yl)butoxy]-dimethyl-silane (2.2 g, 64.0% yield, 97% purity) as a white solid.

¹H NMR (400 MHz, chloroform-d) δ 8.56 (s, 1H), 7.36 (s, 1H), 4.83-4.91 (m, 1H), 3.56-3.61 (m, 1H), 3.07-3.13 (m, 1H), 2.17-2.21 (m, 1H), 2.00-2.05 (m, 1H), 1.60 (d, J=6.4 Hz, 3H), 0.88 (s, 9H), −0.03 (s, 3H), −0.07 (s, 3H).

LCMS [M+H]⁺ m/z: calcd 466.1, found 466.0.

The regio-chemistry was confirmed by NOE.

### Step 9: Synthesis of 3-[3-(azetidin-1-yl)-6-chloro-pyrazolo[4,3-c]pyridin-1-yl]butoxy-tert-butyl-dimethyl-silane

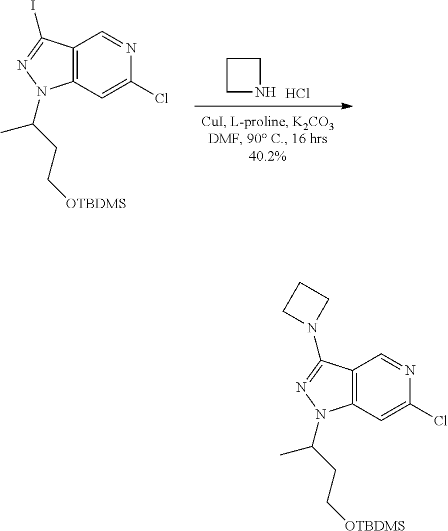

A mixture of tert-butyl-[3-(6-chloro-3-iodo-pyrazolo[4,3-c]pyridin-1-yl)butoxy]-dimethyl-silane (2.2 g, 4.72 mmol, 1 eq), azetidine; hydrochloride (1.77 g, 18.9 mmol, 4 eq), CuI (1.44 g, 7.56 mmol, 1.6 eq), L-proline (978.7 mg, 8.50 mmol, 1.8 eq) and K₂CO₃ (4.57 g, 33.1 mmol, 7 eq) in DMF (30 mL) was degassed and purged with nitrogen for 3 times, and then the mixture was stirred at 90° C. for 16 hours under nitrogen atmosphere. The reaction mixture was diluted with H₂O (100 mL) and extracted with EtOAc (150 mL*2). The combined organic layers were washed with brine (100 mL*2), dried over anhydrous Na₂SO₄, filtered and concentrated under reduced pressure. The residue was purified by flash silica gel chromatography (ISCO®; 12 g SepaFlash® Silica Flash Column, petroleum ether/EtOAc with EtOAc from 0-30%, flow rate=20 mL/min) to afford 3-[3-(azetidin-1-yl)-6-chloro-pyrazolo[4,3-c]pyridin-1-yl]butoxy-tert-butyl-dimethyl-silane (950 mg, 40.2% yield, 79% purity) as yellow solid.

¹H NMR (400 MHz, chloroform-d) δ 8.53 (s, 1H), 7.13 (s, 1H), 4.62-4.65 (m, 1H), 4.18-4.23 (m, 4H), 3.51-3.55 (m, 1H), 3.12-3.18 (m, 1H), 2.49-2.55 (m, 2H), 2.13-2.17 (m, 1H), 1.91-1.95 (m, 1H), 1.50 (d, J=6.8 Hz, 3H), 0.89 (s, 9H), −0.03 (s, 3H), −0.06 (s, 3H).

### Step 10: Synthesis of 4-[4-[[3-(azetidin-1-yl)-1-[3-[tert-butyl(dimethyl)silyl]oxy-1-methyl-propyl]pyrazolo[4,3-c]pyridin-6-yl]amino]pyrimidin-2-yl]-2-methyl-1-(2-trimethylsilylethoxymethyl)pyrazol-3-one

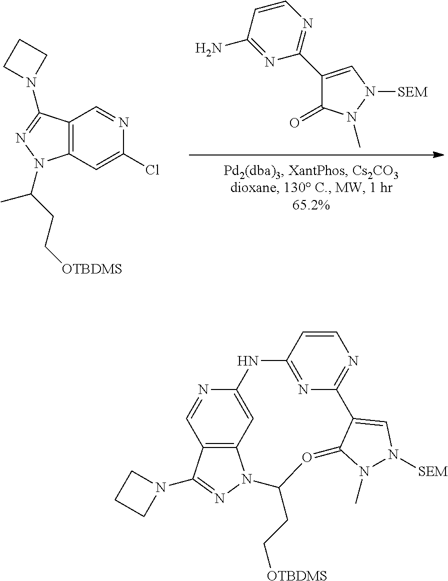

A mixture of 3-[3-(azetidin-1-yl)-6-chloro-pyrazolo[4,3-c]pyridin-1-yl]butoxy-tert-butyl-dimethyl-silane (294.9 mg, 0.747 mmol, 2 eq), 4-(4-aminopyrimidin-2-yl)-2-methyl-1-(2-trimethylsilylethoxymethyl)pyrazol-3-one (120 mg, 0.373 mmol, 1 eq), Pd₂(dba)₃ (68.4 mg, 74.7 μmol, 0.2 eq), Xantphos (43.2 mg, 74.7 μmol, 0.2 eq) and Cs₂CO₃ (364.9 mg, 1.12 mmol, 3 eq) in dioxane (2 mL) was degassed and purged with nitrogen for 3 times, and then the mixture was stirred at 130° C. for 1 hour under nitrogen atmosphere. The reaction mixture was concentrated under reduced pressure and the residue was purified by preparative TLC (silica, DCM/MeOH=10:1, 254 nm) to afford 4-[4-[[3-(azetidin-1-yl)-1-[3-[tert-butyl(dimethyl)silyl]oxy-1-methyl-propyl]pyrazolo[4,3-c]pyridin-6-yl]amino]pyrimidin-2-yl]-2-methyl-1-(2-trimethylsilylethoxymethyl)pyrazol-3-one (180 mg, 65.2% yield, 92% purity) as a yellow solid.

LCMS [M+H]⁺ m/z: calcd 680.4, found 680.6.

### Step 11: Synthesis of 4-[4-[[3-(azetidin-1-yl)-1-(3-hydroxy-1-methyl-propyl)pyrazolo[4,3-c]pyridin-6-yl]amino]pyrimidin-2-yl]-2-methyl-1H-pyrazol-3-one

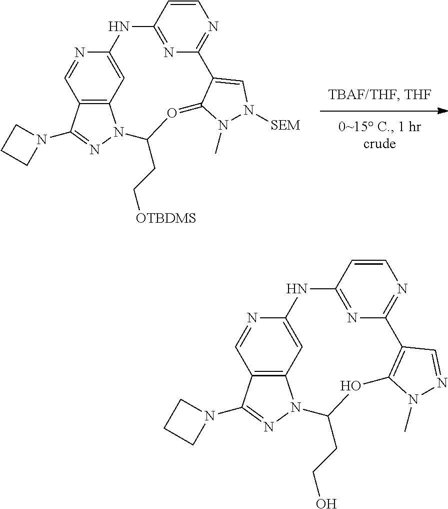

A mixture of 4-[4-[[3-(azetidin-1-yl)-1-[3-[tert-butyl(dimethyl)silyl]oxy-1-methyl-propyl]pyrazolo[4,3-c]pyridin-6-yl]amino]pyrimidin-2-yl]-2-methyl-1-(2-trimethylsilylethoxymethyl)pyrazol-3-one (70 mg, 0.103 mmol, 1 eq) in THF (3 mL) was added 1M TABF/THF (210 μL, 0.210 mmol, 2 eq) at 0° C., and then the mixture was stirred at 15° C. for 1 hour. The reaction mixture was concentrated under reduced pressure to afford 4-[4-[[3-(azetidin-1-yl)-1-(3-hydroxy-1-methyl-propyl)pyrazolo[4,3-c]pyridin-6-yl]amino]pyrimidin-2-yl]-2-methyl-1H-pyrazol-3-one (60 mg, crude) as yellow oil.

### Step 12: Synthesis of 17-(azetidin-1-yl)-20,27-dimethyl-30-oxa-21,22,23,24,25,26,27,28-octazapentacyclopentacosa-2,4(14),5(22),6(23),12(24),13(15),16(18),17(25),19(21)-nonaene (Compound (33))

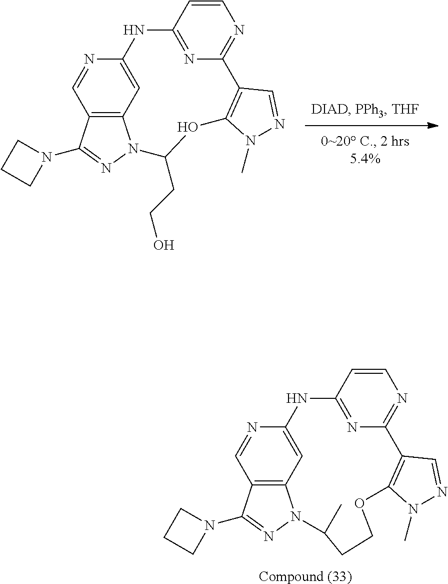

A mixture of 4-[4-[[3-(azetidin-1-yl)-1-(3-hydroxy-1-methyl-propyl)pyrazolo[4,3-c]pyridin-6-yl]amino]pyrimidin-2-yl]-2-methyl-1H-pyrazol-3-one (60.0 mg, 0.138 mmol, 1 eq) and PPh₃ (108.4 mg, 0.414 mmol, 3 eq) in THF (2 mL) was degassed and purged with nitrogen for 3 times, then cooled to 0° C. DIAD (83.6 mg, 0.414 mmol, 3 eq) was added dropwise at 0° C. and the mixture was stirred at 20° C. for 2 hours under nitrogen atmosphere. The reaction mixture was diluted with H₂O (10 mL) and extracted with EtOAc (15 mL*2). The combined organic layers were washed with brine (10 mL), dried over anhydrous Na₂SO₄, filtered and concentrated under reduced pressure. The residue was purified by preparative TLC (silica, DCM/MeOH=10/1, 254 nm) to give a crude product which was purified by preparative HPLC (Welch Xtimate C18 150*25 mm*5 μm; mobile phase: [water (0.05% NH₃—H₂O)-MeCN]; B %: 33%-63%, 7.8 min) to afford 17-(azetidin-1-yl)-20,27-dimethyl-30-oxa-21,22,23,24,25,26,27,28-octazapentacyclopentacosa-2,4(14),5(22),6(23),12(24),13(15),16(18),17(25),19(21)-nonaene (3.3 mg, 5.4% yield, 94% purity) as off white solid.

¹H NMR (400 MHz, chloroform-d) δ 8.87 (s, 1H), 8.47 (s, 1H), 8.33 (d, J=6.4 Hz, 1H), 8.13 (s, 1H), 7.13 (s, 1H), 7.66 (br s, 1H), 6.40 (d, J=6.4 Hz, 1H), 5.01-5.09 (m, 1H), 4.55-4.62 (m, 1H), 4.18-4.28 (m, 4H), 3.80-4.19 (m, 4H), 2.48-2.56 (m, 2H), 2.31-2.35 (m, 1H), 2.18-2.22 (m, 1H), 1.80 (d, J=7.2 Hz, 3H).

LCMS [M+H]⁺ m/z: calcd 418.1, found 418.1.

### Example 2: Preparation of Compound (4)

### Synthesis of 19-(azetidin-1-yl)-11,16,16-trimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaene (compound (4))

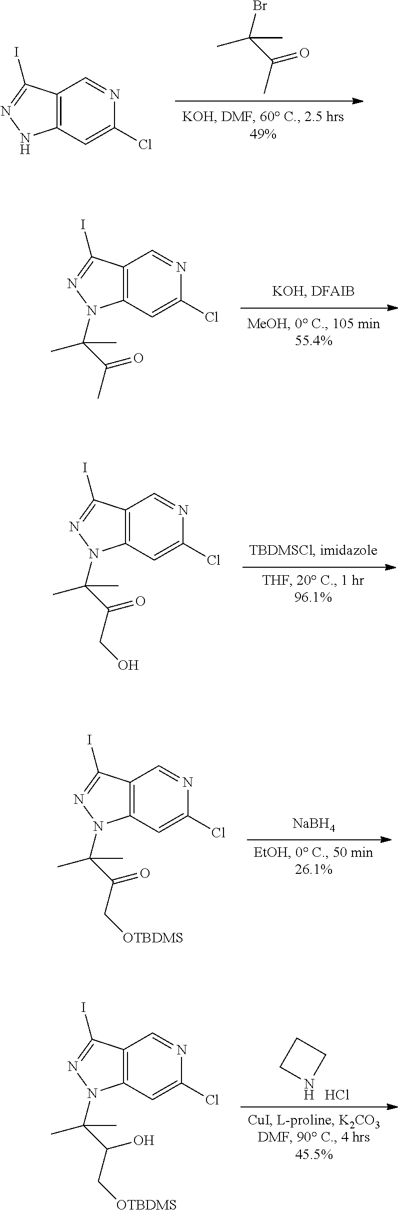

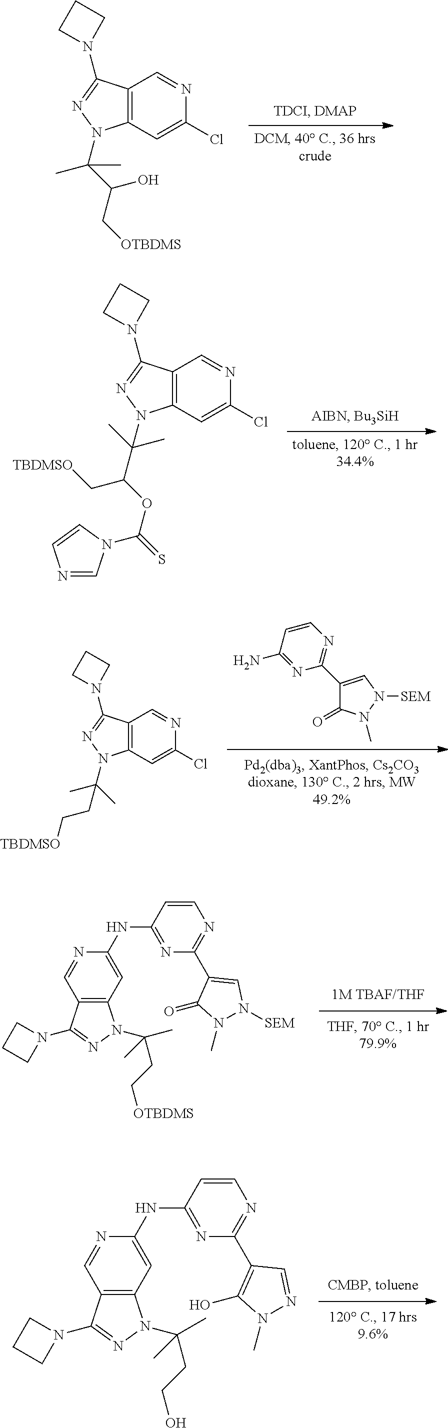

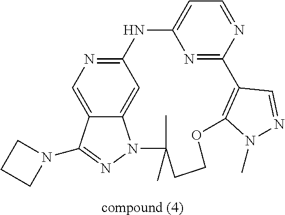

### Step 1: Synthesis of 3-(6-chloro-3-iodo-pyrazolo[4,3-c]pyridin-1-yl)-3-methyl-butan-2-one

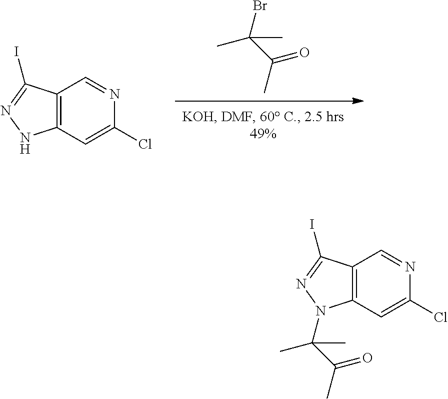

A mixture of 6-chloro-3-iodo-1H-pyrazolo[4,3-c]pyridine (2 g, 7.16 mmol, 1.0 eq), KOH (1.2 g, 21.4 mmol, 3.0 eq) and 3-bromo-3-methyl-butan-2-one (3.5 g, 21.2 mmol, 3.0 eq) in DMF (30.0 mL) was stirred at 60° C. for 2.5 hours. The reaction mixture was partitioned between H₂O (50 mL) and EtOAc (80 mL). The organic phase was separated, washed with brine (80 mL*3), dried over Na₂SO₄, filtered and concentrated under reduced pressure. The residue was purified by column chromatography (SiO₂, petroleum ether/EtOAc=50/1 to 10/1, 254 nm) to afford 3-(6-chloro-3-iodo-pyrazolo[4,3-c]pyridin-1-yl)-3-methyl-butan-2-one (1.3 g, 49.0% yield, 98% purity) as a white solid.

LCMS [M+H]⁺ m/z: calcd 364.0, found 363.8.

¹H NMR (400 MHz, chloroform-d) δ ppm 8.63 (d, J=0.8 Hz, 1H), 7.17 (d, J=0.8 Hz, 1H), 1.96 (s, 3H), 1.87 (s, 6H).

The regio-chemistry was confirmed by NOE.

### Step 2: Synthesis of 3-(6-chloro-3-iodo-pyrazolo[4,3-c]pyridin-1-yl)-1-hydroxy-3-methyl-butan-2-one

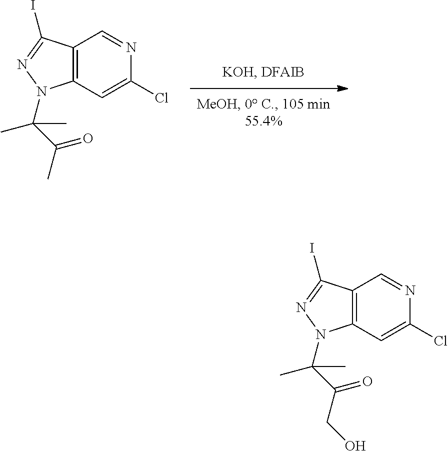 [SMILES: CC(=O)C(C)(C)n1nc(I)c2cnc(Cl)cc21 ; CC(C)(C(=O)CO)n1nc(I)c2cnc(Cl)cc21]

A mixture of 3-(6-chloro-3-iodo-pyrazolo[4,3-c]pyridin-1-yl)-3-methyl-butan-2-one (1.3 g, 3.58 mmol, 1.0 eq) and KOH (1.08 g, 19.31 mmol, 5.4 eq) in MeOH (15.0 mL) was stirred at 0° C. for 15 minutes. Then [phenyl-(2,2,2-trifluoroacetyl)oxy-λ3-iodanyl] 2,2,2-trifluoroacetate (3.12 g, 7.26 mmol, 2.0 eq) was added and the mixture was stirred at 0° C. for 1.5 hours. The reaction mixture was quenched by addition of 5 wt % aqueous solution of H₂SO₄ (20 mL) and stirred at 0° C. for 90 minutes, and extracted with EtOAc (20 mL*3). The combined organic layers were washed with brine (20 mL*3), dried over Na₂SO₄, filtered and concentrated under reduced pressure. The residue was purified by column chromatography (SiO₂, petroleum ether/EtOAc=10/1 to 1/1, 254 nm) to afford 3-(6-chloro-3-iodo-pyrazolo[4,3-c]pyridin-1-yl)-1-hydroxy-3-methyl-butan-2-one (800 mg, 55.4% yield, 94% purity) as a white solid.

LCMS [M+H]⁺ m/z: calcd 380.0, found 379.8.

¹H NMR (400 MHz, chloroform-d) δ ppm 8.63 (d, J=0.8 Hz, 1H), 7.21 (d, J=1.0 Hz, 1H), 4.14 (s, 2H), 2.80 (br s, 1H), 1.96 (s, 6H).

### Step 3: Synthesis of 1-[tert-butyl(dimethyl)silyl]oxy-3-(6-chloro-3-iodo-pyrazolo[4,3-c]pyridin-1-yl)-3-methyl-butan-2-one

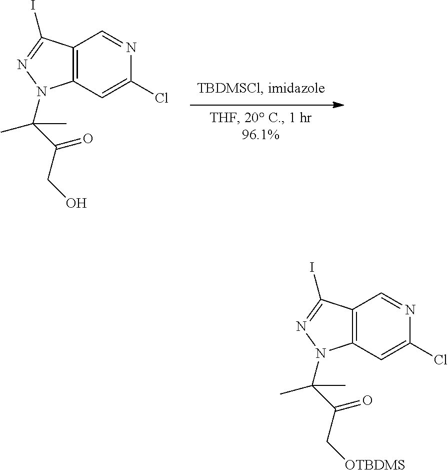 [SMILES: CC(C)(C(=O)CO)n1nc(I)c2cnc(Cl)cc21 ; "THF, 20° C., 1 hr96.1%" ; CC(C)(C(=O)CO[Si](C)(C)C(C)(C)C)n1nc(I)c2cnc(Cl)cc21]

A mixture of 3-(6-chloro-3-iodo-pyrazolo[4,3-c]pyridin-1-yl)-1-hydroxy-3-methyl-butan-2-one (800 mg, 2.11 mmol, 1.0 eq), TBDMSCl (660 mg, 4.38 mmol, 2.1 eq) and imidazole (400 mg, 5.88 mmol, 2.8 eq) in THF (20.0 mL) was stirred at 20° C. for 1 hour. The reaction mixture was partitioned between EtOAc (50 mL) and H₂O (30 mL). The organic phase was separated, washed with brine (30 mL*3), dried over Na₂SO₄, filtered and concentrated under reduced pressure. The residue was purified by column chromatography (SiO₂, petroleum ether/EtOAc=80/1 to 10/1, 254 nm) to afford 1-[tert-butyl(dimethyl)silyl]oxy-3-(6-chloro-3-iodo-pyrazolo[4,3-c]pyridin-1-yl)-3-methyl-butan-2-one (1 g, 96.1% yield, 100% purity) as a colorless oil.

LCMS [M+H]⁺ m/z: calcd 494.0, found 493.9.

¹H NMR (400 MHz, chloroform-d) δ ppm 8.61 (d, J=0.8 Hz, 1H), 7.23 (d, J=0.8 Hz, 1H), 4.24 (s, 2H), 1.91 (s, 6H), 0.80 (s, 9H), −0.07 (s, 6H).

### Step 4: Synthesis of 1-[tert-butyl(dimethyl)silyl]oxy-3-(6-chloro-3-iodo-pyrazolo[4,3-c]pyridin-1-yl)-3-methyl-butan-2-ol

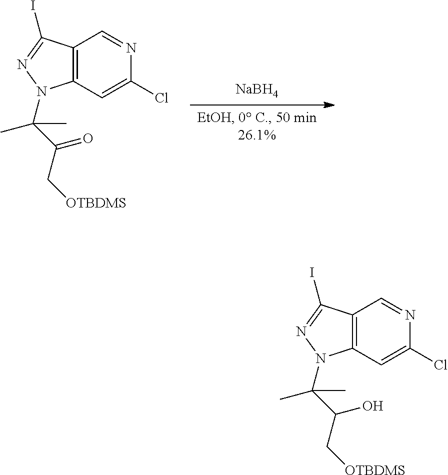 [SMILES: CC(C)(C(=O)CO[Si](C)(C)C(C)(C)C)n1nc(I)c2cnc(Cl)cc21 ; "EtOH, 0° C., 50 min26.1%" ; "NaBH4" ; CC(C)(C(O)CO[Si](C)(C)C(C)(C)C)n1nc(I)c2cnc(Cl)cc21]

A mixture of 1-[tert-butyl(dimethyl)silyl]oxy-3-(6-chloro-3-iodo-pyrazolo[4,3-c]pyridin-1-yl)-3-methyl-butan-2-one (1 g, 2.02 mmol, 1.0 eq) and NaBH₄ (160 mg, 4.23 mmol, 2.1 eq) in EtOH (15.0 mL) was stirred at 0° C. for 50 minutes. The reaction mixture was quenched by H₂O (25 mL), and extracted with EtOAc (20 mL*3). The combined organic layers were washed with brine (30 mL), dried over Na₂SO₄, filtered and concentrated under reduced pressure. The residue was purified by column chromatography (SiO₂, petroleum ether/EtOAc=50/1 to 3/1, 254 nm) to afford 1-[tert-butyl(dimethyl)silyl]oxy-3-(6-chloro-3-iodo-pyrazolo[4,3-c]pyridin-1-yl)-3-methyl-butan-2-ol (270 mg, 26.1% yield, 97% purity) as a colorless oil.

LCMS [M+H]⁺ m/z: calcd 496.1, found 495.9.

¹H NMR (400 MHz, chloroform-d) δ ppm 8.55 (s, 1H), 7.72 (s, 1H), 4.02-3.95 (m, 1H), 3.65 (dd, J=3.8, 10.2 Hz, 1H), 3.35 (dd, J=7.9, 9.6 Hz, 1H), 2.99 (br d, J=3.0 Hz, 1H), 1.83 (s, 6H), 0.84 (d, J=1.1 Hz, 9H), 0.01 (d, J=7.1 Hz, 6H).

### Step 5: Synthesis of 3-[3-(azetidin-1-yl)-6-chloro-pyrazolo[4,3-c]pyridin-1-yl]-1-[tert-butyl(dimethyl)silyl]oxy-3-methyl-butan-2-ol

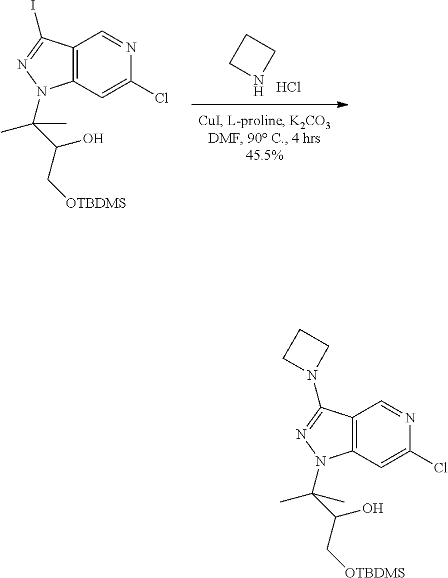

1-[tert-butyl(dimethyl)silyl]oxy-3-(6-chloro-3-iodo-pyrazolo[4,3-c]pyridin-1-yl)-3-methyl-butan-2-ol (270 mg, 0.54 mmol, 1.0 eq), azetidine; hydrochloride (160 mg, 1.71 mmol, 3.1 eq), L-proline (100 mg, 0.87 mmol, 1.6 eq), K₂CO₃ (400 mg, 2.89 mmol, 5.3 eq) and CuI (155 mg, 0.81 mmol, 1.5 eq) in DMF (10.0 mL) was de-gassed and then heated to 90° C. for 4 hours under nitrogen. The reaction was cooled to room temperature, filtered, and extracted with EtOAc (20 mL*3). The combined organic phases were washed with brine (30 mL*3), dried over anhydrous Na₂SO₄, filtered, and concentrated in vacuo. The residue was purified by preparative TLC (SiO₂, petroleum ether/EtOAc=3/1, 254 nm) to afford 3-[3-(azetidin-1-yl)-6-chloro-pyrazolo[4,3-c]pyridin-1-yl]-1-[tert-butyl(dimethyl)silyl]oxy-3-methyl-butan-2-ol (130 mg, 45.5% yield, 81% purity) as a yellow oil.

LCMS [M+H]⁺ m/z: calcd 425.2, found 425.1.

### Step 6: Synthesis of O-[2-[3-(azetidin-1-yl)-6-chloro-pyrazolo[4,3-c]pyridin-1-yl]-1-[[tert-butyl(dimethyl)silyl]oxymethyl]-2-methyl-propyl] imidazole-1-carbothioate

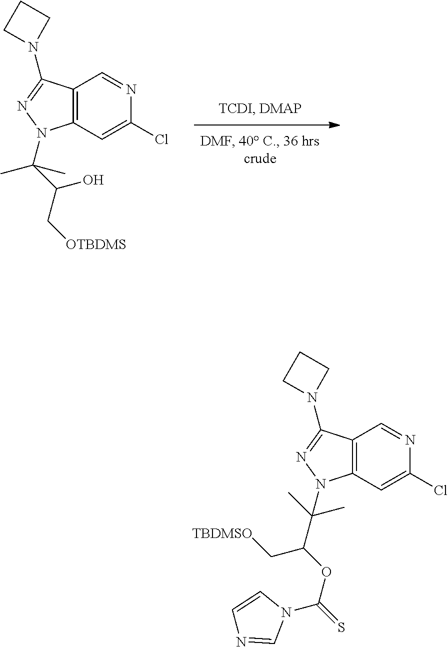 [SMILES: CC(C)(C(O)CO[Si](C)(C)C(C)(C)C)n1nc(N2CCC2)c2cnc(Cl)cc21 ; CC(C)(C(CO[Si](C)(C)C(C)(C)C)OC(=S)n1ccnc1)n1nc(N2CCC2)c2cnc(Cl)cc21]

A mixture of 3-[3-(azetidin-1-yl)-6-chloro-pyrazolo[4,3-c]pyridin-1-yl]-1-[tert-butyl(dimethyl)silyl]oxy-3-methyl-butan-2-ol (130 mg, 0.306 mmol, 1.0 eq), TCDI (130 mg, 0.729 mmol, 2.4 eq) and DMAP (40 mg, 0.327 umol, 1.1 eq) in DCM (10.0 mL) was stirred at 40° C. for 36 hours under nitrogen. The reaction mixture was partitioned between DCM (20 mL) and H₂O (20 mL). The organic phase was separated, washed with brine (20 mL*3), dried over Na₂SO₄, filtered and concentrated under reduced pressure to give O-[2-[3-(azetidin-1-yl)-6-chloro-pyrazolo[4,3-c]pyridin-1-yl]-1-[[tert-butyl(dimethyl)silyl]oxymethyl]-2-methyl-propyl]imidazole-1-carbothioate (500 mg, crude) as a yellow solid.

LCMS [M+H]⁺ m/z: calcd 535.2, found 535.1.

### Step 7: Synthesis of [3-[3-(azetidin-1-yl)-6-chloro-pyrazolo[4,3-c]pyridin-1-yl]-3-methyl-butoxy]-tert-butyl-dimethyl-silane

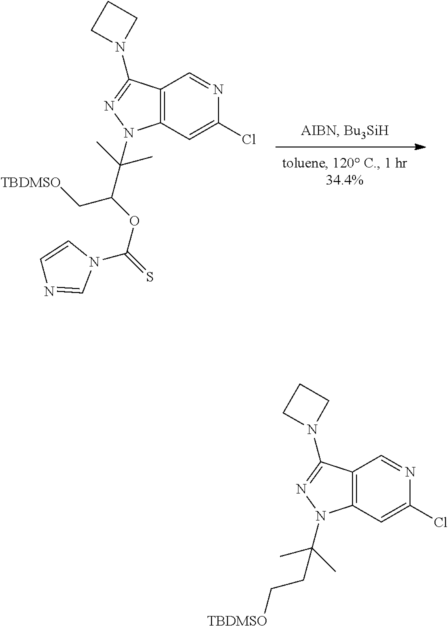 [SMILES: CC(C)(C(CO[Si](C)(C)C(C)(C)C)OC(=S)n1ccnc1)n1nc(N2CCC2)c2cnc(Cl)cc21 ; CC(C)(CCO[Si](C)(C)C(C)(C)C)n1nc(N2CCC2)c2cnc(Cl)cc21]

A mixture of 0-[2-[3-(azetidin-1-yl)-6-chloro-pyrazolo[4,3-c]pyridin-1-yl]-1-[[tert-butyl(dimethyl)silyl]oxymethyl]-2-methyl-propyl] imidazole-1-carbothioate (500 mg, 0.934 mmol, 1.0 eq), AIBN (160 mg, 0.974 mmol, 1.0 eq) and tributylstannane (1 mL, 3.78 mmol, 4.1 eq) in toluene (8.0 mL) was stirred at 120° C. for 1 hour under nitrogen. The reaction mixture was quenched by addition H₂O (15 mL), and extracted with EtOAc (20 mL*3). The combined organic layers were washed with brine (30 mL*3), dried over Na₂SO₄, filtered and concentrated under reduced pressure. The residue was purified by preparative TLC (SiO₂, petroleum ether/EtOAc=5/1, 254 nm) to afford [3-[3-(azetidin-1-yl)-6-chloro-pyrazolo[4,3-c]pyridin-1-yl]-3-methyl-butoxy]-tert-butyl-dimethyl-silane (140 mg, 34.4% yield, 94% purity) as a yellow oil.

LCMS [M+H]⁺ m/z: calcd 409.2, found 409.1.

¹H NMR (400 MHz, chloroform-d) δ ppm 8.53 (s, 1H), 7.35 (s, 1H), 4.18 (t, J=7.4 Hz, 4H), 3.49 (t, J=6.8 Hz, 2H), 2.50 (quin, J=7.4 Hz, 2H), 2.19 (t, J=6.8 Hz, 2H), 1.71 (s, 6H), 0.84 (s, 9H), −0.04 (s, 6H).

### Step 8: Synthesis of 4-[4-[[3-(azetidin-1-yl)-1-[3-[tert-butyl(dimethyl)silyl]oxy-1,1-dimethyl-propyl]pyrazolo[4,3-c]pyridin-6-yl]amino]pyrimidin-2-yl]-2-methyl-1-(2-trimethylsilylethoxymethyl)pyrazol-3-one

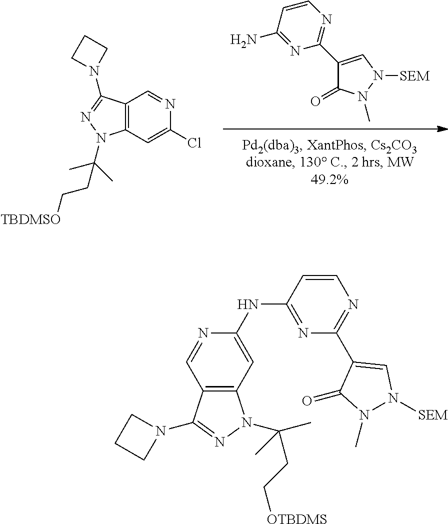 [SMILES: CC(C)(CCO[Si](C)(C)C(C)(C)C)n1nc(N2CCC2)c2cnc(Cl)cc21 ; Cn1c(=O)c(-c2nccc(N)n2)cn1COCC[Si](C)(C)C ; Cn1c(=O)c(-c2nccc(Nc3cc4c(cn3)c(N3CCC3)nn4C(C)(C)CCO[Si](C)(C)C(C)(C)C)n2)cn1COCC[Si](C)(C)C]

[3-[3-(azetidin-1-yl)-6-chloro-pyrazolo[4,3-c]pyridin-1-yl]-3-methyl-butoxy]-tert-butyl-dimethyl-silane (140 mg, 0.342 mmol, 1.0 eq), 4-(4-aminopyrimidin-2-yl)-2-methyl-1-(2-trimethylsilylethoxymethyl)pyrazol-3-one (110 mg, 0.342 mmol, 1.0 eq), XantPhos (20 mg, 0.035 mmol, 0.1 eq), Cs₂CO₃ (230 mg, 0.706 mmol, 2.1 eq) and Pd₂(dba)₃ (33 mg, 0.036 mmol, 0.1 eq) were taken up into a microwave tube in dioxane (5.0 mL). The sealed tube was heated at 130° C. for 2 hours under microwave under nitrogen. The reaction mixture was concentrated under reduced pressure and the residue was purified by preparative TLC (SiO₂, DCM/MeOH=10/1, 254 nm) to afford 4-[4-[[3-(azetidin-1-yl)-1-[3-[tert-butyl(dimethyl)silyl]oxy-1,1-dimethyl-propyl]pyrazolo[4,3-c]pyridin-6-yl]amino]pyrimidin-2-yl]-2-methyl-1-(2-trimethylsilylethoxymethyl)pyrazol-3-one (160 mg, 49.2% yield, 73% purity) as a yellow oil.

LCMS [M+H]⁺ m/z: calcd 694.4, found 694.3.

### Step 9: Synthesis of 4-[4-[[3-(azetidin-1-yl)-1-(3-hydroxy-1,]-dimethyl-propyl)pyrazolo[4,3-c]pyridin-6-yl]amino]pyrimidin-2-yl]-2-methyl-pyrazol-3-ol

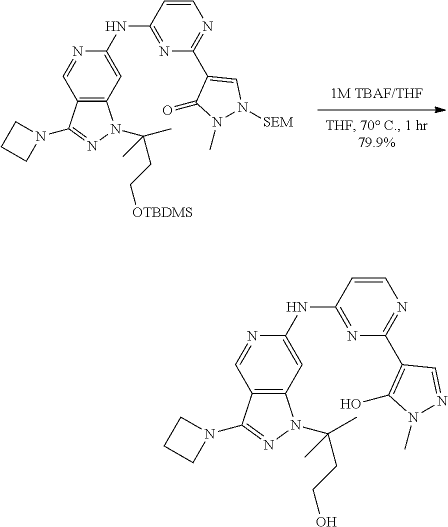 [SMILES: Cn1c(=O)c(-c2nccc(Nc3cc4c(cn3)c(N3CCC3)nn4C(C)(C)CCO[Si](C)(C)C(C)(C)C)n2)cn1COCC[Si](C)(C)C ; Cn1ncc(-c2nccc(Nc3cc4c(cn3)c(N3CCC3)nn4C(C)(C)CCO)n2)c1O]

A mixture of 4-[4-[[3-(azetidin-1-yl)-1-[3-[tert-butyl(dimethyl)silyl]oxy-1,1-dimethyl-propyl]pyrazolo[4,3-c]pyridin-6-yl]amino]pyrimidin-2-yl]-2-methyl-1-(2-trimethylsilylethoxymethyl)pyrazol-3-one (160 mg, 0.230 mmol, 1.0 eq) and 1M TBAF (0.7 mL, 0.7 mmol, 3.0 eq) in THF (10.0 mL) was stirred at 70° C. for 1 hour. The reaction mixture was concentrated under reduced pressure and the residue was purified by flash chromatography (Biotage®; Column: SepaFlash® Sphercial C18, 40 g, 40-60 μm, 120 Å 40 g, 40-60 μm, 120 Å, SepaFlash® Spherical C18 Column, MeCN/water (0.05% NH₃—H₂O) with MeCN from 0-43%, 30 mL/min, 254 nm) to afford 4-[4-[[3-(azetidin-1-yl)-1-(3-hydroxy-1,1-dimethyl-propyl)pyrazolo[4,3-c]pyridin-6-yl]amino]pyrimidin-2-yl]-2-methyl-pyrazol-3-ol (90 mg, 79.9% yield, 92% purity) as a yellow solid.

LCMS [M+H]⁺ m/z: calcd 450.2, found 450.1.

¹H NMR (400 MHz, methanol-d₄) δ ppm 8.63 (br s, 1H), 8.01 (br s, 1H), 7.82 (s, 2H), 5.99 (s, 1H), 4.20 (t, J=7.4 Hz, 4H), 3.49 (s, 3H), 3.48-3.44 (m, 2H), 2.53-2.46 (m, 2H), 2.30 (t, J=7.4 Hz, 2H), 1.76 (s, 6H).

### Step 10: Synthesis of 19-(azetidin-1-yl)-11,16,16-trimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaene

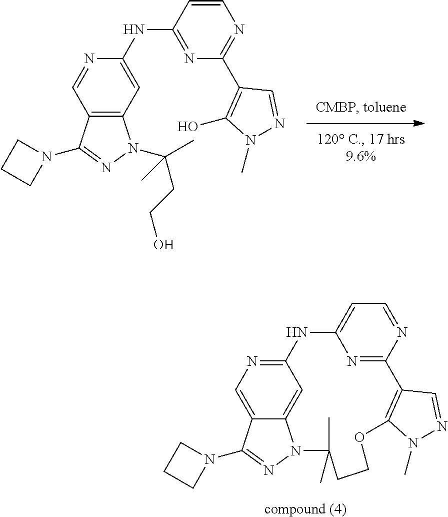

A mixture of 4-[4-[[3-(azetidin-1-yl)-1-(3-hydroxy-1,1-dimethyl-propyl)pyrazolo[4,3-c]pyridin-6-yl]amino]pyrimidin-2-yl]-2-methyl-pyrazol-3-ol (90 mg, 0.20 mmol, 1.0 eq) and 2-(tributyl-X⁵-phosphanylidene)acetonitrile (270 mg, 1.12 mmol, 5.6 eq) in toluene (20.0 mL) was stirred at 120° C. for 17 hours under nitrogen. The reaction mixture was concentrated under reduced pressure. The residue was purified by preparative TLC (SiO₂, DCM:MeOH=10:1, 254 nm) to afford a crude product which was further purified by preparative HPLC (Column: Waters Xbridge 150*25 mm*5 μm; Mobile phase: [water (0.05% NH₃—H₂O+10 mM NH₄HCO₃)-ACN]; B %: 34-64%, 9.5 min, Column Temp.: 30° C.) to afford desired product which is impure. This product was purified by preparative HPLC (Column: Waters Xbridge 150*25 mm*5 μm; Mobile phase: [water (0.05% NH₃H₂O+10 mM NH₄HCO₃)-ACN]; B %: 36-66%, 7.8 min, Column Temp.: 30° C.) to afford 19-(azetidin-1-yl)-11,16,16-trimethyl-13-oxa-2,6,10,11,17,18,22,25-octazapentacyclo[15.5.2.1³⁷.0^{8,12}.0^{20,24}]pentacosa-1(22),3,5,7(25),8(12),9,18,20,23-nonaene (8.4 mg, 9.6% yield, 99% purity) as a white solid.

LCMS [M+H]⁺ m/z: calcd 432.2, found 432.1.

¹H NMR (400 MHz, methanol-d₄) δ ppm 8.85 (s, 1H), 8.50 (s, 1H), 8.24 (d, J=6.0 Hz, 1H), 7.94 (s, 1H), 6.71 (d, J=6.0 Hz, 1H), 4.64-4.61 (m, 2H), 4.18 (t, J=7.4 Hz, 4H), 3.82 (s, 3H), 2.67 (t, J=7.0 Hz, 2H), 2.49 (quin, J=7.4 Hz, 2H), 1.76 (s, 6H).

### Example 3: Preparation of Compound (12)

### Synthesis of 2-[2-(11,16-dimethyl-13-oxa-2,6,10,11,17,18,22,25-octazapentacyclo[15.5.2.13,7.08,12.020,24]pentacosa-1(22),3,5,7(25),8(12),9,18,20,23-nonaen-19-yl)pyrrol-1-yl]-N,N-dimethyl-ethanamine (compound (12))

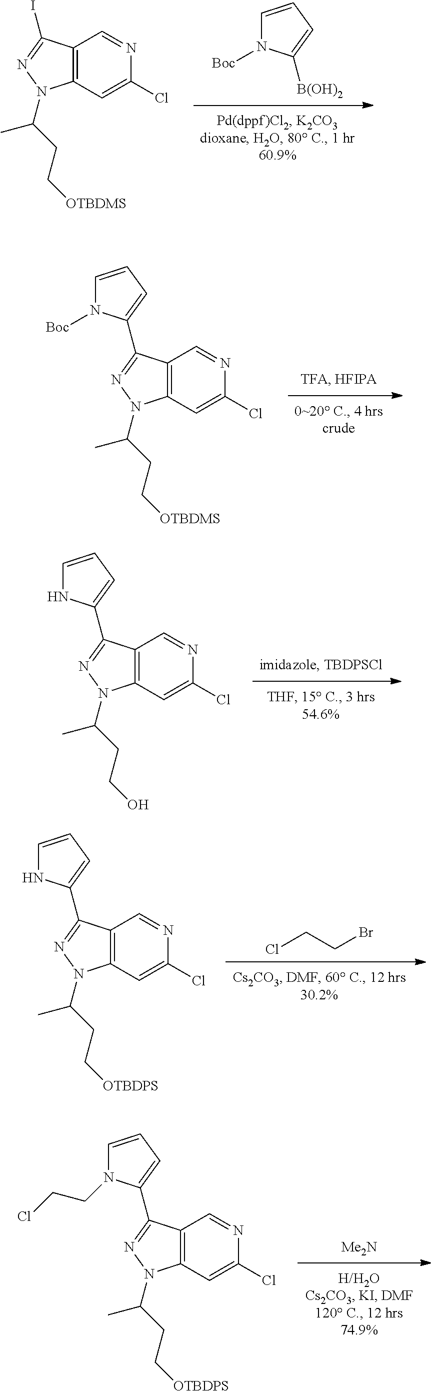

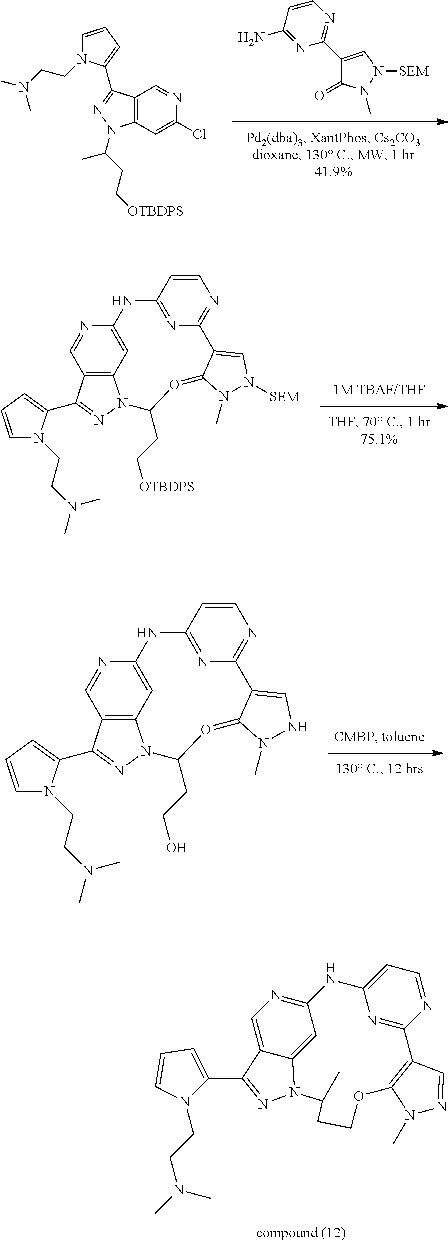

### Step 1: Synthesis of tert-butyl 2-[1-[3-[tert-butyl(dimethyl)silyl]oxy-1-methyl-propyl]-6-chloro-pyrazolo[4,3-c]pyridin-3-yl]pyrrole-1-carboxylate

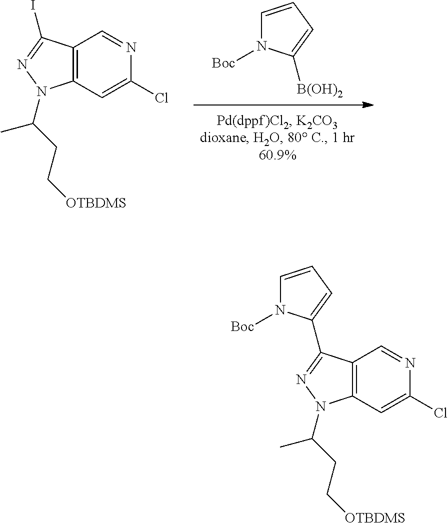 [SMILES: CC(CCO[Si](C)(C)C(C)(C)C)n1nc(I)c2cnc(Cl)cc21 ; CC(C)(C)OC(=O)n1cccc1B(O)O ; CC(CCO[Si](C)(C)C(C)(C)C)n1nc(-c2cccn2C(=O)OC(C)(C)C)c2cnc(Cl)cc21]

A solution of tert-butyl-[3-(6-chloro-3-iodo-pyrazolo[4,3-c]pyridin-1-yl)butoxy]-dimethyl-silane (1.5 g, 3.22 mmol, 1.0 eq), (1-tert-butoxycarbonylpyrrol-2-yl)boronic acid (750 mg, 3.55 mmol, 1.10 eq), Pd(dppf)Cl₂ (500 mg, 0.68 mmol, 0.2 eq) and K₂CO₃ (1.3 g, 9.41 mmol, 2.92 eq) in dioxane (24.0 mL) and H₂O (4.0 mL) was disturbed with nitrogen for 3 times and then stirred at 80° C. for 1 hour. The reaction mixture was diluted with water (30.0 mL) and extracted with EtOAc (50.0 mL*3). The combined organic layers were washed with brine (50.0 mL), dried over Na₂SO₄ and filtered. The filtrate was concentrated and the residue was purified by flash silica gel chromatography (ISCO®; 80 g SepaFlash® Silica Flash Column, petroleum ether/EtOAc with EtOAc from 0-10%, flow=60 mL/min) to affod tert-butyl 2-[1-[3-[tert-butyl(dimethyl)silyl]oxy-1-methyl-propyl]-6-chloro-pyrazolo[4,3-c]pyridin-3-yl]pyrrole-1-carboxylate (1.1 g, 60.9% yield, 90% purity) was obtained as colourless oil.

LCMS [M+H]⁺ m/z: calcd 505.2, found 505.1.

¹H NMR (400 MHz, CDCl₃) δ 8.63 (s, 1H), 7.49 (dd, J=1.8, 3.2 Hz, 1H), 7.39 (s, 1H), 6.51 (dd, J=1.8, 3.3 Hz, 1H), 6.34 (t, J=3.6 Hz, 1H), 4.95-4.83 (m, 1H), 3.64-3.55 (m, 1H), 3.22 (dt, J=3.6, 10.0 Hz, 1H), 3.29-3.17 (m, 1H), 2.31-2.20 (m, 1H), 2.04 (tdd, J=4.8, 9.6, 14.1 Hz, 1H), 1.63 (d, J=6.8 Hz, 3H), 1.32-1.23 (m, 9H), 0.94-0.86 (m, 9H), −0.03 (d, J=15.2 Hz, 6H).

### Step 2: Synthesis of 3-[6-chloro-3-(1H-pyrrol-2-yl)pyrazolo[4,3-c]pyridin-1-yl]butan-1-ol

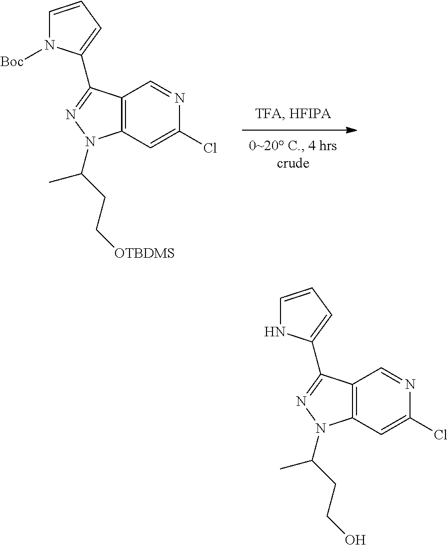 [SMILES: CC(CCO[Si](C)(C)C(C)(C)C)n1nc(-c2cccn2C(=O)OC(C)(C)C)c2cnc(Cl)cc21 ; CC(CCO)n1nc(-c2ccc[nH]2)c2cnc(Cl)cc21]

To a solution of tert-butyl 2-[1-[3-[tert-butyl(dimethyl)silyl]oxy-1-methyl-propyl]-6-chloro-pyrazolo[4,3-c]pyridin-3-yl]pyrrole-1-carboxylate (1.1 g, 2.18 mmol, 1.0 eq) in HFIPA (15.0 mL) was added TFA (1.5 mL, 20.3 mmol, 9.30 eq) at 0° C. The mixture was then stirred at 20° C. for 4 hours. The reaction mixture was quenched with saturated NaHCO₃ aqueous solution to pH=8 and extracted with DCM (50.0 mL*3). The combined organic layers were washed with brine (50.0 mL), dried over Na₂SO₄ and filtered. The filtrate was concentrated to give 3-[6-chloro-3-(1H-pyrrol-2-yl)pyrazolo[4,3-c]pyridin-1-yl]butan-1-ol (800 mg, crude) as a white solid.

### Step 3: Synthesis of tert-butyl-[3-[6-chloro-3-(1H-pyrrol-2-yl)pyrazolo[4,3-c]pyridin-1-yl]butoxy]-diphenyl-silane

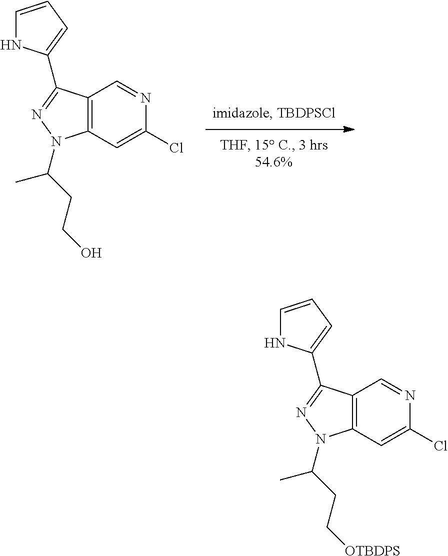

To a solution of 3-[6-chloro-3-(1H-pyrrol-2-yl)pyrazolo[4,3-c]pyridin-1-yl]butan-1-ol (800 mg, 2.75 mmol, 1.0 eq) in THF (10.0 mL) were added imidazole (450 mg, 6.61 mmol, 2.40 eq) and TBDPSCl (0.75 mL, 2.92 mmol, 1.06 eq) at 15° C. After addition, the mixture was stirred at 15° C. for 3 hours. The reaction mixture was diluted with water (30.0 mL) and extracted with EtOAc (50.0 mL*3). The combined organic layers were washed with brine (50.0 mL), dried over Na₂SO₄ and filtered. The filtrate was concentrated under reduced pressure and the residue was purified by column chromatography (silica, petroleum ether/EtOAc=1/0 to 15/1, 254 nm) to afford tert-butyl-[3-[6-chloro-3-(1H-pyrrol-2-yl)pyrazolo[4,3-c]pyridin-1-yl]butoxy]-diphenyl-silane (820 mg, 54.6% yield, 97% purity) as colourless oil.

LCMS [M+H]⁺ m/z: calcd 529.2, found 529.1.

¹H NMR (400 MHz, CDCl₃) δ 9.04 (s, 1H), 7.60 (dd, J=1.6, 8.0 Hz, 2H), 7.49-7.45 (m, 2H), 7.45-7.40 (m, 1H), 7.39-7.32 (m, 4H), 7.27-7.22 (m, 3H), 6.96-6.91 (m, 1H), 6.83 (br s, 1H), 6.38 (q, J=2.8 Hz, 1H), 5.02-4.84 (m, 1H), 3.71-3.50 (m, 1H), 3.28 (dt, J=4.0, 10.2 Hz, 1H), 2.33-2.19 (m, 1H), 2.11-2.01 (m, 1H), 1.59 (s, 3H), 1.06 (s, 9H).

### Step 4: Synthesis of tert-butyl-[3-[6-chloro-3-(1H-pyrrol-2-yl)pyrazolo[4,3-c]pyridin-1-yl]butoxy]-diphenyl-silane

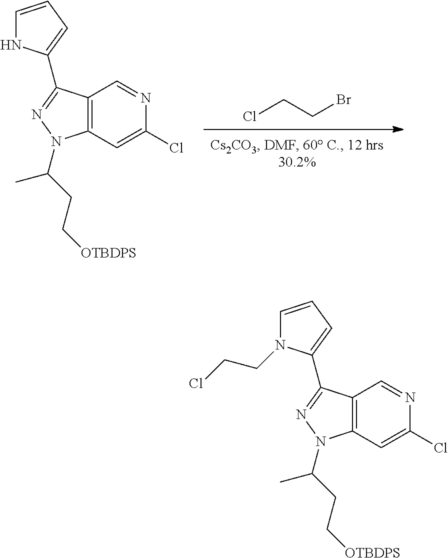

To a solution of tert-butyl-[3-[6-chloro-3-(1H-pyrrol-2-yl)pyrazolo[4,3-c]pyridin-1-yl]butoxy]-diphenyl-silane (400 mg, 0.76 mmol, 1.0 eq) in DMF (8.0 mL) were added Cs₂CO₃ (700 mg, 2.15 mmol, 2.84 eq) and 1-bromo-2-chloro-ethane (0.4 mL, 4.83 mmol, 6.38 eq). The mixture was then stirred at 60° C. for 12 hours. The reaction mixture was diluted with water (30.0 mL) and extracted with EtOAc (50.0 mL*3). The combined organic layers were washed with brine (50.0 mL*3), dried over Na₂SO₄ and filtered. The filtrate was concentrated under reduced pressure and the residue was purified by preparative TLC (silica, petroleum ether/EtOAc=5/1, 254 nm) to afford tert-butyl-[3-[6-chloro-3-[1-(2-chloroethyl)pyrrol-2-yl]pyrazolo[4,3-c]pyridin-1-yl]butoxy]-diphenyl-silane (150 mg, 30.2% yield, 90% purity) as a white solid.

LCMS [M+H]⁺ m/z: calcd 591.2, found 591.1.

### Step 5: Synthesis of 2-[2-[1-[3-[tert-butyl(diphenyl)silyl]oxy-1-methyl-propyl]-6-chloro-pyrazolo[4,3-c]pyridin-3-yl]pyrrol-1-yl]-N,N-dimethyl-ethanamine

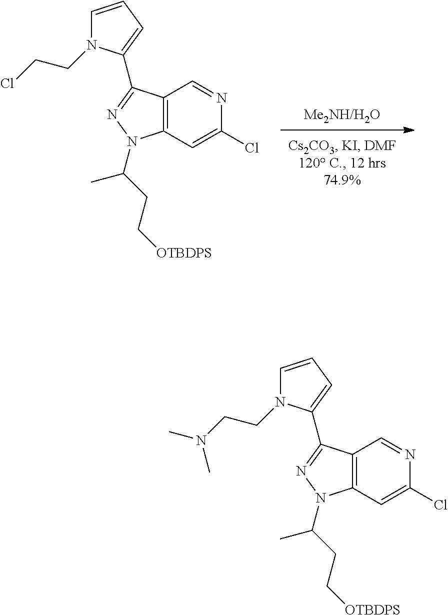

Tert-butyl-[3-[6-chloro-3-[1-(2-chloroethyl)pyrrol-2-yl]pyrazolo[4,3-c]pyridin-1-yl]butoxy]-diphenyl-silane (150 mg, 0.25 mmol, 1.0 eq), N-methylmethanamine/H₂O (1.33 g, 11.8 mmol, 1.5 mL, 40 wt %, 46.7 eq) and KI (120 mg, 0.72 mmol, 2.85 eq) in DMF (15.0 mL) was heated at 120° C. for 12 hours. The reaction mixture was diluted with water (15.0 mL) and extracted with EtOAc (20.0 mL*3). The combined organic layers were washed with brine (20 mL*3), dried over Na₂SO₄ and filtered. The filtrate was concentrated under reduced pressure and the residue was purified by preparative TLC (silica, 100% EtOAc, 254 nm) to afford 2-[2-[1-[3-[tert-butyl(diphenyl)silyl]oxy-1-methyl-propyl]-6-chloro-pyrazolo[4,3-c]pyridin-3-yl]pyrrol-1-yl]-N,N-dimethyl-ethanamine (120 mg, 74.9% yield, 95% purity) was obtained as brown oil.

LCMS [M+H]⁺ m/z: calcd 600.3, found 600.1.

### Step 6: Synthesis of 4-[4-[[ ]-[3-[tert-butyl(diphenyl)silyl]oxy-1-methyl-propyl]-3-[1-[2-(dimethylamino)ethyl]pyrrol-2-yl]pyrazolo[4,3-c]pyridin-6-yl]amino]pyrimidin-2-yl]-2-methyl-1-(2-trimethylsilylethoxymethyl)pyrazol-3-one

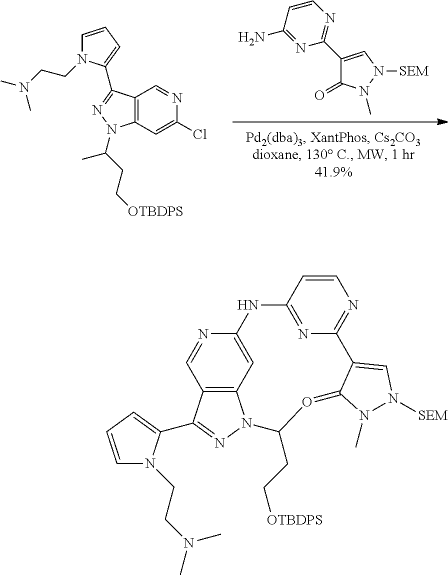

A suspension of 4-(4-aminopyrimidin-2-yl)-2-methyl-1-(2-trimethylsilylethoxymethyl)pyrazol-3-one (85 mg, 0.26 mmol, 1.13 eq), 2-[2-[1-[3-[tert-butyl(diphenyl)silyl]oxy-1-methyl-propyl]-6-chloro-pyrazolo[4,3-c]pyridin-3-yl]pyrrol-1-yl]-N,N-dimethyl-ethanamine (140 mg, 0.23 mmol, 1.0 eq), Pd₂(dba)₃ (50 mg, 0.05 mmol, 0.2 eq), Xantphos (60 mg, 0.1 mmol, 0.4 eq) and Cs₂CO₃ (230 mg, 0.7 mmol, 3.0 eq) in dioxane (3.0 mL) were taken up into a microwave tube. The mixture was distured with nitrogen for 2 minutes. The sealed tube was heated at 130° C. for 1 hour under microwave. The reaction mixture was diluted with water (20.0 mL) and extracted with DCM (30.0 mL*3). The combined organic layers were dried over Na₂SO₄, filtered and concentrated under reduced pressure. The residue was purified by preparative TLC (silica, DCM/MeOH/NH₃—H₂O=10/1/0.25) to afford 4-[4-[[1-[3-[tert-butyl(diphenyl)silyl]oxy-1-methyl-propyl]-3-[1-[2-(dimethylamino)ethyl]pyrrol-2-yl]pyrazolo[4,3-c]pyridin-6-yl]amino]pyrimidin-2-yl]-2-methyl-1-(2-trimethylsilylethoxymethyl)pyrazol-3-one (90 mg, 41.9% yield, 96% purity) as brown oil.

LCMS [M+H]⁺ m/z: calcd 885.5, found 885.4.

### Step 7: Synthesis of 4-[4-[[3-[1-[2-(dimethylamino)ethyl]pyrrol-2-yl]-1-(3-hydroxy-1-methyl-propyl)pyrazolo[4,3-c]pyridin-6-yl]amino]pyrimidin-2-yl]-2-methyl-pyrazol-3-ol

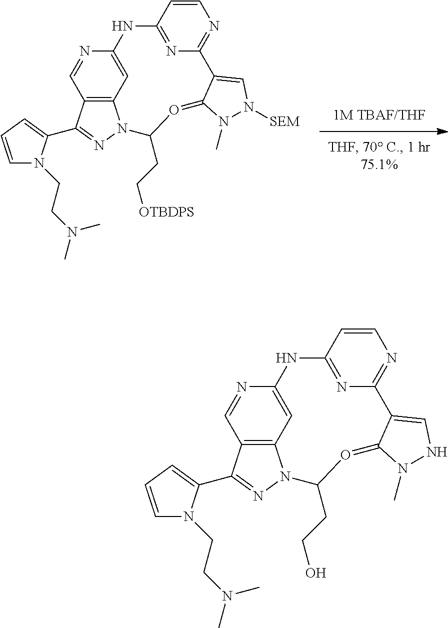

To a solution of 4-[4-[[1-[3-[tert-butyl(diphenyl)silyl]oxy-1-methyl-propyl]-3-[1-[2-(dimethylamino)ethyl]pyrrol-2-yl]pyrazolo[4,3-c]pyridin-6-yl]amino]pyrimidin-2-yl]-2-methyl-1-(2-trimethylsilylethoxymethyl)pyrazol-3-one (130 mg, 0.15 mmol, 1.0 eq) in THF (5.0 mL) was added 1M TBAF/THF (0.3 mL, 0.3 mmol, 2.04 eq). The mixture was stirred at 70° C. for 1 hour. The reaction mixture was concentrated under reduced pressure and the residue was purified by flash chromatography (Biotage®; 25 g SepaFlash® C18, 40-60 μm, 120 Å; MeCN/water (0.5 v % NH₃—H₂O) with MeCN from 0-40%, 25 mL/min, 254 nm) to afford 4-[4-[[3-[1-[2-(dimethylamino)ethyl]pyrrol-2-yl]-1-(3-hydroxy-1-methyl-propyl)pyrazolo[4,3-c]pyridin-6-yl]amino]pyrimidin-2-yl]-2-methyl-pyrazol-3-ol (60 mg, 75.1% yield, 95% purity) as a yellow solid.

LCMS [M+H]⁺ m/z: calcd 517.3, found 517.2.

1H NMR (400 MHz, CDCl₃) δ 8.89 (s, 1H), 8.55 (br s, 1H), 8.32 (br s, 1H), 7.94 (s, 1H), 7.78 (d, J=6.8 Hz, 1H), 6.91-6.84 (m, 1H), 6.72 (dd, J=1.6, 3.6 Hz, 1H), 6.40 (br s, 1H), 6.34-6.25 (m, 1H), 5.07-4.95 (m, 1H), 4.59-4.42 (m, 2H), 3.68-3.56 (m, 4H), 3.37 (dt, J=4.8, 10.0 Hz, 1H), 2.69 (br d, J=5.6 Hz, 1H), 2.48-2.38 (m, 1H), 2.29 (s, 6H), 2.21 (dt, J=5.2, 9.2 Hz, 1H), 1.71 (d, J=6.8 Hz, 3H).

### Step 8: Synthesis of 2-[2-(11,16-dimethyl-13-oxa-2,6,10,11,17,18,22,25-octazapentacyclo[15.5.2.13,7.08,12.020,24]pentacosa-1(22),3,5,7(25),8(12),9,18,20,23-nonaen-19-yl)pyrrol-1-yl]-N,N-dimethyl-ethanamine (compound (12))

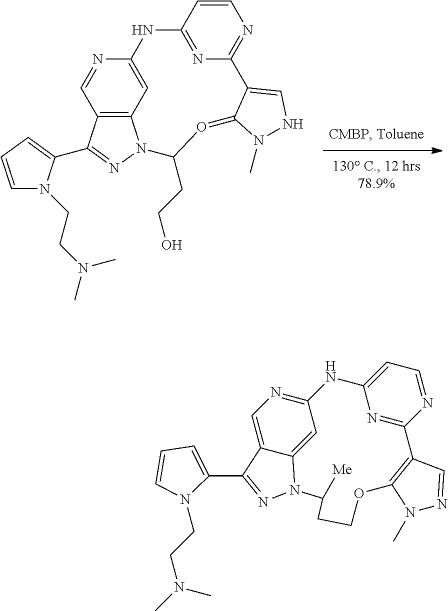

To a solution of 4-[4-[[3-[1-[2-(dimethylamino)ethyl]pyrrol-2-yl]-1-(3-hydroxy-1-methyl-propyl)pyrazolo[4,3-c]pyridin-6-yl]amino]pyrimidin-2-yl]-2-methyl-pyrazol-3-ol (65 mg, 0.13 mmol, 1.0 eq) in toluene (10.0 mL) was added 2-(tributyl-λ5-phosphanylidene)acetonitrile (200 mg, 0.82 mmol, 6.6 eq). The mixture was disturbed with nitrogen for 3 times and stirred at 130° C. for 12 hours. The reaction mixture was concentrated under reduced pressure and the residue was purified by preparative TLC (silica, DCM/MeOH=9/1, 254 nm) to afford crude product (55 mg, 78.9% yield, 90% purity) which was combined with another batch to afford the product (62 mg). This product was purified by preparative HPLC (column: Welch Xtimate C18 150*25 mm*5 μm; mobile phase A: water (0.04% NH₃H₂O+10 mM NH₄HCO₃); mobile phase MeCN; B %: 39%-69%, 7.8 min; Temp: 30° C.) to afford 2-[2-(11,16-dimethyl-13-oxa-2,6,10,11,17,18,22,25-octazapentacyclo[15.5.2.13,7.08,12.020,24]pentacosa-1(22),3,5,7(25),8(12),9,18,20,23-nonaen-19-yl)pyrrol-1-yl]-N,N-dimethyl-ethanamine (20.9 mg, 33.4% yield, 99% purity) as a white solid.

LCMS [M+H]⁺ m/z: calcd 499.3, found 499.1.

¹H NMR (400 MHz, CDCl₃) δ 9.11 (s, 1H), 8.92 (d, J=0.8 Hz, 1H), 8.34 (d, J=5.8 Hz, 1H), 8.15 (s, 1H), 8.05 (br s, 1H), 6.93-6.86 (m, 1H), 6.76 (dd, J=2.0, 4.0 Hz, 1H), 6.43 (d, J=6.0 Hz, 1H), 6.31 (dd, J=2.8, 3.6 Hz, 1H), 5.32-5.20 (m, 1H), 4.72-4.64 (m, 1H), 4.63-4.46 (m, 2H), 3.85-3.77 (m, 4H), 2.76 (br d, J=3.2 Hz, 2H), 2.54-2.43 (m, 1H), 2.32 (s, 6H), 2.22-2.11 (m, 1H), 1.92 (d, J=6.8 Hz, 3H);

### Example 4: Preparation of Compound (14)—Typical Procedure for Making Compound I-C-1

These compounds were prepared via the procedures described in Example 1 by substituting 3-bromo-1-(2-trimethylsilylethoxymethyl)pyridin-4-one (A) for 4-bromo-2-methyl-1-((2-(trimethylsilyl)ethoxy)methyl)-1,2-dihydro-3H-pyrazol-3-one. Synthesis of intermediate (A) was illustrated below.

### Synthesis of 3-bromo-1-(2-trimethylsilylethoxymethyl)pyridin-4-one

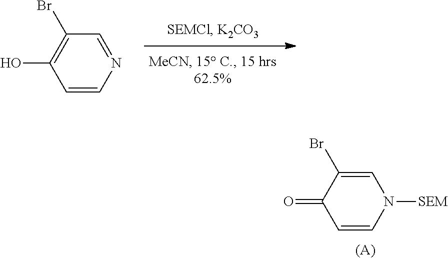

A mixture of 3-bromopyridin-4-ol (500 mg, 2.87 mmol, 1.0 eq), 2-(chloromethoxy)ethyl-trimethyl-silane (8.62 mmol, 1.5 mL, 3.0 eq) and K₂CO₃ (1.99 g, 14.4 mmol, 5.0 eq) in MeCN (20.0 mL) was stirred at 15° C. for 15 hours. The reaction mixture was filtered and concentrated under reduced pressure and the residue was purified by flash silica gel chromatography (ISCO®; 25 g SepaFlash® Silica Flash Column, EtOAc/MeOH with MeOH from 0-10%, flow rate=30 mL/min, 254 nm) to afford 3-bromo-1-(2-trimethylsilylethoxymethyl)pyridin-4-one (A) (600 mg, 62.5% yield, 91% purity) as a yellow solid.

LCMS [M+H]⁺ m/z: calcd 304.0, found 303.8.

¹H NMR (400 MHz, CDCl₃) δ 7.86 (d, J=2.4 Hz, 1H), 7.42 (dd, J=2.4, 7.2 Hz, 1H), 6.50 (d, J=7.6 Hz, 1H), 5.07 (s, 2H), 3.59-3.53 (m, 2H), 0.98-0.94 (m, 2H), 0.03 (s, 9H).

Regio-chemistry was confirmed by NOE.

### 20-(azetidin-1-yl)-17-methyl-14-oxa-2,6,10,18,19,23,26-heptazapentacyclo[16.5.2.13,7.08,13.021,25]hexacosa-1(23),3,5,7(26),8(13),9,11,19,21,24-decaene (compound (14))

LCMS [M+H]⁺ m/z: calcd 415.2, found 415.1.

¹H NMR (400 MHz, CDCl₃) δ 9.57 (s, 1H), 9.47 (s, 1H), 8.58 (br d, J=5.2 Hz, 1H), 8.54-8.49 (m, 2H), 7.63 (s, 1H), 7.04 (d, J=6.0 Hz, 1H), 6.61 (d, J=5.6 Hz, 1H), 4.69-4.61 (m, 1H), 4.37 (t, J=9.2 Hz, 1H), 4.28-4.18 (m, 4H), 4.09-4.02 (m, 1H), 2.52 (quin, J=7.2 Hz, 2H), 2.45-2.36 (m, 1H), 2.25-2.12 (m, 1H), 1.81 (d, J=6.8 Hz, 3H).

### Example 5: Preparation of Compound (145)—Typical Procedure for Making Compound I-A-1 (R¹=Substituted Phenyls or 2 General Heteroaryls)

### Synthesis of (16S)-11,16-dimethyl-19-[3-(2-morpholinoethoxy)phenyl]-13-oxa-2,6,10,11,17,18,22,25-octazapentacyclo[15.5.2.1³.0^{8,12} 0^{20,24}]pentacosa-1(22),3,5,7(25),8(12),9,18,20,23-nonaene (compound (145))

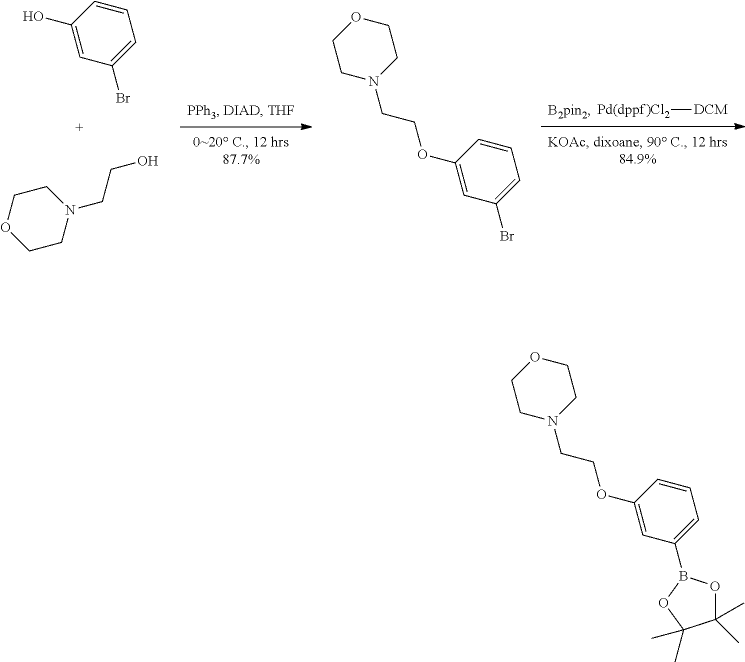

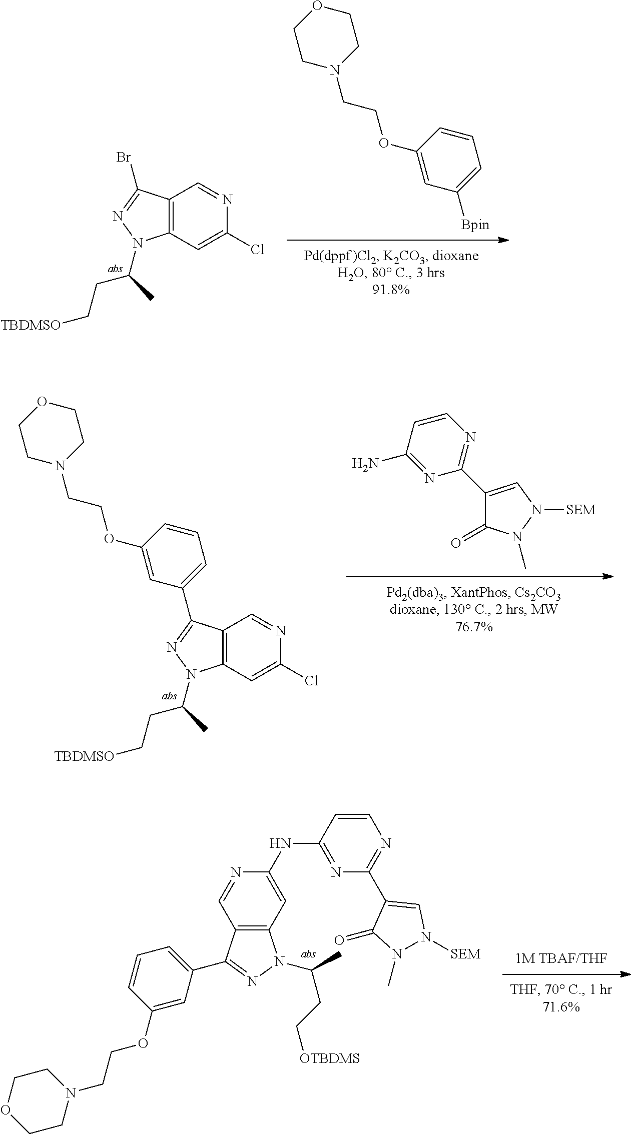

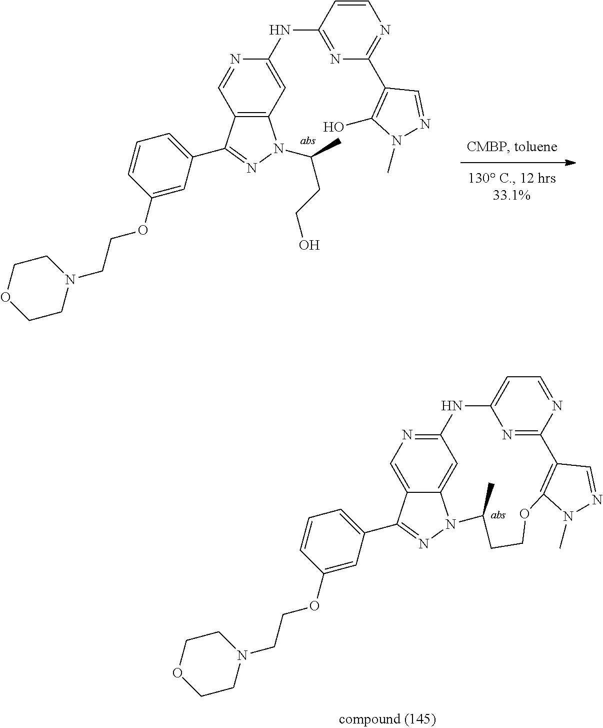 [SMILES: C[C@@H](CCO)n1nc(-c2cccc(OCCN3CCOCC3)c2)c2cnc(Nc3ccnc(-c4cnn(C)c4O)n3)cc21 ; C[C@H]1CCOc2c(cnn2C)-c2nccc(n2)Nc2cc3c(cn2)c(-c2cccc(OCCN4CCOCC4)c2)nn31]

### Step 1: Synthesis of 4-[2-(3-bromophenoxy)ethyl]morpholine

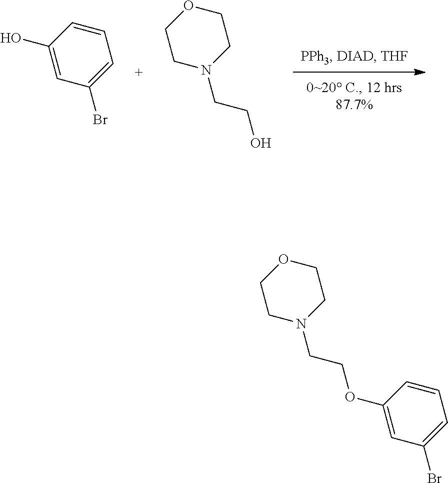 [SMILES: Oc1cccc(Br)c1 ; OCCN1CCOCC1 ; Brc1cccc(OCCN2CCOCC2)c1]

To a mixture of 3-bromophenol (600 mg, 3.47 mmol, 1.0 eq),2-morpholinoethanol (700 mg, 5.34 mmol, 1.5 eq) and PPh₃ (1.5 g, 5.72 mmol, 1.7 eq) in THF (10.0 mL) was added DIAD (1.14 g, 5.66 mmol, 1.6 eq) at 0° C. and the mixture was stirred at 20° C. for 12 hours under N₂. The reaction mixture was concentrated under reduced pressure and the residue was purified by flash chromatography (ISCO®; 40 g SepaFlash® Silica Flash Column, petroleum ether/EtOAc with EtOAc from 0-100%, 100 mL/min, 254 nm) to afford 4-[2-(3-bromophenoxy)ethyl]morpholine (1 g, 87.7% yield, 87% purity) as a colorless oil.

LCMS [M+H]⁺ m/z: calcd 286.0, found 285.8.

¹H NMR (400 MHz, chloroform-d) δ ppm 7.17-7.11 (m, 1H), 7.11-7.06 (m, 2H), 6.88-6.81 (m, 1H), 4.09 (t, J=5.8 Hz, 2H), 3.77-3.72 (m, 4H), 2.80 (t, J=5.6 Hz, 2H), 2.61-2.54 (m, 1H), 2.61-2.54 (m, 4H).

### Step 2: Synthesis of 4-[2-[3-(4,4,5,5-tetramethyl-1,3,2-dioxaborolan-2-yl)phenoxy]ethyl]morpholine

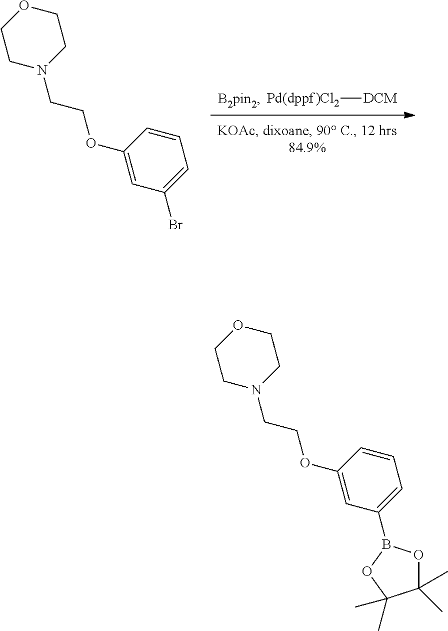

4-[2-(3-bromophenoxy)ethyl]morpholine (1 g, 3.49 mmol, 1.0 eq), Pin₂B₂ (1.1 g, 4.33 mmol, 1.2 eq), Pd(dppf)Cl₂-DCM (300 mg, 0.37 mmol, 0.1 eq) and KOAc (700 mg, 7.13 mmol, 2.0 eq) in dioxane (15.0 mL) was de-gassed and then heated to 90° C. for 12 hours under N₂. The reaction mixture was filtered and concentrated under reduced pressure to give 4-[2-[3-(4,4,5,5-tetramethyl-1,3,2-dioxaborolan-2-yl)phenoxy]ethyl]morpholine (2.3 g, 84.9% yield, 43% purity) as a brown oil.

LCMS [M+H]⁺ m/z: calcd 334.2, found 334.0.

### Step 3: Synthesis of tert-butyl-[(3S)-3-[6-chloro-3-[3-(2-morpholinoethoxy)phenyl]pyrazolo[4,3-c]pyridin-1-yl]butoxy]-dimethyl-silane

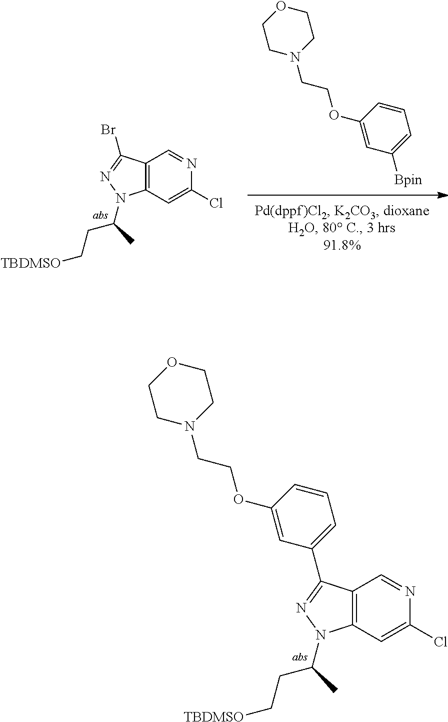

[(3S)-3-(3-bromo-6-chloro-pyrazolo[4,3-c]pyridin-1-yl)butoxy]-tert-butyl-dimethyl-silane (700 mg, 1.67 mmol, 1.0 eq), 4-[2-[3-(4,4,5,5-tetramethyl-1,3,2-dioxaborolan-2-yl)phenoxy]ethyl]morpholine (2.3 g, 2.97 mmol, 43% purity, 1.8 eq), Pd(dppf)Cl₂ (130 mg, 0.18 mmol, 0.1 eq) and K₂CO₃ (600 mg, 4.34 mmol, 2.6 eq) in dioxane (20.0 mL) and H₂O (4.0 mL) was de-gassed and then heated to 80° C. for 3 hours under N₂. The resulting mixture was filtered and washed with EtOAc (20 mL*3). The combined filtrate diluted with saturated Na₂CO₃ aqueous solution (30 mL) and water (30 mL), and then extracted with EtOAc (60 mL). The combined organic layers were washed with brine (60 mL*2), dried over Na₂SO₄, filtered and concentrated under reduced pressure. The residue was purified by flash chromatography (ISCO®; 40 g SepaFlash® Silica Flash Column, petroleum ether/EtOAc with EtOAc from 0-100%, 100 mL/min, 254 nm) to afford tert-butyl-[(3S)-3-[6-chloro-3-[3-(2-morpholinoethoxy)phenyl]pyrazolo[4,3-c]pyridin-1-yl]butoxy]-dimethyl-silane (1.1 g, 91.8% yield, 76% purity) as a yellow oil.

LCMS [M+H]⁺ m/z: calcd 545.3, found 545.1.

¹H NMR (400 MHz, chloroform-d) δ ppm 9.07 (s, 1H), 7.56-7.50 (m, 2H), 7.45 (br d, J=8.1 Hz, 1H), 7.42 (s, 1H), 7.01 (br d, J=5.8 Hz, 1H), 4.91 (br t, J=10.4 Hz, 1H), 4.22 (t, J=5.7 Hz, 2H), 3.77-3.74 (m, 4H), 3.64-3.55 (m, 1H), 3.16 (dt, J=3.3, 10.1 Hz, 1H), 2.87 (t, J=5.6 Hz, 2H), 2.61 (br d, J=4.6 Hz, 4H), 2.33-2.20 (m, 1H), 2.11-2.02 (m, 1H), 1.65 (d, J=6.8 Hz, 3H), 0.89 (s, 9H), −0.04 (d, J=15.3 Hz, 6H).

### Step 4: Synthesis of 4-[4-[[1-[(1S)-3-[tert-butyl(dimethyl)silyl]oxy-1-methyl-propyl]-3-[3-(2-morpholinoethoxy)phenyl]pyrazolo[4,3-c]pyridin-6-yl]amino]pyrimidin-2-yl]-2-methyl-1-(2-trimethylsilylethoxymethyl)pyrazol-3-one

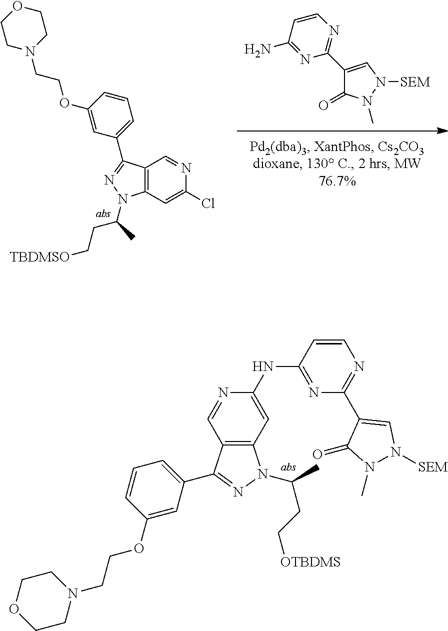 [SMILES: C[C@@H](CCO[Si](C)(C)C(C)(C)C)n1nc(-c2cccc(OCCN3CCOCC3)c2)c2cnc(Cl)cc21 ; Cn1c(=O)c(-c2nccc(N)n2)cn1COCC[Si](C)(C)C ; C[C@@H](CCO[Si](C)(C)C(C)(C)C)n1nc(-c2cccc(OCCN3CCOCC3)c2)c2cnc(Nc3ccnc(-c4cn(COCC[Si](C)(C)C)n(C)c4=O)n3)cc21]

Tert-butyl-[(3S)-3-[6-chloro-3-[3-(2-morpholinoethoxy)phenyl]pyrazolo[4,3-c]pyridin-1-yl]butoxy]-dimethyl-silane (350 mg, 0.64 mmol, 1.5 eq), 4-(4-aminopyrimidin-2-yl)-2-methyl-1-(2-trimethylsilylethoxymethyl)pyrazol-3-one (140 mg, 0.44 mmol, 1.0 eq), XantPhos (26 mg, 0.045 mmol, 0.1 eq), Cs₂CO₃ (280 mg, 0.86 mmol, 2.0 eq) and Pd₂(dba)₃ (40 mg, 0.044 mmol, 0.1 eq) were taken up into a microwave tube in dioxane (15.0 mL). The sealed tube was heated at 130° C. for 2 hours under microwave under N₂. The reaction mixture was filtered and concentrated under reduced pressure. The residue was purified by flash chromatography (ISCO®; 20 g SepaFlash® Silica Flash Column, DCM/MeOH (0.05% TEA) with MeOH (0.05% TEA) from 0-10%, 80 mL/min, 254 nm) to afford 4-[4-[[1-[(1S)-3-[tert-butyl(dimethyl)silyl]oxy-1-methyl-propyl]-3-[3-(2-morpholinoethoxy)phenyl]pyrazolo[4,3-c]pyridin-6-yl]amino]pyrimidin-2-yl]-2-methyl-1-(2-trimethylsilylethoxymethyl)pyrazol-3-one (330 mg, 76.7% yield, 84% purity) as a yellow solid.

LCMS [M+H]⁺ m/z: calcd 830.5, found 830.5.

¹H NMR (400 MHz, chloroform-d) δ ppm 9.23 (br s, 1H), 8.99 (s, 1H), 8.32 (d, J=5.8 Hz, 1H), 8.20 (s, 1H), 7.80 (br s, 1H), 7.61-7.55 (m, 2H), 7.43 (br t, J=7.8 Hz, 1H), 6.98 (br d, J=8.1 Hz, 1H), 6.67 (br d, J=5.3 Hz, 1H), 5.53 (br d, J=7.5 Hz, 1H), 5.18 (s, 2H), 4.24 (br t, J=5.6 Hz, 2H), 3.79-3.75 (m, 4H), 3.62 (br t, J=6.4 Hz, 2H), 3.57 (s, 3H), 3.56-3.52 (m, 2H), 2.87 (br t, J=5.5 Hz, 2H), 2.63 (br s, 4H), 2.53-2.41 (m, 1H), 2.27-2.12 (m, 1H), 1.67 (br d, J=6.4 Hz, 3H), 0.94 (br t, J=8.0 Hz, 2H), 0.80 (s, 9H), −0.08 (d, J=11.6 Hz, 6H).

### Step 5: Synthesis of 4-[4-[[1-[(1S)-3-hydroxy-1-methyl-propyl]-3-[3-(2-morpholinoethoxy)-phenyl]pyrazolo[4,3-c]pyridin-6-yl]amino]pyrimidin-2-yl]-2-methyl-pyrazol-3-ol

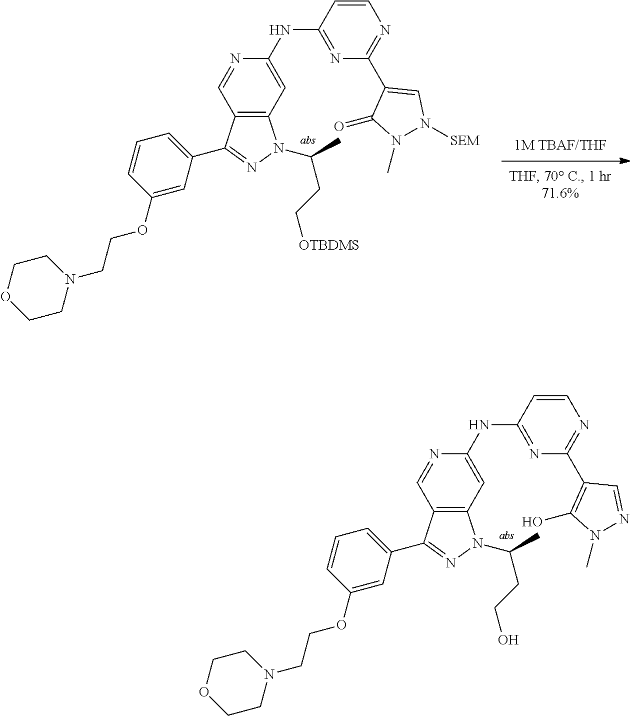 [SMILES: C[C@@H](CCO[Si](C)(C)C(C)(C)C)n1nc(-c2cccc(OCCN3CCOCC3)c2)c2cnc(Nc3ccnc(-c4cn(COCC[Si](C)(C)C)n(C)c4=O)n3)cc21 ; C[C@@H](CCO)n1nc(-c2cccc(OCCN3CCOCC3)c2)c2cnc(Nc3ccnc(-c4cnn(C)c4O)n3)cc21]

A mixture of 4-[4-[[1-[(1S)-3-[tert-butyl(dimethyl)silyl]oxy-1-methyl-propyl]-3-[3-(2-morpholinoethoxy)phenyl]pyrazolo[4,3-c]pyridin-6-yl]amino]pyrimidin-2-yl]-2-methyl-1-(2-trimethylsilylethoxymethyl)pyrazol-3-one (330 mg, 0.40 mmol, 1.0 eq) and 1M TBAF/THF (0.6 mL, 0.6 mmol) in THF (10.0 mL) was stirred at 70° C. for 1 hour. The reaction mixture was concentrated under reduced pressure and the residue was purified by flash chromatography (ISCO®; SepaFlash® Spherical C18 Column, 40 g, 40-60 μm, 120 Å, Eluent of 0-36% ACN/H₂O (0.05% NH₃—H₂O) gradient @ 50 mL/min, 254 nm) to afford 4-[4-[[1-[(1S)-3-hydroxy-1-methyl-propyl]-3-[3-(2-morpholinoethoxy)phenyl]pyrazolo[4,3-c]pyridin-6-yl]amino]pyrimidin-2-yl]-2-methyl-pyrazol-3-ol (170 mg, 71.6% yield, 98% purity) as a yellow solid.

LCMS [M+H]⁺ m/z: calcd 586.3, found 586.2.

### Step 6: Synthesis of (16S)-11,16-dimethyl-19-[3-(2-morpholinoethoxy)phenyl]-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaene (compound (145))

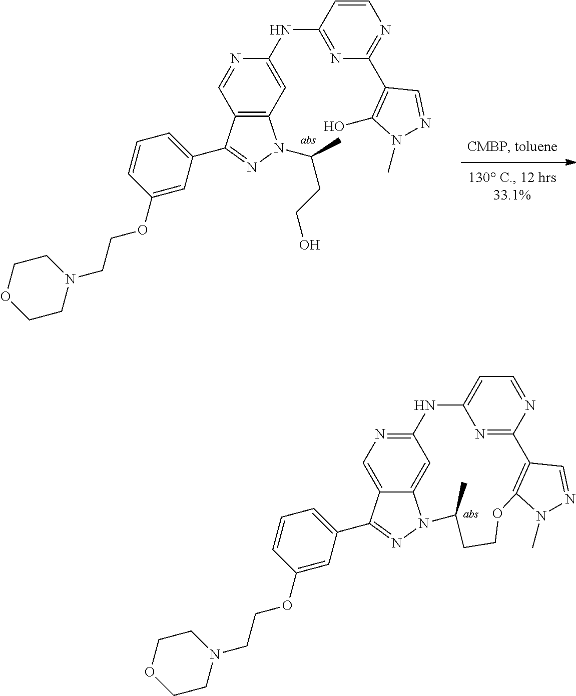 [SMILES: C[C@@H](CCO)n1nc(-c2cccc(OCCN3CCOCC3)c2)c2cnc(Nc3ccnc(-c4cnn(C)c4O)n3)cc21 ; C[C@H]1CCOc2c(cnn2C)-c2nccc(n2)Nc2cc3c(cn2)c(-c2cccc(OCCN4CCOCC4)c2)nn31]

A mixture of 4-[4-[[1-[(1S)-3-hydroxy-1-methyl-propyl]-3-[3-(2-morpholinoethoxy)phenyl]-pyrazolo[4,3-c]pyridin-6-yl]amino]pyrimidin-2-yl]-2-methyl-pyrazol-3-ol (170 mg, 0.29 mmol, 1.0 eq) and 2-(tributyl-X⁵-phosphanylidene)acetonitrile (400 mg, 1.66 mmol, 5.7 eq) in toluene (15.0 mL) was stirred at 130° C. for 12 hours under N₂. The reaction mixture was concentrated under reduced pressure and the residue was purified by flash chromatography (ISCO®; 20 g SepaFlash® Silica Flash Column, DCM/MeOH (0.05 v % TEA) with MeOH from 0-15%, 80 mL/min, 254 nm) to afford a crude product which was purified by preparative HPLC (Column: Phenomenex Gemini-NX 80*40 mm*3 μm; Mobile phase: [water (0.05% NH₃H₂O+10 mM NH₄HCO₃)-ACN]; B %: 34%-64%, 7.8 min, Column Temp.: 30° C., 254 nm) to afford (16S)-11,16-dimethyl-19-[3-(2-morpholinoethoxy)phenyl]-13-oxa-2,6,10,11,17,18,22,25-octazapentacyclo[15.5.2.1³⁷.0^{8,12}.0^{20,24}]pentacosa-1(22),3,5,7(25),8(12),9,18,20,23-nonaene (54.6 mg, 33.1% yield) as a white solid.

¹H NMR (400 MHz, methanol-d₄) δ ppm 9.08 (s, 1H), 9.01 (s, 1H), 8.24 (d, J=6.0 Hz, 1H), 8.02 (s, 1H), 7.63-7.56 (m, 2H), 7.45 (t, J=7.9 Hz, 1H), 7.04 (dd, J=2.3, 7.8 Hz, 1H), 6.69 (d, J=6.0 Hz, 1H), 5.25 (br dd, J=7.5, 10.0 Hz, 1H), 4.57-4.50 (m, 1H), 4.26 (t, J=5.4 Hz, 2H), 3.94 (br d, J=10.3 Hz, 1H), 3.82 (s, 3H), 3.78-3.72 (m, 4H), 2.90 (t, J=5.3 Hz, 2H), 2.68 (br s, 4H), 2.64-2.55 (m, 1H), 2.20-2.11 (m, 1H), 1.91 (d, J=6.8 Hz, 3H).

LCMS [M+H]⁺ m/z: calcd 568.3, found 568.1.

### Example 6: Preparation of Compound (63)

The exemplary synthetic procedure of this Example is a typical procedure for making compound I-A-1 (R¹=substituted furan, 1,2,4-trisubstituted pyrroles) Synthesis of 11,16-dimethyl-19-[5-[(4-methylpiperazin-1-yl)methyl]-2-furyl]-13-oxa-2,6,10,11,17,18,22,25-octazapentacyclo[15.5.2.13,7.08,12.020,24]pentacosa-1(22),3,5,7(25),8(12),9,18,20,23-nonaene (compound (63))

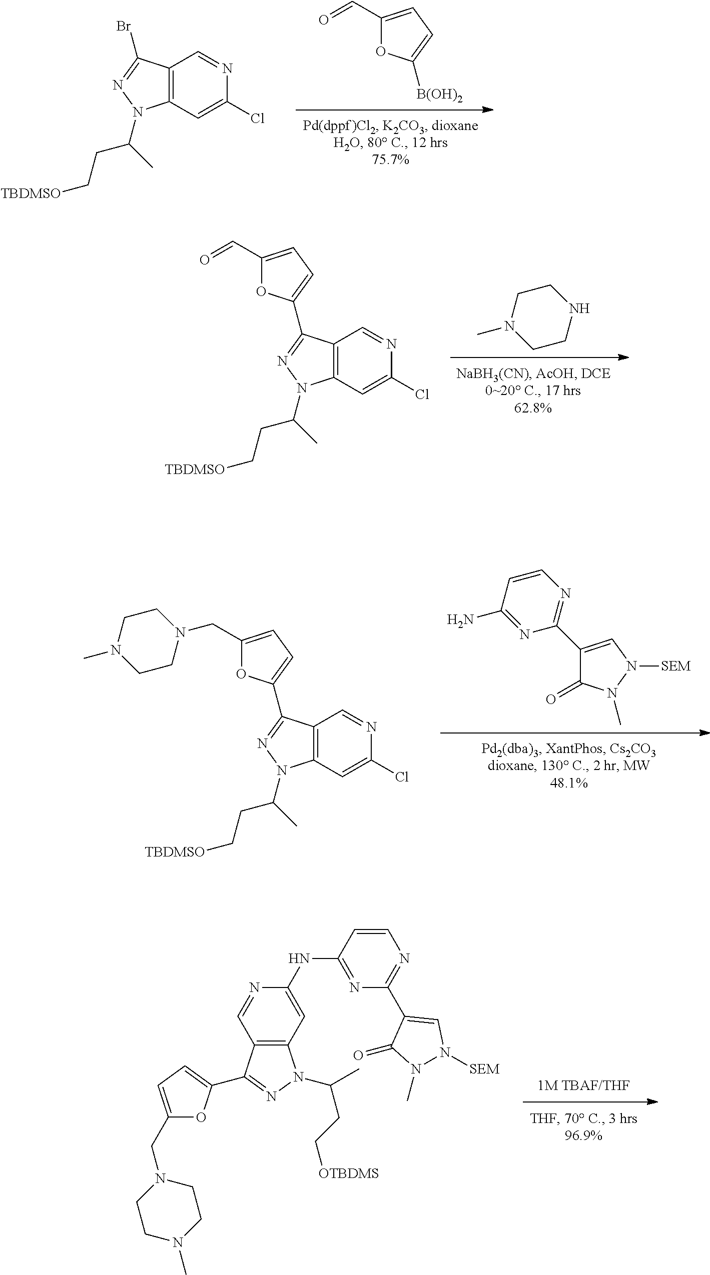

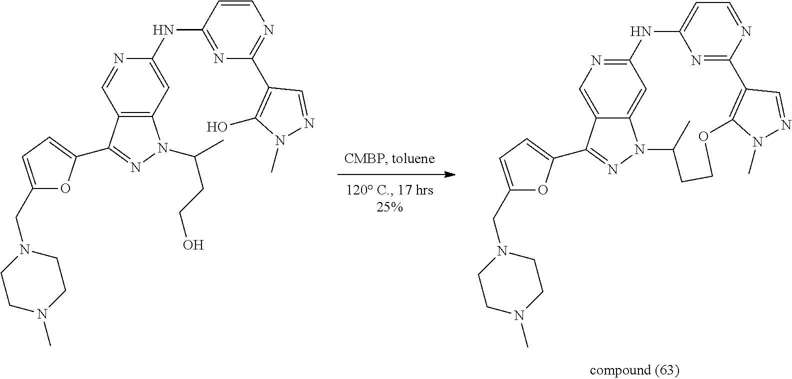 [SMILES: CC(CCO)n1nc(-c2ccc(CN3CCN(C)CC3)o2)c2cnc(Nc3ccnc(-c4cnn(C)c4O)n3)cc21 ; CC1CCOc2c(cnn2C)-c2nccc(n2)Nc2cc3c(cn2)c(-c2ccc(CN4CCN(C)CC4)o2)nn31]

### Step 1: Synthesis of 5-[1-[3-[tert-butyl(dimethyl)silyl]oxy-1-methyl-propyl]-6-chloro-pyrazolo[4,3-c]pyridin-3-yl]furan-2-carbaldehyde

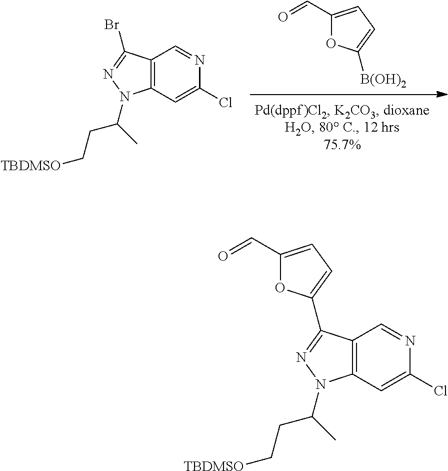 [SMILES: CC(CCO[Si](C)(C)C(C)(C)C)n1nc(Br)c2cnc(Cl)cc21 ; O=Cc1ccc(B(O)O)o1 ; CC(CCO[Si](C)(C)C(C)(C)C)n1nc(-c2ccc(C=O)o2)c2cnc(Cl)cc21]

3-(3-bromo-6-chloro-pyrazolo[4,3-c]pyridin-1-yl)butoxy-tert-butyl-dimethyl-silane (500 mg, 1.19 mmol, 1.0 eq), (5-formyl-2-furyl)boronic acid (350 mg, 2.50 mmol, 2.1 eq), Pd(dppf)Cl₂ (100 mg, 0.137 mmol, 0.1 eq) and K₂CO₃ (350 mg, 2.53 mmol, 2.1 eq) in dioxane (15.0 mL) and H₂O (3.0 mL) was de-gassed and then heated to 80° C. for 12 hours under nitrogen. The reaction was filtered and the filter cake was washed with DCM (15 mL*3). The combined filtrate was concentrated under reduced pressure. The residue was purified by column chromatography (SiO₂, petroleum ether/EtOAc=30:1 to 3/1, 254 nm) to afford 5-[1-[3-[tert-butyl(dimethyl)silyl]oxy-1-methyl-propyl]-6-chloro-pyrazolo[4,3-c]pyridin-3-yl]furan-2-carbaldehyde (400 mg, 75.7% yield, 98% purity) as a yellow oil.

LCMS [M+H]⁺ m/z: calcd 434.2, found 434.0.

¹H NMR (400 MHz, chloroform-d) δ ppm 9.79 (s, 1H), 9.31 (d, J=0.8 Hz, 1H), 7.45 (d, J=1.0 Hz, 1H), 7.41 (d, J=3.5 Hz, 1H), 7.15 (d, J=3.8 Hz, 1H), 5.00-4.90 (m, 1H), 3.65-3.55 (m, 1H), 3.14 (dt, J=3.5, 10.3 Hz, 1H), 2.31-2.21 (m, 1H), 2.07 (tdd, J=4.7, 9.5, 14.1 Hz, 1H), 1.65 (d, J=6.8 Hz, 3H), 0.89 (s, 9H), −0.02 (s, 3H), −0.06 (s, 3H).

### Step 2: Synthesis of tert-butyl-[3-[6-chloro-3-[5-[(4-methylpiperazin-1-yl)methyl]-2-furyl]pyrazolo[4,3-c]pyridin-1-yl]butoxy]-dimethyl-silane

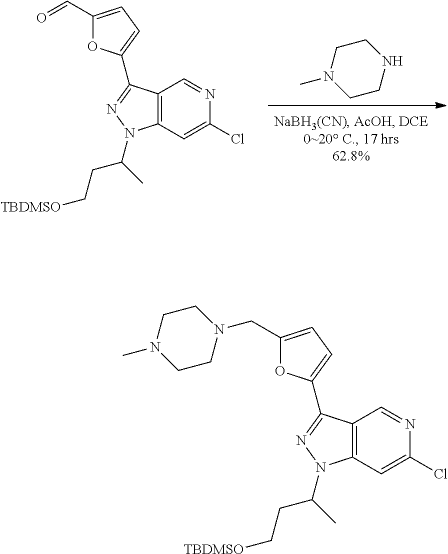

A mixture of 5-[1-[3-[tert-butyl(dimethyl)silyl]oxy-1-methyl-propyl]-6-chloro-pyrazolo[4,3-c]pyridin-3-yl]furan-2-carbaldehyde (400 mg, 0.922 mmol, 1.0 eq), 1-methylpiperazine (300 mg, 3.00 mmol, 3.3 eq) and AcOH (300 mg, 5.0 mmol, 5.4 eq) in DCE (15.0 mL) was stirred at 20° C. for 16.5 hours. Then NaBH₃CN (400 mg, 6.37 mmol, 6.9 eq) was added at 0° C. and the mixture was stirred at 0° C. for 30 minutes. The reaction mixture was quenched by addition of H₂O (20 mL), and extracted with DCM (20 mL*3). The combined organic layers were washed with brine (30 mL*3), dried over Na₂SO₄, filtered and concentrated under reduced pressure. The residue was purified by preparative TLC (silica, DCM/MeOH=10:1, 254 nm) to afford tert-butyl-[3-[6-chloro-3-[5-[(4-methylpiperazin-1-yl)methyl]-2-furyl]pyrazolo[4,3-c]pyridin-1-yl]butoxy]-dimethyl-silane (300 mg, 62.8% yield, 100% purity) as a yellow oil.

LCMS [M+H]⁺ m/z: calcd 518.3, found 518.1.

¹H NMR (400 MHz, chloroform-d) δ ppm 9.16 (d, J=0.8 Hz, 1H), 7.37 (d, J=0.8 Hz, 1H), 6.90 (d, J=3.3 Hz, 1H), 6.40 (d, J=3.3 Hz, 1H), 4.93-4.85 (m, 1H), 3.73 (s, 2H), 3.62-3.54 (m, 1H), 3.13 (dt, J=3.3, 10.2 Hz, 1H), 2.73-2.40 (m, 7H), 2.31 (s, 3H), 2.29-2.19 (m, 2H), 2.04 (tdd, J=4.7, 9.5, 14.1 Hz, 1H), 1.62 (d, J=6.8 Hz, 3H), 0.89 (s, 9H), −0.02 (s, 3H), −0.06 (s, 3H).

### Step 3: Synthesis of 4-[4-[[1-[3-[tert-butyl(dimethyl)silyl]oxy-1-methyl-propyl]-3-[5-[(4-methylpiperazin-1-yl)methyl]-2-furyl]pyrazolo[4,3-c]pyridin-6-yl]amino]pyrimidin-2-yl]-2-methyl-1-(2-trimethylsilylethoxymethyl)pyrazol-3-one

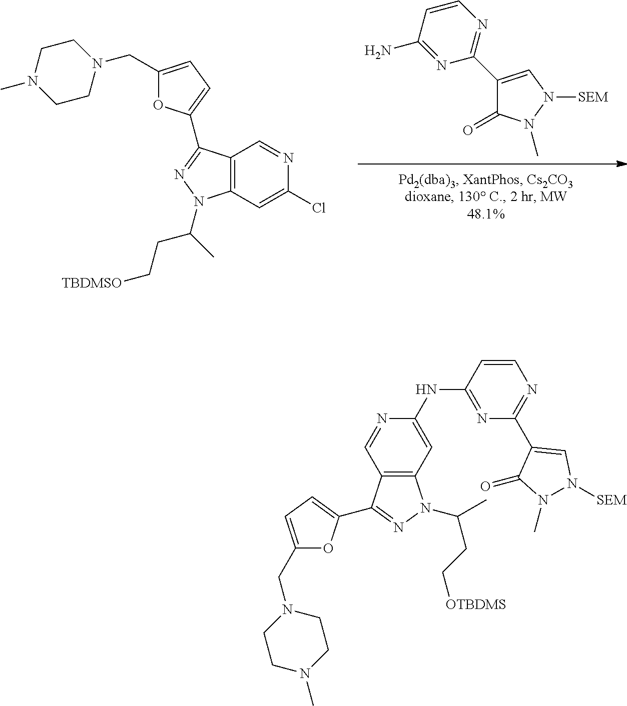 [SMILES: CC(CCO[Si](C)(C)C(C)(C)C)n1nc(-c2ccc(CN3CCN(C)CC3)o2)c2cnc(Cl)cc21 ; Cn1c(=O)c(-c2nccc(N)n2)cn1COCC[Si](C)(C)C ; CC(CCO[Si](C)(C)C(C)(C)C)n1nc(-c2ccc(CN3CCN(C)CC3)o2)c2cnc(Nc3ccnc(-c4cn(COCC[Si](C)(C)C)n(C)c4=O)n3)cc21]

Tert-butyl-[3-[6-chloro-3-[5-[(4-methylpiperazin-1-yl)methyl]-2-furyl]pyrazolo[4,3-c]pyridin-1-yl]butoxy]-dimethyl-silane (300 mg, 0.579 mmol, 1.0 eq), 4-(4-aminopyrimidin-2-yl)-2-methyl-1-(2-trimethylsilylethoxymethyl)pyrazol-3-one (200 mg, 0.622 mmol, 1.1 eq), XantPhos (34 mg, 0.0587 mmol, 0.1 eq), Cs₂CO₃ (400 mg, 1.23 mmol, 2.1 eq) and Pd₂(dba)₃ (51 mg, 0.0557 mmol, 0.1 eq) were taken up into a microwave tube in dioxane (15.0 mL). The sealed tube was heated at 130° C. for 2 hours under microwave and nitrogen. The reaction mixture was filtered and concentrated under reduced pressure. The residue was purified by preparative TLC (silica, DCM/MeOH=5:1, 254 nm) to afford 4-[4-[[1-[3-[tert-butyl(dimethyl)silyl]oxy-1-methyl-propyl]-3-[5-[(4-methylpiperazin-1-yl)methyl]-2-furyl]pyrazolo[4,3-c]pyridin-6-yl]amino]pyrimidin-2-yl]-2-methyl-1-(2-trimethylsilylethoxymethyl)pyrazol-3-one (260 mg, 48.1% yield, 86% purity) as a yellow oil.

LCMS [M+H]⁺ m/z: calcd 803.5, found 803.4.

¹H NMR (400 MHz, chloroform-d) δ ppm 9.08 (br s, 1H), 9.05 (s, 1H), 8.32 (d, J=5.8 Hz, 1H), 8.20 (s, 1H), 7.89 (br s, 1H), 6.88 (d, J=3.3 Hz, 1H), 6.74 (br d, J=5.8 Hz, 1H), 6.39 (d, J=3.3 Hz, 1H), 5.45 (br s, 1H), 3.72 (s, 2H), 3.60-3.54 (m, 6H), 2.71-2.39 (m, 9H), 2.31 (s, 3H), 2.23-2.11 (m, 1H), 1.64 (br d, J=6.5 Hz, 6H), 0.96-0.91 (m, 2H), 0.80 (s, 9H), 0.01 (s, 9H), −0.07 (s, 3H), −0.10 (s, 3H).

### Step 4: Synthesis of 4-[4-[[1-(3-hydroxy-1-methyl-propyl)-3-[5-[(4-methylpiperazin-1-yl)methyl]-2-furyl]pyrazolo[4,3-c]pyridin-6-yl]amino]pyrimidin-2-yl]-2-methyl-pyrazol-3-ol

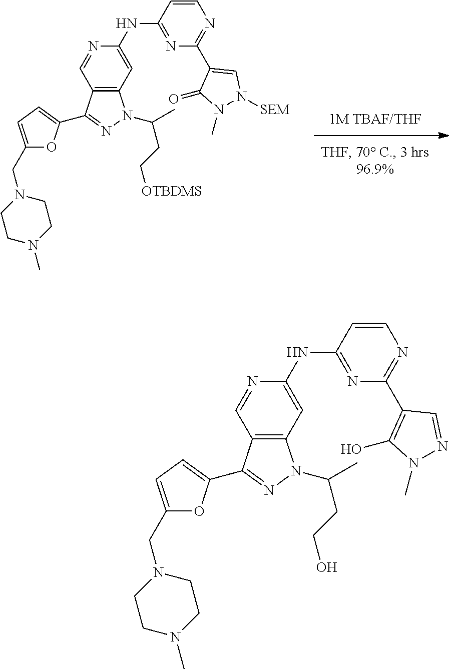 [SMILES: CC(CCO[Si](C)(C)C(C)(C)C)n1nc(-c2ccc(CN3CCN(C)CC3)o2)c2cnc(Nc3ccnc(-c4cn(COCC[Si](C)(C)C)n(C)c4=O)n3)cc21 ; CC(CCO)n1nc(-c2ccc(CN3CCN(C)CC3)o2)c2cnc(Nc3ccnc(-c4cnn(C)c4O)n3)cc21]

A mixture of 4-[4-[[1-[3-[tert-butyl(dimethyl)silyl]oxy-1-methyl-propyl]-3-[5-[(4-methylpiperazin-1-yl)methyl]-2-furyl]pyrazolo[4,3-c]pyridin-6-yl]amino]pyrimidin-2-yl]-2-methyl-1-(2-trimethylsilylethoxymethyl)pyrazol-3-one (320 mg, 0.398 mmol, 1.0 eq) and 1M TBAF/THF (0.8 mL, 0.8 mmol, 2.0 eq) in THF (10.0 mL) was stirred at 70° C. for 3 hours. The reaction mixture was concentrated under reduced pressure and the residue was purified by flash chromatography (Column: SepaFlash® Sphercial C18, 40 g, 40-60 μm, 120 Å; MeCN/water (0.5% NH₃—H₂O) with MeCN from 0-45%, 30 mL/min, 254 nm) to afford 4-[4-[[1-(3-hydroxy-1-methyl-propyl)-3-[5-[(4-methylpiperazin-1-yl)methyl]-2-furyl]pyrazolo[4,3-c]pyridin-6-yl]amino]pyrimidin-2-yl]-2-methyl-pyrazol-3-ol (220 mg, 96.9% yield, 98% purity) as a yellow solid.

LCMS [M+H]⁺ m/z: calcd 559.3, found 559.1.

¹H NMR (400 MHz, methanol-d₄) δ ppm 9.11 (s, 1H), 8.06 (br d, J=6.0 Hz, 1H), 7.85 (s, 1H), 6.98 (d, J=3.3 Hz, 1H), 6.64 (br d, J=5.3 Hz, 1H), 6.52 (d, J=3.3 Hz, 1H), 5.41 (br d, J=4.8 Hz, 1H), 3.75 (s, 2H), 3.61-3.54 (m, 1H), 3.53 (s, 2H), 3.45 (ddd, J=4.0, 8.0, 11.5 Hz, 1H), 2.76-2.44 (m, 6H), 2.40-2.31 (m, 1H), 2.28 (s, 3H), 2.16-2.06 (m, 1H), 1.68-1.64 (m, 4H), 1.61-1.58 (m, 3H).

### Step 5: Synthesis of 11,16-dimethyl-19-[5-[(4-methylpiperazin-1-yl)methyl]-2-furyl]-13-oxa-2,6,10,11,17,18,22,25-octazapentacyclo[15.5.2.13,7.08,12.020,24]pentacosa-1(22),3,5,7(25),8(12),9,18,20,23-nonaene

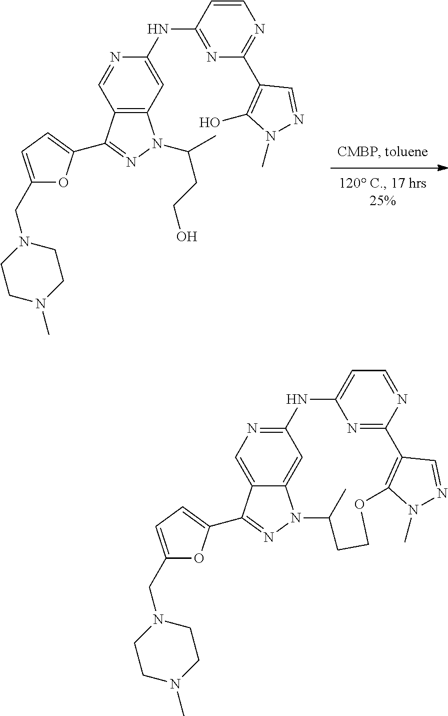 [SMILES: CC(CCO)n1nc(-c2ccc(CN3CCN(C)CC3)o2)c2cnc(Nc3ccnc(-c4cnn(C)c4O)n3)cc21 ; CC1CCOc2c(cnn2C)-c2nccc(n2)Nc2cc3c(cn2)c(-c2ccc(CN4CCN(C)CC4)o2)nn31]

A mixture of 4-[4-[[1-(3-hydroxy-1-methyl-propyl)-3-[5-[(4-methylpiperazin-1-yl)methyl]-2-furyl]pyrazolo[4,3-c]pyridin-6-yl]amino]pyrimidin-2-yl]-2-methyl-pyrazol-3-ol (120 mg, 0.215 mmol, 1.0 eq) and 2-(tributyl-λ5-phosphanylidene)acetonitrile (260 mg, 1.08 mmol, 5.0 eq) in toluene (20.0 mL) was stirred at 120° C. for 17 hours under nitrogen. The reaction mixture was concentrated under reduced pressure and the residue was purified by preparative TLC (silica, DCM:MeOH=5:1, 254 nm) to afford a crude product which was further purified by preparative HPLC (Column: Waters Xbridge 150*25 mm*5 μm; Mobile phase: [water (0.05% NH₃—H₂O)-ACN]; B %: 32%-62%, 7.8 min, Column Temp.: 30° C.) to afford 11,16-dimethyl-19-[5-[(4-methylpiperazin-1-yl)methyl]-2-furyl]-13-oxa-2,6,10,11,17,18,22,25-octazapentacyclo[15.5.2.13,7.08,12.020,24]pentacosa-1(22),3,5,7(25),8(12),9,18,20,23-nonaene (29 mg, 25.0% yield, 100% purity) as a white solid.

LCMS [M+H]⁺ m/z: calcd 541.3, found 541.1.

1H NMR (400 MHz, methanol-d₄) δ 9.06 (s, 1H), 9.00 (s, 1H), 8.20 (d, J=6.0 Hz, 1H), 8.00 (s, 1H), 6.96 (d, J=3.4 Hz, 1H), 6.64 (d, J=5.9 Hz, 1H), 6.51 (d, J=3.3 Hz, 1H), 5.21-5.13 (m, 1H), 4.47 (br t, J=11.1 Hz, 1H), 3.91-3.83 (m, 1H), 3.78 (s, 3H), 3.73 (s, 2H), 2.78-2.39 (m, 9H), 2.28 (s, 3H), 2.14-2.04 (m, 1H), 1.84 (d, J=6.9 Hz, 3H).

### Example 7: Preparation of Compound (101)—Typical Procedure for Making Compound I-A-1 (R¹=Substituted Pyrazoles and 1,2,5-Trisubstituted Pyrroles)

### Synthesis of (16S)-11,16-dimethyl-19-[1-methyl-5-[(4-methylpiperazin-1-yl)methyl]pyrrol-2-yl]-13-oxa-2,6,10,11,17,18,22,25-octazapentacyclo[15.5.2.13,7.08,12.020,24]pentacosa-1(22),3,5,7(25),8(12),9,18,20,23-nonaene (compound (101))

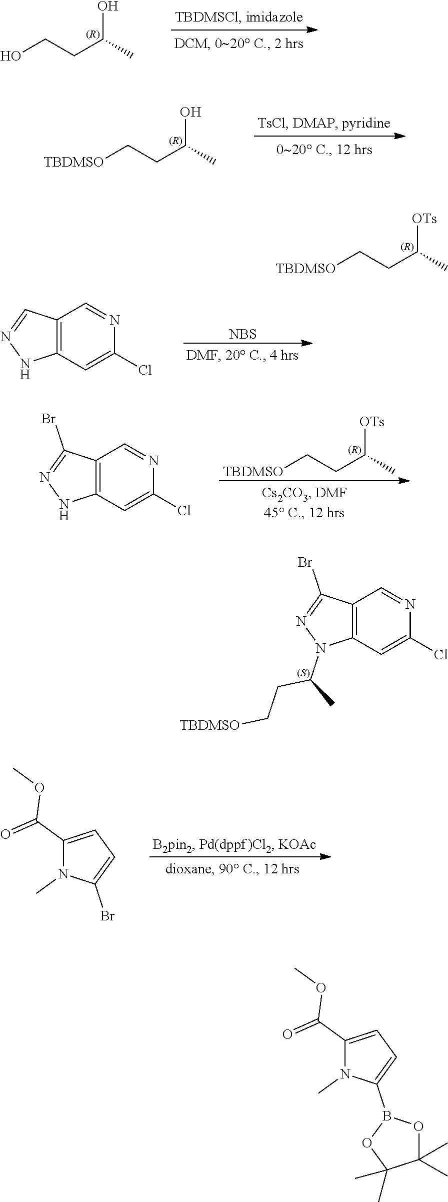

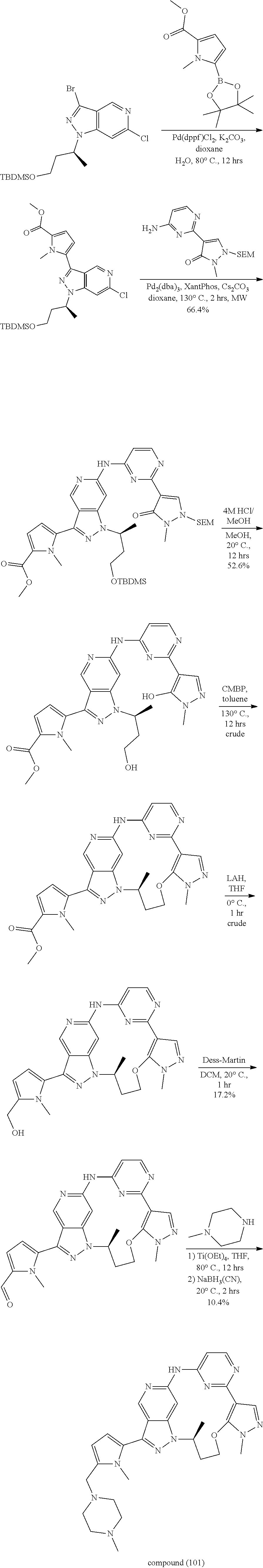

### Step 1: Synthesis of (2R)-4-[tert-butyl(dimethyl)silyl]oxybutan-2-ol

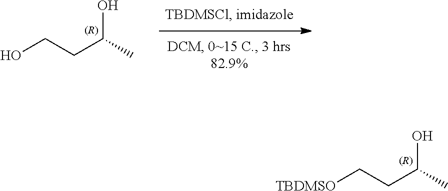

To a solution of (3R)-butane-1,3-diol (5 g, 55.5 mmol, 1.0 eq) and imidazole (4.15 g, 61.0 mmol, 1.1 eq) in DCM (80.0 mL) was added TBDMSCl (8.36 g, 55.5 mmol, 1.0 eq) at 0° C. The mixture was stirred at 15° C. for 3 hours. The reaction mixture was diluted with H₂O (100 mL and extracted with DCM (100 mL*2). The combined organic layers were washed with brine (100 mL*1), dried over anhydrous Na₂SO₄, filtered and concentrated under reduced pressure. The residue was purified by flash chromatography (ISCO®; 80 g SepaFlash® Silica Flash Column, petroleum ether/EtOAc with EtOAc from 0-30%, 100 mL/min, PMA) to afford (2R)-4-[tert-butyl(dimethyl)silyl]oxybutan-2-ol (9.4 g, 82.9% yield) as colourless oil.

¹H NMR (400 MHz, CDCl₃) δ ppm 4.08-3.96 (m, 1H), 3.93-3.86 (m, 1H), 3.85-3.78 (m, 1H), 3.41 (s, 1H), 1.72-1.58 (m, 2H), 1.19 (d, J=6.4 Hz, 3H), 0.92-0.88 (m, 9H), 0.08 (s, 6H).

### Step 2: Synthesis of [(1R)-3-[tert-butyl(dimethyl)silyl]oxy-1-methyl-propyl]-4-methylbenzenesulfonate

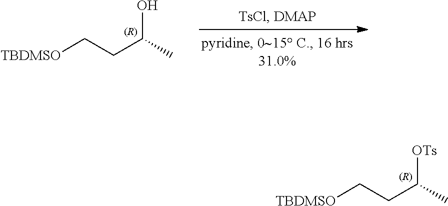

To a solution of (2R)-4-[tert-butyl(dimethyl)silyl]oxybutan-2-ol (6 g, 29.4 mmol, 1.0 eq) in pyridine (60.0 mL) added 4-methylbenzenesulfonyl chloride (12.31 g, 64.6 mmol, 2.2 eq) and DMAP (1.08 g, 8.81 mmol, 0.3 eq) at 0° C. The mixture was stirred at 15° C. for 16 hours. The reaction mixture was diluted with H₂O (100 mL) and extracted with DCM (100 mL*2). The combined organic layers were washed with brine (100 mL), dried over anhydrous Na₂SO₄, filtered and concentrated under reduced pressure. The residue was purified by flash chromatography (ISCO®; 25 g SepaFlash® Silica Flash Column, petroleum ether/EtOAc with EtOAc from 0-30%, 30 mL/min, 254 nm) to afford [(1R)-3-[tert-butyl(dimethyl)silyl]oxy-1-methyl-propyl]-4-methylbenzenesulfonate (3.4 g, 31.0% yield) as colorless oil.

LCMS [M+H]⁺ m/z: calcd 359.2, found 359.0.

¹H NMR (400 MHz, CDCl₃) δ ppm 7.86-7.76 (m, 2H), 7.33 (br d, J=7.6 Hz, 2H), 4.83-4.72 (m, 1H), 3.58-3.45 (m, 2H), 2.44 (s, 3H), 1.89-1.80 (m, 1H), 1.71-1.62 (m, 1H), 1.32 (dd, J=1.2, 6.4 Hz, 3H), 0.85 (d, J=1.6 Hz, 9H), 0.03-−0.05 (m, 6H).

### Step 3: Synthesis of [(3S)-3-(3-bromo-6-chloro-pyrazolo[4,3-c]pyridin-1-yl)butoxy]-tert-butyl-dimethyl-silane

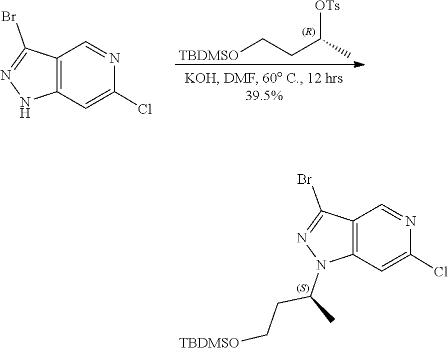

A mixture of 3-bromo-6-chloro-1H-pyrazolo[4,3-c]pyridine (1.24 g, 5.33 mmol, 1.0 eq), [(1R)-3-[tert-butyl(dimethyl)silyl]oxy-1-methyl-propyl]-4-methylbenzenesulfonate (2.10 g, 5.87 mmol, 1.1 eq), KOH (599 mg, 10.7 mmol, 2.0 eq) in DMF (20.0 mL) was stirred at 60° C. for 12 hours. The reaction mixture was diluted with H₂O (50 mL) and extracted with EtOAc (50 mL*2). The combined organic layers were washed with brine (50 mL), dried over anhydrous Na₂SO₄, filtered and concentrated under reduced pressure. The residue was purified by flash chromatography (ISCO®; 25 g SepaFlash® Silica Flash Column, petroleum ether/EtOAc with EtOAc from 0-10%, 30 mL/min, 254 nm) to afford [(3S)-3-(3-bromo-6-chloro-pyrazolo[4,3-c]pyridin-1-yl)butoxy]-tert-butyl-dimethyl-silane (1.28 g, 39.5% yield) as yellow oil.

LCMS [M+H]⁺ m/z: calcd 418.1, found 419.7.

¹H NMR (400 MHz, CDCl₃) δ ppm 8.71 (d, J=0.8 Hz, 1H), 7.39 (d, J=0.8 Hz, 1H), 4.91-4.80 (m, 1H), 3.63-3.55 (m, 1H), 3.15-3.06 (m, 1H), 2.24-2.14 (m, 1H), 2.06-1.96 (m, 1H), 1.61-1.59 (m, 3H), 0.88 (s, 9H), −0.04 (d, J=17.2 Hz, 6H).

The regio-chemistry was confirmed by NOE.

### Step 4: Synthesis of methyl 1-methyl-5-(4,4,5,5-tetramethyl-1,3,2-dioxaborolan-2-yl)-1H-pyrrole-2-carboxylate

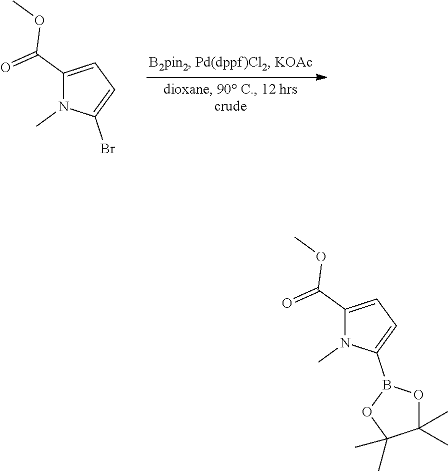 [SMILES: COC(=O)c1ccc(Br)n1C ; COC(=O)c1ccc(B2OC(C)(C)C(C)(C)O2)n1C]

A mixture of methyl 5-bromo-1-methyl-pyrrole-2-carboxylate (3.0 g, 13.8 mmol, 1.0 eq), 4,4,5,5-tetramethyl-2-(4,4,5,5-tetramethyl-1,3,2-dioxaborolan-2-yl)-1,3,2-dioxaborolane (10.0 g, 39.4 mmol, 2.9 eq), KOAc (3.0 g, 30.6 mmol, 2.2 eq) and Pd(dppf)Cl₂ (1.2 g, 1.64 mmol, 0.1 eq) in dioxane (30.0 mL) was degassed and purged with nitrogen for 3 times, and then the mixture was stirred at 90° C. for 12 hours under nitrogen atmosphere. The reaction mixture was diluted with water (20 mL) and extracted with EtOAc (20 mL*3). The combined organic layers were washed with brine (30 mL), dried over Na₂SO₄, filtered and concentrated under reduced pressure. The residue was purified by flash chromatography (ISCO®; 40 g SepaFlash® Silica Flash Column, Eluent of 0-4% EtOAc/petroleum ether gradient @ 50 mL/min, 254 nm) to afford methyl 1-methyl-5-(4,4,5,5-tetramethyl-1,3,2-dioxaborolan-2-yl)pyrrole-2-carboxylate (7.8 g, crude) as a white solid.

LCMS (ESI) [M+H]⁺ m/z: calcd 266.2, found 265.9.

¹H NMR (400 MHz, CDCl₃) δ ppm 6.91-6.85 (m, 1H), 6.65 (d, J=4.0 Hz, 1H), 4.13-4.06 (m, 3H), 3.77 (s, 3H), 1.28 (s, 12H).

### Step 5: Synthesis of methyl 5-[1-[(1S)-3-[tert-butyl(dimethyl)silyl]oxy-1-methyl-propyl]-6-chloro-pyrazolo[4,3-c]pyridin-3-yl]-1-methyl-pyrrole-2-carboxylate

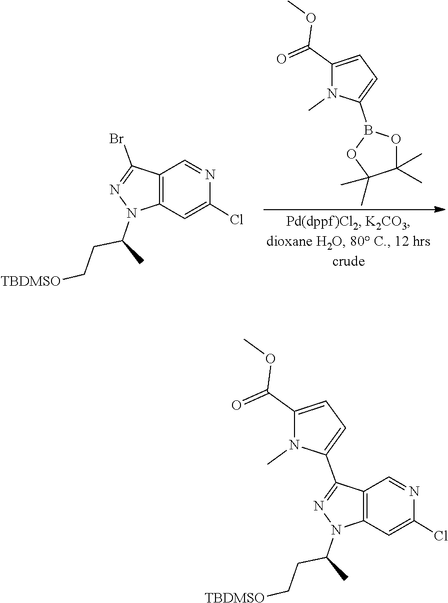 [SMILES: C[C@@H](CCO[Si](C)(C)C(C)(C)C)n1nc(Br)c2cnc(Cl)cc21 ; COC(=O)c1ccc(B2OC(C)(C)C(C)(C)O2)n1C ; COC(=O)c1ccc(-c2nn([C@@H](C)CCO[Si](C)(C)C(C)(C)C)c3cc(Cl)ncc23)n1C]

A mixture of [(3S)-3-(3-bromo-6-chloro-pyrazolo[4,3-c]pyridin-1-yl)butoxy]-tert-butyl-dimethyl-silane (500 mg, 1.19 mmol, 1.0 eq), methyl 1-methyl-5-(4,4,5,5-tetramethyl-1,3,2-dioxaborolan-2-yl)pyrrole-2-carboxylate (2.0 g, 4.0 mmol, 3.4 eq), K₂CO₃ (500 mg, 3.62 mmol, 3.0 eq) and Pd(dppf)Cl₂ (150 mg, 0.205 mmol, 0.2 eq) in dioxane (10.0 mL) and H₂O (2.0 mL) was degassed and purged with nitrogen for 3 times, and then the mixture was stirred at 80° C. for 12 hours under nitrogen atmosphere. The reaction mixture was diluted with water (30 mL) and extracted with EtOAc (30 mL*3). The combined organic layers were washed with brine (20 mL), dried over Na₂SO₄, filtered and concentrated under reduced pressure. The residue was purified by preparative TLC (SiO₂, petroleum ether/EtOAc=5:1, 254 nm) to afford methyl 5-[1-[(1S)-3-[tert-butyl(dimethyl)silyl]oxy-1-methyl-propyl]-6-chloro-pyrazolo[4,3-c]pyridin-3-yl]-1-methyl-pyrrole-2-carboxylate (1.2 g, crude) as a white solid. LCMS (ESI) [M+H]⁺ m/z: calcd 477.2, found 477.1.

### Step 6: Synthesis of methyl 5-[1-[(1S)-3-[tert-butyl(dimethyl)silyl]oxy-1-methyl-propyl]-6-chloro-pyrazolo[4,3-c]pyridin-3-yl]-1-methyl-pyrrole-2-carboxylate

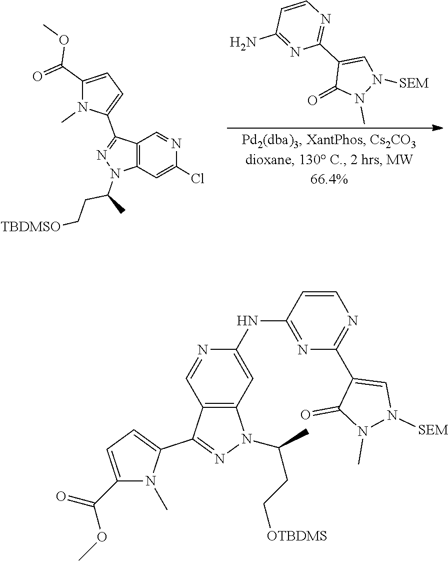 [SMILES: COC(=O)c1ccc(-c2nn([C@@H](C)CCO[Si](C)(C)C(C)(C)C)c3cc(Cl)ncc23)n1C ; Cn1c(=O)c(-c2nccc(N)n2)cn1COCC[Si](C)(C)C ; COC(=O)c1ccc(-c2nn([C@@H](C)CCO[Si](C)(C)C(C)(C)C)c3cc(Nc4ccnc(-c5cn(COCC[Si](C)(C)C)n(C)c5=O)n4)ncc23)n1C]

A mixture of methyl 5-[1-[(1S)-3-[tert-butyl(dimethyl)silyl]oxy-1-methyl-propyl]-6-chloro-pyrazolo[4,3-c]pyridin-3-yl]-1-methyl-pyrrole-2-carboxylate (550 mg, 1.15 mmol, 1.9 eq), 4-(4-aminopyrimidin-2-yl)-2-methyl-1-(2-trimethylsilylethoxymethyl)pyrazol-3-one (200 mg, 0.622 mmol, 1.0 eq), Cs₂CO₃ (500 mg, 1.53 mmol, 2.5 eq), XantPhos (70 mg, 0.121 mmol, 0.2 eq) and Pd₂(dba)₃ (70 mg, 0.0764 mmol, 0.1 eq) in dioxane (10.0 mL) was taken up into a microwave tube (two parallel batches were set up). The sealed tube was heated at 130° C. for 2 hours under microwave. Two batches were combined and the reaction mixture was filtered and then diluted with water (20 mL) and extracted with EtOAc (20 mL*3). The combined organic layers were dried over Na₂SO₄, filtered and concentrated under reduced pressure. The residue was purified by preparative TLC (SiO₂, DCM/MeOH=10/1, 254 nm) to afford methyl 5-[1-[(1S)-3-[tert-butyl(dimethyl)silyl]oxy-1-methyl-propyl]-6-[[2-[2-methyl-3-oxo-1-(2-trimethylsilylethoxymethyl)pyrazol-4-yl]pyrimidin-4-yl]amino]pyrazolo[4,3-c]pyridin-3-yl]-1-methyl-pyrrole-2-carboxylate (630 mg, 66.4% yield) as a white solid. LCMS (ESI) [M+H]⁺ m/z: calcd 762.4, found 762.3.

### Step 7: Synthesis of methyl 5-[1-[(1S)-3-hydroxy-1-methyl-propyl]-6-[[2-(5-hydroxy-1-methyl-pyrazol-4-yl)pyrimidin-4-yl]amino]pyrazolo[4,3-c]pyridin-3-yl]-1-methyl-pyrrole-2-carboxylate

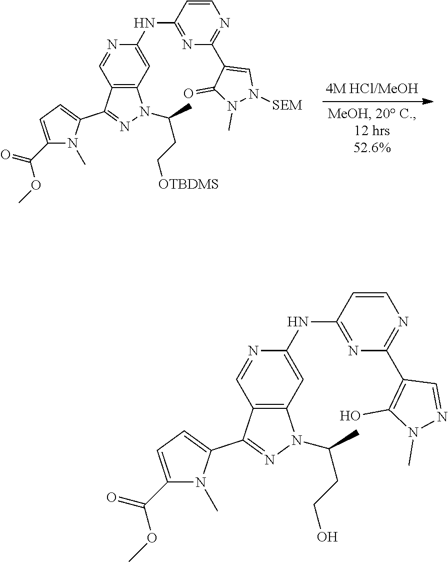 [SMILES: COC(=O)c1ccc(-c2nn([C@@H](C)CCO[Si](C)(C)C(C)(C)C)c3cc(Nc4ccnc(-c5cn(COCC[Si](C)(C)C)n(C)c5=O)n4)ncc23)n1C ; COC(=O)c1ccc(-c2nn([C@@H](C)CCO)c3cc(Nc4ccnc(-c5cnn(C)c5O)n4)ncc23)n1C]

To a solution of methyl 5-[1-[(1S)-3-[tert-butyl(dimethyl)silyl]oxy-1-methyl-propyl]-6-[[2-[2-methyl-3-oxo-1-(2-trimethylsilylethoxymethyl)pyrazol-4-yl]pyrimidin-4-yl]amino]pyrazolo[4,3-c]pyridin-3-yl]-1-methyl-pyrrole-2-carboxylate (560 mg, 0.735 mmol, 1.0 eq) in MeOH (5.0 mL) was added 4M HCl/MeOH (5.0 mL, 20 mmol). The mixture was stirred at 20° C. for 12 hours. The reaction mixture was quenched by addition saturated Na₂CO₃ aqueous solution to pH-8 at 0° C., and then diluted with DCM (20 mL), dried over Na₂SO₄, filtered and concentrated under reduced pressure. The residue was purified by flash chromatography (Biotage®, Column: SepaFlash® Sphercial C18, 40 g, 40-60 μm, 120 Å; MeCN/water (0.05% NH₃—H₂O) with MeCN from 0-36%, 50 mL/min, 254 nm) to afford methyl 5-[1-[(1S)-3-hydroxy-1-methyl-propyl]-6-[[2-(5-hydroxy-1-methyl-pyrazol-4-yl)pyrimidin-4-yl]amino]pyrazolo[4,3-c]pyridin-3-yl]-1-methyl-pyrrole-2-carboxylate (200 mg, 52.6% yield) as a white solid. LCMS (ESI) [M+H]⁺ m/z: calcd 518.2, found 518.1.

### Step 8: Synthesis of methyl 5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrole-2-carboxylate

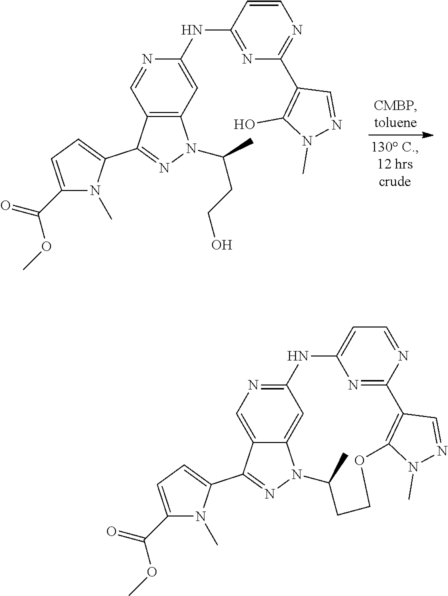 [SMILES: COC(=O)c1ccc(-c2nn([C@@H](C)CCO)c3cc(Nc4ccnc(-c5cnn(C)c5O)n4)ncc23)n1C ; COC(=O)c1ccc(-c2nn3c4cc(ncc24)Nc2ccnc(n2)-c2cnn(C)c2OCC[C@@H]3C)n1C]

A mixture of methyl 5-[1-[(1S)-3-hydroxy-1-methyl-propyl]-6-[[2-(5-hydroxy-1-methyl-pyrazol-4-yl)pyrimidin-4-yl]amino]pyrazolo[4,3-c]pyridin-3-yl]-1-methyl-pyrrole-2-carboxylate (200 mg, 0.386 mmol, 1.0 eq), 2-(tributyl-X⁵-phosphanylidene)acetonitrile (500 mg, 2.07 mmol, 5.4 eq) in toluene (10.0 mL) was degassed and purged with nitrogen for 3 times, and then the mixture was stirred at 130° C. for 12 hours under nitrogen atmosphere. The reaction mixture was concentrated under reduced pressure and the residue was purified by flash chromatography (ISCO®; 40 g SepaFlash® Silica Flash Column, Eluent of 0-10% MeOH/DCM @ 50 mL/min, 254 nm) to afford methyl 5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrole-2-carboxylate (900 mg, crude) as a white solid. LCMS (ESI) [M+H]⁺ m/z: calcd 500.2, found 500.1.

### Step 9: Synthesis of [5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrol-2-yl]methanol

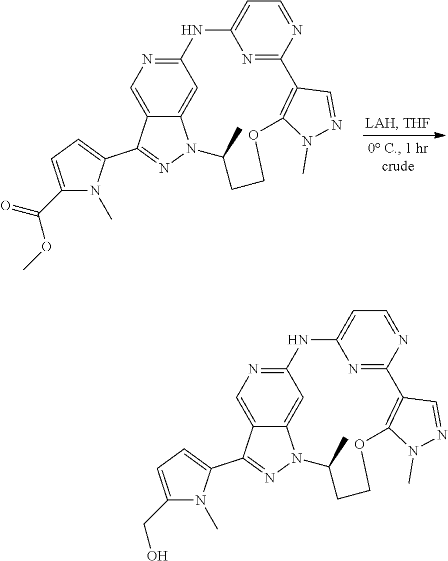 [SMILES: COC(=O)c1ccc(-c2nn3c4cc(ncc24)Nc2ccnc(n2)-c2cnn(C)c2OCC[C@@H]3C)n1C ; C[C@H]1CCOc2c(cnn2C)-c2nccc(n2)Nc2cc3c(cn2)c(-c2ccc(CO)n2C)nn31]

To a solution of methyl 5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrole-2-carboxylate (800 mg, 1.60 mmol, 1.0 eq) in THF (10.0 mL) was added LiAlH₄ (80 mg, 2.11 mmol, 1.32 eq). The mixture was stirred at 0° C. for 1 hr. The reaction mixture was diluted with water (30 mL) and extracted with EtOAc (30 mL*3). The combined organic layers were washed with brine (30 mL), dried over Na₂SO₄, filtered and concentrated under reduced pressure to give [5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrol-2-yl]methanol (700 mg, crude) as a white solid. LCMS (ESI) [M+H]⁺ m/z: calcd 472.2, found 472.1.

### Step 10: Synthesis of 5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrole-2-carbaldehyde

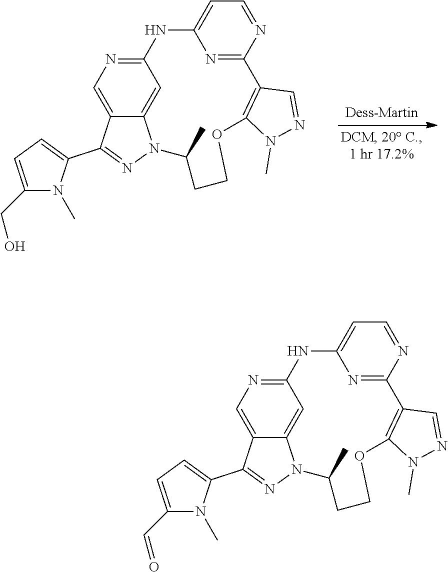 [SMILES: C[C@H]1CCOc2c(cnn2C)-c2nccc(n2)Nc2cc3c(cn2)c(-c2ccc(CO)n2C)nn31 ; C[C@H]1CCOc2c(cnn2C)-c2nccc(n2)Nc2cc3c(cn2)c(-c2ccc(C=O)n2C)nn31]

To a solution of [5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrol-2-yl]methanol (650 mg, 1.38 mmol, 1.0 eq) in DCM (8.0 mL) was added Dess-Martin (250 mg, 0.589 mmol, 0.4 eq). The mixture was stirred at 20° C. for 1 hour. The reaction mixture was quenched by saturated Na₂CO₃ aqueous solution (20 mL) and extracted with DCM (20 mL*3). The combined organic layers were washed with brine (20 mL), dried over Na₂SO₄, filtered and concentrated under reduced pressure. The residue was purified by preparative TLC (SiO₂, EtOAc/DCM=2/3, 254 nm) to afford 5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrole-2-carbaldehyde (130 mg, 17.2% yield, 86% purity) as a white solid. LCMS (ESI) [M+H]⁺ m/z: calcd 470.2, found 470.1.

### Step 11: Synthesis of (16S)-11,16-dimethyl-19-[1-methyl-5-[(4-methylpiperazin-1-yl)methyl]pyrrol-2-yl]-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaene

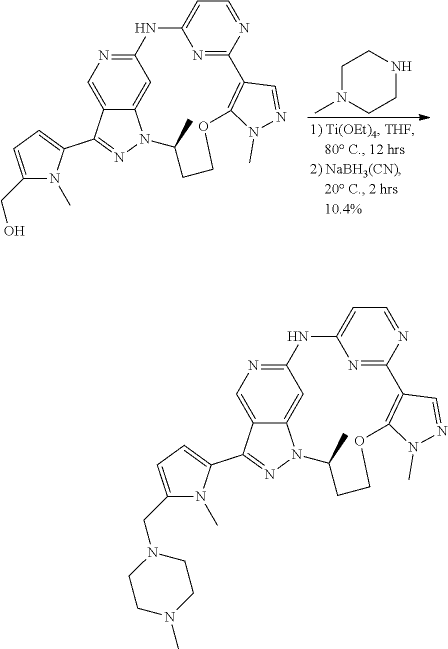

A mixture of 5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrole-2-carbaldehyde (130 mg, 0.277 mmol, 1.0 eq), 1-methylpiperazine (100 mg, 0.998 mmol, 3.6 eq) and Ti(OEt)₄ (440 mg, 1.93 mmol, 7.0 eq) in THF (5.0 mL) was degassed and purged with nitrogen for 3 times, and then the mixture was stirred at 80° C. for 12 hours under nitrogen atmosphere. Then NaBH₃(CN) (100 mg, 1.59 mmol, 5.8 eq) was added and the mixture was stirred at 20° C. for 2 hours. The reaction mixture was quenched by H₂O (0.2 mL), and then diluted with EtOAc (30 mL). Then silica powder (˜2 g) was added and the mixture was stirred at 20° C. for 30 minutes. The mixture was filtered and concentrated under reduced pressure. The residue was purified by preparative HPLC (Column: Waters Xbridge 150*25 mm*10 μm; mobile phase: [water (0.05% NH₃—H₂O+10 mM NH₄HCO₃)-ACN]; B %: 23%-53%, 9.5 min; Column Temp: 20° C.) to afford (16S)-11,16-dimethyl-19-[1-methyl-5-[(4-methylpiperazin-1-yl)methyl]pyrrol-2-yl]-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaene (16.5 mg, 10.4% yield, 97% purity) as a white solid.

LCMS (ESI) [M+H]⁺ m/z: calcd 554.3, found 554.1.

¹H NMR (400 MHz, CD₃OD) δ ppm 9.02 (s, 1H), 8.81 (s, 1H), 8.21 (d, J=6.0 Hz, 1H), 8.00 (s, 1H), 6.66 (d, J=6.0 Hz, 1H), 6.61 (d, J=3.6 Hz, 1H), 6.16 (d, J=3.6 Hz, 1H), 5.22-5.11 (m, 1H), 4.57-4.46 (m, 2H), 3.95 (s, 3H), 3.92-3.85 (m, 1H), 3.80 (s, 3H), 3.58 (s, 2H), 2.74-2.37 (m, 8H), 2.33 (s, 3H), 2.14-2.01 (m, 1H), 1.85 (d, J=6.8 Hz, 3H).

### Example 8: Preparation of Compound (184)—Typical Procedure for Making Compound I-A-1 (R¹=Substituted Pyrrole)

The synthetic procedure described in this example is also adapted for the preparation of still further compounds of the invention.

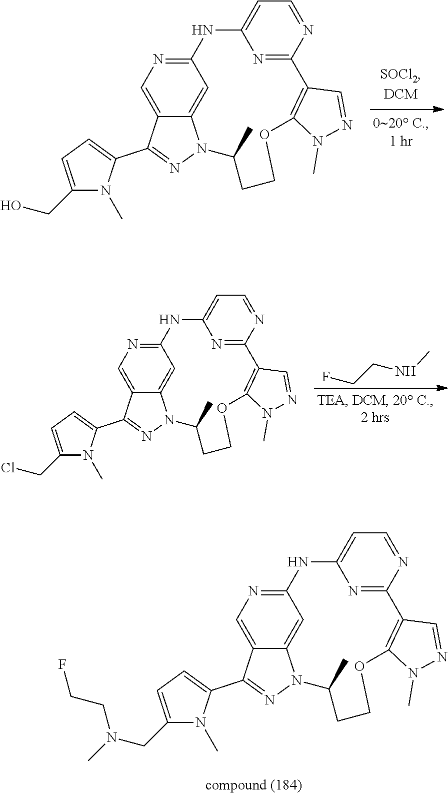

### Step 1: (16S)-19-[5-(chloromethyl)-1-methyl-pyrrol-2-yl]-11,16-dimethyl-13-oxa-2,6,10,11,17,18,22,25-octazapentacyclo[15.5.2.13,7.08,12.020,24]pentacosa-1(22),3,5,7(25),8(12),9,18,20,23-nonaene

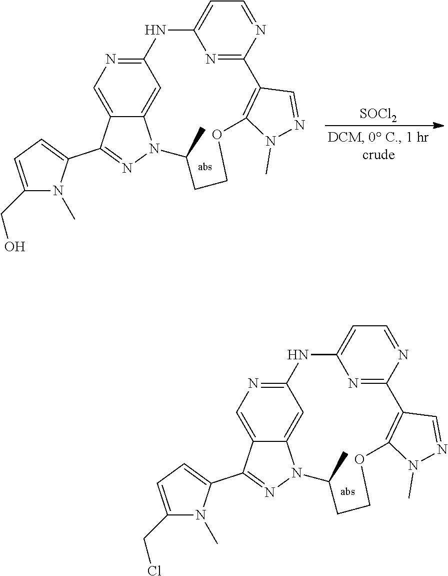

To a solution of [5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrol-2-yl]methanol (15 mg, 0.03 mmol, 1.0 eq) in DCM (3.0 mL) was added SOCl₂ (30 mg, 0.25 mmol, 8.0 eq). The mixture was stirred at 0° C. for 1 hour. The reaction mixture was concentrated in vacuum to give (16S)-19-[5-(chloromethyl)-1-methyl-pyrrol-2-yl]-11,16-dimethyl-13-oxa-2,6,10,11,17,18,22,25-octazapentacyclo[15.5.2.1³.0^{8,12}.0^{20,24}]pentacosa-1(22),3,5,7(25),8(12),9,18,20,23-nonaene (16 mg, crude) as a yellow solid which was directly used into the next step without purification.

### Step 2: (16S)-11,16-dimethyl-19-[1-methyl-5-[(4-methylpiperazin-1-yl)methyl]pyrrol-2-yl]-13-oxa-2,6,10,11,17,18,22,25-octazapentacyclo[15.5.2.13,7.08,12.020,24]pentacosa-1(22),3,5,7(25),8(12),9,18,20,23-nonaene (compound (184))

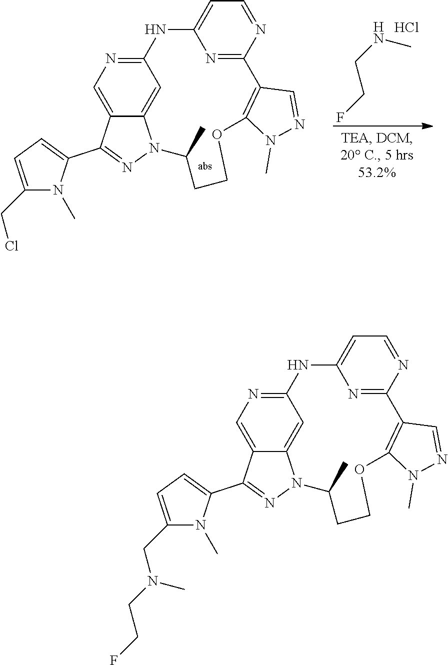

To a solution of (16S)-19-[5-(chloromethyl)-1-methyl-pyrrol-2-yl]-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaene (15 mg, 0.03 mmol, 1.0 eq) and 2-fluoro-N-methyl-ethanamine; hydrochloride (40 mg, 0.35 mmol, 11.5 eq) in DCM (2.0 mL) was added TEA (31.0 mg, 0.31 mmol, 10.0 eq) at 0° C. The mixture was stirred at 20° C. for 5 hours. The reaction mixture was concentrated under reduced pressure and the residue was purified by preparative HPLC (column: Phenomenex Gemini-NX 80*40 mm*3 μm; mobile phase: [water (10 mM NH₄HCO₃)-ACN]; B %: 33%-63%, 9.5 min, 254 nm) to give N-[[5-[(16S)-11,16-dimethyl-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaen-19-yl]-1-methyl-pyrrol-2-yl]methyl]-2-fluoro-N-methyl-ethanamine (9 mg, 53.2% yield) as an off-white solid.

¹H NMR (400 MHz, CD₃OD) δ ppm 9.07 (br s, 1H), 8.85 (br s, 1H), 8.25 (d, J=5.8 Hz, 1H), 8.03 (s, 1H), 6.77-6.59 (m, 2H), 6.27 (d, J=3.3 Hz, 1H), 5.29-5.20 (m, 1H), 4.70 (t, J=4.6 Hz, 1H), 4.62-4.58 (m, 2H), 3.99 (s, 3H), 3.93 (br d, J=10.3 Hz, 1H), 3.83 (s, 5H), 3.00-2.89 (m, 2H), 2.60 (br t, J=11.9 Hz, 1H), 2.46 (s, 3H), 2.20-2.08 (m, 1H), 1.89 (d, J=6.8 Hz, 3H).

LCMS [M+H]⁺ m/z: calcd 531.2, found 531.0.

### Example 9: Preparation of Compound (107)—Typical Procedure for Making Compound I-A-1 (R¹=Substituted Thiazoles)

### Synthesis of (16S)-11,16-dimethyl-9-[5-[(4-methylpiperazin-1-yl)methyl]thiazol-2-yl]-13-oxa-2,6,10,11,17,18,22,25-octazapentacyclo[15.5.2.1^{3,7}.0^{8,12}.0^{20,24}]pentacosa-1(22),3,5,7(25),8(12),9,18,20,23-nonaene (compound (107))

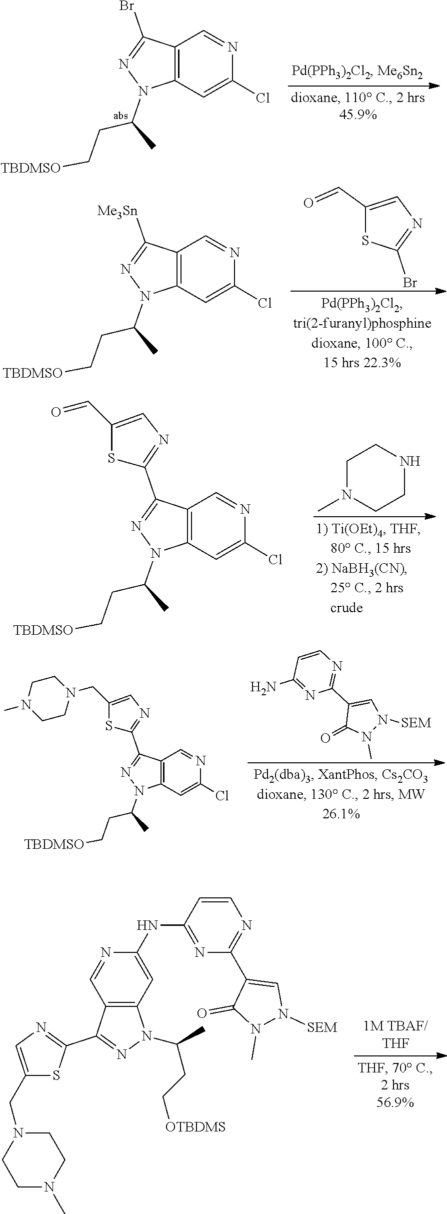

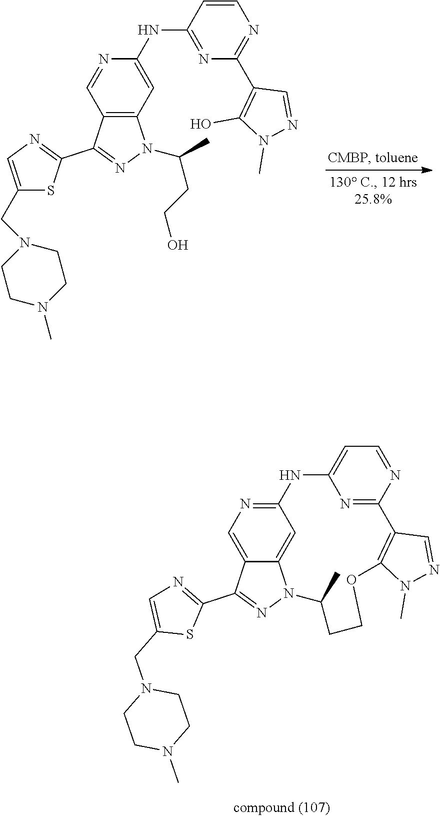 [SMILES: C[C@@H](CCO)n1nc(-c2ncc(CN3CCN(C)CC3)s2)c2cnc(Nc3ccnc(-c4cnn(C)c4O)n3)cc21 ; C[C@H]1CCOc2c(cnn2C)-c2nccc(n2)Nc2cc3c(cn2)c(-c2ncc(CN4CCN(C)CC4)s2)nn31]

### Step 1: Synthesis of tert-butyl-[(3S)-3-(6-chloro-3-trimethylstannyl-pyrazolo[4,3-c]pyridin-1-yl)butoxy]-dimethyl-silane

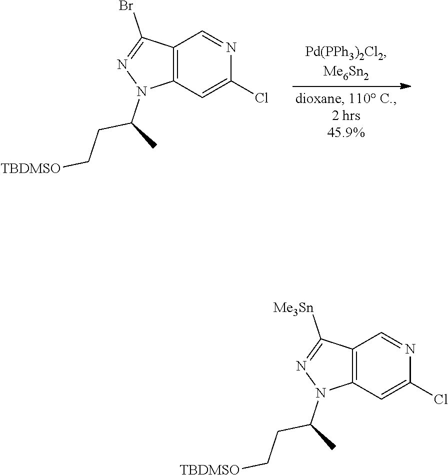

A mixture of [(3S)-3-(3-bromo-6-chloro-pyrazolo[4,3-c]pyridin-1-yl)butoxy]-tert-butyl-dimethyl-silane (2.5 g, 5.97 mmol, 1.0 eq), Pd(PPh₃)₂Cl₂ (420 mg, 0.598 mmol, 0.1 eq) and trimethyl(trimethylstannyl)stannane (5.35 g, 16.3 mmol, 2.7 eq) in dioxane (30.0 mL) was degassed and purged with nitrogen for 3 times, and then the mixture was stirred at 110° C. for 2 hours under nitrogen atmosphere. The reaction mixture was filtered and the filter cake was washed with EtOAc (50 mL*2). The combined filtrate were washed with brine (40 mL*2), dried over Na₂SO₄, filtered and concentrated under reduced pressure. The residue was purified by flash chromatography (ISCO®; 40 g SepaFlash® Silica Flash Column, petroleum ether/EtOAc with EtOAc form 0-3%, flow rate=80 mL/min, 254 nm) to afford tert-butyl-[(3S)-3-(6-chloro-3-trimethylstannyl-pyrazolo[4,3-c]pyridin-1-yl)butoxy]-dimethyl-silane (2.0 g, 45.9% yield, 69% purity) as a yellow oil.

LCMS [M+H]⁺ m/z: calcd 504.1, found 504.0.

¹H NMR (400 MHz, CDCl₃) δ ppm 8.75 (d, J=0.8 Hz, 1H), 7.36 (s, 1H), 4.91-4.82 (m, 1H), 3.58-3.52 (m, 1H), 3.15 (dt, J=4.0, 9.8 Hz, 1H), 2.31-2.22 (m, 1H), 2.07-2.00 (m, 1H), 1.60 (d, J=6.8 Hz, 3H), 0.88 (s, 9H), 0.47 (s, 9H), −0.05 (d, J=12.0 Hz, 6H).

### Step 2: Synthesis of 2-[1-[(1S)-3-[tert-butyl(dimethyl)silyl]oxy-1-methyl-propyl]-6-chloro-pyrazolo[4,3-c]pyridin-3-yl]thiazole-5-carbaldehyde

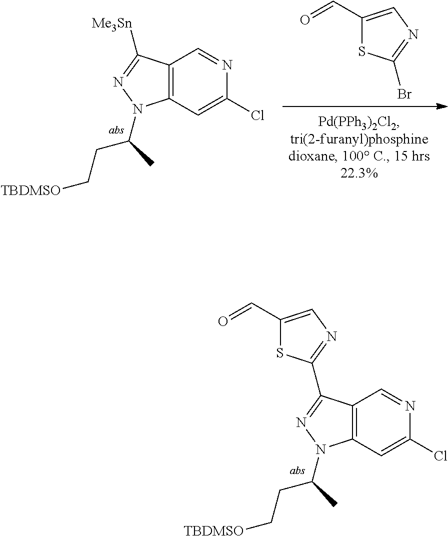

A mixture of tert-butyl-[(3S)-3-(6-chloro-3-trimethylstannyl-pyrazolo[4,3-c]pyridin-1-yl)butoxy]-dimethyl-silane (500 mg, 0.995 mmol, 1.0 eq), 2-bromothiazole-5-carbaldehyde (300 mg, 1.56 mmol, 1.6 eq), Pd(PPh₃)₂Cl₂ (75 mg, 0.107 mmol, 0.1 eq) and tris(2-furyl)phosphane (25 mg, 0.108 mmol, 0.1 eq) in dioxane (10.0 mL) was degassed and purged with nitrogen for 3 times, and then the mixture was stirred at 100° C. for 15 hours under nitrogen atmosphere. The reaction mixture was filtered, and the filter cake was washed with DCM (40 mL). The combined filtrate concentrated under reduced pressure. The residue was purified by flash chromatography (ISCO®; 25 g SepaFlash® Silica Flash Column, petroleum ether/EtOAc with EtOAc from 0-4%, flow rate=50 mL/min, 254 nm) to afford 2-[1-[(1S)-3-[tert-butyl(dimethyl)silyl]oxy-1-methyl-propyl]-6-chloro-pyrazolo[4,3-c]pyridin-3-yl]thiazole-5-carbaldehyde (110 mg, 22.3% yield, 91% purity) as a white solid.

LCMS [M+H]⁺ m/z: calcd 451.1, found 451.0.

¹H NMR (400 MHz, CDCl₃) δ ppm 10.12 (s, 1H), 9.53 (d, J=0.8 Hz, 1H), 8.54 (s, 1H), 7.48 (d, J=0.8 Hz, 1H), 5.01-4.92 (m, 1H), 3.66-3.58 (m, 1H), 3.16 (dt, J=3.4, 10.2 Hz, 1H), 2.32-2.23 (m, 1H), 2.13-2.05 (m, 1H), 1.67 (d, J=6.8 Hz, 3H), 0.89 (s, 9H), −0.03 (d, J=16.8 Hz, 6H).

### Step 3: Synthesis of tert-butyl-[(3S)-3-[6-chloro-3-[5-[(4-methylpiperazin-1-yl)methyl]thiazol-2-yl]pyrazolo[4,3-c]pyridin-1-yl]butoxy]-dimethyl-silane

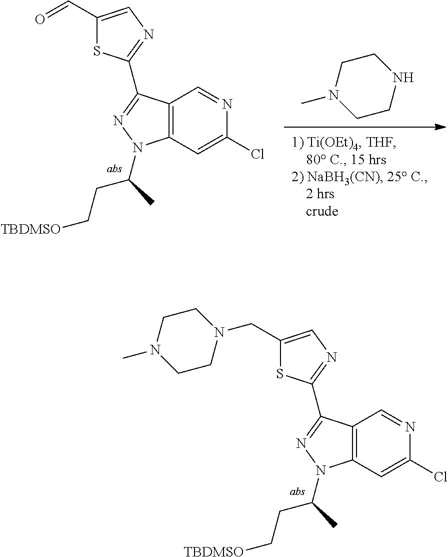

A mixture of 2-[1-[(1S)-3-[tert-butyl(dimethyl)silyl]oxy-1-methyl-propyl]-6-chloro-pyrazolo[4,3-c]pyridin-3-yl]thiazole-5-carbaldehyde (110 mg, 0.244 mmol, 1.0 eq), 1-methylpiperazine (80 mg, 0.799 mmol, 3.3 eq) and Ti(OEt)₄ (170 mg, 0.745 mmol, 3.1 eq) in THF (5.0 mL) was stirred at 80° C. for 15 hours, and then NaBH₃CN (400 mg, 6.37 mmol, 26.1 eq) was added. The mixture was stirred at 25° C. for 2 hours. The reaction mixture was quenched by addition water (2 mL) at 0° C., and then silica powder (3 g) was added. The mixture was filtered and washed with a mixture of DCM/MeOH (30 mL, v/v=10/1). The combined filtrate concentrated under reduced pressure and the residue was purified by flash chromatography (ISCO®; 25 g SepaFlash® Silica Flash Column, DCM/MeOH with MeOH from 0-15%, flow rate=40 mL/min, 254 nm) to afford tert-butyl-[(3S)-3-[6-chloro-3-[5-[(4-methylpiperazin-1-yl)methyl]thiazol-2-yl]pyrazolo[4,3-c]pyridin-1-yl]butoxy]-dimethyl-silane (150 mg, crude) as a yellow oil.

LCMS [M+H]⁺ m/z: calcd 535.2, found 535.1.

¹H NMR (400 MHz, CDCl₃) δ ppm 9.47 (s, 1H), 7.75 (s, 1H), 7.41 (s, 1H), 4.91 (br s, 1H), 3.80 (s, 2H), 3.64-3.57 (m, 1H), 3.17 (dt, J=3.2, 10.2 Hz, 1H), 2.48 (br s, 2H), 2.30 (s, 3H), 2.25 (br dd, J=4.2, 9.8 Hz, 2H), 2.06 (dt, J=4.8, 9.4 Hz, 2H), 1.64 (br s, 3H), 0.89 (s, 9H), 0.63-0.57 (m, 4H), −0.04 (d, J=15.4 Hz, 6H).

### Step 4: Synthesis of 4-[4-[[1-[(1S)-3-[tert-butyl(dimethyl)silyl]oxy-1-methyl-propyl]-3-[5-[(4-methylpiperazin-1-yl)methyl]thiazol-2-yl]pyrazolo[4,3-c]pyridin-6-yl]amino]pyrimidin-2-yl]-2-methyl-1-(2-trimethylsilylethoxymethyl)pyrazol-3-one

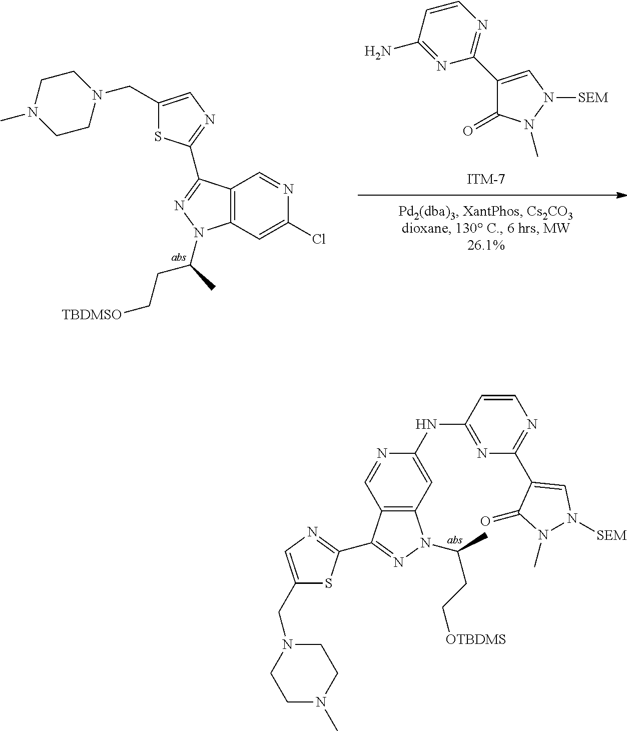 [SMILES: C[C@@H](CCO[Si](C)(C)C(C)(C)C)n1nc(-c2ncc(CN3CCN(C)CC3)s2)c2cnc(Cl)cc21 ; Cn1c(=O)c(-c2nccc(N)n2)cn1COCC[Si](C)(C)C ; C[C@@H](CCO[Si](C)(C)C(C)(C)C)n1nc(-c2ncc(CN3CCN(C)CC3)s2)c2cnc(Nc3ccnc(-c4cn(COCC[Si](C)(C)C)n(C)c4=O)n3)cc21]

Tert-butyl-[(3S)-3-[6-chloro-3-[5-[(4-methylpiperazin-1-yl)methyl]thiazol-2-yl]pyrazolo[4,3-c]pyridin-1-yl]butoxy]-dimethyl-silane (90 mg, 0.168 mmol, 1.0 eq), 4-(4-aminopyrimidin-2-yl)-2-methyl-1-(2-trimethylsilylethoxymethyl)pyrazol-3-one (70 mg, 0.218 mmol, 1.3 eq), Pd₂(dba)₃ (20 mg, 0.0218 mmol, 0.1 eq), XantPhos (10 mg, 0.0173 mmol, 0.1 eq) and Cs₂CO₃ (180 mg, 0.552 mmol, 3.3 eq) were taken up into a microwave tube in dioxane (3.0 mL). The sealed tube was heated at 130° C. for 6 hours under microwave. The reaction mixture was filtered and the filter cake was washed with DCM (50 mL). The combined filtrate concentrated under reduced pressure and the residue was purified by preparative TLC (silica, DCM/MeOH=10/1, 254 nm) to afford 4-[4-[[1-[(1S)-3-[tert-butyl(dimethyl)silyl]oxy-1-methyl-propyl]-3-[5-[(4-methylpiperazin-1-yl)methyl]thiazol-2-yl]pyrazolo[4,3-c]pyridin-6-yl]amino]pyrimidin-2-yl]-2-methyl-1-(2-trimethylsilylethoxymethyl)pyrazol-3-one (50 mg, 26.1% yield, 72% purity) as a yellow solid.

LCMS [M+H]⁺ m/z: calcd 820.4, found 820.4.

### Step 5: Synthesis of 4-[4-[[1-[(1S)-3-hydroxy-1-methyl-propyl]-3-[5-[(4-methylpiperazin-1-yl)methyl]thiazol-2-yl]pyrazolo[4,3-c]pyridin-6-yl]amino]pyrimidin-2-yl]-2-methyl-pyrazol-3-ol

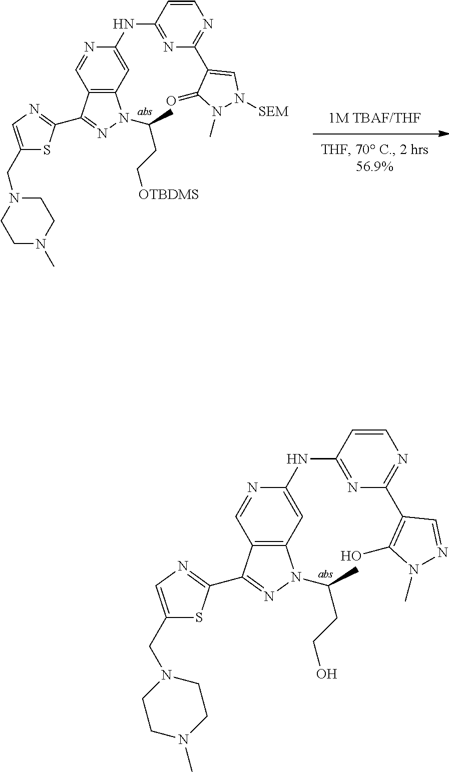 [SMILES: C[C@@H](CCO[Si](C)(C)C(C)(C)C)n1nc(-c2ncc(CN3CCN(C)CC3)s2)c2cnc(Nc3ccnc(-c4cn(COCC[Si](C)(C)C)n(C)c4=O)n3)cc21 ; C[C@@H](CCO)n1nc(-c2ncc(CN3CCN(C)CC3)s2)c2cnc(Nc3ccnc(-c4cnn(C)c4O)n3)cc21]

To a mixture of 4-[4-[[1-[(1S)-3-[tert-butyl(dimethyl)silyl]oxy-1-methyl-propyl]-3-[5-[(4-methylpiperazin-1-yl)methyl]thiazol-2-yl]pyrazolo[4,3-c]pyridin-6-yl]amino]pyrimidin-2-yl]-2-methyl-1-(2-trimethylsilylethoxymethyl)pyrazol-3-one (50 mg, 0.0610 mmol, 1.0 eq) in THF (5.0 mL) was added 1M TBAF/THF (0.2 mL, 0.2 mmol, 3.3 eq). The mixture was stirred at 70° C. for 2 hours. The reaction mixture was concentrated under reduced pressure and the residue was purified by flash chromatography (Column: SepaFlash® Sphercial C18, 40 g, 40-60 μm, 120 Å; MeCN/water (0.05% NH₃—H₂O) with MeCN from 0-35%, 50 mL/min, 254 nm) to afford 4-[4-[[1-[(1S)-3-hydroxy-1-methyl-propyl]-3-[5-[(4-methylpiperazin-1-yl)methyl]thiazol-2-yl]pyrazolo[4,3-c]pyridin-6-yl]amino]pyrimidin-2-yl]-2-methyl-pyrazol-3-ol (20 mg, 56.9% yield, 100% purity) as a yellow solid.

LCMS [M+H]⁺ m/z: calcd 576.2, found 576.1.

### Step 6: Synthesis of (16S)-11,16-dimethyl-19-[5-[(4-methylpiperazin-1-yl)methyl]thiazol-2-yl]-13-oxa-2,6,10,11,17,18,22,25-octazapentacyclo[15.5.2.13,7.08,12.020,24]pentacosa-1(22),3,5,7(25),8(12),9,18,20,23-nonaene

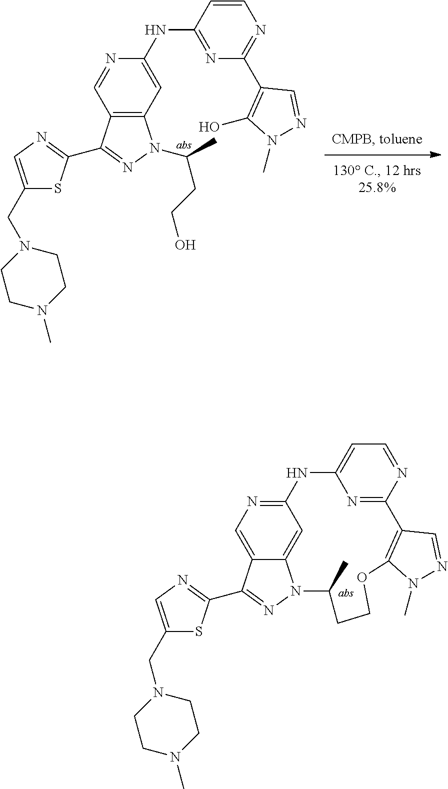 [SMILES: C[C@@H](CCO)n1nc(-c2ncc(CN3CCN(C)CC3)s2)c2cnc(Nc3ccnc(-c4cnn(C)c4O)n3)cc21 ; C[C@H]1CCOc2c(cnn2C)-c2nccc(n2)Nc2cc3c(cn2)c(-c2ncc(CN4CCN(C)CC4)s2)nn31]

A mixture of 4-[4-[[1-[(1S)-3-hydroxy-1-methyl-propyl]-3-[5-[(4-methylpiperazin-1-yl)methyl]thiazol-2-yl]pyrazolo[4,3-c]pyridin-6-yl]amino]pyrimidin-2-yl]-2-methyl-pyrazol-3-ol (20 mg, 0.0347 mmol, 1.0 eq) and 2-(tributyl-λ5-phosphanylidene)acetonitrile (50 mg, 0.207 mmol, 6.0 eq) in toluene (5.0 mL) was degassed and purged with nitrogen for 3 times, and then the mixture was stirred at 130° C. for 12 hours under nitrogen atmosphere. The reaction mixture was concentrated under reduced pressure. The residue was purified by preparative TLC (silica, DCM/MeOH=10/1, 254 nm) to give a crude product which was purified by preparative HPLC (Column: Phenomenex Gemini 150*25 mm*10 μm; Mobile phase: [water (0.05% NH₃—H₂O+10 mM NH₄HCO₃)-ACN]; B %: 27%-57%, 7.8 min, Column Temp: 30° C., 254 nm) to afford (16S)-11,16-dimethyl-19-[5-[(4-methylpiperazin-1-yl)methyl]thiazol-2-yl]-13-oxa-2,6,10,11,17,18,22,25-octazapentacyclo[15.5.2.13,7.08,12.020,24]pentacosa-1(22),3,5,7(25),8(12),9,18,20,23-nonaene (5.0 mg, 25.8% yield, 100% purity) as off-white solid.

LCMS [M+H]⁺ m/z: calcd 558.2, found 558.1.

¹H NMR (400 MHz, CD₃OD) δ ppm 9.27 (d, J=1.0 Hz, 1H), 9.16 (s, 1H), 8.26 (d, J=6.0 Hz, 1H), 8.03 (s, 1H), 7.81 (s, 1H), 6.72 (d, J=6.0 Hz, 1H), 5.35-5.23 (m, 2H), 4.54 (s, 2H), 3.96 (br d, J=10.4 Hz, 1H), 3.87 (s, 2H), 3.83 (s, 3H), 2.72-2.50 (m, 7H), 2.32 (s, 3H), 2.22-2.11 (m, 1H), 1.91 (d, J=6.8 Hz, 3H).

### Example 10: In Vitro Assays

The biological activity of compounds described herein can be studied according to standard methods known in the art. Methods can be used to study inhibition of EGFR, including mutant forms of EGFR comprising L858R, T790M, C797S, and/or Del19 mutations, or any combination thereof (e.g., L858R single, double, or triple mutants). Exemplary, non-limiting methods are described herein.

### Kinase Assays

Assays using an in vitro kinase assay kit (HTRF KinEASE-TK kit) can be used to study the inhibitory activity of compounds described herein with respect to EGFR mutants such as EGFR^{L858R}, EGFR^{L58R/T790M}, and EGFR^{L858R/T790M}/C^{797S}.

Ba/F3 Viability Assays

Inhibition of cell proliferation can be studied using Ba/F3 viability assays, including the Promega CellTiter-Glo cell viability assay. This assay can be used to study the effect of compounds described herein in the following assays: (1) Ba/F3 Parental; (2) Ba/F3 EGFR-Del19/T790M; (3); Ba/F3 EGFR-Del19/C797S; and (4) Ba/F3 EGFR-Del19/T790M/C797S.

### P-EGFR Signaling Assays

Phosphorylation of EGFR can be studied using multiplex immunoassay kits such as Phospho-EGFR (Tyr1068) Total EGFR MULTI-SPOT® 96 HB 4-Spot Custom EGFR Duplex ANALYTES assay.

Exemplary kinase inhibition (Kinase) and anti-proliferation activity (Ba/F3) data are shown in Table 1 for certain compounds of the invention as described herein.

TABLE 1
In vitro Assay Data

| | kinase | | | BaF3 | | | |
|---|---|---|---|---|---|---|---|
| cmpnd # | LTC | LT | L | DTC | DT | DC | parental |
| (1) | A | A | A | A | A | B | C |
| (2) | A | A | B | B | B | B | C |
| (3) | A | A | B | C | B | C | C |
| (4) | A | A | C | C | C | C | D |
| (5) | A | A | B | B | B | B | D |
| (6) | A | A | B | B | B | B | C |
| (8) | A | A | B | B | B | B | C |
| (10) | A | A | C | C | C | C | C |
| (11) | A | A | C | C | C | C | C |
| (12) | A | A | B | B | B | B | C |
| (13) | A | A | B | B | B | C | C |
| (14) | A | A | A | A | A | A | B |
| (15) | A | A | B | B | B | B | C |
| (16) | A | A | B | B | B | B | C |
| (33) | A | A | B | B | B | B | D |
| (37) | A | A | B | B | B | B | D |
| (57) | A | A | B | B | B | B | C |
| (58) | A | A | B | B | B | B | D |
| (61) | B | A | C | C | C | C | D |
| (62) | A | A | C | C | C | C | C |
| (63) | A | A | A | B | B | A | C |
| (64) | A | A | B | B | B | B | C |
| (65) | A | A | C | C | C | C | C |
| (66) | A | A | B | C | C | C | D |
| (67) | C | B | D | D | D | D | D |
| (68) | B | A | C | C | C | C | D |
| (69) | A | A | B | B | B | B | D |
| (70) | A | A | A | B | A | B | C |
| (71) | A | A | A | B | B | B | C |
| (73) | A | A | B | B | B | B | D |
| (74) | A | A | C | C | C | C | D |
| (75) | A | A | B | B | B | B | C |
| (76) | A | A | A | A | A | A | B |
| (77) | C | B | D | C | D | C | D |
| (78) | A | A | B | B | B | B | C |
| (79) | A | A | B | B | B | B | D |
| (80) | A | A | A | A | A | A | B |
| (82) | A | A | C | B | B | B | B |
| (83) | A | A | B | B | B | B | B |
| (84) | A | A | B | B | B | C | C |
| (85) | A | A | B | A | A | B | D |
| (86) | A | A | B | B | B | B | D |
| (87) | A | A | B | A | B | B | C |
| (88) | A | A | B | C | C | C | C |
| (89) | A | A | B | B | B | B | C |
| (90) | A | A | A | A | A | A | C |
| (91) | A | A | B | B | B | B | D |
| (92) | A | A | B | B | B | B | C |
| (93) | A | A | A | A | A | A | B |
| (94) | A | A | C | C | C | C | D |
| (95) | A | A | A | B | B | B | C |
| (96) | A | A | A | B | B | B | C |
| (97) | B | A | C | C | C | C | D |
| (98) | A | A | A | B | B | B | C |
| (99) | A | A | B | B | B | B | C |
| (100) | A | A | B | B | B | B | C |
| (101) | A | A | A | A | A | A | C |
| (102) | A | A | A | B | B | B | C |
| (103) | A | A | A | A | A | B | C |
| (104) | A | A | B | B | B | B | C |
| (105) | A | A | A | A | A | B | D |
| (106) | A | A | A | B | B | A | C |
| (107) | A | A | A | A | A | A | C |
| (108) | A | A | A | A | B | B | C |
| (109) | A | A | A | A | A | A | C |
| (110) | A | A | A | A | A | A | C |
| (111) | A | A | A | A | A | B | C |
| (112) | A | A | A | A | A | B | C |
| (113) | A | A | A | A | A | A | C |
| (114) | A | A | B | B | B | B | C |
| (115) | A | A | B | B | B | B | C |
| (116) | A | A | B | B | B | B | C |
| (117) | A | A | A | A | A | B | B |
| (118) | A | A | B | B | C | C | D |
| (119) | A | A | A | A | A | A | C |
| (120) | A | A | A | A | A | B | C |
| (121) | A | A | A | A | A | A | C |
| (122) | A | A | A | A | A | A | C |
| (123) | A | A | A | A | A | A | C |
| (124) | A | A | A | A | A | A | C |
| (125) | A | A | B | A | A | B | C |
| (126) | A | A | A | A | A | A | C |
| (127) | A | A | A | A | A | A | C |
| (128) | A | A | B | B | B | B | C |
| (129) | A | A | B | B | B | B | C |
| (130) | A | A | B | B | B | B | D |
| (131) | A | A | A | A | A | A | C |
| (132) | A | A | B | B | B | B | C |
| (133) | A | A | B | B | B | B | C |
| (134) | A | A | A | B | B | B | C |
| (135) | A | A | A | A | A | A | C |
| (136) | A | A | A | A | A | A | C |
| (137) | A | A | A | A | A | A | C |
| (138) | A | A | A | A | A | A | C |
| (139) | A | A | A | A | A | A | C |
| (140) | A | A | A | A | A | A | C |
| (141) | A | A | A | B | B | B | C |
| (142) | A | A | A | A | B | B | C |
| (143) | A | A | B | B | B | B | D |
| (144) | A | A | A | A | A | A | B |
| (145) | A | A | A | A | A | B | C |
| (146) | A | A | A | A | A | A | C |
| (147) | A | A | A | A | A | A | C |
| (148) | A | A | A | A | A | A | C |
| (149) | A | A | A | A | A | B | C |
| (150) | A | A | B | B | B | B | D |
| (151) | A | A | A | A | A | B | C |
| (152) | A | A | A | A | A | A | C |
| (153) | A | A | A | B | B | B | C |
| (154) | A | A | A | A | B | B | C |
| (155) | A | A | A | B | B | B | D |
| (156) | A | A | A | A | A | A | C |
| (157) | A | A | A | A | B | B | C |
| (158) | A | A | C | C | C | C | C |
| (159) | B | B | C | C | C | C | C |
| (160) | A | A | A | A | A | B | C |
| (161) | A | A | A | A | A | B | C |
| (162) | A | A | A | A | A | A | C |
| (163) | A | A | A | A | B | B | C |
| (164) | A | A | A | A | A | B | C |
| (165) | A | A | A | A | A | A | C |
| (166) | A | A | A | A | A | A | C |
| (167) | A | A | A | A | A | A | C |
| (168) | A | A | A | A | A | A | C |
| (169) | A | A | A | A | A | A | C |
| (170) | A | A | A | A | B | B | C |
| (171) | A | A | A | B | B | B | C |
| (172) | A | A | A | A | A | A | B |
| (173) | A | A | A | A | A | A | C |
| (174) | A | A | A | A | A | A | C |
| (175) | A | A | A | A | A | A | B |
| (176) | A | A | A | A | A | B | C |
| (177) | A | A | B | A | B | B | C |
| (178) | A | A | B | A | B | B | C |
| (179) | A | A | A | A | A | B | C |
| (180) | A | A | B | A | B | B | C |
| (181) | A | A | A | B | B | B | C |
| (182) | A | A | B | A | B | B | C |
| (183) | A | A | A | A | B | B | C |
| (184) | A | A | A | A | A | B | C |
| (185) | A | A | A | B | B | B | C |
| (186) | A | A | A | A | A | A | C |
| (187) | A | A | A | A | A | A | C |
| (188) | A | A | A | A | A | A | C |
| (189) | A | A | A | A | A | A | C |
| (190) | A | A | A | A | A | A | C |
| (191) | A | A | A | A | B | A | C |
| (192) | A | A | A | A | A | A | C |
| (193) | A | A | A | A | A | A | C |

Legend:
A = IC₅₀ < 10 nM
B = 10 nM ≤ IC₅₀ < 100 nM
C = 100 nM ≤ IC₅₀ < 1000 nM
D = IC₅₀ ≥ 1000 nM

From the foregoing description, one skilled in the art can easily ascertain the essential characteristics of this invention, and without departing from the spirit and scope thereof, can make various changes and modifications of the invention to adapt it to various usages and conditions.

All references, patents or applications, U.S. or foreign, cited in the application are hereby incorporated by reference as if written herein in their entireties. Where any inconsistencies arise, material literally disclosed herein controls.

What is claimed is:

1. A compound of Formula I: 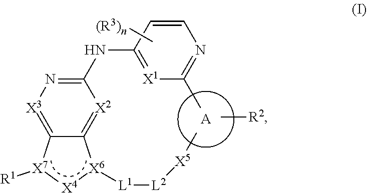
or a pharmaceutically acceptable salt thereof,
wherein:
A is C₆₋₁₀ arylene, 5-12-membered heteroarylene, or 5-12-membered heterocycloalkylene;
X¹ is N or CR^{X};
X² is N or CR^{X};
X³ is N or CR^{X};
X⁴ is N or CR^{X};
X⁶ is N or CR^{X′};
X⁷ is N or CR^{X′}; [symbol]
represents an optional double bond between X⁷ and X⁴ or X⁴ and X⁶, wherein one and only one double bond is present;
X⁵ is a covalent bond, CH₂, O, NR⁴, C(O)NR⁴, or NR⁴C(O);
L¹ is a covalent bond or C(R⁵)₂, and L² is C₁₋₄ alkylene, or L¹ and L² combine to form a C₃₋₆ cycloalkyl or a 4- to 6-membered heterocycloalkyl;
R¹ is halogen, C₁₋₆ alkyl, C₃₋₇ cycloalkyl, C₆₋₁₀ aryl, 5- to 10-membered heteroaryl, 3- to 10-membered heterocycloalkyl, CN, NR⁶R⁷, NR⁶C(O)R⁷, NR⁶C(O)NH₂, OR⁸, or C(O)NR⁶R⁷;
R² is absent, H, C₁₋₆ alkyl, halogen, CN, or C₁₋₆ alkoxy;
each R³, when present, is independently OH, CN, halogen, C₁₋₆ alkyl, or C₁₋₆ alkoxy;
n is 0, 1, or 2;
each R^{X} is independently H, OR^{X1}, CN, halogen, or C₁₋₆ alkyl, wherein R^{X1} is H or C₁₋₆ alkyl;
each R^{X′} is independently H, OR^{X1}, CN, halogen, or C₁₋₆ alkyl, wherein R^{X1} is H or C₁₋₆ alkyl, or R^{X′} is absent if the carbon to which it is attached is part of a double bond;
each R⁴ and R⁵ is independently H or C₁₋₆ alkyl;
each R⁶ and R⁷ is independently H, C₁₋₆ alkyl, C₃₋₇ cycloalkyl, or 3- to 10-membered heterocycloalkyl; or R⁶ and R⁷ together with the nitrogen atom to which they are attached form a 3- to 8-membered heterocycloalkyl ring; and
R⁸ is independently H, C₁₋₆ alkyl, or 4- to 6-membered heterocycloalkyl.

2. The compound of claim 1, having a structure according to Formula I′: 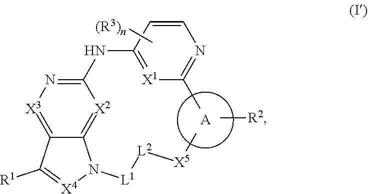
or a pharmaceutically acceptable salt thereof.

3. The compound of claim 1 or 2, wherein n is 0.

4. The compound of claim any one of claims 1-3, wherein X³ is CH.

5. The compound of any one of claims 1-4, wherein X² is N or CH.

6. The compound of any one of claims 1-5, wherein X¹ is N or CH.

7. The compound of any one of claims 1-6, wherein one of X¹ and X² is N and the other is CH.

8. The compound of any one of claims 1-7, wherein X⁴ is N or CH.

9. The compound of any one of claims 1-8, wherein L¹ is CHR⁵, and R⁵ is H, CH₃, or CH₂CH₃.

10. The compound of any one of claims 1-8, wherein L¹ is C(CH₃)₂ or CHCH₃.

11. The compound of any one of claims 1-10, wherein L² is unsubstituted C₁₋₄ alkylene, or C₁₋₄ alkylene substituted by unsubstituted C₁₋₃ alkyl.

12. The compound of claim 11, wherein L² is (CH₂)₂, (CH₂)₃, CH(CH₃)CH₂, or CH₂CH(CH₃).

13. The compound of any one of claims 1-8, wherein L¹ and L² combine to form cyclopropyl, cyclobutyl, cyclopentyl, or cyclohexyl.

14. The compound of any one of claims 1-13, wherein X⁵ is O or NR⁴.

15. The compound of claim 14, wherein X⁵ is O, NH, or NCH₃.

16. The compound of any one of claims 1-15, wherein A is C₆₋₁₀ arylene or 5-12-membered heteroarylene.

17. The compound of any one of claims 1-15, wherein A is 5-12-membered heteroarylene or 5-12-membered heterocycloalkylene.

18. The compound of any one of claims 1-15, wherein A is pyridyl, pyrazolyl, thiazolyl, oxazolyl, imidazyolyl, 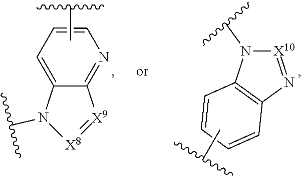
wherein each X⁸, X⁹, and X¹⁰ is CH or N.

19. The compound of claim 18, wherein A is pyrazolyl optionally substituted by methyl.

20. The compound of any one of claims 1-15, wherein A is phenyl.

21. The compound of any one of claims 1-20, wherein R² is absent, H, unsubstituted C₁_3 alkyl, or C₁₋₃ alkyl substituted by unsubstituted C₃₋₆ cycloalkyl.

22. The compound of any one of claims 1-21, wherein R¹ is F, CN, NH₂, O-(oxetan-3-yl), NH-(oxetan-3-yl), O-(tetrahydrofuran-3-yl), O-(1-N,N-dimethylaminocyclohexan-4-yl), NH-(tetrahydrofuran-3-yl), NH(C₁₋₆ alkyl), NCH₃(C₁₋₆ alkyl), and wherein said C₁₋₆ alkyl comprises one or two substituents selected from OH, NH₂, piperidinyl, and CONH₂.

23. The compound of any one of claim 1-21, wherein R¹ is an N-linked group that is azetidine, pyrrolidine, pyrrolyl, or piperazinyl, and wherein said N-linked group is unsubstituted or substituted with a substituent that is OH, CN, oxo, C₁₋₄ alkyl, —NR^{1A}R^{1B} or —C(O)NR^{1A}R^{1B}, wherein
said C₁₋₄ alkyl is unsubstituted or substituted with at least one group that is OH, CN, NH₂, NHCH₃, N(CH₃)₂, N-methylpiperazinyl, C(O)NH₂, C(O)NHCH₃, C(O)N(CH₃)₂,
each R^{1A} and R^{1B} is independently H, C₁₋₆ alkyl, C₃₋₇ cycloalkyl, or 3- to 10-membered heterocycloalkyl; or R^{1A} and R^{1B} together with the nitrogen atom to which they are attached form a 3- to 8-membered heterocycloalkyl ring, wherein said C₁₋₆ alkyl is unsubstituted or substituted with a group that is alkoxy.

24. The compound of any one of claims 1-21, wherein R¹ is C(O)NHR⁷, and R⁷ is a cyclic group that is cyclopentyl, cyclohexyl,
wherein said cyclic group is unsubstituted or substituted by a group that is CN, OH, oxo, C₁₋₄ alkyl, —NR^{1A}R^{1B} or —C(O)NR^{1A}R^{1B}, wherein
said C₁₋₄ alkyl is unsubstituted or substituted with a group that is OH, NH₂, NHCH₃, N(CH₃)₂, N-methylpiperazinyl, C(O)NH₂, C(O)NHCH₃, C(O)N(CH₃)₂,
each R^{1A} and R^{1B} is independently H, C₁₋₆ alkyl, C₃₋₇ cycloalkyl, or 3- to 10-membered heterocycloalkyl; or R^{1A} and R^{1B} together with the nitrogen atom to which they are attached form a 3- to 8-membered heterocycloalkyl ring.

25. The compound of any one of claims 1-21, wherein R¹ is NR⁶R⁷, wherein
R⁶ is independently H or unsubstituted C₁₋₃ alkyl; and
R⁸ is independently C₁₋₆ alkyl, wherein said C₁₋₆ alkyl is unsubstituted or comprises one or two substituent groups selected from —OH and —C(O)NH₂.

26. The compound of any one of claims 1-21, wherein
R¹ is a substituted or unsubstituted 5- or 6-membered heteroarylene; a substituted or unsubstituted 5- or 6-membered heterocycloalkyl, C₁₋₆ alkyl substituted by a 5- or 6-membered heteroarylene that is substituted or unsubstituted; or C₁₋₆ alkyl substituted by a 5- or 6-membered heterocycloalkyl that is substituted or unsubstituted, or substituted phenyl.

27. The compound of claim 1, having a structure according to Formula (I-A), 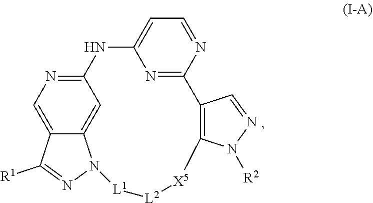
or a pharmaceutically acceptable salt thereof, wherein
R² is unsubstituted C₁₋₆ alkyl or C₁₋₆ alkyl substituted by a group that is unsubstituted C₃₋₆ cycloalkyl; and
L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄, or
L¹-L²-X⁵ is

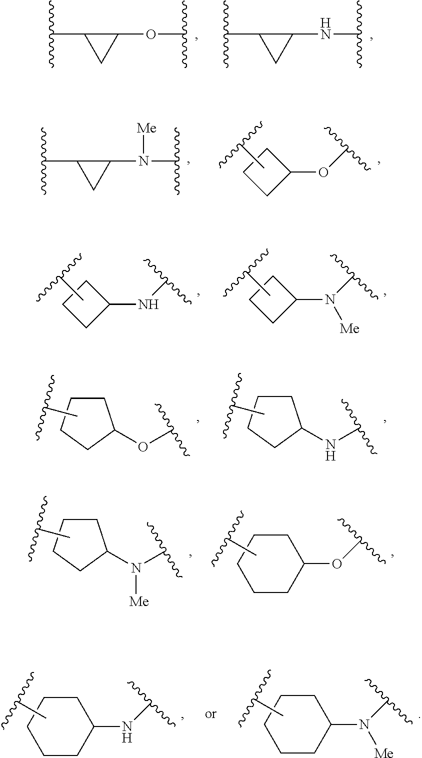

28. The compound of claim 27, wherein R² is CH₃ or C₁₋₃ alkyl substituted by unsubstituted C₃₋₆ cycloalkyl.

29. The compound of claim 27 or 28, wherein L¹-L²-X⁵ is CH(CH₃)—(CH₂)₂—O, CH(CH₃)—(CH₂)₃—O, or (CH₂)₃—O.

30. The compound of any one of claims 27-29, having a structure according to Formula (I-A-1), 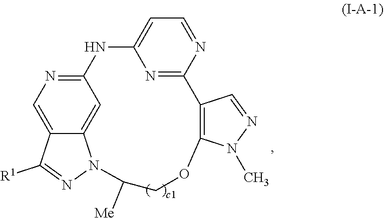
or a pharmaceutically acceptable salt thereof, wherein
c1 is 2 or 3.

31. The compound of claim 30, having a structure according to Formula (I-A-1′), 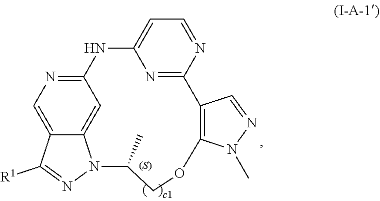
or a pharmaceutically acceptable salt thereof.

32. The compound of claim 30, having a structure according to Formula (I-A-1″), 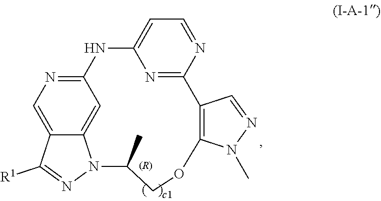
or a pharmaceutically acceptable salt thereof.

33. The compound of any one of claims 27-29, having a structure according to Formula (I-A-2), 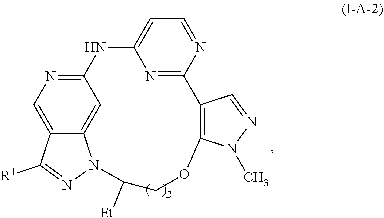
or a pharmaceutically acceptable salt thereof.

34. The compound of any one of claims 27-29, having a structure according to Formula (I-A-3), 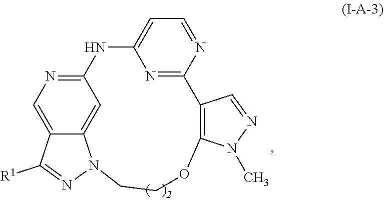
or a pharmaceutically acceptable salt thereof.

35. The compound of claim 1, having a structure according to Formula (I-B), 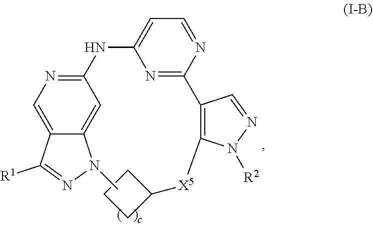
or a pharmaceutically acceptable salt thereof, wherein
R² is unsubstituted C₁₋₆ alkyl or C₁₋₆ alkyl substituted by a group that is unsubstituted C₃₋₆ cycloalkyl;
X⁵ is O; and
c is 0, 1, 2, or 3.

36. The compound of claim 35, wherein R² is CH₃.

37. The compound of claim 35 or 36 having a structure according to Formula (I-B-1), 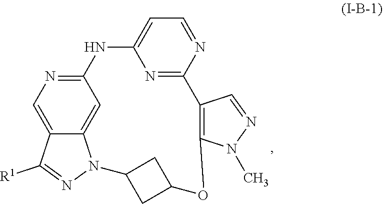
or a pharmaceutically acceptable salt thereof.

38. The compound of claim 35 or 36, having a structure according to Formula (I-B-2), 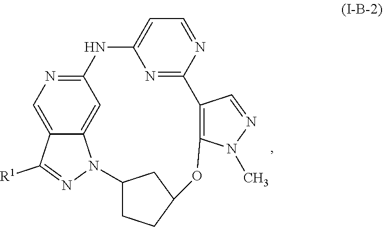
or a pharmaceutically acceptable salt thereof.

39. The compound of claim 35 or 36, having a structure according to Formula (I-B-3), 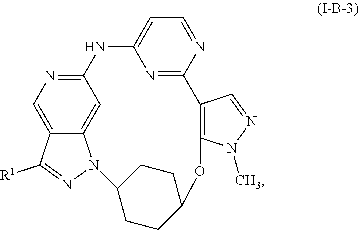
or a pharmaceutically acceptable salt thereof.

40. The compound of claim 1, having a structure according to Formula (I-C), 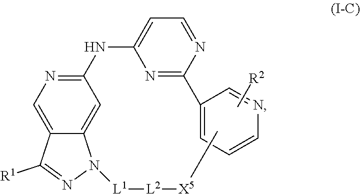
or a pharmaceutically acceptable salt thereof, wherein
R² is H, unsubstituted C₁₋₆ alkyl or C₁₋₆ alkyl substituted by a group that is unsubstituted C₃₋₆ cycloalkyl;
L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, CH(CH₃)—(CH₂)₄, CH(CH₃)—(CH₂)₂—NHC(O), CH(CH₃)—(CH₂)₂—NCH₃C(O), CH(CH₃)—(CH₂)₃—NHC(O), CH(CH₃)—(CH₂)₃—NCH₃C(O), CH(CH₃)—(CH₂)₂—C(O)NH, CH(CH₃)—(CH₂)₂—C(O)NCH₃, CH(CH₃)—(CH₂)₃—C(O)NH, or CH(CH₃)—(CH₂)₃—C(O)NCH₃; or
L¹-L¹-X⁵ is

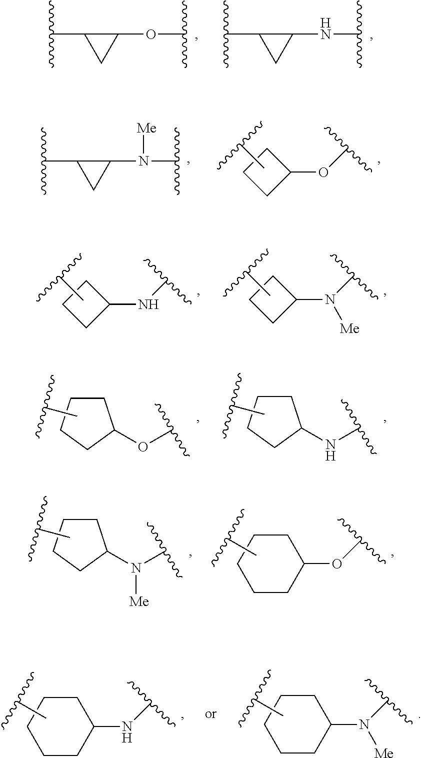

41. The compound of claim 40, wherein R² is H or CH₃.

42. The compound of claim 41, wherein L¹-L²-X⁵ is CH(CH₃)—(CH₂)₂—O, CH(CH₃)—(CH₂)₃—O, (CH₂)₃—O, CH(CH₃)—(CH₂)₂—NHC(O), CH(CH₃)—(CH₂)₂—NCH₃C(O), CH(CH₃)—(CH₂)₃—NHC(O), CH(CH₃)—(CH₂)₃—NCH₃C(O), CH(CH₃)—(CH₂)₂—C(O)NH, CH(CH₃)—(CH₂)₂—C(O)NCH₃, CH(CH₃)—(CH₂)₃—C(O)NH, or CH(CH₃)—(CH₂)₃—C(O)NCH₃.

43. The compound of any one of claims 40-42, having a structure according to Formula (I-C-1), 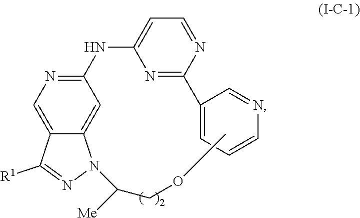
or a pharmaceutically acceptable salt thereof.

44. The compound of any one of claims 40-42, having a structure according to Formula (I-C-2), 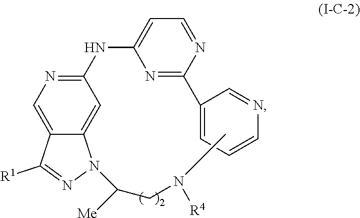
or a pharmaceutically acceptable salt thereof, wherein
R⁴ is H or CH₃.

45. The compound of any one of claims 40-42, having a structure according to Formula (I-C-3), 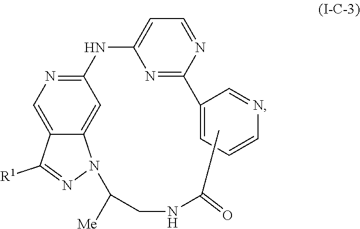
or a pharmaceutically acceptable salt thereof.

46. The compound of any one of claims 40-42, having a structure according to Formula (I-C-4), 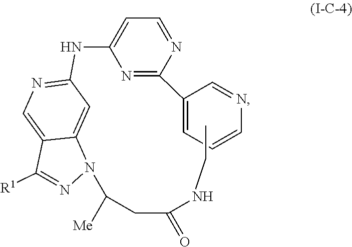
or a pharmaceutically acceptable salt thereof.

47. The compound of claim 1, having a structure according to Formula (I-D), 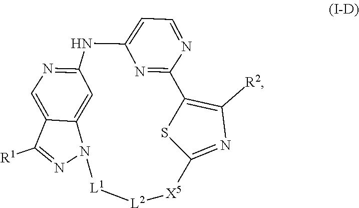
or a pharmaceutically acceptable salt thereof, wherein
R² is H or unsubstituted C₁₋₆ alkyl; and
L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄, or
L¹-L²-X⁵ is

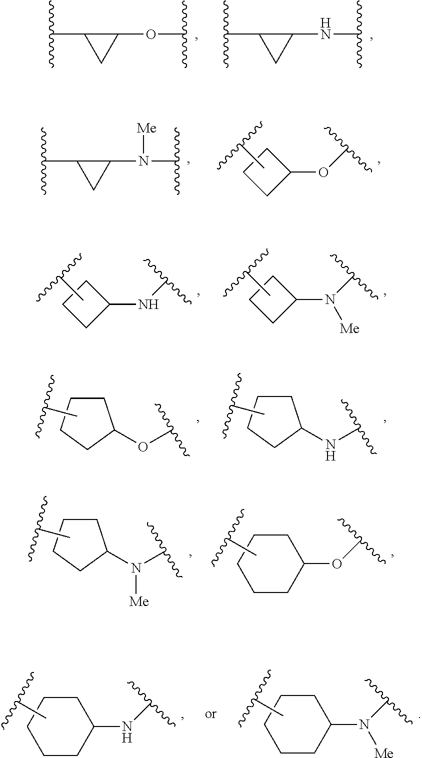

48. The compound of claim 47, wherein R² is H or CH₃.

49. The compound of claim 47 or 48, wherein L¹-L²-X⁵ is CH(CH₃)—(CH₂)₂—NH, CH(CH₃)—(CH₂)₂—NCH₃, CH(CH₃)—(CH₂)₃—NH, or CH(CH₃)—(CH₂)₃—NCH₃.

50. The compound of any one of claims 47-49, having a structure according to Formula (I-D-1), 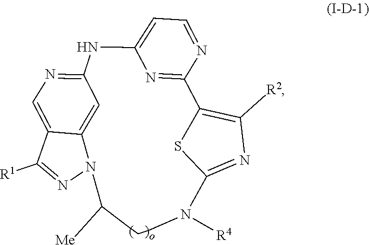
or a pharmaceutically acceptable salt thereof, wherein
R² is H or CH₃;
R⁴ is H or CH₃; and
is 1 or 2.

51. The compound of claim 1, having a structure according to Formula (I-E), 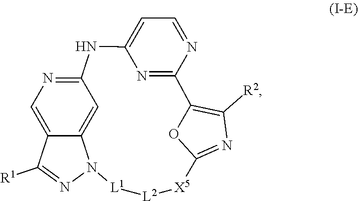
or a pharmaceutically acceptable salt thereof, wherein
R² is H or unsubstituted C₁₋₆ alkyl; and
L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄; or
L¹-L¹-X⁵ is

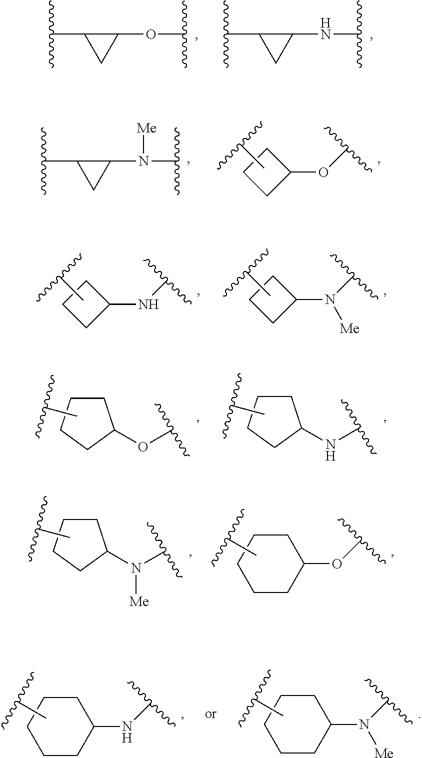

52. The compound of claim 51, wherein R² is H or CH₃.

53. The compound of claim 51 or 52, wherein L¹-L²-X⁵ is CH(CH₃)—(CH₂)₃, or CH(CH₃)—(CH₂)₄.

54. The compound of any one of claims 51-53, having a structure according to Formula (I-E-1), 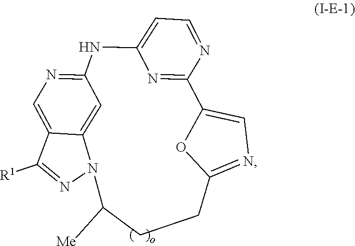
or a pharmaceutically acceptable salt thereof, wherein
is 2 or 3.

55. The compound of claim 1, having a structure according to Formula (I-F), 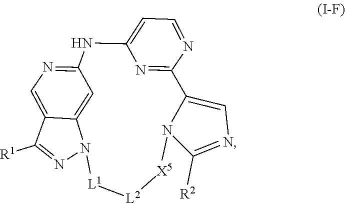
or a pharmaceutically acceptable salt thereof, wherein
R² is H or unsubstituted C₁₋₆ alkyl; and
L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄; or
L¹-L²-X⁵ is

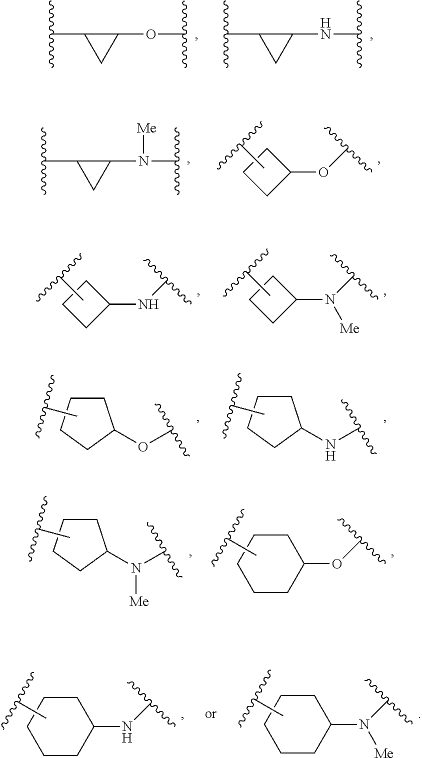

56. The compound of claim 55, wherein R² is H or CH₃.

57. The compound of claim 55 or 56, wherein L¹-L²-X⁵ is CH(CH₃)—(CH₂)₃, or CH(CH₃)—(CH₂)₄.

58. The compound of any one of claims 55-57, having a structure according to Formula (I-F-1), 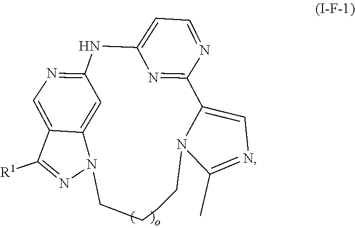
or a pharmaceutically acceptable salt thereof, wherein
o is 1 or 2.

59. The compound of claim 1, having a structure according to Formula (I-G), 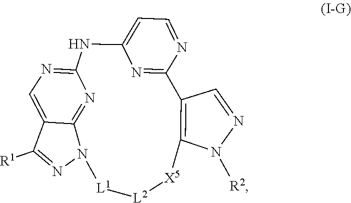
or a pharmaceutically acceptable salt thereof, wherein
R² is unsubstituted C₁₋₆ alkyl or C₁₋₆ alkyl substituted by a group that is unsubstituted C₃₋₆ cycloalkyl; and
L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄; or
L¹-L²-X⁵ is

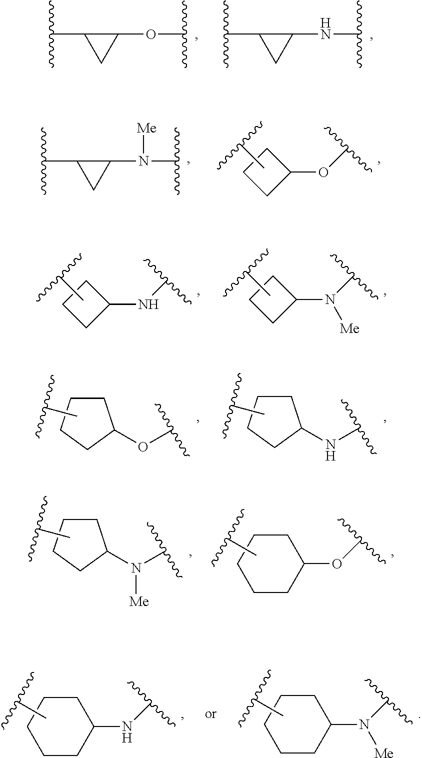

60. The compound of claim 59, wherein R² is H or CH₃.

61. The compound of claim 59 or 60, wherein L¹-L²-X⁵ is CH(CH₃)—(CH₂)₂—O, CH(CH₃)—(CH₂)₃—O, or (CH₂)₃—O.

62. The compound of any one of claims 59-61, having a structure according to Formula (I-G-1), 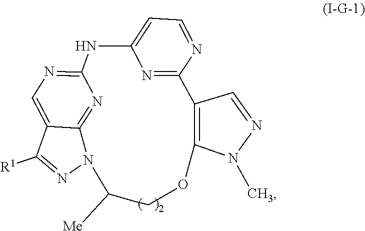
or a pharmaceutically acceptable salt thereof.

63. The compound of claim 1, having a structure according to Formula (I-H), 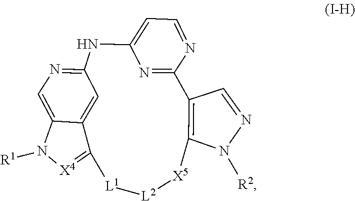
or a pharmaceutically acceptable salt thereof, wherein
X⁴ is CH or N;
R² is unsubstituted C₁₋₆ alkyl or C₁₋₆ alkyl substituted by a group that is unsubstituted C₃₋₆ cycloalkyl; and
L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH(CH₃)—(CH₂)₃, or CH(CH₃)—(CH₂)₄; or
L¹-L²-X⁵ is

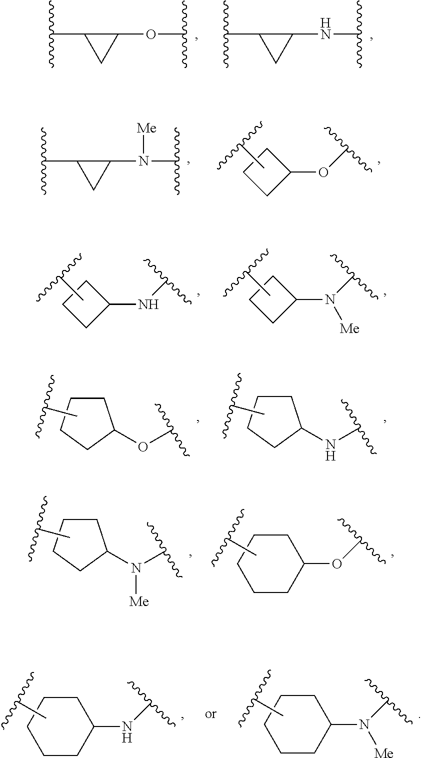

64. The compound of claim 63, wherein R² is H or CH₃.

65. The compound of claim 63 or 64, wherein L¹-L²-X⁵ is CH(CH₃)—(CH₂)₂—O, CH(CH₃)—(CH₂)₃—O, or (CH₂)₃—O.

66. The compound of any one of claims 63-65, having a structure according to Formula (I-H-1), 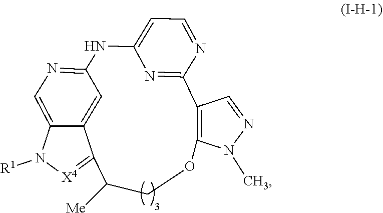
or a pharmaceutically acceptable salt thereof.

67. The compound of claim 1, having a structure according to Formula (I-I), 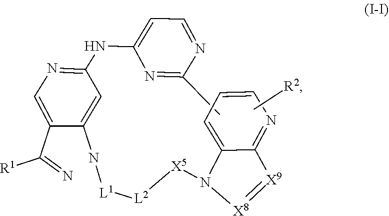
or a pharmaceutically acceptable salt thereof, wherein
R² is H or unsubstituted C₁₋₆ alkyl;
each X⁸ and X⁹ is CH or N; and
L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH₂CH₂, CH(CH₃)—(CH₂)₃, or CH(CH₃)—(CH₂)₄; or
L¹-L²-X⁵ is

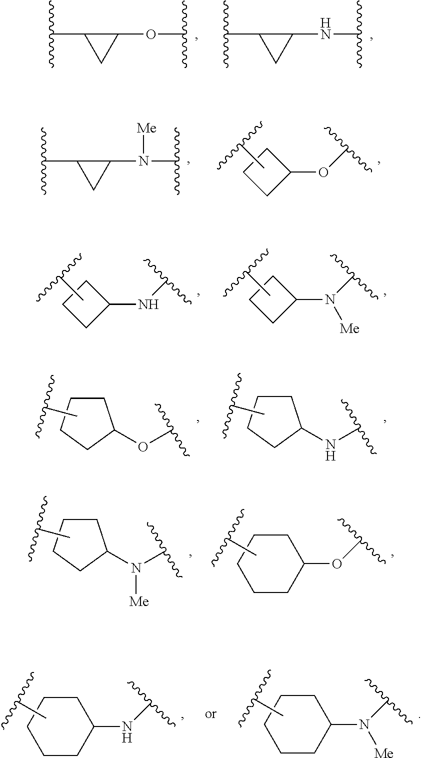

68. The compound of claim 67, wherein R² is H or CH₃.

69. The compound of claim 67 or 68, wherein L¹-L²-X⁵ is CH₂CH₂, CH(CH₃)—(CH₂)₃, or CH(CH₃)—(CH₂)₄.

70. The compound of any one of claims 67-69, having a structure according to Formula (I-I-1), 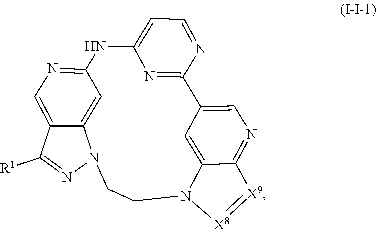
or a pharmaceutically acceptable salt thereof, wherein
each X⁸ and X⁹ is CH or N.

71. The compound of claim 1, having a structure according to Formula (I-J), 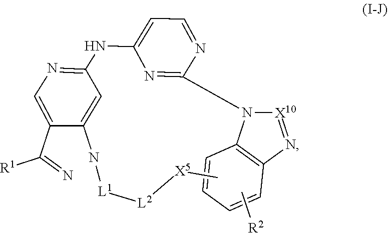
or a pharmaceutically acceptable salt thereof, wherein
R² is H or unsubstituted C₁₋₆ alkyl;
X¹⁰ is CH or N; and
L¹-L²-X⁵ is CH(CH₃)—(CH₂)₂—O, CH(CH₃)—(CH₂)₃—O, CH(CH₂CH₃)—(CH₂)₂—O, C(CH₃)₂—(CH₂)₂—O, (CH₂)₃—O, CH₂—CH(CH₃)CH₂—O, CH₂—CH₂CH(CH₃)—O, CH(CH₃)—(CH₂)₂—NH, CH(CH₃)—(CH₂)₂—NCH₃, CH(CH₃)—(CH₂)₃—NH, CH(CH₃)—(CH₂)₃—NCH₃, CH₂CH₂, CH(CH₃)—(CH₂)₃, or CH(CH₃)—(CH₂)₄; or
L¹-L2-X⁵ is

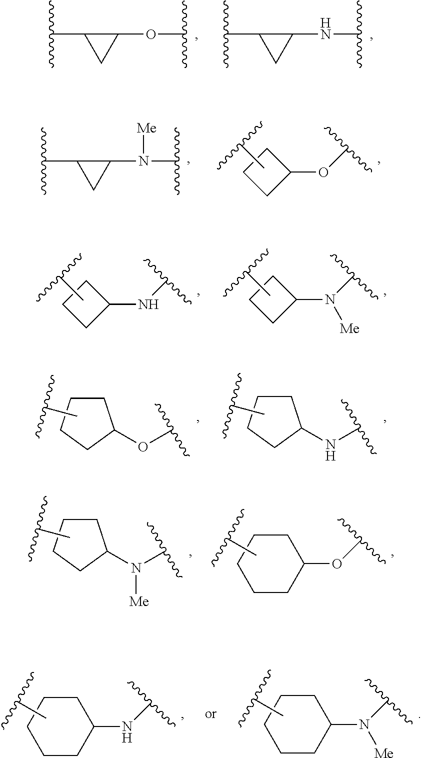

72. The compound of claim 71, wherein R² is H or CH₃.

73. The compound of claim 71 or 72, wherein L¹-L²-X⁵ is CH₂CH₂, CH(CH₃)—(CH₂)₃, or CH(CH₃)—(CH₂)₄.

74. The compound of any one of claims 71-73, having a structure according to Formula (I-J-1), 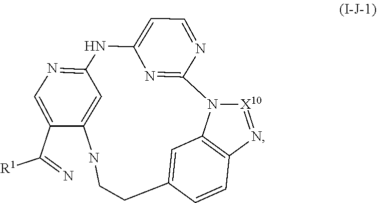
or a pharmaceutically acceptable salt thereof, wherein
X¹⁰ is CH or N.

75. The compound of any one of claims 1-21 and 27-74, wherein R¹ is F, CN, or NH₂.

76. The compound of any one of claims 1-21 and 27-74, wherein R¹ has a structure according to Substructure 1, 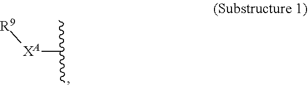
wherein
X^{A} is NH, NCH₃, or O;
R⁹ is a 3- to 6-membered oxygen-containing or nitrogen-containing heterocycloalkyl, C₃₋₇ cycloalkyl, or C₁₋₆ alkyl, and wherein said C₃₋₇ cycloalkyl or C₁₋₆ alkyl comprises one or two substituents selected from OH, NH₂, NMe₂, piperidinyl, and CONH₂.

77. The compound of claim 76, wherein R¹ is

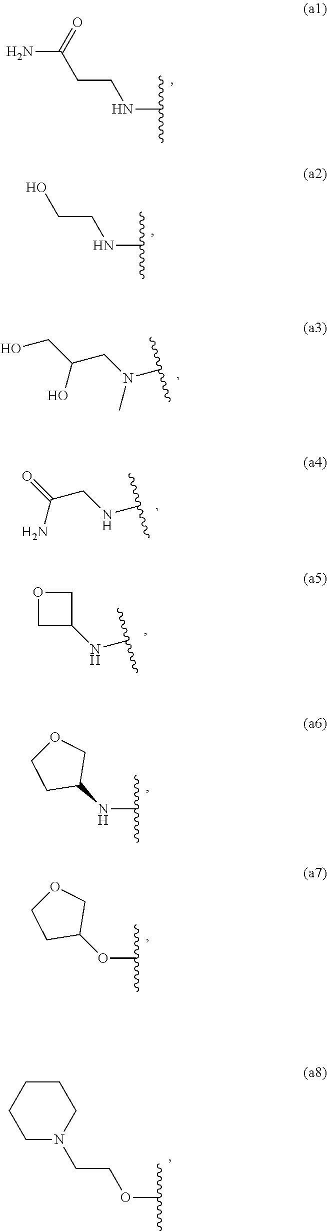

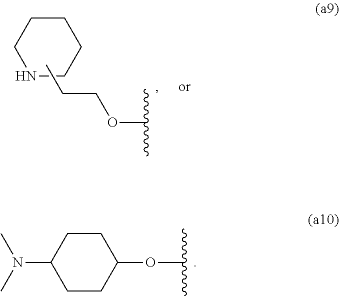

78. The compound of any one of claims 1-21 and 27-74, wherein R¹ has a structure according to Substructure 2, 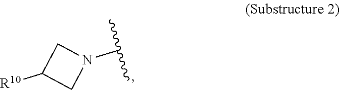
wherein
R¹⁰ is H, OH, C₁₋₆ alkyl, or CONR^{10A}R^{10B} and wherein said C₁₋₆ alkyl comprises one or two substituents selected from OH and CN;
each R^{10A} and R^{10B} is independently H, unsubstituted C₁₋₆ alkyl, C₁₋₆ alkyl substituted by alkoxy, or R^{10A} and R^{10B} together with the nitrogen atom to which they are attached form an unsubstituted 3- to 8-membered heterocycloalkyl ring.

79. The compound of claim 78, wherein R¹ is

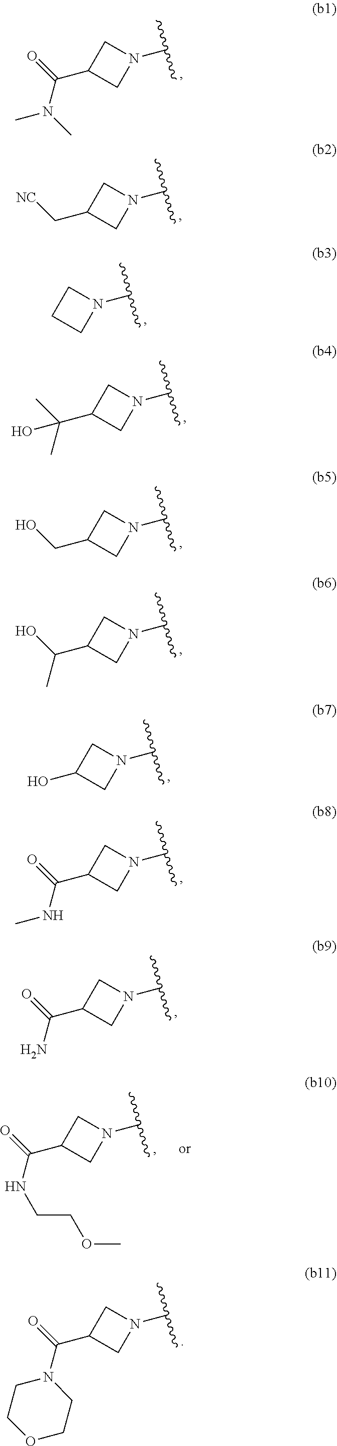

80. The compound of any one of claims 1-21 and 27-74, wherein R¹ has a structure according to Substructure 3, 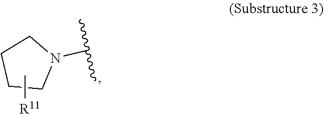
wherein
R¹¹ is H, OH, amino, mono(C₁₋₆ alkyl)amino, di(C₁₋₆ alkyl)amino, —CH₂-[di(C₁₋₆ alkyl)amino], CN, C₁₋₆ alkyl, CONH₂, CONHMe, COOH, CO₂Me, or CONR^{11A}R^{11B}; and wherein said C₁₋₆ alkyl comprises one or two substituents selected from OH, F, and NR^{11A}R^{11B};
each R^{11A} and R^{11B} is independently unsubstituted C₁₋₆ alkyl, or R^{11A} and R^{11B} together with the nitrogen atom to which they are attached form a methyl or isopropyl substituted 3- to 8-membered heterocycloalkyl ring.

81. The compound of claim 80, wherein R¹ is

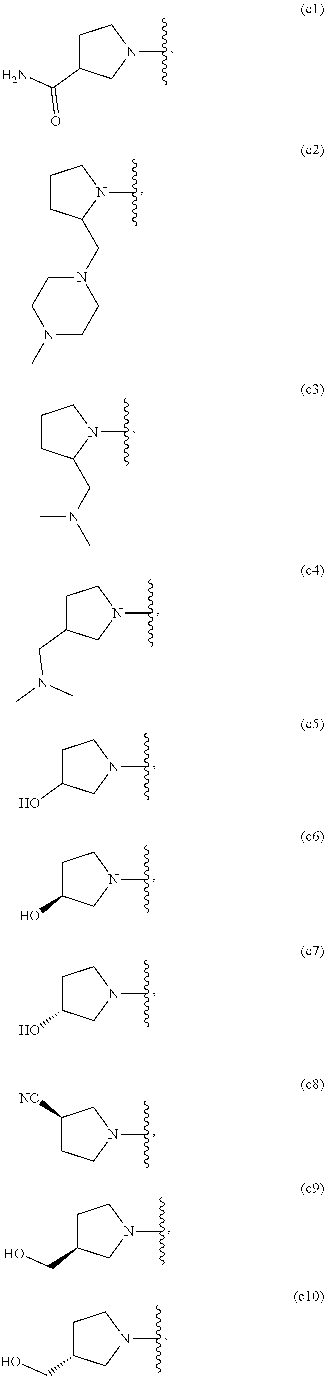

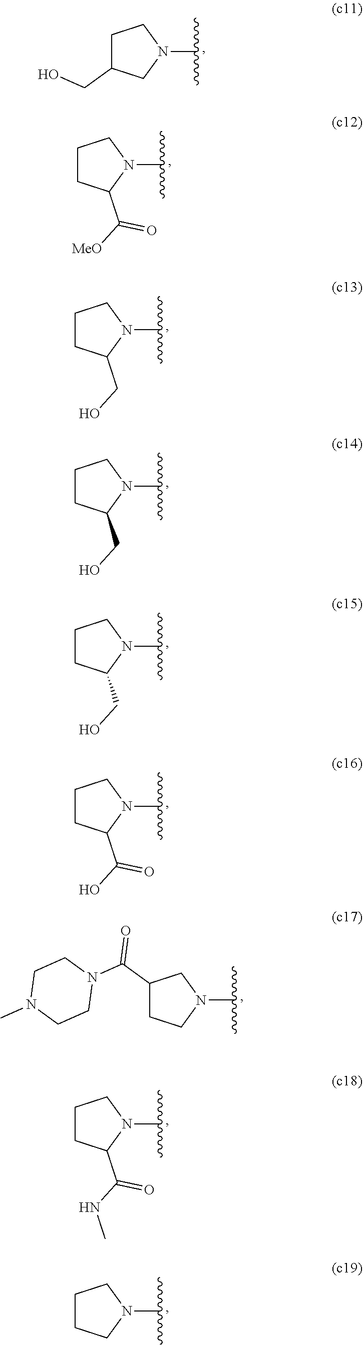

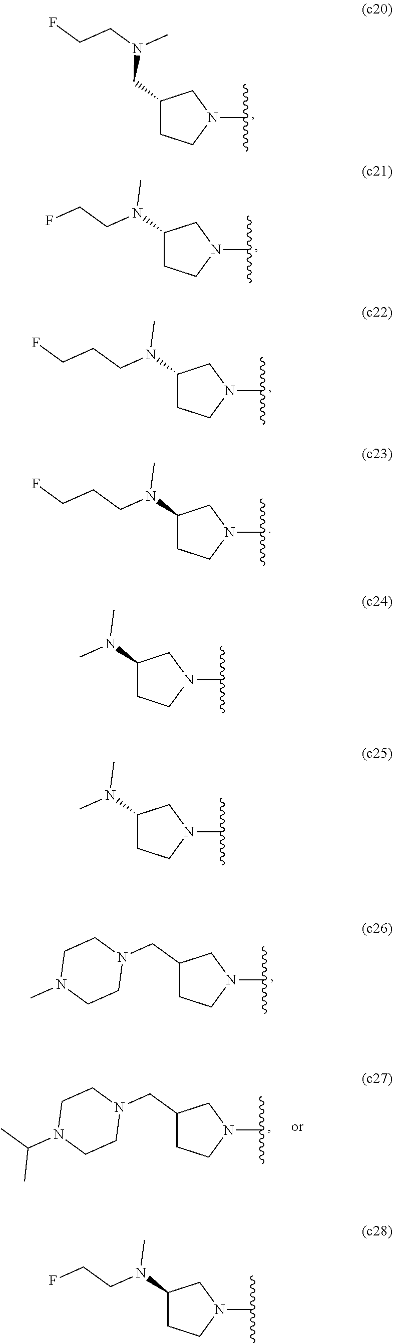

82. The compound of any one of claims 1-21 and 27-74, wherein R¹ has a structure according to Substructure 4, 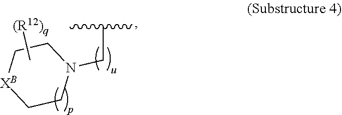
X^{B} is N, O, S, SO, or SO₂;
each R¹², when present, is oxo, methyl, or cyclopropyl;
p is 0 or 1;
q is 0, 1, or 2; and
u is 0 or 1.

83. The compound of claim 82, wherein R¹ is

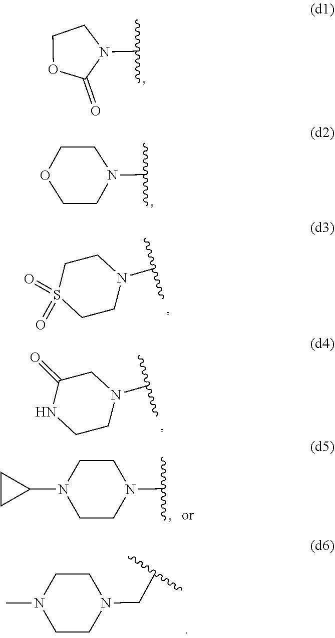

84. The compound of any one of claims 1-21 and 27-74, wherein R¹ has a structure according to Substructure 5, 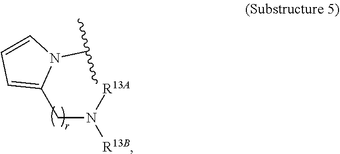
wherein
r is 1 or 2; and
each R^{13A} and R^{13B} is independently unsubstituted C₁₋₆ alkyl, or R^{13A} and R^{13B} together with the nitrogen atom to which they are attached form a N-methyl 3- to 8-membered heterocycloalkyl ring.

85. The compound of claim 84, wherein R¹ is

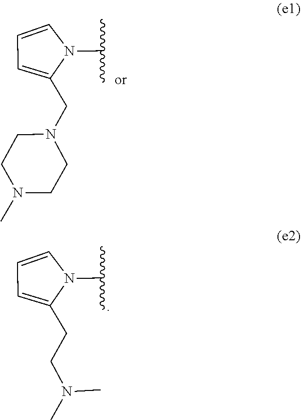

86. The compound of any one of claims 1-21 and 27-74, wherein R¹ has a structure according to Substructure 6, 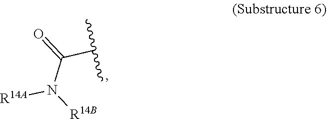
wherein
each R^{14A} and R^{14B} is independently H, unsubstituted C₁₋₆ alkyl, or 5- to 6-membered cycloalkyl ring substituted with CN.

87. The compound of claim 86, wherein R¹ is

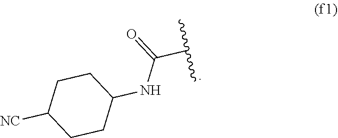 [SMILES: *C(=O)NC1CCC(C#N)CC1]

88. The compound of any one of claims 1-21 and 27-74, wherein R¹ has a structure according to Substructure 7, 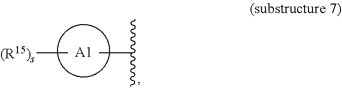
wherein
s is 0, 1, 2, or 3;
v is 0, 1, 2, or 3;
A1 is phenyl, 5- to 6-membered heteroarylene or 5- to 6-membered heterocycloalkyl;
R¹⁵ is independently
halogen
unsubstituted C₁₋₆ alkyl;
C₃₋₆ cycloalkyl;
C₁₋₆ alkyl substituted by OH or OMe;
C₁₋₆ alkyl substituted by halo, amino, monoalkylamino, or dialkylamino;
C₁₋₆ alkoxyl substituted by halo, amino, monoalkylamino, or dialkylamino;
8- to 9-membered heterocycloalkyl;
—(CH₂)ᵥ-(5- to 6-membered heterocycloalkyl);
—(CH₂)ᵥ-(5- to 6-membered heteroaryl);
—(CO)-(5- to 6-membered heterocycloalkyl);
—(CO)-(5- to 6-membered heteroaryl);
—O-(5- to 6-membered heterocycloalkyl);
—O-(5- to 6-membered heteroaryl);
—(CH₂)ᵥ—NH—(C₁₋₆ alkyl substituted by halo, OH, OMe, amino, monoalkylamino, or dialkylamino);
—(CH₂)ᵥ—NMe-(C₁₋₆ alkyl substituted by halo, OH, OMe, amino, monoalkylamino, or dialkylamino).

89. The compound of claim 88, wherein A1 is furan, pyrazole, pyrrole, thiazole, oxazole, phenyl, pyridyl, or a bicyclic nitrogen-containing 8- to 9-membered heterocycloalkyl.

90. The compound of claim 88 or 89, wherein substructure 7 is

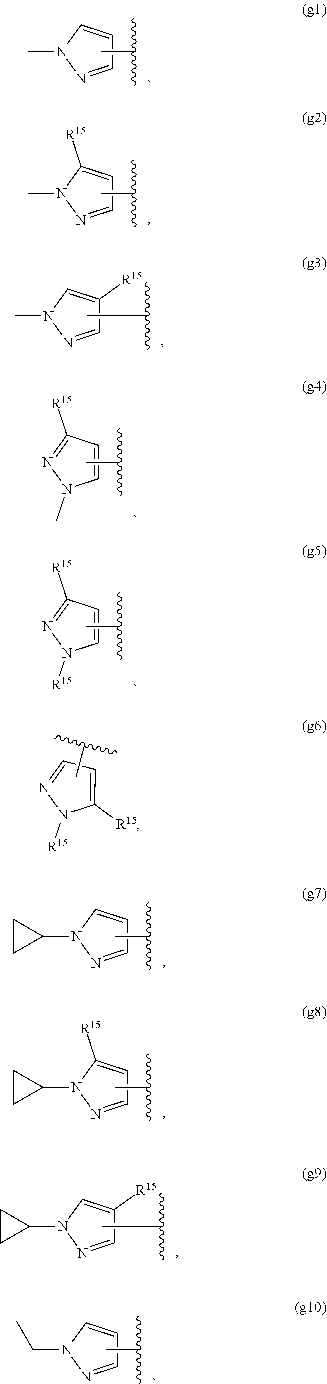

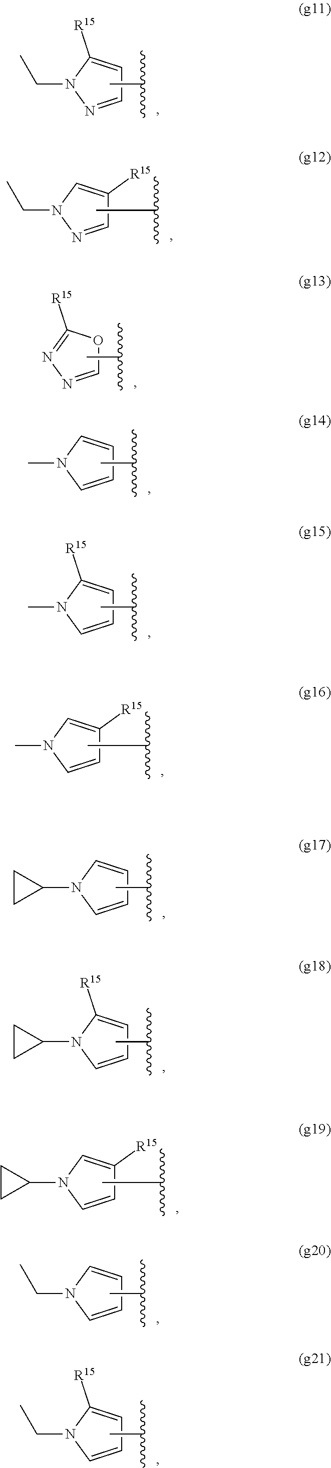

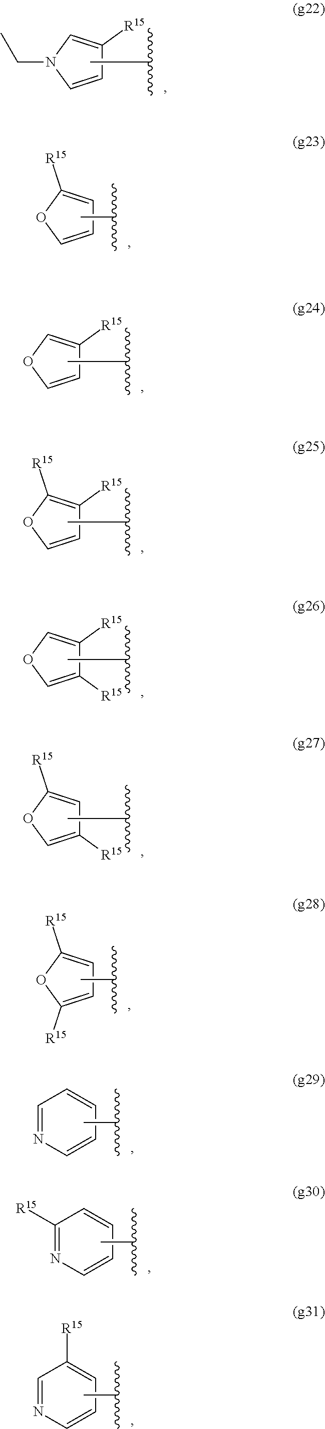

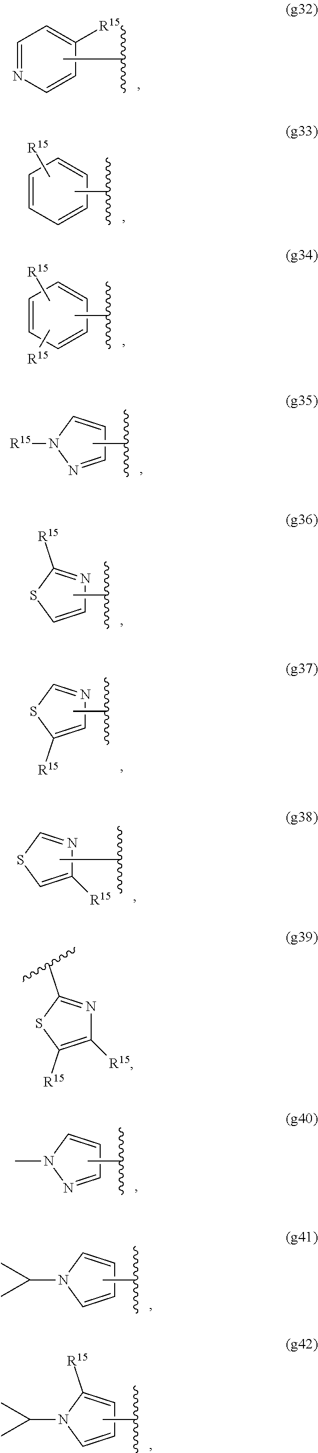

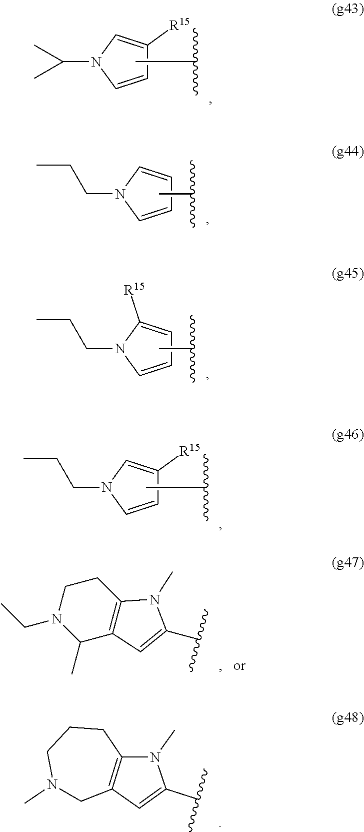

91. The compound of any one of claims 88-90, wherein each R¹⁵ is independently
—F, —Cl, —CH₃, —CH₂CH₃, —CH(CH₃)₂,

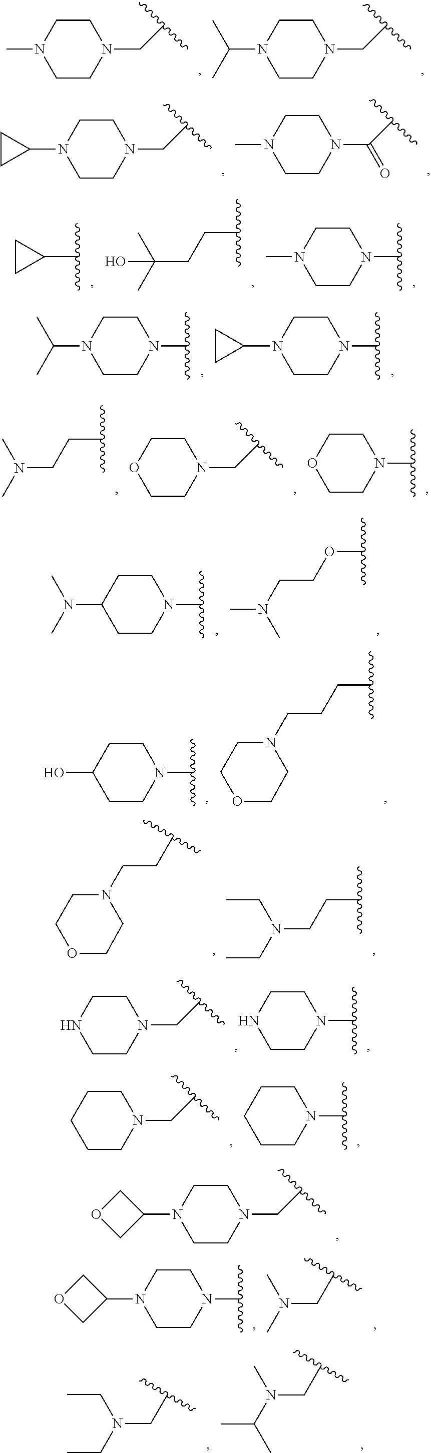

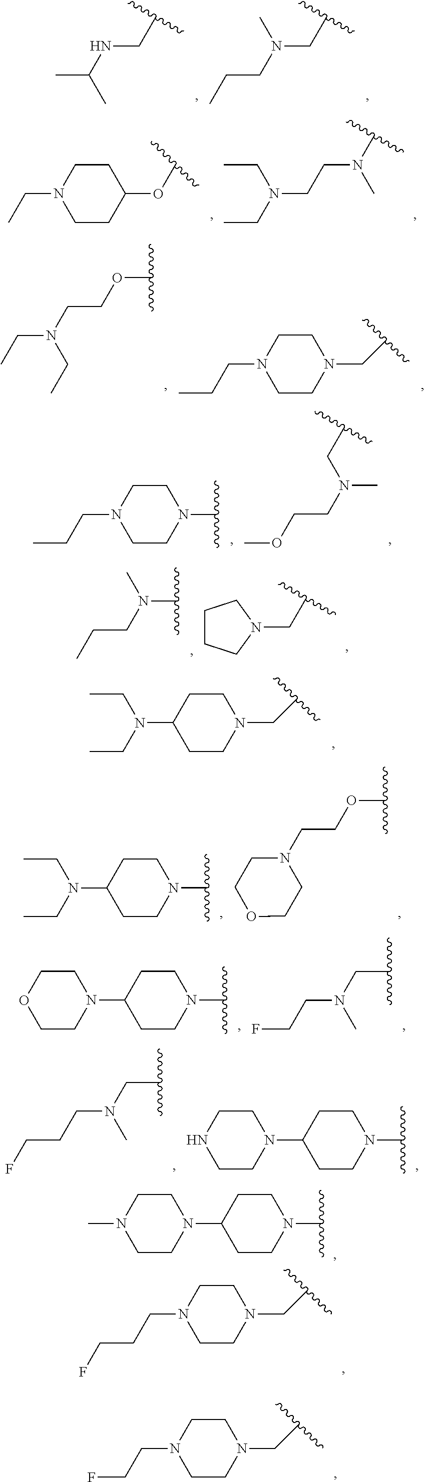

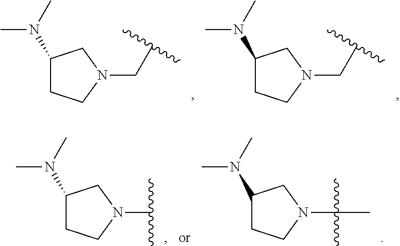

92. The compound of claim 1, having a structure that is selected from the group consisting of any one of Compounds (1)-(58), (61)-(71), (73)-(80), and (82)-(193), or a pharmaceutically acceptable salt thereof.

93. A pharmaceutical composition comprising a compound according to any one of claims 1-92, or a pharmaceutically acceptable salt thereof.

94. A method of treating cancer comprising administering to a human in need thereof an effective amount of a compound according to any one of claims 1-92 or a pharmaceutically acceptable salt thereof in a pharmaceutical composition.

95. The method of claim 94, wherein said cancer is a lung cancer.

96. The method of claim 94 or 95, wherein said cancer is non-small cell lung cancer.

97. The method of any one of claims 94-96, wherein said cancer is an EGFR-driven cancer.

98. The method of any one of claims 94-97, wherein said cancer is characterized by an EGFR mutation.
